(19) United States
(12) Patent Application Publication
(10) Pub. No.: US 2024/0035087 A1
(43) Pub. Date: Feb. 1, 2024

(54) METHODS AND SYSTEMS FOR DROPLET MANIPULATION

(71) Applicant: Volta Labs, Inc., Cambridge, MA (US)

(72) Inventors: William Kai LANGFORD, Cambridge, MA (US); Akim LENNHOFF, Cambridge, MA (US); Devin Laier JUNKINS, Boston, MA (US); Ishaan GOVINDARAJAN, Cambridge, MA (US); Abdul Majeed MOHAMMED, Somerville, MA (US); Raphael MERAND, Somerville, MA (US); Liam MASTERS, Cambridge, MA (US); Udayan UMAPATHI, Somerville, MA (US)

(21) Appl. No.: 18/462,365

(22) Filed: Sep. 6, 2023

Related U.S. Application Data
(63) Continuation of application No. PCT/US2022/018549, filed on Mar. 2, 2022.
(60) Provisional application No. 63/287,412, filed on Dec. 8, 2021.
(60) Provisional application No. 63/255,721, filed on Oct. 14, 2021.
(60) Provisional application No. 63/250,101, filed on Sep. 29, 2021.
(60) Provisional application No. 63/155,692, filed on Mar. 2, 2021.

(51) Int. Cl. C12Q 1/6874; B01L 3/00

(52) U.S. Cl. CPC C12Q 1/6874; B01L 3/502792; B01L 3/502707; B01L 2400/0427; B01L 2400/0433; B01L 2300/0645; B01L 2400/043

(57) ABSTRACT

Described herein are systems and methods of for conducting various biological assays on arrays utilizing electrowetting on dielectric (EWOD). The systems and methods may process the biological sample, or plurality thereof, using at least one droplet. The droplet, or plurality thereof, may be manipulated using the systems and methods described herein. Further described herein are improvements to arrays for facilitating the execution of biological assays on the arrays.

### CROSS-REFERENCE

This application is a continuation of International Application No. PCT/US2022/018549, filed Mar. 2, 2022, which claims benefit of U.S. Provisional Application No. 63/155,692 filed Mar. 2, 2021, U.S. Provisional Application No. 63/250,101 filed Sep. 29, 2021, U.S. Provisional Application No. 63/255,721 filed Oct. 14, 2021, and U.S. Provisional Application No. 63/287,412 filed Dec. 8, 2021, which are herein incorporated by reference in their entirety.

### BACKGROUND

Biological samples may be processed for various applications. For example, a deoxyribonucleic acid (DNA) molecule or a ribonucleic acid (RNA) molecule may be processed (e.g., sequenced) to identify genetic variants, which may be useful to identify a disease, such as cancer. Such biological samples may be processed in partitions, such as droplets. Sequences of DNA or RNA may be determined by sequence identification, such as nucleic acid sequencing.

Droplets containing biological samples may be manipulated by using electrowetting, which may employ electric fields from electrodes to move a droplet adjacent to a surface.

### SUMMARY

Some aspects of the present disclosure provide method for circularizing a nucleic acid sample, comprising: providing a droplet adjacent to an electrowetting array, wherein the sample droplet comprises the nucleic acid sample; and using the electrowetting array to process the droplet to circularize the nucleic acid sample. In some embodiments, the electrowetting array comprises a dielectric substrate. In some embodiments, the electrowetting array further comprises one or more reagent droplets. In some embodiments, the one or more reagent droplets comprises one or more reagents for circularizing the nucleic acid sample. In some embodiments, the method further comprises: combining the sample droplet with the one or more reagent droplets; separating the sample droplet from the one or more reagent droplets; and combining the one or more reagent droplets with a second droplet. In some embodiments, the droplet comprises one or more reagents for circularizing the nucleic acid sample. In some embodiments, (b) further comprises performing one or more droplet operations on the electrowetting array to process the droplet, wherein the one or more droplet operations comprise contacting the one or more reagent droplets with the droplet. In some embodiments, the electrowetting array comprises one or more electrodes beneath a surface of the electrowetting array, and wherein the one or more droplet operations comprise applying a voltage to at least one electrode of the one or more electrodes to manipulate the one or more reagent droplets, the sample droplet, or both. In some embodiments, the one or more droplet operations comprise applying a vibration to the one or more reagent droplets, the sample droplet, or both. In some embodiments, the one or more droplet operations comprise applying a vibration to the electrowetting array. In some embodiments, the method further comprises using a single polymerizing enzyme to subject the nucleic acid sample to a sequencing reaction. In some embodiments, the method further comprises yielding a sequencing read having a length of at least 70 kilobase (kb). In some embodiments, the method further comprises yielding a sequencing read having a length of at least 80 kilobase (kb). In some embodiments, the method further comprises yielding a sequencing read having a length of about 200 kilobase (kb). In some embodiments, at least 100 (Gb) of sequencing data is produced. In some embodiments, at least 500 Gb of sequencing data is produced. In some embodiments, at least 512 Gb of sequencing data is produced. In some embodiments, at least 10 Gb of data is produced. In some embodiments, at least 30 Gb of data is produced. In some embodiments, the sequencing reaction comprises repeated passes. In some embodiments, one or more subreads of the sequencing read are produced. In some embodiments, a consensus sequence is produced from the subreads of sequencing reads. In some embodiments, the sequencing read comprises an A260/A280 ratio of less than about 1.84. In some embodiments, the method further comprises generating a circularized nucleic acid sample. In some embodiments, the circularized nucleic acid sample comprises a target sequence. In some embodiments, the circularized nucleic acid sample comprises a plurality of sequences comprising the target sequence. In some embodiments, at least 80% of the plurality of sequences comprises the target sequence. In some embodiments, the method further comprises, prior to (a), deriving the nucleic acid sample from a biological sample on the electrowetting array.

Another aspect of the present disclosure is a method of sequencing a nucleic acid sample, comprising (a) providing a droplet adjacent to an electrowetting array, which droplet comprises the nucleic acid sample, (b) using the electrowetting array to process the droplet to circularize the nucleic acid sample, and (c) using a single polymerizing enzyme to subject the circularized nucleic acid sample to a sequencing reaction. A method for sequencing a circular nucleic acid sample, comprising using a single polymerizing enzyme to subject the nucleic acid sample to a sequencing reaction to yield a sequencing read having a length of at least 70 kilobase. In some embodiments, the method further comprises using a waveguide to detect bases incorporated into the nucleic acid sample during the sequencing reaction. In some embodiments, the electrowetting array comprises a dielectric substrate. In some embodiments, the electrowetting array further comprises one or more reagent droplets. In some embodiments, the one or more reagent droplets comprises one or more reagents for circularizing the nucleic acid sample. In some embodiments, the method further comprises combining the sample droplet with the one or more reagent droplets; separating the sample droplet from the one or more reagent droplets; and combining the one or more reagent droplets with a second droplet. In some embodiments, the droplet comprises one or more reagents for circularizing the nucleic acid sample. In some embodiments, (b) further comprises performing one or more droplet operations on the electrowetting array to process the droplet, wherein the one or more droplet operations comprise contacting the one or more reagent droplets with the droplet. In some embodiments, the electrowetting array comprises one or more electrodes beneath a surface of the electrowetting array, and wherein the one or more droplet operations comprise applying a voltage to at least one electrode of the one or more electrodes to manipulate the one or more reagent droplets, the sample droplet, or both. In some embodiments, the one or more droplet operations comprise applying a vibration to the one or more reagent droplets, the sample droplet, or both. In some embodiments, the one or more droplet operations comprise applying a vibration to the electrowetting array. In some embodiments, the method further comprises using a single polymerizing enzyme to subject the nucleic acid sample to a sequencing reaction. In some embodiments, the method further comprises yielding a sequencing read having a length of at least 70 kilobase (kb). In some embodiments, the method further comprises yielding a sequencing read having a length of at least 80 kilobase (kb). In some embodiments, the method further comprises yielding a sequencing read having a length of about 200 kilobase (kb). In some embodiments, at least 100 (Gb) of sequencing data is produced. In some embodiments, at least 500 Gb of sequencing data is produced. In some embodiments, at least 512 Gb of sequencing data is produced. In some embodiments, at least 10 Gb of data is produced. In some embodiments, at least 30 Gb of data is produced. In some embodiments, the sequencing reaction comprises repeated passes. In some embodiments, one or more subreads of the sequencing read are produced. In some embodiments, a consensus sequence is produced from the subreads of sequencing reads. In some embodiments, the sequencing read comprises an A260/A280 ratio of less than about 1.84. In some embodiments, the method further comprises generating a circularized nucleic acid sample. In some embodiments, the circularized nucleic acid sample comprises a target sequence. In some embodiments, the circularized nucleic acid sample comprises a plurality of sequences comprising the target sequence. In some embodiments, at least 80% of the plurality of sequences comprises the target sequence. In some embodiments, the method further comprises, prior to (a), deriving the nucleic acid sample from a biological sample on the electrowetting array.

Another aspect of the present disclosure is a method of producing a circularized nucleic acid sample with a longer insert size, comprising (a) providing a droplet adjacent to an electrowetting array, which droplet comprises the nucleic acid sample, (b) using the electrowetting array to process the droplet to circularize the nucleic acid sample, and (c) using a single polymerizing enzyme to subject the circularized nucleic acid sample to a sequencing reaction. In some embodiments, the electrowetting array comprises a dielectric substrate. In some embodiments, the electrowetting array further comprises one or more reagent droplets. In some embodiments, the one or more reagent droplets comprises one or more reagents for circularizing the nucleic acid sample. In some embodiments, the method further comprises combining the sample droplet with the one or more reagent droplets; separating the sample droplet from the one or more reagent droplets; and combining the one or more reagent droplets with a second droplet. In some embodiments, the droplet comprises one or more reagents for circularizing the nucleic acid sample. In some embodiments, (b) further comprises performing one or more droplet operations on the electrowetting array to process the droplet, wherein the one or more droplet operations comprise contacting the one or more reagent droplets with the droplet. In some embodiments, the electrowetting array comprises one or more electrodes beneath a surface of the electrowetting array, and wherein the one or more droplet operations comprise applying a voltage to at least one electrode of the one or more electrodes to manipulate the one or more reagent droplets, the sample droplet, or both. In some embodiments, the one or more droplet operations comprise applying a vibration to the one or more reagent droplets, the sample droplet, or both. In some embodiments, the one or more droplet operations comprise applying a vibration to the electrowetting array. In some embodiments, the method further comprises using a single polymerizing enzyme to subject the nucleic acid sample to a sequencing reaction. In some embodiments, the method further comprises yielding a sequencing read having a length of at least 70 kilobase (kb). In some embodiments, the method further comprises yielding a sequencing read having a length of at least 80 kilobase (kb). In some embodiments, the method further comprises yielding a sequencing read having a length of about 200 kilobase (kb). In some embodiments, at least 100 (Gb) of sequencing data is produced. In some embodiments, at least 500 Gb of sequencing data is produced. In some embodiments, at least 512 Gb of sequencing data is produced. In some embodiments, at least 10 Gb of data is produced. In some embodiments, at least 30 Gb of data is produced. In some embodiments, the sequencing reaction comprises repeated passes. In some embodiments, one or more subreads of the sequencing read are produced. In some embodiments, a consensus sequence is produced from the subreads of sequencing reads. In some embodiments, the sequencing read comprises an A260/A280 ratio of less than about 1.84. In some embodiments, the method further comprises generating a circularized nucleic acid sample. In some embodiments, the circularized nucleic acid sample comprises a target sequence. In some embodiments, the circularized nucleic acid sample comprises a plurality of sequences comprising the target sequence. In some embodiments, at least 80% of the plurality of sequences comprises the target sequence. In some embodiments, the method further comprises, prior to (a), deriving the nucleic acid sample from a biological sample on the electrowetting array.

Another aspect of the present disclosure is a method for generating a sequencing library, comprising (a) providing a nucleic acid sample comprising a plurality of nucleic acid molecules comprising a plurality of sequences, and (b) using the nucleic acid sample to generate the sequencing library, wherein the sequencing library comprises at least 80% of the plurality of sequences of complements thereof; (c) using said electrowetting array to process said droplet to circularize said nucleic acid sample; (d) separating said droplet from said one or more reagent droplets; and; (e) combining said one or more reagent droplets with said sample droplet to yield a circularized nucleic acid sample. In some embodiments, the electrowetting array comprises a dielectric substrate. In some embodiments, the electrowetting array further comprises one or more reagent droplets. In some embodiments, the one or more reagent droplets comprises one or more reagents for circularizing the nucleic acid sample. In some embodiments, the method further comprises combining the sample droplet with the one or more reagent droplets; separating the sample droplet from the one or more reagent droplets; and combining the one or more reagent droplets with a second droplet. In some embodiments, the droplet comprises one or more reagents for circularizing the nucleic acid sample. In some embodiments, (b) further comprises performing one or more droplet operations on the electrowetting array to process the droplet, wherein the one or more droplet operations comprise contacting the one or more reagent droplets with the droplet. In some embodiments, the electrowetting array comprises one or more electrodes beneath a surface of the electrowetting array, and wherein the one or more droplet operations comprise applying a voltage to at least one electrode of the one or more electrodes to manipulate the one or more reagent droplets, the sample droplet, or both. In some embodiments, the one or more droplet operations comprise applying a vibration to the one or more reagent droplets, the sample droplet, or both. In some embodiments, the one or more droplet operations comprise applying a vibration to the electrowetting array. In some embodiments, the method further comprises using a single polymerizing enzyme to subject the nucleic acid sample to a sequencing reaction. In some embodiments, the method further comprises yielding a sequencing read having a length of at least 70 kilobase (kb). In some embodiments, the method further comprises yielding a sequencing read having a length of at least 80 kilobase (kb). In some embodiments, the method further comprises yielding a sequencing read having a length of about 200 kilobase (kb). In some embodiments, at least 100 (Gb) of sequencing data is produced. In some embodiments, at least 500 Gb of sequencing data is produced. In some embodiments, at least 512 Gb of sequencing data is produced. In some embodiments, at least 10 Gb of data is produced. In some embodiments, at least 30 Gb of data is produced. In some embodiments, the sequencing reaction comprises repeated passes. In some embodiments, one or more subreads of the sequencing read are produced. In some embodiments, a consensus sequence is produced from the subreads of sequencing reads. In some embodiments, the sequencing read comprises an A260/A280 ratio of less than about 1.84. In some embodiments, the method further comprises generating a circularized nucleic acid sample. In some embodiments, the circularized nucleic acid sample comprises a target sequence. In some embodiments, the circularized nucleic acid sample comprises a plurality of sequences comprising the target sequence. In some embodiments, at least 80% of the plurality of sequences comprises the target sequence. In some embodiments, the method further comprises, prior to (a), deriving the nucleic acid sample from a biological sample on the electrowetting array.

Another aspect of the present disclosure is a method for circularizing a nucleic acid sample, comprising: providing a droplet adjacent to an electrowetting array, wherein the droplet comprises the nucleic acid sample; combining the droplet with one or more reagent droplets; using the electrowetting array to process the droplet to circularize the nucleic acid sample; separating the droplet from the one or more reagent droplets; and combining the one or more reagent droplets with the sample droplet to yield a circularized nucleic acid sample. In some embodiments, the electrowetting array comprises a dielectric substrate. In some embodiments, the electrowetting array further comprises one or more reagent droplets. In some embodiments, the one or more reagent droplets comprises one or more reagents for circularizing the nucleic acid sample. In some embodiments, the method further comprises: combining the sample droplet with the one or more reagent droplets; separating the sample droplet from the one or more reagent droplets; and combining the one or more reagent droplets with a second droplet. In some embodiments, the droplet comprises one or more reagents for circularizing the nucleic acid sample. In some embodiments, (b) further comprises performing one or more droplet operations on the electrowetting array to process the droplet, wherein the one or more droplet operations comprise contacting the one or more reagent droplets with the droplet. In some embodiments, the electrowetting array comprises one or more electrodes beneath a surface of the electrowetting array, and wherein the one or more droplet operations comprise applying a voltage to at least one electrode of the one or more electrodes to manipulate the one or more reagent droplets, the sample droplet, or both. In some embodiments, the one or more droplet operations comprise applying a vibration to the one or more reagent droplets, the sample droplet, or both. In some embodiments, the one or more droplet operations comprise applying a vibration to the electrowetting array. In some embodiments, the method further comprises using a single polymerizing enzyme to subject the nucleic acid sample to a sequencing reaction. In some embodiments, the method further comprises yielding a sequencing read having a length of at least 70 kilobase (kb). In some embodiments, the method further comprises yielding a sequencing read having a length of at least 80 kilobase (kb). In some embodiments, the method further comprises yielding a sequencing read having a length of about 200 kilobase (kb). In some embodiments, at least 100 (Gb) of sequencing data is produced. In some embodiments, at least 500 Gb of sequencing data is produced. In some embodiments, at least 512 Gb of sequencing data is produced. In some embodiments, at least 10 Gb of data is produced. In some embodiments, at least 30 Gb of data is produced. In some embodiments, the sequencing reaction comprises repeated passes. In some embodiments, one or more subreads of the sequencing read are produced. In some embodiments, a consensus sequence is produced from the subreads of sequencing reads. In some embodiments, the sequencing read comprises an A260/A280 ratio of less than about 1.84. In some embodiments, the method further comprises generating a circularized nucleic acid sample. In some embodiments, the circularized nucleic acid sample comprises a target sequence. In some embodiments, the circularized nucleic acid sample comprises a plurality of sequences comprising the target sequence. In some embodiments, at least 80% of the plurality of sequences comprises the target sequence. In some embodiments, the method further comprises, prior to (a), deriving the nucleic acid sample from a biological sample on the electrowetting array.

Another aspect of the present disclosure is a method of generating a biopolymer, comprising: providing a plurality of droplets adjacent to a surface, wherein said plurality of droplets comprises a first droplet comprising a first reagent and a second droplet comprising a second reagent; subjecting said first droplet and said second droplet to motion relative to one another to (i) bring said first droplet in contact with said second droplet and (ii) form a merged droplet comprising said first reagent and said second reagent; and in said merged droplet, using at least (i) said first reagent and (ii) said second reagent to form at least a portion of said biopolymer, wherein (b)-(c) are performed in a time period of 10 minutes or less. In some embodiments, said biopolymer is a polynucleotide. In some embodiments, said biopolymer is a polypeptide. In some embodiments, where said polynucleotide comprises about 10 to about 250 bases. In some embodiments, where said polynucleotide comprises about 260 to about 1 kb. In some embodiments, said polynucleotide comprises about 1 kb to about 10,000 kb. In some embodiments, a vibration is applied to said synthesis droplet during (b), (c), or both. In some embodiments, the method further comprises, one or more washing steps comprising subjecting a wash droplet to motion to contact said merged droplet. In some embodiments, a vibration is applied to said one or more washing steps. In some embodiments, said surface is dielectric. In some embodiments, said surface comprises a dielectric layer disposed over one or more electrodes. In some embodiments, said surface is the surface of a polymeric film. In some embodiments, the surface comprises one or more oligonucleotides bound to the surface. In some embodiments, said surface is the surface of a lubricating liquid layer. In some embodiments, said plurality of droplets comprises a third droplet comprising a third reagent. In some embodiments, said first reagent, said second reagent, said third reagent, or any combination thereof, comprises one or more functionalized beads. In some embodiments, said functional beads comprise one or more oligonucleotides immobilized thereto. In some embodiments, a vibration is applied to either said first droplet, said second droplet, said third droplet, a wash droplet, or the mixtures thereof. In some embodiments, said first reagent, said second reagent, said third reagent or any combination thereof comprises a polymerase. In some embodiments, said first reagent, said second reagent, said third reagent or any combination thereof comprises a bio-monomer. In some embodiments, said bio-monomer is an amino acid. In some embodiments, said bio-monomer is a nucleic acid molecule. In some embodiments, said nucleic acid molecule comprises of adenine, cytosine, guanine, thymine, or uracil. In some embodiments, said first reagent, said second reagent, said third reagent, or any combination thereof, comprises one or more functionalized discs. In some embodiments, said functionalized disc comprise one or more oligonucleotides immobilized thereto. In some embodiments, said first reagent, said second reagent, said third reagent, or any combination thereof comprises an enzyme that mediates synthesis or polymerization. In some embodiments, said enzyme is from the group consisting of Polynucleotide Phosphorylase (PNPase), Terminal Denucleotidyl Transferas (TdT), DNA polymerase Beta, DNA polymerase lambda, DNA polymerase mu and other enzymes from X family of DNA polymerases. In some embodiments, at least one nucleic acid molecule of said polynucleotide is generated in 20 minutes or less within said merged droplet. In some embodiments, at least one nucleic acid molecule of said polynucleotide is generated in 15 minutes or less within said merged droplet. In some embodiments, at least one nucleic acid molecule of said polynucleotide is generated in 10 minutes or less within said merged droplet. In some embodiments, at least one nucleic acid molecule of said polynucleotide is generated in 1 minute or less within said merged droplet. In some embodiments, said merged droplet is temperature-controlled. In some embodiments, said first droplet, said second droplet, said third droplet, or said merged droplet is subjected to a magnetic field. In some embodiments, said first droplet, said second droplet, said third droplet, or said merged droplet is subjected to light. “In some embodiments, said first droplet, said second droplet, said third droplet, or said merged droplet is subjected to pH change. In some embodiments, said first droplet, said second droplet, said third droplet, or said merged droplet comprises of deoxynucleoside triphosphate (dNTP). In some embodiments, said deoxynucleoside triphosphate may have a protective group. In some embodiments, said protective group can be removed during the reaction. In some embodiments, said first droplet, said second droplet, said third droplet, or said merged droplet make contact with a surface only on one side. In some embodiments, volumes of said first droplet, said second droplet, said third droplet, or said merged droplet is between 1 nanoliter (1 nl) and 500 microliters (500 μl). In some embodiments, volumes of said first droplet, said second droplet, said third droplet, or said merged droplet is between 1 microliter (1 μl) and 500 microliters (500 μl). In some embodiments, volumes of said first droplet, said second droplet, said third droplet, or said merged droplet is between 1 microliter (1 μl) and 200 microliters (200 μl). In some embodiments, the method further comprises ligating said biopolymer to a second biopolymer. In some embodiments, said second biopolymer was generated using any method as disclosed herein.

Another aspect of the present disclosure provides a method of generating a biopolymer, comprising: providing a plurality of droplets adjacent to a surface, wherein said plurality of droplets comprises a first droplet comprising a first reagent and a second droplet comprising a second reagent; subjecting said first droplet and said second droplet to motion relative to one another to (i) bring said first droplet in contact with said second droplet and (ii) form a merged droplet comprising said first reagent and said second reagent; and in said merged droplet, using at least (i) said first reagent and (ii) said second reagent to form at least a portion of said biopolymer, wherein a vibration is applied to (b), (c), or both. In some embodiments, said biopolymer is a polynucleotide. In some embodiments, said biopolymer is a polypeptide. In some embodiments, said polynucleotide comprises 2 to 10,000,000 nucleic acid molecules. In some embodiments, the method further comprises, one or more washing steps comprising subjecting a wash droplet to motion to contact said merged droplet. In some embodiments, a vibration is applied to said one or more washing steps. In some embodiments, at least one nucleic acid molecule of said polynucleotide is generated in 30 minutes or less within said merged droplet. In some embodiments, said surface is dielectric. In some embodiments, said surface comprises a dielectric layer disposed over one or more electrodes. In some embodiments, said surface is the surface of a polymeric film. In some embodiments, the surface comprises one or more oligonucleotides bound to the surface. In some embodiments, said surface is the surface of a lubricating liquid layer. In some embodiments, said plurality of droplets comprises a third droplet comprising a third reagent. In some embodiments, said first reagent, said second reagent, said third reagent, or any combination thereof comprises one or more functionalized beads. In some embodiments, said functional beads comprise one or more oligonucleotides immobilized thereto. In some embodiments, said first reagent, said second reagent, said third reagent, or any combination thereof comprises a polymerase. In some embodiments, said first reagent, said second reagent, said third reagent or any combination thereof comprises a bio-monomer. In some embodiments, said bio-monomer is an amino acid. In some embodiments, said bio-monomer is a nucleic acid molecule. In some embodiments, said nucleic acid molecule is adenine, cytosine, guanine, thymine, or uracil. In some embodiments, said first reagent comprises one or more functionalized discs. In some embodiments, said functionalized disc comprise one or more oligonucleotides immobilized thereto. In some embodiments, said first droplet, second droplet, third droplet, or both comprises an enzyme that mediate synthesis or polymerization. In some embodiments, said enzyme is from the group consisting of Polynucleotide Phosphorylase (PNPase), Terminal Denucleotidyl Transferas (TdT), DNA polymerase Beta, DNA polymerase lambda, DNA polymerase mu and other enzymes from X family of DNA polymerases. In some embodiments, at least one nucleic acid molecule of said polynucleotide is generated in 20 minutes or less within said merged droplet. In some embodiments, at least one nucleic acid molecule of said polynucleotide is generated in 15 minutes or less within said merged droplet. In some embodiments, at least one nucleic acid molecule of said polynucleotide is generated in 10 minutes or less within said merged droplet. In some embodiments, said merged droplet is heated. In some embodiments, said first droplet, said second droplet, said third droplet, or said merged droplet is subjected to magnetic field. In some embodiments, said first droplet, said second droplet, said third droplet, or said merged droplet is subjected to light. In some embodiments, said first droplet, said second droplet, said third droplet, or said merged droplet is subjected to pH change. In some embodiments, said first droplet, said second droplet, said third droplet, or said merged droplet comprises of deoxynucleoside triphosphate (dNTP). In some embodiments, said deoxynucleoside triphosphate may have a protective group. In some embodiments, said protective group can be removed during the reaction. In some embodiments, said first droplet, said second droplet, said third droplet, or said merged droplet make contact with a surface only on one side. In some embodiments, volumes of said first droplet, said second droplet, said third droplet, or said merged droplet is between 1 nanoliter (1 nl) and 500 microliters (500 μl). In some embodiments, volumes of said first droplet, said second droplet, said third droplet, or said merged droplet is between 1 microliter (1 μl) and 500 microliters (500 μl). In some embodiments, volumes of said first droplet, said second droplet, said third droplet, or said merged droplet is between 1 microliter (1 μl) and 200 microliters (200 μl).

Another aspect of the present disclosure comprises a method for processing a nucleic acid sample, comprising: providing a biological sample adjacent to an electrowetting array, wherein said sample droplet comprises said nucleic acid sample; and extracting said nucleic acid sample from said biological sample adjacent to said electrowetting array wherein said nucleic acid sample comprises a sequencing read having a length of at least about 70 kilobases (kb) In some embodiments, said length is at least about 80 kilobases (kb). In some embodiments, said length is at least about 200 kilobases (kb). In some embodiments, said sequencing read comprises an A260/A280 ratio of less than about 1.84.

Another aspect of the present disclosure provides a system for inducing motion in a droplet, comprising: (a) a surface configured to support said droplet comprising at least one bead formed of a material configured to couple to a magnetic field; (b) an actuator coupled a magnet, wherein said magnet is configured to supply said magnetic field, and wherein said actuator is configured to subject said magnetic field to translation along a plane parallel to said surface; and (c) a controller operatively coupled to said actuator, wherein said controller is configured to direct said actuator to subject said magnetic field to translation along said plane, such that while said magnetic field translates along said plane, said droplet undergoes motion along said surface. In some embodiments, said actuator is a switch. In some embodiments, said actuator comprises a motor coupled to said magnet, wherein said motor is configured to translate said magnet along a direction parallel to said surface. In some embodiments, the system further comprises an electrode configured to supply an electric field to said surface, wherein said electric field and said magnetic field are sufficient to subject said droplet to said motion. In some embodiments, said actuator is configured to motion said magnet to translate along at least two axes parallel to said plane. In some embodiments, said magnetic comprises a permanent magnet. In some embodiments, said magnet comprises at least one electromagnet. In some embodiments, said actuator comprises a pivot, wherein said pivot is coupled to said surface. In some embodiments, said surface comprises a dielectric disposed over one or more electrodes. In some embodiments, said one or more magnets are disposed below said surface. In some embodiments, said surface comprises a liquid layer. In some embodiments, said liquid layer comprises a liquid comprising an affinity for said surface.

Another aspect of the present disclosure provides a system for processing a sample, comprising: (a) a plurality of electrodes; (b) a dielectric layer disposed over said plurality of electrodes, wherein said dielectric layer comprises a surface configured to support a droplet comprising said sample; and (c) a liquid disposed in an interspace adjacent to said plurality of electrodes and said dielectric layer. In some embodiments, said liquid generates an adhesion between said plurality of electrodes and said dielectric layer. In some embodiments, said liquid comprises a dielectric material. In some embodiments, said liquid prevents or reduces electrical conductivity of air disposed in said interspace. In some embodiments, said dielectric layer comprises a natural polymeric material, a synthetic polymeric material, a fluorinated material, a surface modification, or any combination thereof. In some embodiments, said natural polymeric material comprises shellac, amber, wool, silk, natural rubber, cellulose, wax, chiton, or any combination thereof. In some embodiments, said synthetic polymeric material comprises polyethylene, polypropylene, polystyrene, polyetheretherketone (PEEK), polyimide, polyacetal, polysilfone, polyphenulene ether, polyphenylene Sulfide (PPS), polyvinyl chloride, synthetic rubber, neoprene, nylon, polyacrylonitrile, polyvinyl butyral, silicone, parafilm, polyethylene terephthalate, polybutylene terephthalate, polyamides, polyoxymethlyene, polycarbonate, polymethylpentene, polyphenylene oxide (Polyphenyl ether), polyphthalamide (PPA), polylactic acid, synthetic cellulose ethers (e.g., methyl cellulose, ethyl cellulose, propyl cellulose, hydroxyethyl cellulose, hydroxyethyl cellulose, hydroxypropyl cellulose (HPC), hydroxyethyl methyl cellulose, hydroxypropyl methyl cellulose (HPMC), ethyl hydroxyethyl cellulose), paraffins, microcrystalline wax, epoxy, or any combination thereof. In some embodiments, said fluorinated material comprises polytetrafluoroethylene (PTFE), tetrafluoroethylene (TFE), fluorinated ethylenepropylene copolymer (FEP), polyvinylidene fluoride (PVDF), perfluoroalkoxytetrafluoroethylene copolymer (PFA), perfluoromethyl vinylether copolymer (MFA), ethylenechlorotrifluoroethylene copolymer (ECTFE), ethylene-tetrafluoroethylene copolymer (ETFE), perfluoropolyether (PFPE), polychlorotetrafluoroethylene (PCTFE), or any combination thereof. In some embodiments, said surface modification comprises silicone, silane, fluoro-polymer treatment, parylene coating, any other suitable surface chemistry modification process, ceramic, clay minerals, bentonite, kaolinite, vermiculite, graphite, molybdenum disulfide, mica, boron nitride, sodium formate, sodium oleate, sodium palmitate, sodium sulfate, sodium alginate, or any combination thereof. In some embodiments, said liquid comprises silicone oils, fluorinated oils, ionic liquids, mineral oils, ferrofluids, polyphenyl ether, vegetable oil, esters of saturated fatty and dibasic acids, grease, fatty acids, triglycerides, polyalphaolefin, polyglycol hydrocarbons, other Non-hydrocarbon synthetic oils, or any combination thereof. In some embodiments, said liquid further comprises surfactants, electrolytes, rheology modifier, wax, graphite, graphene, molybdenum disulfide, PTFE particles, or any combination thereof. In some embodiments, said surface comprises a liquid layer. In some embodiments, said liquid layer comprises silicone oils, fluorinated oils, ionic liquids, mineral oils, ferrofluids, polyphenyl ether, vegetable oil, esters of saturated fatty and dibasic acids, grease, fatty acids, triglycerides, polyalphaolefin, polyglycol hydrocarbons, other Non-hydrocarbon synthetic oils, or any combination thereof. In some embodiments, said liquid layer further comprises surfactants, electrolytes, rheology modifier, wax, graphite, graphene, molybdenum disulfide, PTFE particles, or any combination thereof. In some embodiments, said dielectric layer is removable. In some embodiments, said adhesion is sufficient to immobilize said liquid onto said surface and wherein said liquid is resistant to gravity. In some embodiments, said liquid is selected to preferentially wet said surface to facilitate a motion of said droplet on said surface.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings (also “Figure” and “FIG.” herein), of which:

FIG. 1 shows a side section view for printed circuit boards with various steps in application of dielectric coating and steps in the process of planarization.

FIG. 2 represents a cartoon of computer systems used for arrays described herein.

FIG. 3A-3B illustrates example systems and methods for reference electrode placement on an electrode array. FIG. 3A represents a liquid coating that functions as a reference electrode. FIG. 3B represents electrically conductive ionized particles.

FIG. 4 depicts data for the distribution of the size of DNA isolated using systems and methods described herein.

FIG. 5A-5C depicts a configuration for the synthesis and assembly of biopolymers (e.g., DNA) using systems and methods described herein. FIG. 5A and FIG. 5B show example workflows to afford the synthesis of DNA. FIG. 5C shows a schematic diagram for a single reaction site that performs step by step addition of nucleotides to synthesize a long molecule of DNA.

FIG. 6 shows the library size distribution for on-chip vs. off-chip experiments of a NGS library preparation using systems and methods described herein.

FIG. 7 depicts the quality for sequencing libraries for on-chip vs. off-chip experiments of a NGS library preparation using systems and methods described herein.

FIG. 8 depicts the level of duplicates for sequencing libraries for on-chip vs. off-chip experiments of a NGS library preparation using systems and methods described herein.

FIG. 9A-9B depicts levels of adapter contamination for experiments of a NGS library preparation.

FIG. 10 depicts the level coverage across the human genome for experiments of a NGS library preparation using systems and methods described herein.

FIG. 11 depicts the single nucleotide polymorphism (SNP) sensitivity for experiments of a NGS library preparation using systems and methods described herein.

FIG. 12 depicts an example schematic NGS workflow using systems and methods described herein. The example workflow comprises manipulating (e.g., lysing cells, digesting protein, and DNA clean-up) biological samples on an array described herein.

FIGS. 13A-13B depict an array according to some embodiments described herein.

FIGS. 14A-14B depict an array according to some embodiments described herein.

FIG. 15 depicts a circuit map of a system without a dedicated reference electrode as described herein.

FIGS. 16A-16B illustrate one application of vibration assisted electrowetting on dielectric for the extraction of DNA using magnetic beads.

FIGS. 17A-17B show that high contact angle droplets (FIG. 17A) tend to experience greater response to vibration than droplets with lower contact angle (FIG. 17B).

FIGS. 18A-18B depict embodiments of electro-mechanical actuators.

FIGS. 19A-19B depict additional embodiments of electro-mechanical actuators.

FIG. 20 shows an embodiment of efficiently coupling the actuation force of the electro-mechanical actuator into droplet vibration and, ultimately, to effective mixing.

FIG. 21 shows an embodiment of tuning a vibration assisted EWOD system's natural frequency to be low relative to the desired frequency range.

FIG. 22 shows an embodiment of the present disclosure comprising the utilization of an oil for evaporation control of one or more droplets of interest on the arrays described herein.

FIG. 23 shows an embodiment of the present disclosure comprising a nucleic acid sequencing assay (e.g. nanopore sequencing) integrated into the arrays described herein.

FIG. 24 shows the results of high molecular weight (HMW) DNA extraction from GM12878 cells using the methods and devices of the present disclosure.

FIG. 25(A-C) shows the results of high molecular weight (HMW) DNA extraction from whole human blood samples using the methods and devices of the present disclosure.

FIG. 26A-26B shows the improved gDNA extraction results using the methods and devices of the present disclosure compared to manual sample and/or reagent handling.

FIG. 27 shows the improved gDNA extraction results using the methods and devices of the present disclosure compared to manual sample and/or reagent handling.

FIG. 28 shows the improved sequencing results on a MinION sequencing system using gDNA extracted using the methods and devices of the present disclosure compared to manual sample and/or reagent handling.

FIG. 29 shows improved sequencing data generated on a MinION sequencing system using gDNA extracted using the methods and devices of the present disclosure.

FIG. 30 shows improved sequencing data generated on a MinION sequencing system using gDNA extracted using the methods and devices of the present disclosure.

FIG. 31A-31B shows the distribution of the library fragment size for one GM12878 and one whole blood sample extracted using the methods and devices of the present disclosure.

FIG. 32A-32B shows the distribution of read lengths on a MinION sequencing system using gDNA extracted using the methods and devices of the present disclosure.

FIG. 33A-33E shows the sequencing results on a Pacific Biosciences of California HiFi sequencing system using gDNA extracted using the methods and devices of the present disclosure; including read length (33A), subread length (33B), read quality distribution (33C), HiFi read length distribution (33D), and a model of predicted accuracy v. read length (33E).

FIG. 34 shows the increase in the average concentration/purity of DNA extracted using the methods and devices of the present disclosure.

FIG. 35A-35B shows the increasing experimentation and increasing workflow robustness of the methods and devices of the present disclosure as the methods and devices are further utilized.

FIG. 36A-36B shows embodiments of the systems and devices described herein comprising a movable magnet to induce motion in magnetically responsive materials contained in droplets on the arrays/substrates described herein.

### DETAILED DESCRIPTION

While various embodiments of the invention have been shown and described herein, it will be obvious to those skilled in the art that such embodiments are provided by way of example only. Numerous variations, changes, and substitutions may occur to those skilled in the art without departing from the invention. It should be understood that various alternatives to the embodiments of the invention described herein may be employed.

Whenever the term “at least,” “greater than,” or “greater than or equal to” precedes the first numerical value in a series of two or more numerical values, the term “at least,” “greater than” or “greater than or equal to” applies to each of the numerical values in that series of numerical values. For example, greater than or equal to 1, 2, or 3 is equivalent to greater than or equal to 1, greater than or equal to 2, or greater than or equal to 3.

Whenever the term “no more than,” “less than,” or “less than or equal to” precedes the first numerical value in a series of two or more numerical values, the term “no more than,” “less than,” or “less than or equal to” applies to each of the numerical values in that series of numerical values. For example, less than or equal to 3, 2, or 1 is equivalent to less than or equal to 3, less than or equal to 2, or less than or equal to 1.

The term “slide angle”, as used herein, generally refers to the angle from horizontal at which a droplet of a given size begins to move under the force of gravity. For example, a surface that holds a 5 microliter (μl) droplet at 4° but allows it to slide at 5° may be the to have a 5 μl slide angle of 5°. For various applications, 5 μl slide angles of less than or equal to 70°, 60°, 50°, 40°, 30°, 25°, 20°, 15°, 10° 5°, 3°, 2°, 1° or less may be used. The smaller the slide angle, the more slippery the surface, and generally the lower the voltage required to move droplets across the surface.

The term “contact angle hysteresis”, as used herein, generally refers to the observed differences between advancing and receding contact angles. For example, in a surface with lower surface adhesion, as a liquid droplet moves across the surface, the contact angle between the leading edge and the surface vs. the trailing edge and the surface can be close the same. However, in a surface with higher adhesion, the difference between the leading and trailing contract angles can become larger. Low surface roughness, high surface hydrophobicity, and low surface energy can result in less difference in this angle. Contact angle hysteresis (that is, the difference between leading and trailing contact angles) of less than or equal to 70°, 60°, 50°, 40°, 30°, 25°, 20°, 15°, 10⁰, 7⁰, 5⁰, 3⁰, 2° or less may be used.

The term “droplet”, as used herein, generally refers to a discrete or finite volume of a fluid (e.g., a liquid). A droplet may be generated by one phase separated from another phase by an interface. The droplet may be a first phase phase-separated from another phase. The droplet me include a single phase or multiple phases (e.g., an aqueous phase containing a polymer). The droplet may be a liquid phase disposed adjacent to a surface and in contact with a separate phase (e.g., gas phase, such as air).

The term “biological sample,” as used herein, generally refers to a biological material. Such biological material may display bioactivity or be bioactive. Such biological material may be, or may include, a deoxyribonucleic acid (DNA) molecule, a ribonucleic acid (RNA) molecule, a polypeptide (e.g., protein), or any combination thereof. A biological sample (or sample) may be a tissue sample, such as a biopsy, core biopsy, needle aspirate, or fine needle aspirate. The sample may be a fluid sample, such as a blood sample, urine sample, stool sample, or saliva sample. The sample may be a skin sample. The sample may be a cheek swab. The sample may be a plasma or serum sample. The sample may be a plant derived sample, water sample or soil sample. The sample may be extraterrestrial. The extraterrestrial sample may contain biological material. The sample may be a cell-free (or cell free) sample. A cell-free sample may include extracellular polynucleotides. Extracellular polynucleotides may be isolated from a bodily sample that may be selected from a group consisting of blood, plasma, serum, urine, saliva, mucosal excretions, sputum, stool and tears. The sample may include a eukaryotic cell or a plurality thereof. The sample may include a prokaryotic cell or a plurality thereof. The sample may include a virus. The sample may include a compound derived from an organism. The sample may be from a plant. The sample may be from an animal. The sample may be from an animal suspected of having or carrying a disease. The sample may be from a mammal.

The term “% glycerol,” as used herein, generally refers to the viscosity of a solution as compared to a glycerol in water solution wherein the amount of glycerol in water (by volume) is determined by the value of the percentage. For example, a solution described herein with a viscosity of about “30% glycerol” expresses that the viscosity of the solution is the equivalent of a glycerol in water solution comprising about 30% glycerol.

The term “subject,” as used herein, generally refers to an animal, such as a mammal (e.g., human) or avian (e.g., bird), or other organism, such as a plant. The subject can be a vertebrate, a mammal, a rodent (e.g., a mouse), a primate, a simian or a human. Animals may include, but are not limited to, farm animals, sport animals, and pets. A subject can be a healthy or asymptomatic individual, an individual that has or is suspected of having a disease (e.g., cancer) or a pre-disposition to the disease, an individual that needs therapy or suspected of needing therapy, or any combination thereof. A subject can be a patient

The term “electro-mechanical actuator,” as used herein, generally refers to a non-human structure that can be utilized to apply vibration and/or acoustic forces to the arrays described herein. By way of non-limiting examples, electro-mechanical actuators include an oscillating mechanism or cantilever, motor-driven linkages, and/or rotating masses. In some embodiments, the electro-mechanical actuators described herein are flexible structures comprising various flexible elements (e.g. a linear flexure) or with traditional bearings

The term “coefficient of variation,” as used herein, generally refers to repeatability and precision. This may be given by Equation 1, where s is the standard deviation of the responsivities of the different materials and x is the mean responsivity of all materials.

$$CV = \frac{s}{x} \times 100 \qquad Equation 1$$

The term “cross-talk,” as used herein, generally refers to contamination of a droplet. Cross-talk may refer to a percentage of a droplet, a biological sample, or a combination thereof acquired from another droplet. If p1 represents the material of interest in the droplet and p₂ is the total material from other droplets present in the droplet of interest, the cross-talk may be given by Equation 2.

$$CT = \frac{{p}_{2}}{{p}_{1} + {p}_{2}} \qquad Equation 2$$

### Electrowetting Devices and Systems

An electrowetting device may be used to move individual droplets of water (or other aqueous, polar, or conducting solution) from place to place. The surface tension and wetting properties of water may be altered by electric field strength using the electrowetting effect. The electrowetting effect may arise from the change in solid-liquid contact angle due to an applied potential difference between the solid and the liquid. Differences in wetting surface tension that may vary over the width of the droplet, and corresponding change in contact angle, may provide motive force to cause the droplets to move, without moving parts or physical contact. The electrowetting device may include a grid of electrodes with a dielectric layer with appropriate electrical and surface priorities overlaying electrodes, all laid on a rigid insulating substrate. Additional examples of electrowetting devices can be found in WO2021041709, which is hereby incorporated by reference in its entirety.

The surface of the electrode grid may be prepared so that it has low adhesion with water. This may allow water droplets to be moved along the surface by small forces generated by gradients in electric field and surface tension across the width of the droplet. A surface with low adhesion may reduce the trail left behind from a droplet. A smaller trail may reduce droplet cross contamination, and may reduce sample loss during droplet movement. Low adhesion to surface may also allow for low actuation voltage for droplet motion and repeatable behavior of droplet motion. There are several ways to measure low adhesion between a surface and a droplet including slide angle and contact angle hysteresis, such as, for example, using a contact angle goniometer or a charge-coupled device (CCD) camera.

There may be several ways to achieve low surface adhesion; for example, mechanically polishing, chemically etching, or a combination thereof until smooth within a few nanometers, applying coating to fill surface irregularities, applying liquids to fill surface irregularities, chemically modifying the surface to create desirable surface properties (hydrophobic, hydrophilic, resistance to biofouling, varying with electric field strength, etc.).

### Liquid-On-Liquid Electrowetting (LLEW) for Electrowetting

An electrowetting mechanism called “liquid-on-liquid-electrowetting” (LLEW) takes advantage of an electrowetting phenomenon that occurs at a liquid-liquid-gas interface. A droplet riding on the surface of a layer of a low surface energy liquid (such as oil) and substantially surrounded by gas (such as air, nitrogen, argon, etc.) creates a liquid-liquid-gas interface at the contact line. The oil may be stabilized in place on the solid substrate by a textured surface of the solid substrate, and the conductive layer of metal electrodes may be embedded in the body of this solid. In some embodiments, when an electric potential is applied across the height of droplet, the liquid-liquid-gas interface may cause droplet to wet the oil and spread across the surface while still riding on the oil.

In some embodiments, the liquid-on-liquid electrowetting technique may be used to manipulate droplets that may contain biological and chemical samples. In some embodiments, a droplet may be in motion from left to right, and can been attracted onto the left-most of three electrodes by a positive voltage on that leftmost electrode, with consequent addition of electric field at the liquid-liquid surface and enhanced wetting. In some embodiments, the voltage is withdrawn from the leftmost electrode and applied to the center electrode. In some embodiments, because of the enhanced wetting over the center electrode, the droplet may be attracted to the center position. In some embodiments, the voltage is withdrawn from the left and center electrodes and applied to the right electrode, and the enhanced wetting over the right electrode has attracted the droplet to the right.

In some embodiments, differential wetting may be used to merge two droplets on a LLEW surface over an electrode array. In some embodiments, two droplets have been attracted to the leftmost and rightmost electrodes. In some embodiments, the voltage is removed from the left and right electrodes and applied to the center electrode. The two droplets may be attracted from left and right to center and begin to merge.

In some embodiments, such a microfluidic selective wetting device may be capable of performing, for example, microfluidic droplet actuation such as droplet transport, droplet merging, droplet mixing, droplet splitting, droplet dispensing, droplet shape change, or a combination thereof. This LLEW droplet actuation may then be used for a microfluidic device to automate biological experiments such as liquid assays, in devices for medical diagnostics and in many lab-on-a-chip applications.

Additional examples LLEW droplet actuation can be found in WO2021041709, which is hereby incorporated by reference in its entirety.

In some embodiments, the low surface energy liquid (e.g., oil) may be stabilized in place on the solid surface without texturing the solid surface. In these embodiments, stabilization of the liquid layer relies on chemical affinity between the surface of the underlying surface and the liquid layer. In some embodiments, the liquid layer is a lubricant film. In some embodiments, the lubricant film is thermodynamically stable such that it preferentially wets the surface of the underlying surface. In some embodiments, the underlying surface is a solid substrate. In some embodiments, the solid substrate is a dielectric. In some embodiments, the underlying surface is a film. In some embodiments, the film is a dielectric film. In some embodiments, achieving this stability is important and is governed by the affinity of the lubricant liquid to the surface of the dielectric. In some embodiments, for fluorinated surfaces of dielectric, it may be advantageous to use fluorinated lubricant liquids. The similar chemical structure leads to a greater affinity and therefore the lubricant is more likely to wet the surface in a stable way. In some embodiments, when using dielectrics with hydrocarbon based surfaces, or siliconized surfaces, (such as silicones and untreated polymer plastics), it may be advantageous to use a hydrocarbon based lubricant liquid (such as silicone oil).

An aspect of the present disclosure comprises a system for processing a sample, comprising: a plurality of electrodes; a dielectric layer disposed over the plurality of electrodes, wherein the dielectric layer comprises a surface configured to support a droplet comprising the sample; a liquid adjacent to the surface, wherein the liquid comprises a chemical affinity to the surface, and wherein the chemical affinity is sufficient to immobilize the liquid onto the surface and wherein the liquid is resistant to gravity. In some embodiments, the dielectric layer comprises a natural polymeric material, a synthetic polymeric material, a fluorinated material, a surface modification, or any combination thereof. In some embodiments, the natural polymeric material comprises shellac, amber, wool, silk, natural rubber, cellulose, wax, chiton, or any combination thereof. In some embodiments, the synthetic polymeric material comprises polyethylene, polypropylene, polystyrene, polyetheretherketone (PEEK), polyimide, polyacetal, polysilfone, polyphenulene ether, polyphenylene Sulfide (PPS), polyvinyl chloride, synthetic rubber, neoprene, nylon, polyacrylonitrile, polyvinyl butyral, silicone, parafilm, polyethylene terephthalate, polybutylene terephthalate, polyamides, polyoxymethlyene, polycarbonate, polymethylpentene, polyphenylene oxide (Polyphenyl ether), polyphthalamide (PPA), polylactic acid, synthetic cellulose ethers (e.g., methyl cellulose, ethyl cellulose, propyl cellulose, hydroxyethyl cellulose, hydroxyethyl cellulose, hydroxypropyl cellulose (HPC), hydroxyethyl methyl cellulose, hydroxypropyl methyl cellulose (HPMC), ethyl hydroxyethyl cellulose), paraffins, microcrystalline wax, epoxy, or any combination thereof. In some embodiments, the fluorinated material comprises polytetrafluoroethylene (PTFE), tetrafluoroethylene (TFE), fluorinated ethylenepropylene copolymer (FEP), polyvinylidene fluoride (PVDF), perfluoroalkoxytetrafluoroethylene copolymer (PFA), perfluoromethyl vinylether copolymer (MFA), ethylenechlorotrifluoroethylene copolymer (ECTFE), ethylene-tetrafluoroethylene copolymer (ETFE), perfluoropolyether (PFPE), polychlorotetrafluoroethylene (PCTFE), or any combination thereof. In some embodiments, the surface modification comprises silicone, silane, fluoro-polymer treatment, parylene coating, any other suitable surface chemistry modification process, ceramic, clay minerals, bentonite, kaolinite, vermiculite, graphite, molybdenum disulfide, mica, boron nitride, sodium formate, sodium oleate, sodium palmitate, sodium sulfate, sodium alginate, or any combination thereof. In some embodiments, the liquid comprises silicone oils, fluorinated oils, ionic liquids, mineral oils, ferrofluids, polyphenyl ether, vegetable oil, esters of saturated fatty and dibasic acids, grease, fatty acids, triglycerides, polyalphaolefin, polyglycol hydrocarbons, other Non-hydrocarbon synthetic oils, or any combination thereof. In some embodiments, the liquid further comprises surfactants, electrolytes, rheology modifier, wax, graphite, graphene, molybdenum disulfide, PTFE particles, or any combination thereof. In some embodiments, the surface comprises a liquid layer. In some embodiments, the liquid layer comprises silicone oils, fluorinated oils, ionic liquids, mineral oils, ferrofluids, polyphenyl ether, vegetable oil, esters of saturated fatty and dibasic acids, grease, fatty acids, triglycerides, polyalphaolefin, polyglycol hydrocarbons, other Non-hydrocarbon synthetic oils, or any combination thereof. In some embodiments, the liquid layer further comprises surfactants, electrolytes, rheology modifier, wax, graphite, graphene, molybdenum disulfide, PTFE particles, or any combination thereof. In some embodiments, the dielectric layer is removable.

### Electrowetting on a Dielectric (EWOD) for Droplet Manipulation

In some embodiments, Electrowetting on Dielectric (EWOD) is a phenomenon in which the wettability of an aqueous, polar, or conducting liquid (L) may be modulated through an electric field across a dielectric film between the droplet and conducting electrode. Adding or subtracting charge from electrode may change the wettability of an insulating dielectric layer, and that wettability change is reflected in a change to contact angle of the droplet. The contact angle change may in turn cause the droplet to change shape, to move, to split into smaller droplets, or to merge with another droplet. Additional examples EWOD droplet actuation can be found in WO2021041709, which is hereby incorporated by reference in its entirety.

### Properties of Arrays

Described herein are arrays and substrates configured to facilitate EWOD to induce droplet actuation. The array may comprise a plurality of elements which may comprise: a plurality of heaters, a plurality of coolers, a plurality of magnetic field generators, a plurality of electroporation units, a plurality of light sources, a plurality of radiation sources, a plurality of nucleic acids sequencers, a plurality of biological protein channels, a plurality of solid state nanopores, a plurality of protein sequencers, a plurality of acoustic transducers, a plurality of microelectromechanical system (MEMS) transducers, a plurality of capillary tubes as liquid dispensers, a plurality of holes for dispensing or transferring liquids using gravity, a plurality of electrodes in a hole to dispense or transfer liquids using electric field, a plurality of holes for optical inspection, a plurality of holes for liquids to interact through membranes, or any combination thereof. The plurality of elements may comprise less than or equal to about 100, 90, 80, 70, 60, 50, 40, 30, 20, 10, 5, 4, 3, 2 or less of each element. The plurality of elements may comprise greater than or equal to about 2, 3, 4, 5, 6, 7, 8, 9, 10, 20, 30, 40, 50, 60, 70, 80, 90, 100 or more of each element.

The heater may have a maximum temperature less than or equal to about 150° C., 125° C., 100° C., 75° C., 50° C., 25° C., or less. The heater may be thermoelectric, resistive, or heated by a heat transfer medium (e.g., a recirculated hot water loop). The cooler may have a minimum temperature greater than or equal to about −50° C., −25° C., −10° C., −5° C., 0° C., 10° C., or more. The cooler may be thermoelectric, evaporative, or cooled by a heat transfer medium (e.g., a water chiller).

The magnetic field generator may be for magnetic bead based operations or for other operations requiring magnetic field. The magnetic field generator may be electromagnets.

The electroporation unit may be two or more electrodes on either side of the droplet.

The light source may be broadband, monochromatic, or a combination thereof. The light source may be an incandescent source, a light emitting diode (LED), a laser, or a combination thereof. The light source may emit polarized light, collimated light, or a combination thereof. The plurality of radiation sources may emit ultraviolet light (light of a wavelength from 10 nm to 400 n), x-rays, gamma rays, alpha particles, beta particles, or a combination thereof. The radiation source may be collimated.

### Substrates for Electrowetting

An electrowetting microfluidic device may be formed by creating a slippery (in the sense of low surface energy) surface directly on the electrode array (120). Electrode arrays may consist of conductive plates that charge electrically to actuate the droplets. Electrodes in an array may be arranged in an arbitrary layout, for example a rectangular grid, or a collection of discrete paths. The electrodes themselves may be made of one or more conductive metals (including gold, silver, copper, nickel, aluminum, platinum, titanium), one or more conductive oxides (including indium tin oxide, aluminum doped zinc oxide), one or more conductive organic compounds (including PEDOT and polyacetylene), one or more semiconductors (including, silicon dioxide), or any combination thereof. The substrates for laying out the electrode array may be any insulating materials of any thickness and any rigidity.

The electrode arrays may be fabricated on standard rigid and flexible printed circuit board substrates. The substrate for the PCB may be FR4 (glass-epoxy), FR2 (glass-epoxy), Rogers material (hydrocarbon-ceramic), or insulated metal substrate (IMS), polyimide film (example commercial brands include Kapton, Pyralux), polyethylene terapthalate (PET), ceramic or other commercially available substrates of thickness from 1 μm to 10,000 μm. Thicknesses from 500 μm to 2000 μm may be utilized in some embodiments.

The electrode arrays may also be made of conductive elements, semiconductive elements, or any combination thereof which may be fabricated with active matrix technologies and passive matrix technologies such as thin film transistor (TFT) technology. The electrode arrays may also be made of arrays of pixels fabricated with traditional CMOS or HV-CMOS fabrication techniques.

The electrode arrays may also be fabricated with transparent conductive materials such as indium tin oxide (ITO), aluminum doped zinc oxide (AZO), fluorine doped tin oxide (FTO) deposited on sheets of glass, polyethylene terapthalate (PET) and any other insulating substrates.

The electrode arrays may also be fabricated with metal deposited on glass, polyethylene terapthalate (PET) and any other insulating substrates.

In constructing the electrowetting microfluidic device, many layers of laminations (from 1 to 50 layers) may be used to isolate multiple layers of electrical interconnect routing (from 2 to 50 layers). One of the outermost layers of lamination may contain electrode pads for actuating droplets and may contain reference electrodes. The interconnects may connect the electrical pads to high voltages for actuation and for capacitive sensing. The actuation voltage may be from 1V to 350V. This actuation voltage may be an AC signal or DC signal.

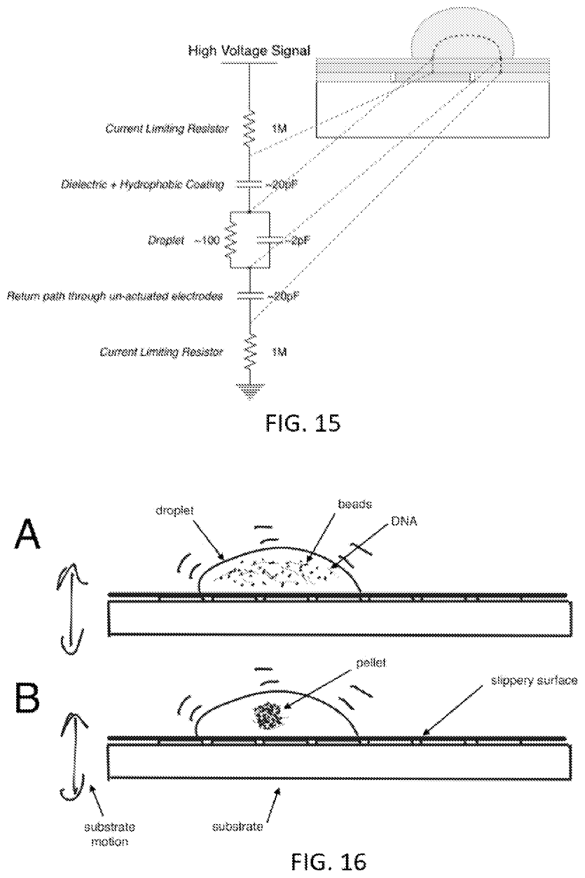

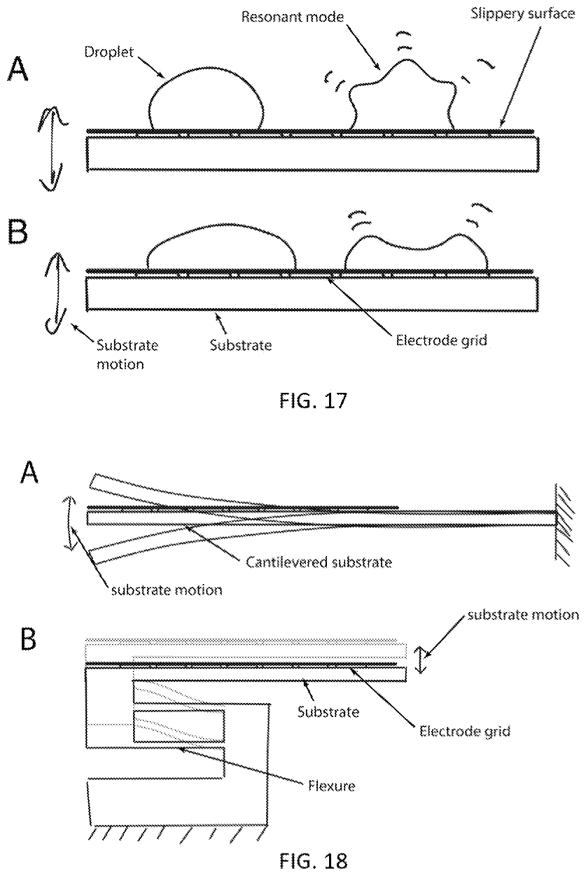

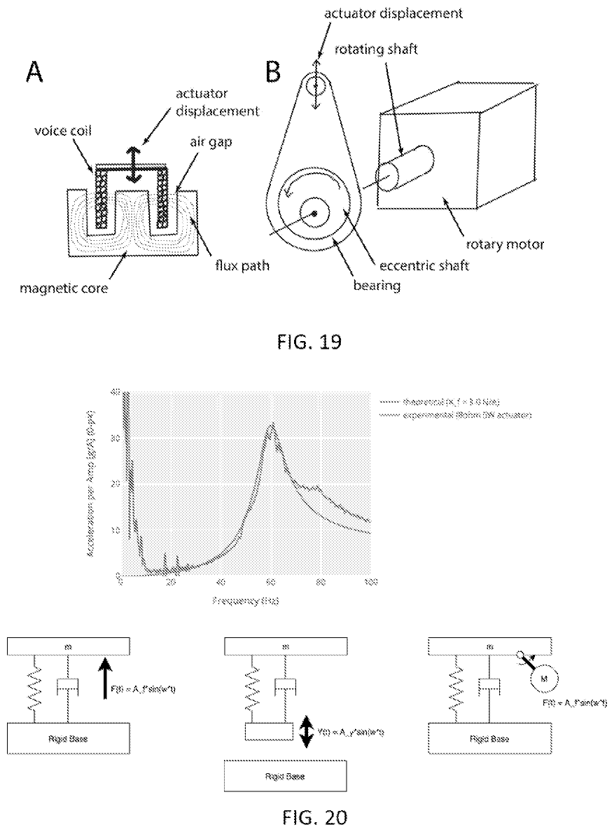

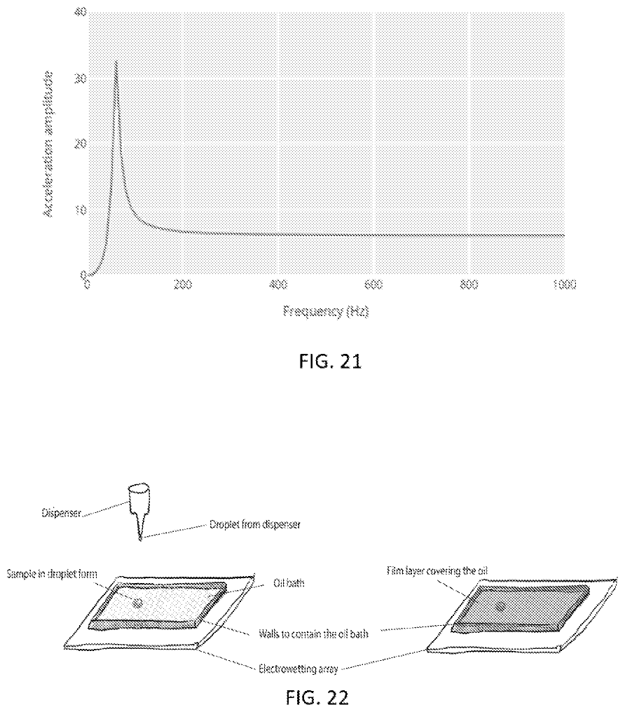

In further embodiments, the substrate does not have a dedicated reference electrode. The circuit with a conventional dedicated reference electrode includes a resistive return path which acts to ground the droplet. Without a dedicated reference electrode, the return path includes a capacitive element formed between the inactive electrode(s) and the droplet across the dielectric membrane (FIG. 15). For this reason, activating the electrodes with a time-varying voltage is necessary in order for this current-return path to be effective. This time varying voltage may be bipolar in which case the high voltage signal is both positive and negative relative to the “0V” inactive electrodes. In another embodiment, the time varying voltage may be unipolar in which case the high voltage signal is only positive and neighboring electrodes are driven antagonistically such that the electric field across the droplet flips direction periodically.

Additional examples of substrates for EWOD droplet actuation can be found in WO2021041709, which is hereby incorporated by reference in its entirety.

Some aspects of the present disclosure provide for different surface. In some embodiments, the surface is dielectric. In some embodiments, the surface comprises a dielectric layer disposed over one or more electrodes. In some embodiments, the surface is the surface of a polymeric film. In some embodiments, the surface comprises one or more nucleotides bound to the surface. In some embodiments, the surface is the surface is the surface of a lubricating liquid layer.

### Creating Smooth Dielectric Surface on the Electrode Array

In order to isolate the droplet electrically from the electrode array, a layer of dielectric may be applied on the top surface of the electrode array. The top surface of this dielectric layer may be formed with a top surface that offers little to no resistance to droplet motion, so that droplets may be moved with low actuation voltages (less than 100V DC, less than 80V, less than 50V, less than 40V, less than 30V, less than 20V, less than 15V, less than 10V, less than 8V, or less, depending on the degree of smoothness, slipperiness, hydrophobicity, or any combination thereof). To achieve a low resistance slippery surface, the dielectric surface may have a smooth surface topography and may be hydrophobic or otherwise offer low adherence to the droplet. A chemical treatment may also be applied directly to the dielectric surface.

A smooth topography surface is typically characterized by its roughness value. By experimentation, it has been found that the voltages required to effect droplet motion may vary as the surface becomes smoother. The smoothness may be less than 2 μm, 1 μm, 500 nm, or less. Examples of methods of creating smooth dielectric surfaces for EWOD droplet actuation can be found in WO2021041709, which is hereby incorporated by reference in its entirety.

### Surface Chemistry Modification (Functionalization)

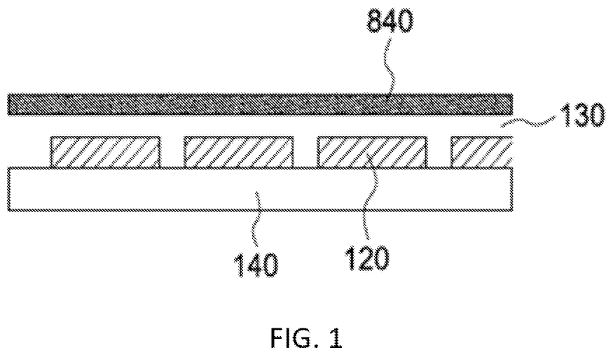

Referring to FIG. 1, the surface energy may be reduced by chemical modification, for example, by coating over the electrodes (120), dielectric (130), or any combination thereof with hydrophobic or low-surface energy materials (840) such as, for example, fluorocarbon based polymers (fluoropolymers), polyethylenes, polypropylenes, or other hydrophobic surface coatings.

The surface coating may be applied by one or more methods, including spin coating, dip coating, spray coating, drop coating, chemical vapor deposition, or other methods.

In some cases, it may be desirable to choose a conformal coating that may act as both a dielectric (to insulate the droplets from the charge of the electrical pads while allowing the electric field to propagate) and as a hydrophobic or hydrophilic or both coating (to reduce adhesion and allow smooth droplet motion).

Droplet Motion, Merging and Splitting

A droplet may be moved, merged, split, or any combination thereof on an open surface electrowetting device. The same principles apply to two plate configuration (droplet sandwiched).

In some embodiments, applying a voltage to an electrode may make the overlying surface hydrophilic and a droplet can then wet it. When voltage is applied on two neighboring electrodes, the droplet may spread across both actuated electrodes. When voltage is removed from electrode and applied to another adjacent electrode, the surface returns to original hydrophobic state and the droplet may be pushed out. By sequentially controlling the voltage applied to an electrode grid, a droplet's position on a surface may be precisely controlled.

In some embodiments, when two droplets are pulled towards the same electrode, they may naturally merge due to surface tension. This principle may be applied to merge a number of droplets to create a larger volume droplet spreading across multiple electrodes.

In some embodiments, a droplet may be split into two smaller ones through a sequence of voltages, applied across multiple electrodes (at least three electrodes). In some embodiments, a single large droplet is consolidated above a single electrode. In some embodiments, an equal voltage is applied to three adjacent electrodes simultaneously, and this may cause the single droplet to spread across the three adjacent electrodes. In some embodiments, turning off the center electrode may force the droplet to move out to the two outer electrodes. Due to the equal potential on both of the two outer electrodes, the droplet may then split into two smaller droplets.

Lab in a Box (Desktop Digital Wetlab)

Any combination of the manufacturing methods described so far may be used for the application described in this section.

In some embodiments, described herein is a device that may provide a general-purpose machine that may automate a large variety of biological protocols/assays/tests. The device may comprise a box that may have a lid that can be opened and closed. The lid may have a clear window to view the motion of droplets on the electrode array, which may be formed as a digital microfluidic chip. The box may house a digital microfluidic chip capable of moving, merging, splitting droplets, in which the droplets may carry biological reagents. The microfluidic chip may also have one or more heaters or chillers that may be able to heat droplets from as high as 1500 Celsius or more or cool the droplets from as low as −20° Celsius or less.

Aspects of the present disclosure provide for thermal control. In some embodiments, there is on-chip thermal control.

Droplets may be dispensed onto the chip through one or more “liquid dispenser(s)”. Each liquid dispenser may be, for example, an electro-fluidic pump, syringe pump, simple tube, robotic pipettor, inkjet nozzle, acoustic ejection device, or other pressure or non-pressure driven device. Droplets may be fed in to the liquid dispenser from a reservoir labeled “reagent cartridge”. The “lab-in-a-box” may have up to a several hundred reagent cartridges interfacing directly with the microfluidic chip.

Droplets may be moved from the digital microfluidic chip on to micro plates. Microplates may be plates with wells that can hold samples. Microplates may have anywhere from one to a million wells on a single plate. Multiple microplates may interface with the chip in the box. To dispense droplets from the microfluidic chip to the microplate, electrowetting chips with various geometries may be used. In some cases, the dispensing chip may be in the form of a cone resembling a pipette tip. In another embodiment, the dispensing aperture may be a cylinder. In another embodiment, the dispensing apparatus may be two parallel plates with a gap in between. In another embodiment, the dispensing apparatus may be a single open surface with at least one droplet moving on the open surface. The dispensing mechanism may also use a number of other mechanisms such as, for example, electrofluidic pumps, syringe pump, tubes, capillaries, paper, wicks or even simple holes in the chip.

An aspect of the disclosure presents microfluidic dispense chips.

The “lab-in-a-box” may be climate controlled to regulate the internal temperature, humidity, lighting conditions, droplet size, pressure, droplet coating, oxygen concentration, or any combination thereof. The inside of the box may be at vacuum. The inside of the box may be purged with a combination of a variety of gasses. The gasses may include air, argon, nitrogen, or carbon dioxide.

The digital microfluidic chip at the center of the box may be removed, washed and replaced.

The digital microfluidic chip at the center of the box may be disposable.

The digital microfluidic device may include sensors to perform various assays, for example optical spectroscopy, or sonic transducers.

The digital microfluidic device may include a magnetic bead-based separation unit for DNA size selection, DNA purification, protein purification, plasmid extraction and any other biological workflow that uses magnetic beads. The device may perform a number of simultaneous magnetic bead-based operations—from one to a million on a single chip.

The box may be equipped with multiple cameras looking at the chip from the top, sides and bottom. The cameras may be used to locate droplets on the chip, to measure volumes of droplets, to measuring mixing, and to analyze reactions in progress. Information from these sensors may be provided as feedback to computers that control the electrical flow to the electrodes, so that the droplets may be accurately controlled to achieve high throughput rates with accurate drop positioning, mixing, etc. Information from these sensors may be provided to a machine learning algorithm or neural network. This machine learning data may also be used to ensure that the assigned protocol has been properly executed. This may be achieved through the establishment of machine learning classifiers that may enable automated monitoring and tracing of events occurring during the assay that may indicate atypical incidents. The machine learning algorithms may also allow for the box to optimize and improve assays. The detection of unusual fluidic phenomenon across a database of run data may help to optimize assay performance. This data may help to improve assay results and reliability on the box.

The lab-in-a-box may be used to perform microplate operations such as plate stamping, serial dilution, plate replicate and plate rearray.

The lab-in-a-box may include equipment for PCR amplification and DNA assembly (Gibson Assembly, Golden Gate Assembly), molecular cloning, DNA library preparation, RNA library preparation DNA sequencing, single cell sorting, cell incubation, cell culture, cell assay, cell lysing, DNA extraction, protein extraction, RNA extraction, RNA and cell-free protein expression.

Processing Stations

An electrowetting chip (with or without a lab-in-a-box enclosure) may include one or more stations for various functions.

Mixing and Partitioning Stations

In some embodiments, an electrowetting device may incorporate one or more mixing stations. In some embodiments, 2×2 collection of electrowetting-based mixing stations may be operated in parallel. A single mixing station may have a 3×3 grid of actuation electrodes. Each mixing station may be used to mix biological samples, chemical reagents, and liquids. For example, droplets of two reagents may be brought together at a mixing station, and then mixed by running the merged droplet around the outer eight electrodes of the 3×3 grid, or running through other patterns designed to mix the two original droplets. The center-to-center spacing between each mixing station may be 9 mm, equivalent to the spacing of a standard 96-well plate.

The mixing stations may be extended to have a number of different configurations. Each single mixer may be comprised of any number of actuation electrodes in an A×B pattern. Additionally, the spacing between mixers is arbitrary and may be altered to fit the application (such as other SDS plates). A parallel mixing station may also have any number of individual mixers in an M×N pattern. Parallel mixing stations may have any configuration of top plate including but not limited to an open face, a closed plate, or a closed plate with liquid entry holes.

The mixing stations may be used as partitioning stations. Partitioning stations may use the electrowetting force to partition one droplet into a plurality of droplets. In addition to the electrowetting force, other methods can be used to partition droplets, including dielectrowetting forces, dielectrophoretic effects, acoustic forces, hydrophobic knives, or any combination thereof. Partitioning may be used for a variety of purposes, such as dispensing reagents or samples. Partitioned droplets may then be mixed with other droplets to execute a reaction in the other droplets. The partitioned droplets may be analyzed by the same sensors and methods as non-partitioned droplets.

Partitioned droplets may be mixed with target droplets to maintain a constant volume of at least one target droplet, where the at least one target droplet has lost volume (for example due to evaporation, being partitioned itself, etc.). The instruction to mix the droplets may come from an attached device such as a computer or smartphone.

Temperature Control Station

In some embodiments, an electrowetting chip may include one or more temperature control station(s). Each station may integrate one or more functions to be applied to liquid samples such as mixing, heating (for example, to temperatures up to and including 1500 Celsius), cooling (for example, down to and including −20° Celsius), compensating for fluid loss due to evaporation as well as homogenizing temperature of a sample. Heating or cooling may be accomplished by metal traces, foil heaters, Peltier elements external to the substrate, or a combination thereof. In some cases, the individualized heating elements may permit each station to be controlled to a separate temperature, for example, −20° C., 25° C., 37° C., and 95° C., depending on the heat transfer power of each element and the heat conduction levels between stations.

A parallel temperature control station may be configured in any of the same configurations as a parallel mixing station.

An aspect of present disclosure provides that the merged droplet is temperature-controlled.

The heater may have a maximum temperature less than or equal to about 150° C., 125° C., 100° C., 75° C., 50° C., 25° C., or less. The heater may be thermoelectric, resistive, or heated by a heat transfer medium (e.g., a recirculated hot water loop). The cooler may have a minimum temperature greater than or equal to about −50° C., −25° C., −10° C., −5° C., 0° C., 10° C., or more. The cooler may be thermoelectric, evaporative, or cooled by a heat transfer medium (e.g., a water chiller).

The temperature control stations as described herein may configured to precisely control and manipulate the temperature applied to the liquid sample. In some embodiments, the temperature control stations are configured to heat/cool the liquid samples by about 0.1° C. to about 1° C. In some embodiments, the temperature control stations are configured to heat/cool the liquid samples by about 0.1° C. to about 0.2° C., about 0.1° C. to about 0.3° C., about 0.1° C. to about 0.4° C., about 0.1° C. to about 0.5° C., about 0.1° C. to about 0.6° C., about 0.1° C. to about 0.7° C., about 0.1° C. to about 0.8° C., about 0.1° C. to about 0.9° C., about 0.1° C. to about 1° C., about 0.2° C. to about 0.3° C., about 0.2° C. to about 0.4° C., about 0.2° C. to about 0.5° C., about 0.2° C. to about 0.6° C., about 0.2° C. to about 0.7° C., about 0.2° C. to about 0.8° C., about 0.2° C. to about 0.9° C., about 0.2° C. to about 1° C., about 0.3° C. to about 0.4° C., about 0.3° C. to about 0.5° C., about 0.3° C. to about 0.6° C., about 0.3° C. to about 0.7° C., about 0.3° C. to about 0.8° C., about 0.3° C. to about 0.9° C., about 0.3° C. to about 1° C., about 0.4° C. to about 0.5° C., about 0.4° C. to about 0.6° C., about 0.4° C. to about 0.7° C., about 0.4° C. to about 0.8° C., about 0.4° C. to about 0.9° C., about 0.4° C. to about 1° C., about 0.5° C. to about 0.6° C., about 0.5° C. to about 0.7° C., about 0.5° C. to about 0.8° C., about 0.5° C. to about 0.9° C., about 0.5° C. to about 1° C., about 0.6° C. to about 0.7° C., about 0.6° C. to about 0.8° C., about 0.6° C. to about 0.9° C., about 0.6° C. to about 1° C., about 0.7° C. to about 0.8° C., about 0.7° C. to about 0.9° C., about 0.7° C. to about 1° C., about 0.8° C. to about 0.9° C., about 0.8° C. to about 1° C., or about 0.9° C. to about 1° C. In some embodiments, the temperature control stations may be configured to heat/cool the liquid samples by about 0.1° C., about 0.2° C., about 0.3° C., about 0.4° C., about 0.5° C., about 0.6° C., about 0.7° C., about 0.8° C., about 0.9° C., or about 1° C. In some embodiments, the temperature control stations may be configured to heat/cool the liquid samples by at least about 0.1° C., about 0.2° C., about 0.3° C., about 0.4° C., about 0.5° C., about 0.6° C., about 0.7° C., about 0.8° C., or about 0.9° C. In some embodiments, the temperature control stations may be configured to heat/cool the liquid samples by at most about 0.2° C., about 0.3° C., about 0.4° C., about 0.5° C., about 0.6° C., about 0.7° C., about 0.8° C., about 0.9° C., or about 1° C. In some embodiments, the temperature control stations may be configured to heat/cool the liquid samples by about 0.5° C. In some embodiments, the temperature control stations are configured to heat/cool to the liquid samples to maintain the temperature of the liquid samples within about 0.1° C. to about 1° C. of a target temperature.

Magnetic Bead Station

In some embodiments, a magnetic bead station may contain samples with nucleic acids, proteins, cells, buffers, magnetic beads, wash buffers, elution buffers, and other liquids on an electrode grid. The station may be configured to mix samples and reagents, apply heating or other processes, in sequential order to perform such actions as nucleic acid isolation, cell isolation, protein isolation, peptide purification, isolation or purification of biopolymers, immunoprecipitation, in vitro diagnostics, exosome isolation, cell activation, cell expansion, isolation, or any combination thereof of a specific biomolecule. In addition to mixing and heating of liquids, each magnetic bead station may have the ability to locally turn on and turn off a strong and varying magnetic field, which in turn may cause magnetic beads to move, for example, to the bottom of the electrowetting chip. Each magnetic bead station may also have the ability to remove excess supernatant liquids and wash liquids through electrowetting forces or through other forces.

In some cases, the sample may be on an open surface with single plate electrowetting device. In some cases, the samples may be sandwiched between two plates. Multiple magnetic bead stations may be configured to be operated in parallel, as described above for parallel mixing stations.

Some aspects of present disclosure provide that the droplet or reagent comprises one or more magnetic beads. In some embodiments, the first droplet or reagent comprises one or more magnetic beads. In some embodiments, the second droplet or reagent is a magnetic bead. In some embodiments, the third droplet or reagent comprises one or more magnetic beads. In some embodiments, the merged droplet or reagent comprises one or more magnetic beads.

In some embodiments, the magnetic bead stations are fixed locations on the array and/or substrate corresponding to fixed, permanent or electro-magnets. In some embodiments, the arrays and/or substrates described herein are operably coupled to a movable magnet. In embodiments wherein the array and/or substrate is coupled to a movable magnet, the magnetic bead station(s) described herein can be moved along the plane of the array and/or substrate.

Movable Magnet

The present disclosure provides a system for inducing motion in a droplet, comprising: (a) a surface configured to support said droplet comprising at least one bead formed of a material configured to couple to a magnetic field; (b) an actuator coupled a magnet, wherein said magnet is configured to supply said magnetic field, and wherein said actuator is configured to subject said magnetic field to translation along a plane parallel to said surface; and (c) a controller operatively coupled to said actuator, wherein said controller is configured to direct said actuator to subject said magnetic field to translation along said plane, such that while said magnetic field translates along said plane, said droplet undergoes motion along said surface. In some embodiments, said actuator is a switch. In some embodiments, said actuator comprises a motor coupled to said magnet, wherein said motor is configured to translate said magnet along a direction parallel to said surface. In some embodiments, the system further comprises an electrode configured to supply an electric field to said surface, wherein said electric field and said magnetic field are sufficient to subject said droplet to said motion. In some embodiments, said actuator is configured to motion said magnet to translate along at least two axes parallel to said plane. In some embodiments, said magnetic comprises a permanent magnet. In some embodiments, said magnet comprises at least one electromagnet. In some embodiments, said actuator comprises a pivot, wherein said pivot is coupled to said surface. In some embodiments, said surface comprises a dielectric disposed over one or more electrodes. In some embodiments, said one or more magnets are disposed below said surface. In some embodiments, said surface comprises a liquid layer. In some embodiments, said liquid layer comprises a liquid comprising an affinity for said surface.

An example of a droplet operation using a magnetic field is provided in FIG. 37. A bead formed of a material configured to couple to a magnetic field is provided in a droplet on a surface (FIG. 37A). A magnetic field is supplied to the surface, and an actuator translates the magnetic field along a plane parallel to the surface and the droplet, thereby moving the bead outside of the droplet (FIG. 37B). The droplet operation can be implemented in any system of the present disclosure that has a magnetic field.

EWOD-Enabled Magnetic Bead Wash

Magnetic particles may be manipulated on the surface of the chip by a controllable, localized magnetic field. The magnetic particles may be made of, for example, microspheres. Controlling the localized magnetic field may be achieved by, for example, placing a solenoid, a magnet, a pair of magnets, or any combination thereof in the vicinity of the particles or by generating a magnetic field within the EWOD chip. Magnetic bead-based separations and washes may be performed on an EWOD-enabled array. The droplet may be manipulated using the actuating electrodes which may also allow positioning of the droplet. The magnetic particles may be concentrated in a small region using the magnetic field. Liquids may be separated from the magnetic particles by EWOD-based, dielectrophoresis-based, or other electromotive force based actuation. Separation is possible in the open-plate and two-plate systems. Since the droplet can be positioned using EWOD actuation, the fluid may also be aspirated from the chip using a liquid handling robot, leaving the magnetic particles on the chip surface. Removal of liquid may be achieved through a hole, or a plurality thereof, in the array by employing capillary forces, pneumatic forces, electromotive forces, such as EWOD or dielectrowetting, or any combination thereof. This waste fluid may be collected in a reservoir positioned under the array. A computer-vision-based algorithm may be used to inform and provide feedback to the liquid handler and/or array for the processes involving magnetic beads. The processes may include, for example, aspiration of the supernatant, resuspension of beads, preventing aspiration of magnetic beads along with the supernatant during removal of supernatant, or any combination thereof.

Nucleic Acid Delivery Station

In some embodiments, an electrowetting chip may include one or more nucleic acid delivery stations. Each nucleic acid delivery station may be designed to insert genetic material, other nucleic acids and biologics into cells through various insertion methods. This insertion may be performed by applying a strong electric field, applying a strong magnetic field, applying ultrasonic waves, applying laser beams, or other techniques. One or more nucleic acid delivery station may be configured as a singleton on an electrowetting device, or multiple nucleic acid delivery stations may be provided to operate in parallel.

Optical Inspection Station

In some embodiments, one or more optical inspection stations that use optical detection and assay methods may be provided on an electrowetting device. A light source (e.g., broad spectrum light, single frequency, etc.) may be passed through optics to condition the light (which may include, for example, filters, diffraction gratings, mirrors, etc.) and illuminate a sample sitting on an electrowetting device. An optical detector, which may be placed on the same or other side of the electrowetting device, may be configured to detect the spectrum of light passing through the sample for analysis. The optical inspection may be used for measuring, for example, concentration of nucleic acids, measuring quality of nucleic acids, measuring density of cells, measuring extent of mixing between two liquids, measuring volume of sample, measuring fluorescence of sample, measuring absorbance of sample, quantification of proteins, colorimetric assays, optical assays, or any combination thereof.

In some embodiments, a sample may be on an open surface with single plate electrowetting device. In some embodiments, the sample may be sandwiched between two plates. In some embodiments, the electrowetting chip and the electrodes may be transparent. In some embodiments, there may be a hole in the electrode on which the sample is located, to allow passing of light from the source through the sample to the optical detector, or to introduce samples, reagents, or reactants.

In some embodiments, the optical detection may be performed on samples arranged in 2×2 sample format or 96 well plate format for optical detection or any M×N format to measure, for example, a million samples. The samples and corresponding measurement units may be arranged in any regular and irregular format.

Liquid Handling Station

In some embodiments, an electrowetting device may include one or more stations for loading biological samples, chemical reagents and liquids from a source well, plate, or reservoir onto an electrowetting chip.

In some embodiments, droplets may be loaded onto the electrowetting surface through acoustic droplet ejection. The source plate may hold liquids in wells and may be coupled with a piezoelectric transducer via an acoustic coupling fluid. Acoustic energy from a piezoelectric acoustic transducer may be focused on to the sample in the well. In some embodiments, an electrowetting chip is on top, and is inverted. The droplet may adhere to electrowetting chip because of the additional wetting force induced by the voltage, which contributes to the droplet-sorting function of apparatus. A droplet ejected from a well by acoustic energy may adhere to the upper electrowetting device or may be incorporated into a droplet that has been moved to the acoustic injection station.

In some embodiments, an electrowetting device may include one or more stations designed to load biological samples, chemical reagents and liquids through a microdiaphragm pump based dispenser onto an electrowetting chip.

Either the acoustic droplet ejection technique or a microdiaphragm pump may be used to dispense fluid droplets of picoliter, nanoliter, or microliter volumes. In some embodiments an electrowetting device placed above the source plate captures the droplets ejected from the well plate and holds the droplets through electrowetting force. In this manner, samples containing, for example, biological reagents, chemical reagents, or a combination thereof may be dispensed onto an electrowetting chip. In some embodiments, the electrowetting plate is on the bottom and the acoustic droplet ejection transducer or microdiaphragm pump is on the top. An input valve and larger microdiaphragm pump may be used to meter fluid flow into microdiaphragm pumps. In this method the dispenser may be used to put samples on to an electrowetting chip on any arbitrary location.

In some cases, the electrowetting chip may be in an open plate configuration (no second plate) and droplets may be loaded directly onto the chip. In some cases, the electrowetting chip may have a second plate that sandwiches the droplet between an electrode array and a ground electrode. In some cases, the second plate (cover plate with or without ground) may have holes to allow the droplets in transit. In some cases, the droplets may be first loaded on an open plate and then a second plate may be added. In some cases, the liquids loaded onto the electrowetting chip is in preparation to execute a workflow when the chip is located inside of an acoustic liquid handler. In some cases, the liquids loaded onto the electrowetting chip is in preparation to execute a workflow when the chip is located external to the acoustic liquid handler or microdiaphragm pump. In some cases, the liquids are loaded onto the electrowetting chip when a workflow is being executed. In some cases, the acoustic droplet injector or microdiaphragm pump may be mounted on a locatable carriage (somewhat like a 3D printer nozzle) capable of motion over the electrowetting device, so that droplets may be injected at a specific point over the electrowetting device.

In some cases, both the source and destination may be electrowetting chips. In this scenario, the chips may be organized with their electrode arrays facing each other. In some cases, droplets may be transferred between the top and bottom electrowetting chips, back and forth between top using acoustic fields or electric fields and differential wetting affinities. Here, there may be acoustic transducers and coupling fluids on both sides of the chips. In some cases, samples on an electrowetting chip may be a source and the destination may be a well plate. Here samples may be transferred from the electrowetting chip on to a well plate using acoustic droplet ejection.

The spacing between the wells in a well plate and hence the format in which the liquids are loaded on to (and transferred away from) the electrowetting chip may be in standard well plate form or any other SDS well plate format or any arbitrary formats. The number of wells in the plate may be any arbitrary number in the range from one to a million.

The electrowetting chips loaded with samples from an acoustic droplet ejection device or microdiaphragm pump device may be combined with one or more of the functionalities of mixing station, incubation station, magnetic bead station, nucleic acid delivery station, optical inspection station, other functionalities, or any combination thereof.

### Dried/Lyophilized Reagents On-Chin

Chemical reagents, biological reagents, or a combination thereof may be lyophilized/dried/spotted on the surface of the array. The reagents may be spotted on the surface of a disposable cartridge that is compatible with the array. The reagent may include, but are not limited to, buffers, salts, surfactants, nucleic acids, proteins, stabilizing agents, microbeads, enzymes, antibiotics or any combination thereof. The reagents may be solubilized or resuspended in the appropriate solution by liquid handling systems, EWOD actuation, manual pipetting, or any combination thereof. Kits, in part or in whole, for myriad molecular biology workflows/processes, may be produced using dried reagents. The kit may comprise refrigerated conditions for storage. Molecular biology processes may include, but are not limited to, preparation of nucleic acid libraries for next generation sequencing and microbial analysis workflows, (e.g., antibiotic-resistant strain detection).

Vibration-Assisted Mixing

Liquid droplets can be mixed in a variety of methods. The present disclosure provides methods by which vibration of a digital microfluidic surface can be used to assist in the mixing of liquids on the surface of the digital microfluidic device. The vibration may produce small-scale fluidic motion within a droplet on the surface of the digital microfluidic device. The motion may encourage diffusion and rapidly speed up the mixing process. An example of the benefits of vibration-assisted droplet mixing is efficient capture of the DNA onto the magnetic microparticles (e.g. beads) and ultimately higher yield DNA extraction. In some embodiments, an electrowetting array comprising an open surface is provided.

A common problem with digital microfluidics platforms is achieving robust mixing with all varieties of reagents and droplets. Highly viscous liquid droplets, for example, can be extremely difficult to mix effectively using a purely electrowetting based motion. These kinds of viscous droplets are important in a wide range of applications including DNA extraction from highly concentrated sample material where DNA needs to be efficiently bound to magnetic beads. Using purely electrowetting based motion to mix in these applications results in very poor mixing and therefore very poor DNA extraction from the sample droplet.

Implementation of devices, systems, and methods by which vibration and/or application of acoustic forces to the digital microfluidic surface can be used to assist in the mixing of liquids on the surface is described herein. The vibration, when tuned to the appropriate frequency and amplitude, produces small-scale fluidic motion within the droplet that encourages diffusion and rapidly speeds up the mixing process. FIG. 72 illustrates one such application of this technique for the extraction of DNA using magnetic beads. In FIG. 72, the viscosity of the DNA sample prohibits efficient mixing unless vibration is used. The result is efficient capture of the DNA onto the magnetic microparticles and ultimately to a higher yield DNA extraction. FIG. 72A shows suspended beads before vibration-assisted mixing. While FIG. 72B shows a pellet of beads and DNA has formed after vibration-assisted mixing.

Vibration also contributes to enhanced mobility of droplets. This is especially true for droplets that contain particulates. Without vibration, large particles can tend to settle at the interface between the droplet and the substrate. When these particles are present at the droplet's contact line they can act to pin the droplet in place, restricting its mobility. The introduction of vibration can help keep particles from settling at the contact line and, in doing so, greatly improves the reliability of electrowetting mobility of particulate-carrying droplets.

Vibration based mixing is synergistic with electrowetting based mixing. While vibration mixing is effective at dispersing particles within portions of a liquid droplet, it is often less effective at macro-scale mixing across the entire droplet, especially for droplets with low contact angle with the surface. Electrowetting-based droplet mixing helps address this problem and with both vibration and electrowetting acting together, mixing of a wide variety of droplets of various compositions can be accomplished rapidly and effectively.

The vibration frequency and amplitude need to be tuned to the droplet and the system dynamics. The resonant dynamics of the droplet depends on a number of factors including the volume, surface tension, and viscosity of the droplet. Droplets with higher contact angles tend to exhibit a greater response to the vibration while droplets that spread more readily on the surface (and have a lower contact angle) require greater amplitude to achieve comparable mixing (FIG. 73). FIG. 73 shows that high contact angle droplets (FIG. 73A) tend to experience greater response to vibration than droplets with lower contact angle (FIG. 73B). In order to achieve sufficient mixing of droplets using vibration the entire digital microfluidics device or parts of it can be displaced anywhere from a few micrometers to few millimeters. A displacement between 0.1 mm and 10 mm is a good range for this purpose. In the vibration assisted mixing schemes described above typical frequencies of vibrations range from 1 hz to 20 khz.

Beyond vibration, in some embodiments, other methods to mix difficult-to-mix droplets may be used. If assistance is needed with resuspending magnetic beads, an alternating magnetic field may be used to resuspend and mix magnetic beads within a droplet. This may be accomplished with the use of rotating permanent magnets or with electromagnetic coils oriented in multiple axes around the droplet. Using the electrode grid itself, it is possible to mix the droplet by exciting a resonance using an alternating current circuit that oscillates the voltage on electrodes beneath the droplet. This actuation may benefit from having a low impedance path to the droplet itself in order to increase the magnitude of the response.

The present disclosure provides methods of electrowetting-based droplet mixing. In some embodiments, electrowetting-based droplet mixing comprises both vibration and electrowetting. In some embodiments, vibration and electrowetting act together and mix a wide variety of droplets. In some embodiments, vibration and electrowetting of various compositions may be accomplished rapidly and effectively. The frequency for electrowetting-based droplet mixing may be modulated. The amplitude for electrowetting-based droplet mixing may be modulated.

The vibration of the digital microfluidic surface may be accomplished through a number of means. In one embodiment, the surface itself may be used as a spring element whereby one end of the surface is fixed while the other is attached to an electro-mechanical actuator. In some embodiments, the electro-mechanical actuator is an oscillating mechanism or cantilever (FIG. 74). The embodiment depicted in FIG. 74A produces a gradient in the vibration energy across the length of the surface. Droplets positioned closer to the vibrating edge of the cantilever will experience much greater amplitude than those closer to the fixed end. For example, electromagnetic actuators, voice coil actuators, piezoelectric actuators, ultrasonic transducers, rotating eccentric masses, motor driven linkage, and brushed/brushless/stepper motors with oscillating linkage mechanisms may be used.

In another embodiment, the whole surface is translated vertically (FIG. 74B). This may be accomplished with an electro-mechanical actuator comprising various flexible elements (e.g. a linear flexure) or with traditional bearings. The embodiment depicted in FIG. 74B produces uniform vibration amplitude across the entire surface (assuming a sufficiently rigid substrate is used).

The vibration mechanisms of constraining the motion of the surface described herein may be actuated through a number of different methods including electromagnetic actuators, piezoelectric actuators, ultrasonic transducers, rotating eccentric masses, as well as brushed/brushless/stepper motors with oscillating linkage mechanisms (FIG. 75A).

Electromagnetic voice coil actuators can be actuated at a wide range of frequencies and amplitudes and these can be controlled independently. Additionally, a variety of waveforms can be used to excite the actuator to achieve different effects. A sine wave can be used, for example, to produce quiet oscillation while a square wave may be used to excite the surface much more aggressively.

The embodiments depicted in FIG. 75 result in a dynamic system that must be characterized and understood in order to efficiently couple the actuation force into droplet vibration and ultimately to effective mixing (FIG. 76). The resulting dynamic system may be modified or augmented through the use of external elements such as passive springs, dampers, or masses. These elements can be used, for example, to shift the resonant frequency of the system to one that is aligned with a resonant frequency of the droplets on the surface. This can be done passively or actively (with actuated spring elements). In some embodiments a disposable widget may be attached to the system between the surface and the system to modify the system for greater stability and reliable performance. This widget may be a sponge clip and may facilitate the vibration of the surface within the system.

In some embodiments the system may be leveled by the user through the use of a digital leveling interface. This digital leveling may increase the functionality of the vibrational mixing and decrease the occurrence of vibrational induced droplet splitting. This leveling interface may instruct the user on how to properly level the system and may ensure that it has been properly leveled through a leveling module configured to detect an angle of the surface.

In some embodiments, a voice coil actuators may be used to generate vibration. The voice coil actuator may comprise a permanent magnetic field assembly and a coil assembly. The voice coil actuator may be actuated at a wide range of frequencies and amplitudes. The voice coil actuator may be excited via a variety of waveforms. A sine wave can be used, for example, to produce quiet oscillation while a square wave may be used to excite the surface much more aggressively.

In some embodiments, a motor driven linkage may be used to generate vibration (FIG. 75B). A conventional brushless, brushed, or stepper motor may used to turn a shaft. The shaft may be connected to a rigid-body or flexural linkage, which when turned, results in oscillatory motion of the output. The motor driven linkage may be augmented with, for example, passive spring, mass, and damping elements, to improve efficiency and allow for larger amplitude oscillations with less input power. For example, spring elements may be placed between the output of the linkage and the oscillating substrate. This embodiment is much less dependent on the system dynamics of the surface and motion constraint mechanisms but, as a result, may require more power input in order to achieve equivalent vibration output to a well-tuned dynamic system.

In some embodiments, a rotating eccentric mass may be used to generate vibration. The rotating eccentric mass may be off-center from the point of rotation. A motor may be mounted to an oscillating substrate (either directly or through coupling springs). The motor may spin an offset mass to create an oscillating acceleration. The operation of the rotating eccentric mass may cause an uneven centripetal force, which may in turn cause the motor to move backwards and forwards. The acceleration amplitude and frequency may be directly linked.

While the resonant frequency of the system may be excited at or close to the resonant peak in order to achieve best efficiency between input and output power. It may also be advantageous in some embodiments to excite the system far from the resonant frequency. This may be beneficial, for example, if it is desirable to excite the droplet with a roughly equivalent amplitude across a wide range of frequencies. This particular case can be readily achieved by tuning the system's natural frequency to be low relative to the desired frequency range and results in a near constant acceleration amplitude across a broad frequency range (FIG. 77).

In another embodiment, a closed loop control can be utilized for fine control of amplitude independently from frequency. This may be accomplished, for example, with the use of an accelerometer mounted to the vibrating platform. A microcontroller may communicate with the sensor and calculate the acceleration amplitude in real time. Given some desired acceleration amplitude, the microcontroller can adjust the amplifier gain and modulate the drive waveform of the vibration actuator in order to precisely control the vibration amplitude.

In addition to mechanically induced vibrations, in some embodiments, droplets can also be vibrated purely with the use of electrowetting or other non-contact forces. In some embodiments, other non-contact forces that may be used for vibration-assisted-mixing include acoustic or ultrasonic waves.

In some embodiments, electrowetting vibration-assisted mixing can be accomplished by switching the amplitude or polarity of the electric field at a certain frequency. For optimal vibration-assisted-mixing this frequency should be close to the resonant frequency (or a multiple of the resonant frequency) of the droplet. For droplets between 10 uL and 200 uL this frequency is often in the range of 10 Hz to 100 Hz. In some embodiments, only the polarity of the field is switched by alternately charging and discharging the electrodes beneath or adjacent to the droplet while surrounding electrodes are driven with the opposite polarity signal.

Some aspects of the present disclosure provide a method of generating a biopolymer, comprising: (a) providing a plurality of droplets adjacent to a surface, wherein said plurality of droplets comprises a first droplet comprising a first reagent and a second droplet comprising a second reagent; (b) subjecting said first droplet and said second droplet to motion relative to one another to (i) bring said first droplet in contact with said second droplet and (ii) form a merged droplet comprising said first reagent and said second reagent; and in said merged droplet, (c) using at least (i) said first reagent and (ii) said second reagent to form at least a portion of said biopolymer, wherein (b)-(c) are performed in a time period of 10 minutes or less. In some embodiments, a vibration is applied to said synthesis droplet during (b), (c), or both.

An aspect of the present disclosure comprises a system for processing a sample, comprising: an array comprising: a plurality of electrodes; and a surface configured to support the sample; an electro-mechanical actuator coupled to the array, wherein the actuator is configured to vibrate the array; and a controller operatively coupled to the plurality of electrodes, or the electro-mechanical actuator, wherein the controller is configured to: direct at least a subset of the plurality of electrodes to supply an electric field to alter a wetting characteristic of the surface; or direct the electro-mechanical actuator to apply a frequency of vibration to the array. In some embodiments, the controller is configured to perform (i) and (ii). In some embodiments, the controller is coupled to the plurality of electrodes and the electro-mechanical actuator. In some embodiments, the sample is a droplet. In some embodiments, the droplet comprises about 1 nanoliter to 1 milliliter. In some embodiments, the droplet comprises a biological material. In some embodiments, the biological sample comprises one or more bio-molecules. In some embodiments, the bio-molecules comprise nucleic acid molecules, proteins, polypeptides, or any combination thereof. In some embodiments, the electro-mechanical actuator comprises a cantilever. In some embodiments, the electro-mechanical actuator comprises one or more coupling members coupled to the array. In some embodiments, the one or more coupling members comprise electromagnetic actuators, piezoelectric actuators, ultrasonic transducers, rotating eccentric masses, one or more motors with oscillating linkage mechanisms, or any combination thereof. In some embodiments, the one or more motors are brushed, brushless, stepper, or any combination thereof. In some embodiments, the electromagnetic actuators comprise electromagnetic voice coil actuators. In some embodiments, the frequency of vibration comprises a gradient. In some embodiments, the gradient ascends from near a site wherein the cantilever is coupled to the array. In some embodiments, the vibration comprises a pattern. In some embodiments, the pattern is sinusoidal. In some embodiments, the pattern is square. In some embodiments, the surface is a top surface of a dielectric wherein the dielectric is disposed over the plurality of electrodes. In some embodiments, the top surface comprises a layer. In some embodiments, the layer comprises a liquid. In some embodiments, the layer comprises a coating. In some embodiments, the coating is hydrophobic. In some embodiments, the layer comprises a film. In some embodiments, the film is a dielectric film. In some embodiments, the dielectric film comprises a natural polymeric material, a synthetic polymeric material, a fluorinated material, a surface modification, or any combination thereof. In some embodiments, the natural polymeric material comprises shellac, amber, wool, silk, natural rubber, cellulose, wax, chiton, or any combination thereof. In some embodiments, the synthetic polymeric material comprises polyethylene, polypropylene, polystyrene, polyetheretherketone (PEEK), polyimide, polyacetal, polysilfone, polyphenulene ether, polyphenylene Sulfide (PPS), polyvinyl chloride, synthetic rubber, neoprene, nylon, polyacrylonitrile, polyvinyl butyral, silicone, parafilm, polyethylene terephthalate, polybutylene terephthalate, polyamides, polyoxymethlyene, polycarbonate, polymethylpentene, polyphenylene oxide (Polyphenyl ether), polyphthalamide (PPA), polylactic acid, synthetic cellulose ethers (e.g., methyl cellulose, ethyl cellulose, propyl cellulose, hydroxyethyl cellulose, hydroxyethyl cellulose, hydroxypropyl cellulose (HPC), hydroxyethyl methyl cellulose, hydroxypropyl methyl cellulose (HPMC), ethyl hydroxyethyl cellulose), paraffins, microcrystalline wax, epoxy, or any combination thereof. In some embodiments, the fluorinated material comprises polytetrafluoroethylene (PTFE), tetrafluoroethylene (TFE), fluorinated ethylenepropylene copolymer (FEP), polyvinylidene fluoride (PVDF), perfluoroalkoxytetrafluoroethylene copolymer (PFA), perfluoromethyl vinylether copolymer (MFA), ethylenechlorotrifluoroethylene copolymer (ECTFE), ethylene-tetrafluoroethylene copolymer (ETFE), perfluoropolyether (PFPE), polychlorotetrafluoroethylene (PCTFE), or any combination thereof. In some embodiments, the surface modification comprises silicone, silane, fluoro-polymer treatment, parylene coating, any other suitable surface chemistry modification process, ceramic, clay minerals, bentonite, kaolinite, vermiculite, graphite, molybdenum disulfide, mica, boron nitride, sodium formate, sodium oleate, sodium palmitate, sodium sulfate, sodium alginate, or any combination thereof. In some embodiments, the liquid comprises silicone oils, fluorinated oils, ionic liquids, mineral oils, ferrofluids, polyphenyl ether, vegetable oil, esters of saturated fatty and dibasic acids, grease, fatty acids, triglycerides, polyalphaolefin, polyglycol hydrocarbons, other Non-hydrocarbon synthetic oils, or any combination thereof. In some embodiments, the liquid further comprises surfactants, electrolytes, rheology modifier, wax, graphite, graphene, molybdenum disulfide, PTFE particles, or any combination thereof. In some embodiments, the first plurality of electrodes, the dielectric, the surface configured to support the droplet comprising the sample, or any combination thereof is removable from the array.

In some embodiments, the electro-mechanical actuator is configured to displace the surface or a portion of the surface from 0.05 millimeters (mm) to 10 mm. In some embodiments, the surface or the portion of the surface, is displaced from about 0.05 mm to about 10 mm. In some embodiments, the surface or the portion of the surface, is displaced from about 0.05 mm to about 0.1 mm, about 0.05 mm to about 0.5 mm, about 0.05 mm to about 1 mm, about 0.05 mm to about 2 mm, about 0.05 mm to about 3 mm, about 0.05 mm to about 4 mm, about 0.05 mm to about 5 mm, about 0.05 mm to about 6 mm, about 0.05 mm to about 7 mm, about 0.05 mm to about 8 mm, about 0.05 mm to about 9 mm, about 0.05 mm to about 10 mm about 0.1 mm to about 0.5 mm, about 0.1 mm to about 1 mm, about 0.1 mm to about 2 mm, about 0.1 mm to about 3 mm, about 0.1 mm to about 4 mm, about 0.1 mm to about 5 mm, about 0.1 mm to about 6 mm, about 0.1 mm to about 7 mm, about 0.1 mm to about 8 mm, about 0.1 mm to about 9 mm, about 0.1 mm to about 10 mm, about 0.5 mm to about 1 mm, about 0.5 mm to about 2 mm, about 0.5 mm to about 3 mm, about 0.5 mm to about 4 mm, about 0.5 mm to about 5 mm, about 0.5 mm to about 6 mm, about 0.5 mm to about 7 mm, about 0.5 mm to about 8 mm, about 0.5 mm to about 9 mm, about 0.5 mm to about 10 mm, about 1 mm to about 2 mm, about 1 mm to about 3 mm, about 1 mm to about 4 mm, about 1 mm to about 5 mm, about 1 mm to about 6 mm, about 1 mm to about 7 mm, about 1 mm to about 8 mm, about 1 mm to about 9 mm, about 1 mm to about 10 mm, about 2 mm to about 3 mm, about 2 mm to about 4 mm, about 2 mm to about 5 mm, about 2 mm to about 6 mm, about 2 mm to about 7 mm, about 2 mm to about 8 mm, about 2 mm to about 9 mm, about 2 mm to about 10 mm, about 3 mm to about 4 mm, about 3 mm to about 5 mm, about 3 mm to about 6 mm, about 3 mm to about 7 mm, about 3 mm to about 8 mm, about 3 mm to about 9 mm, about 3 mm to about 10 mm, about 4 mm to about 5 mm, about 4 mm to about 6 mm, about 4 mm to about 7 mm, about 4 mm to about 8 mm, about 4 mm to about 9 mm, about 4 mm to about 10 mm, about 5 mm to about 6 mm, about 5 mm to about 7 mm, about 5 mm to about 8 mm, about 5 mm to about 9 mm, about 5 mm to about 10 mm, about 6 mm to about 7 mm, about 6 mm to about 8 mm, about 6 mm to about 9 mm, about 7 mm to about 8 mm, about 7 mm to about 9 mm, about 7 mm to about 10 mm, about 8 mm to about 9 mm, or about 9 mm to about 10 mm. In some embodiments, the surface or the portion of the surface, is displaced from about 0.05 mm, about 0.1 mm, about 0.5 mm, about 1 mm, about 2 mm, about 3 mm, about 4 mm, about 5 mm, about 6 mm, about 7 mm, about 8 mm, about 9 mm, or about 10 mm. In some embodiments, the surface or the portion of the surface, is displaced from at least about 0.05 mm, about 0.1 mm, about 0.5 mm, about 1 mm, about 2 mm, about 3 mm, about 4 mm, about 5 mm, about 6 mm, about 7 mm, or about 8 mm. In some embodiments, the surface or the portion of the surface, is displaced from at most about 0.1 mm, about 0.5 mm, about 1 mm, about 2 mm, about 3 mm, about 4 mm, about 5 mm, about 6 mm, about 7 mm, about 8 mm, about 9 mm, or about 10 mm.

In some embodiments, the frequency of the vibration is from 1 Hertz (hz) to 20 kilohertz (khz). In some embodiments, the frequency of the vibration is from about 1 Hz to about 10 Hz. In some embodiments, the frequency of the vibration is from about 1 Hz to about 2 Hz, about 1 Hz to about 3 Hz, about 1 Hz to about 4 Hz, about 1 Hz to about 5 Hz, about 1 Hz to about 6 Hz, about 1 Hz to about 7 Hz, about 1 Hz to about 8 Hz, about 1 Hz to about 9 Hz, about 1 Hz to about 10 Hz, about 2 Hz to about 3 Hz, about 2 Hz to about 4 Hz, about 2 Hz to about 5 Hz, about 2 Hz to about 6 Hz, about 2 Hz to about 7 Hz, about 2 Hz to about 8 Hz, about 2 Hz to about 9 Hz, about 2 Hz to about 10 Hz, about 3 Hz to about 4 Hz, about 3 Hz to about 5 Hz, about 3 Hz to about 6 Hz, about 3 Hz to about 7 Hz, about 3 Hz to about 8 Hz, about 3 Hz to about 9 Hz, about 3 Hz to about 10 Hz, about 4 Hz to about 5 Hz, about 4 Hz to about 6 Hz, about 4 Hz to about 7 Hz, about 4 Hz to about 8 Hz, about 4 Hz to about 9 Hz, about 4 Hz to about 10 Hz, about 5 Hz to about 6 Hz, about 5 Hz to about 7 Hz, about 5 Hz to about 8 Hz, about 5 Hz to about 9 Hz, about 5 Hz to about 10 Hz, about 6 Hz to about 7 Hz, about 6 Hz to about 8 Hz, about 6 Hz to about 9 Hz, about 6 Hz to about 10 Hz, about 7 Hz to about 8 Hz, about 7 Hz to about 9 Hz, about 7 Hz to about 10 Hz, about 8 Hz to about 9 Hz, about 8 Hz to about 10 Hz, or about 9 Hz to about 10 Hz. In some embodiments, the frequency of the vibration is from about 1 Hz, about 2 Hz, about 3 Hz, about 4 Hz, about 5 Hz, about 6 Hz, about 7 Hz, about 8 Hz, about 9 Hz, or about 10 Hz. In some embodiments, the frequency of the vibration is from at least about 1 Hz, about 2 Hz, about 3 Hz, about 4 Hz, about 5 Hz, about 6 Hz, about 7 Hz, about 8 Hz, or about 9 Hz. In some embodiments, the frequency of the vibration is from at most about 2 Hz, about 3 Hz, about 4 Hz, about 5 Hz, about 6 Hz, about 7 Hz, about 8 Hz, about 9 Hz, or about 10 Hz. In some embodiments, the frequency of the vibration is from about 10 Hz to about 1,000 Hz. In some embodiments, the frequency of the vibration is from about 10 Hz to about 100 Hz, about 10 Hz to about 200 Hz, about 10 Hz to about 300 Hz, about 10 Hz to about 400 Hz, about 10 Hz to about 500 Hz, about 10 Hz to about 600 Hz, about 10 Hz to about 700 Hz, about 10 Hz to about 800 Hz, about 10 Hz to about 900 Hz, about 10 Hz to about 1,000 Hz, about 100 Hz to about 200 Hz, about 100 Hz to about 300 Hz, about 100 Hz to about 400 Hz, about 100 Hz to about 500 Hz, about 100 Hz to about 600 Hz, about 100 Hz to about 700 Hz, about 100 Hz to about 800 Hz, about 100 Hz to about 900 Hz, about 100 Hz to about 1,000 Hz, about 200 Hz to about 300 Hz, about 200 Hz to about 400 Hz, about 200 Hz to about 500 Hz, about 200 Hz to about 600 Hz, about 200 Hz to about 700 Hz, about 200 Hz to about 800 Hz, about 200 Hz to about 900 Hz, about 200 Hz to about 1,000 Hz, about 300 Hz to about 400 Hz, about 300 Hz to about 500 Hz, about 300 Hz to about 600 Hz, about 300 Hz to about 700 Hz, about 300 Hz to about 800 Hz, about 300 Hz to about 900 Hz, about 300 Hz to about 1,000 Hz, about 400 Hz to about 500 Hz, about 400 Hz to about 600 Hz, about 400 Hz to about 700 Hz, about 400 Hz to about 800 Hz, about 400 Hz to about 900 Hz, about 400 Hz to about 1,000 Hz, about 500 Hz to about 600 Hz, about 500 Hz to about 700 Hz, about 500 Hz to about 800 Hz, about 500 Hz to about 900 Hz, about 500 Hz to about 1,000 Hz, about 600 Hz to about 700 Hz, about 600 Hz to about 800 Hz, about 600 Hz to about 900 Hz, about 600 Hz to about 1,000 Hz, about 700 Hz to about 800 Hz, about 700 Hz to about 900 Hz, about 700 Hz to about 1,000 Hz, about 800 Hz to about 900 Hz, about 800 Hz to about 1,000 Hz, or about 900 Hz to about 1,000 Hz. In some embodiments, the frequency of the vibration is from about 10 Hz, about 100 Hz, about 200 Hz, about 300 Hz, about 400 Hz, about 500 Hz, about 600 Hz, about 700 Hz, about 800 Hz, about 900 Hz, or about 1,000 Hz. In some embodiments, the frequency of the vibration is from at least about 10 Hz, about 100 Hz, about 200 Hz, about 300 Hz, about 400 Hz, about 500 Hz, about 600 Hz, about 700 Hz, about 800 Hz, or about 900 Hz. In some embodiments, the frequency of the vibration is from at most about 100 Hz, about 200 Hz, about 300 Hz, about 400 Hz, about 500 Hz, about 600 Hz, about 700 Hz, about 800 Hz, about 900 Hz, or about 1,000 Hz. In some embodiments, the frequency of the vibration is from about 1 kHz to about 20 kHz. In some embodiments, the frequency of the vibration is from about 1 kHz to about 2.5 kHz, about 1 kHz to about 5 kHz, about 1 kHz to about 7.5 kHz, about 1 kHz to about 10 kHz, about 1 kHz to about 12.5 kHz, about 1 kHz to about 15 kHz, about 1 kHz to about 17.5 kHz, about 1 kHz to about 20 kHz, about 2.5 kHz to about 5 kHz, about 2.5 kHz to about 7.5 kHz, about 2.5 kHz to about 10 kHz, about 2.5 kHz to about 12.5 kHz, about 2.5 kHz to about 15 kHz, about 2.5 kHz to about 17.5 kHz, about 2.5 kHz to about 20 kHz, about 5 kHz to about 7.5 kHz, about 5 kHz to about 10 kHz, about 5 kHz to about 12.5 kHz, about 5 kHz to about 15 kHz, about 5 kHz to about 17.5 kHz, about 5 kHz to about 20 kHz, about 7.5 kHz to about 10 kHz, about 7.5 kHz to about 12.5 kHz, about 7.5 kHz to about 15 kHz, about 7.5 kHz to about 17.5 kHz, about 7.5 kHz to about 20 kHz, about 10 kHz to about 12.5 kHz, about 10 kHz to about 15 kHz, about 10 kHz to about 17.5 kHz, about 10 kHz to about 20 kHz, about 12.5 kHz to about 15 kHz, about 12.5 kHz to about 17.5 kHz, about 12.5 kHz to about 20 kHz, about 15 kHz to about 17.5 kHz, about 15 kHz to about 20 kHz, or about 17.5 kHz to about 20 kHz. In some embodiments, the frequency of the vibration is from about 1 kHz, about 2.5 kHz, about 5 kHz, about 7.5 kHz, about 10 kHz, about 12.5 kHz, about 15 kHz, about 17.5 kHz, or about 20 kHz. In some embodiments, the frequency of the vibration is from at least about 1 kHz, about 2.5 kHz, about 5 kHz, about 7.5 kHz, about 10 kHz, about 12.5 kHz, about 15 kHz, or about 17.5 kHz. In some embodiments, the frequency of the vibration is from at most about 2.5 kHz, about 5 kHz, about 7.5 kHz, about 10 kHz, about 12.5 kHz, about 15 kHz, about 17.5 kHz, or about 20 kHz.

Another aspect of the present disclosure comprises a method for processing a sample comprising: providing an array comprising: a plurality of electrodes; and a surface configured to support the sample; wherein the array is coupled to an electro-mechanical actuator and the electro-mechanical actuator is configured to vibrate the array; introducing the droplet to the surface; directing the electro-mechanical actuator to apply a frequency of vibration to the array. In some embodiments, the sample is a droplet. In some embodiments, the droplet comprises about 1 nanoliter to 1 milliliter. In some embodiments, the droplet comprises a biological material. In some embodiments, the biological sample comprises one or more bio-molecules. In some embodiments, the bio-molecules comprise nucleic acid molecules, proteins, polypeptides, or any combination thereof. In some embodiments, the droplet comprises about 1 nanoliter to 1 milliliter. In some embodiments, the method further comprises directing at least a subset of the plurality of electrodes to supply an electric field to alter a wetting characteristic of the surface. In some embodiments, the electro-mechanical actuator comprises a cantilever. In some embodiments, the electro-mechanical actuator comprises one or more coupling members coupled to the array. In some embodiments, the one or more coupling members comprise electromagnetic actuators, piezoelectric actuators, ultrasonic transducers, rotating eccentric masses, one or more motors with oscillating linkage mechanisms, or any combination thereof. In some embodiments, the one or more motors are brushed, brushless, stepper, or any combination thereof. In some embodiments, the electromagnetic actuators comprise electromagnetic voice coil actuators. In some embodiments, the frequency of vibration comprises a gradient. In some embodiments, the gradient ascends from near a site wherein the cantilever is coupled to the array. In some embodiments, the vibration comprises a pattern. In some embodiments, the pattern is sinusoidal. In some embodiments, the pattern is square. In some embodiments, the surface is a top surface of a dielectric wherein the dielectric is disposed over the plurality of electrodes. In some embodiments, the surface comprises a layer disposed over a dielectric wherein the dielectric is disposed over the plurality of electrodes. In some embodiments, the layer comprises a liquid. In some embodiments, the layer comprises a coating. In some embodiments, the coating is hydrophobic. In some embodiments, the layer comprises a film. In some embodiments, the film is a dielectric film. In some embodiments, the dielectric film comprises a natural polymeric material, a synthetic polymeric material, a fluorinated material, a surface modification, or any combination thereof. In some embodiments, the natural polymeric material comprises shellac, amber, wool, silk, natural rubber, cellulose, wax, chiton, or any combination thereof. In some embodiments, the synthetic polymeric material comprises polyethylene, polypropylene, polystyrene, polyetheretherketone (PEEK), polyimide, polyacetal, polysilfone, polyphenulene ether, polyphenylene Sulfide (PPS), polyvinyl chloride, synthetic rubber, neoprene, nylon, polyacrylonitrile, polyvinyl butyral, silicone, parafilm, polyethylene terephthalate, polybutylene terephthalate, polyamides, polyoxymethlyene, polycarbonate, polymethylpentene, polyphenylene oxide (Polyphenyl ether), polyphthalamide (PPA), polylactic acid, synthetic cellulose ethers (e.g., methyl cellulose, ethyl cellulose, propyl cellulose, hydroxyethyl cellulose, hydroxyethyl cellulose, hydroxypropyl cellulose (HPC), hydroxyethyl methyl cellulose, hydroxypropyl methyl cellulose (HPMC), ethyl hydroxyethyl cellulose), paraffins, microcrystalline wax, epoxy, or any combination thereof. In some embodiments, the fluorinated material comprises polytetrafluoroethylene (PTFE), tetrafluoroethylene (TFE), fluorinated ethylenepropylene copolymer (FEP), polyvinylidene fluoride (PVDF), perfluoroalkoxytetrafluoroethylene copolymer (PFA), perfluoromethyl vinylether copolymer (MFA), ethylenechlorotrifluoroethylene copolymer (ECTFE), ethylene-tetrafluoroethylene copolymer (ETFE), perfluoropolyether (PFPE), polychlorotetrafluoroethylene (PCTFE), or any combination thereof. In some embodiments, the surface modification comprises silicone, silane, fluoro-polymer treatment, parylene coating, any other suitable surface chemistry modification process, ceramic, clay minerals, bentonite, kaolinite, vermiculite, graphite, molybdenum disulfide, mica, boron nitride, sodium formate, sodium oleate, sodium palmitate, sodium sulfate, sodium alginate, or any combination thereof. In some embodiments, the liquid comprises silicone oils, fluorinated oils, ionic liquids, mineral oils, ferrofluids, polyphenyl ether, vegetable oil, esters of saturated fatty and dibasic acids, grease, fatty acids, triglycerides, polyalphaolefin, polyglycol hydrocarbons, other Non-hydrocarbon synthetic oils, or any combination thereof. In some embodiments, the liquid further comprises surfactants, electrolytes, rheology modifier, wax, graphite, graphene, molybdenum disulfide, PTFE particles, or any combination thereof. In some embodiments, the first plurality of electrodes, the dielectric, the surface configured to support the droplet comprising the sample, or any combination thereof is removable from the array. In some embodiments, the frequency of the vibration displaces the surface or a portion of the surface from 0.05 millimeters (mm) to 10 mm. In some embodiments, the frequency of the vibration is from 1 Hertz (hz) to 20 kilohertz (khz).

An additional aspect of the present disclosure comprises a method of contacting a first sample with a second sample, wherein the first sample is contained in a first droplet and the second sample is contained in a second droplet, the method comprising: providing an array comprising: a plurality of electrodes; and a surface configured to support the first droplet and the second droplet; wherein the array is coupled to an electro-mechanical actuator and the electro-mechanical actuator is configured to vibrate the array; introducing the first droplet and the second droplet to the surface; directing at least a subset of the plurality of electrodes to supply an electric field to alter a wetting characteristic of the surface thereby inducing a motion in the first droplet and the second droplet wherein the motion of the first droplet and the second droplet comprise the first droplet and the second droplet to converge to generate a mixed droplet; and directing the electro-mechanical actuator to apply a frequency of vibration to the surface; thereby contacting the first sample with the second sample. In some embodiments, the first sample, the second sample, or both comprise a viscous fluid. In some embodiments, the first sample, the second sample, or both comprise a biological sample. In some embodiments, there is a third droplet comprising a third reagent. In some embodiments, the biological sample comprises one or more bio-molecules. In some embodiments, the bio-molecules comprise nucleic acid molecules, proteins, polypeptides, or any combination thereof. In some embodiments, the first sample, the second sample, or both comprise reagents for a biological assay. In some embodiments, the first sample, the second sample, or both comprise one or more cell lysis reagents. In some embodiments, the one or more cell lysis reagents comprise a substrate configured to bind to the biological sample or a subset of the biological sample. In some embodiments, the nucleic acid molecule comprises more than 10 kilobases (kb), 20 kb, 30 kb, 40 kb, or 50 kb. In some embodiments, greater than 10%, 20%, 30%, 40%, 50%, 60%, 70%, 80%, or 90% of the biological sample binds to the substrate. In some embodiments, the substrate is a functionalized bead. In some embodiments, the first reagent comprises one or more functionalized beads. In some embodiments, the second reagent comprises one or more functionalized beads. In some embodiments, the third reagent comprises one or more functionalized beads. In some embodiments, a combination of the first, second, and/or third reagent comprises one or more functionalized beads. In some embodiments, the functionalized beads comprise one or more oligonucleotides immobilized thereto. In some embodiments, the first reagent comprises a polymerase. In some embodiments, the second reagent comprises a polymerase. In some embodiments, the third reagent comprises a polymerase. In some embodiments, a combination of the first, second, and/or third reagent comprises a polymerase. In some embodiments, the first reagent comprises a bio-monomer. In some embodiments, the second reagent comprises a bio-monomer. In some embodiments, the third reagent comprises a bio-monomer. In some embodiments, a combination of the first, second, and/or third reagent comprises a bio-monomer. In some embodiments, the bio-monomer is an amino acid. In some embodiments, the bio-monomer is a nucleic acid molecule. In some embodiments, the nucleic acid molecule comprises of adenine, cytosine, guanine, thymine, or uracil. In some embodiments, the substrate is a functionalized disc. In some embodiments, the first reagent comprises one or more functionalized discs. In some embodiments, the second reagent comprises one or more functionalized discs. In some embodiments, the third reagent comprises one or more functionalized discs. In some embodiments, a combination of the first, second, and/or third reagent comprises one or more functionalized discs. In some embodiments, the functionalized disc comprises one or more oligonucleotides immobilized thereto. In some embodiments, the method further comprises, subsequent to (d): removing at least a portion of the mixed droplet by directing at least a subset of the plurality of electrodes to supply an electric field to alter a wetting characteristic of the surface thereby inducing a motion in the at least the portion of the mixed droplet. In some embodiments, the at least the portion of the mixed droplet does not comprise the biological sample. In some embodiments, the method further comprises, prior to or contemporaneously with (e) applying a magnetic field to the surface. In some embodiments, the magnetic field immobilizes the substrate. In some embodiments, the electro-mechanical actuator comprises a cantilever. In some embodiments, the electro-mechanical actuator comprises one or more coupling members coupled to the array. In some embodiments, the one or more coupling members comprise electromagnetic actuators, piezoelectric actuators, ultrasonic transducers, rotating eccentric masses, one or more motors with oscillating linkage mechanisms, or any combination thereof. In some embodiments, the one or more motors are brushed, brushless, stepper, or any combination thereof. In some embodiments, the electromagnetic actuators comprise electromagnetic voice coil actuators. In some embodiments, the frequency of vibration comprises a gradient. In some embodiments, the gradient ascends from near a site wherein the cantilever is coupled to the array. In some embodiments, the vibration comprises a pattern. In some embodiments, the pattern is sinusoidal. In some embodiments, the pattern is square. In some embodiments, the surface is a top surface of a dielectric wherein the dielectric is disposed over the plurality of electrodes. In some embodiments, the surface comprises a layer disposed over a dielectric wherein the dielectric is disposed over the plurality of electrodes. In some embodiments, the layer comprises a liquid. In some embodiments, the layer comprises a coating. In some embodiments, the coating is hydrophobic. In some embodiments, the layer comprises a film. In some embodiments, the film is a dielectric film. In some embodiments, the dielectric film comprises a natural polymeric material, a synthetic polymeric material, a fluorinated material, a surface modification, or any combination thereof. In some embodiments, the natural polymeric material comprises shellac, amber, wool, silk, natural rubber, cellulose, wax, chiton, or any combination thereof. In some embodiments, the synthetic polymeric material comprises polyethylene, polypropylene, polystyrene, polyetheretherketone (PEEK), polyimide, polyacetal, polysilfone, polyphenulene ether, polyphenylene Sulfide (PPS), polyvinyl chloride, synthetic rubber, neoprene, nylon, polyacrylonitrile, polyvinyl butyral, silicone, parafilm, polyethylene terephthalate, polybutylene terephthalate, polyamides, polyoxymethlyene, polycarbonate, polymethylpentene, polyphenylene oxide (Polyphenyl ether), polyphthalamide (PPA), polylactic acid, synthetic cellulose ethers (e.g., methyl cellulose, ethyl cellulose, propyl cellulose, hydroxyethyl cellulose, hydroxyethyl cellulose, hydroxypropyl cellulose (HPC), hydroxyethyl methyl cellulose, hydroxypropyl methyl cellulose (HPMC), ethyl hydroxyethyl cellulose), paraffins, microcrystalline wax, epoxy, or any combination thereof. In some embodiments, the fluorinated material comprises polytetrafluoroethylene (PTFE), tetrafluoroethylene (TFE), fluorinated ethylenepropylene copolymer (FEP), polyvinylidene fluoride (PVDF), perfluoroalkoxytetrafluoroethylene copolymer (PFA), perfluoromethyl vinylether copolymer (MFA), ethylenechlorotrifluoroethylene copolymer (ECTFE), ethylene-tetrafluoroethylene copolymer (ETFE), perfluoropolyether (PFPE), polychlorotetrafluoroethylene (PCTFE), or any combination thereof. In some embodiments, the surface modification comprises silicone, silane, fluoro-polymer treatment, parylene coating, any other suitable surface chemistry modification process, ceramic, clay minerals, bentonite, kaolinite, vermiculite, graphite, molybdenum disulfide, mica, boron nitride, sodium formate, sodium oleate, sodium palmitate, sodium sulfate, sodium alginate, or any combination thereof. In some embodiments, the liquid comprises silicone oils, fluorinated oils, ionic liquids, mineral oils, ferrofluids, polyphenyl ether, vegetable oil, esters of saturated fatty and dibasic acids, grease, fatty acids, triglycerides, polyalphaolefin, polyglycol hydrocarbons, other Non-hydrocarbon synthetic oils, or any combination thereof. In some embodiments, the liquid further comprises surfactants, electrolytes, rheology modifier, wax, graphite, graphene, molybdenum disulfide, PTFE particles, or any combination thereof. In some embodiments, the first plurality of electrodes, the dielectric, the surface configured to support the droplet comprising the sample, or any combination thereof is removable from the array. In some embodiments, the frequency of the vibration displaces the surface or a portion of the surface from 0.05 millimeters (mm) to 10 mm.

In some embodiments, the electro-mechanical actuator is configured to displace the surface or a portion of the surface from 0.05 millimeters (mm) to 10 mm. In some embodiments, the surface or the portion of the surface, is displaced from about 0.05 mm to about 10 mm. In some embodiments, the surface or the portion of the surface, is displaced from about 0.05 mm to about 0.1 mm, about 0.05 mm to about 0.5 mm, about 0.05 mm to about 1 mm, about 0.05 mm to about 2 mm, about 0.05 mm to about 3 mm, about 0.05 mm to about 4 mm, about 0.05 mm to about 5 mm, about 0.05 mm to about 6 mm, about 0.05 mm to about 7 mm, about 0.05 mm to about 8 mm, about 0.05 mm to about 9 mm, about 0.05 mm to about 10 mm about 0.1 mm to about 0.5 mm, about 0.1 mm to about 1 mm, about 0.1 mm to about 2 mm, about 0.1 mm to about 3 mm, about 0.1 mm to about 4 mm, about 0.1 mm to about 5 mm, about 0.1 mm to about 6 mm, about 0.1 mm to about 7 mm, about 0.1 mm to about 8 mm, about 0.1 mm to about 9 mm, about 0.1 mm to about 10 mm, about 0.5 mm to about 1 mm, about 0.5 mm to about 2 mm, about 0.5 mm to about 3 mm, about 0.5 mm to about 4 mm, about 0.5 mm to about 5 mm, about 0.5 mm to about 6 mm, about 0.5 mm to about 7 mm, about 0.5 mm to about 8 mm, about 0.5 mm to about 9 mm, about 0.5 mm to about 10 mm, about 1 mm to about 2 mm, about 1 mm to about 3 mm, about 1 mm to about 4 mm, about 1 mm to about 5 mm, about 1 mm to about 6 mm, about 1 mm to about 7 mm, about 1 mm to about 8 mm, about 1 mm to about 9 mm, about 1 mm to about 10 mm, about 2 mm to about 3 mm, about 2 mm to about 4 mm, about 2 mm to about 5 mm, about 2 mm to about 6 mm, about 2 mm to about 7 mm, about 2 mm to about 8 mm, about 2 mm to about 9 mm, about 2 mm to about 10 mm, about 3 mm to about 4 mm, about 3 mm to about 5 mm, about 3 mm to about 6 mm, about 3 mm to about 7 mm, about 3 mm to about 8 mm, about 3 mm to about 9 mm, about 3 mm to about 10 mm, about 4 mm to about 5 mm, about 4 mm to about 6 mm, about 4 mm to about 7 mm, about 4 mm to about 8 mm, about 4 mm to about 9 mm, about 4 mm to about 10 mm, about 5 mm to about 6 mm, about 5 mm to about 7 mm, about 5 mm to about 8 mm, about 5 mm to about 9 mm, about 5 mm to about 10 mm, about 6 mm to about 7 mm, about 6 mm to about 8 mm, about 6 mm to about 9 mm, about 7 mm to about 8 mm, about 7 mm to about 9 mm, about 7 mm to about 10 mm, about 8 mm to about 9 mm, or about 9 mm to about 10 mm. In some embodiments, the surface or the portion of the surface, is displaced from about 0.05 mm, about 0.1 mm, about 0.5 mm, about 1 mm, about 2 mm, about 3 mm, about 4 mm, about 5 mm, about 6 mm, about 7 mm, about 8 mm, about 9 mm, or about 10 mm. In some embodiments, the surface or the portion of the surface, is displaced from at least about 0.05 mm, about 0.1 mm, about 0.5 mm, about 1 mm, about 2 mm, about 3 mm, about 4 mm, about 5 mm, about 6 mm, about 7 mm, or about 8 mm. In some embodiments, the surface or the portion of the surface, is displaced from at most about 0.1 mm, about 0.5 mm, about 1 mm, about 2 mm, about 3 mm, about 4 mm, about 5 mm, about 6 mm, about 7 mm, about 8 mm, about 9 mm, or about 10 mm.

In some embodiments, the frequency of the vibration is from 1 Hertz (hz) to 20 kilohertz (khz). In some embodiments, the frequency of the vibration is from 1 Hertz (hz) to 20 kilohertz (khz). In some embodiments, the frequency of the vibration is from about 1 Hz to about 10 Hz. In some embodiments, the frequency of the vibration is from about 1 Hz to about 2 Hz, about 1 Hz to about 3 Hz, about 1 Hz to about 4 Hz, about 1 Hz to about 5 Hz, about 1 Hz to about 6 Hz, about 1 Hz to about 7 Hz, about 1 Hz to about 8 Hz, about 1 Hz to about 9 Hz, about 1 Hz to about 10 Hz, about 2 Hz to about 3 Hz, about 2 Hz to about 4 Hz, about 2 Hz to about 5 Hz, about 2 Hz to about 6 Hz, about 2 Hz to about 7 Hz, about 2 Hz to about 8 Hz, about 2 Hz to about 9 Hz, about 2 Hz to about 10 Hz, about 3 Hz to about 4 Hz, about 3 Hz to about 5 Hz, about 3 Hz to about 6 Hz, about 3 Hz to about 7 Hz, about 3 Hz to about 8 Hz, about 3 Hz to about 9 Hz, about 3 Hz to about 10 Hz, about 4 Hz to about 5 Hz, about 4 Hz to about 6 Hz, about 4 Hz to about 7 Hz, about 4 Hz to about 8 Hz, about 4 Hz to about 9 Hz, about 4 Hz to about 10 Hz, about 5 Hz to about 6 Hz, about 5 Hz to about 7 Hz, about 5 Hz to about 8 Hz, about 5 Hz to about 9 Hz, about 5 Hz to about 10 Hz, about 6 Hz to about 7 Hz, about 6 Hz to about 8 Hz, about 6 Hz to about 9 Hz, about 6 Hz to about 10 Hz, about 7 Hz to about 8 Hz, about 7 Hz to about 9 Hz, about 7 Hz to about 10 Hz, about 8 Hz to about 9 Hz, about 8 Hz to about 10 Hz, or about 9 Hz to about 10 Hz. In some embodiments, the frequency of the vibration is from about 1 Hz, about 2 Hz, about 3 Hz, about 4 Hz, about 5 Hz, about 6 Hz, about 7 Hz, about 8 Hz, about 9 Hz, or about 10 Hz. In some embodiments, the frequency of the vibration is from at least about 1 Hz, about 2 Hz, about 3 Hz, about 4 Hz, about 5 Hz, about 6 Hz, about 7 Hz, about 8 Hz, or about 9 Hz. In some embodiments, the frequency of the vibration is from at most about 2 Hz, about 3 Hz, about 4 Hz, about 5 Hz, about 6 Hz, about 7 Hz, about 8 Hz, about 9 Hz, or about 10 Hz. In some embodiments, the frequency of the vibration is from about 10 Hz to about 1,000 Hz. In some embodiments, the frequency of the vibration is from about 10 Hz to about 100 Hz, about 10 Hz to about 200 Hz, about 10 Hz to about 300 Hz, about 10 Hz to about 400 Hz, about 10 Hz to about 500 Hz, about 10 Hz to about 600 Hz, about 10 Hz to about 700 Hz, about 10 Hz to about 800 Hz, about 10 Hz to about 900 Hz, about 10 Hz to about 1,000 Hz, about 100 Hz to about 200 Hz, about 100 Hz to about 300 Hz, about 100 Hz to about 400 Hz, about 100 Hz to about 500 Hz, about 100 Hz to about 600 Hz, about 100 Hz to about 700 Hz, about 100 Hz to about 800 Hz, about 100 Hz to about 900 Hz, about 100 Hz to about 1,000 Hz, about 200 Hz to about 300 Hz, about 200 Hz to about 400 Hz, about 200 Hz to about 500 Hz, about 200 Hz to about 600 Hz, about 200 Hz to about 700 Hz, about 200 Hz to about 800 Hz, about 200 Hz to about 900 Hz, about 200 Hz to about 1,000 Hz, about 300 Hz to about 400 Hz, about 300 Hz to about 500 Hz, about 300 Hz to about 600 Hz, about 300 Hz to about 700 Hz, about 300 Hz to about 800 Hz, about 300 Hz to about 900 Hz, about 300 Hz to about 1,000 Hz, about 400 Hz to about 500 Hz, about 400 Hz to about 600 Hz, about 400 Hz to about 700 Hz, about 400 Hz to about 800 Hz, about 400 Hz to about 900 Hz, about 400 Hz to about 1,000 Hz, about 500 Hz to about 600 Hz, about 500 Hz to about 700 Hz, about 500 Hz to about 800 Hz, about 500 Hz to about 900 Hz, about 500 Hz to about 1,000 Hz, about 600 Hz to about 700 Hz, about 600 Hz to about 800 Hz, about 600 Hz to about 900 Hz, about 600 Hz to about 1,000 Hz, about 700 Hz to about 800 Hz, about 700 Hz to about 900 Hz, about 700 Hz to about 1,000 Hz, about 800 Hz to about 900 Hz, about 800 Hz to about 1,000 Hz, or about 900 Hz to about 1,000 Hz. In some embodiments, the frequency of the vibration is from about 10 Hz, about 100 Hz, about 200 Hz, about 300 Hz, about 400 Hz, about 500 Hz, about 600 Hz, about 700 Hz, about 800 Hz, about 900 Hz, or about 1,000 Hz. In some embodiments, the frequency of the vibration is from at least about 10 Hz, about 100 Hz, about 200 Hz, about 300 Hz, about 400 Hz, about 500 Hz, about 600 Hz, about 700 Hz, about 800 Hz, or about 900 Hz. In some embodiments, the frequency of the vibration is from at most about 100 Hz, about 200 Hz, about 300 Hz, about 400 Hz, about 500 Hz, about 600 Hz, about 700 Hz, about 800 Hz, about 900 Hz, or about 1,000 Hz. In some embodiments, the frequency of the vibration is from about 1 kHz to about 20 kHz. In some embodiments, the frequency of the vibration is from about 1 kHz to about 2.5 kHz, about 1 kHz to about 5 kHz, about 1 kHz to about 7.5 kHz, about 1 kHz to about 10 kHz, about 1 kHz to about 12.5 kHz, about 1 kHz to about 15 kHz, about 1 kHz to about 17.5 kHz, about 1 kHz to about 20 kHz, about 2.5 kHz to about 5 kHz, about 2.5 kHz to about 7.5 kHz, about 2.5 kHz to about 10 kHz, about 2.5 kHz to about 12.5 kHz, about 2.5 kHz to about 15 kHz, about 2.5 kHz to about 17.5 kHz, about 2.5 kHz to about 20 kHz, about 5 kHz to about 7.5 kHz, about 5 kHz to about 10 kHz, about 5 kHz to about 12.5 kHz, about 5 kHz to about 15 kHz, about 5 kHz to about 17.5 kHz, about 5 kHz to about 20 kHz, about 7.5 kHz to about 10 kHz, about 7.5 kHz to about 12.5 kHz, about 7.5 kHz to about 15 kHz, about 7.5 kHz to about 17.5 kHz, about 7.5 kHz to about 20 kHz, about 10 kHz to about 12.5 kHz, about 10 kHz to about 15 kHz, about 10 kHz to about 17.5 kHz, about 10 kHz to about 20 kHz, about 12.5 kHz to about 15 kHz, about 12.5 kHz to about 17.5 kHz, about 12.5 kHz to about 20 kHz, about 15 kHz to about 17.5 kHz, about 15 kHz to about 20 kHz, or about 17.5 kHz to about 20 kHz. In some embodiments, the frequency of the vibration is from about 1 kHz, about 2.5 kHz, about 5 kHz, about 7.5 kHz, about 10 kHz, about 12.5 kHz, about 15 kHz, about 17.5 kHz, or about 20 kHz. In some embodiments, the frequency of the vibration is from at least about 1 kHz, about 2.5 kHz, about 5 kHz, about 7.5 kHz, about 10 kHz, about 12.5 kHz, about 15 kHz, or about 17.5 kHz. In some embodiments, the frequency of the vibration is from at most about 2.5 kHz, about 5 kHz, about 7.5 kHz, about 10 kHz, about 12.5 kHz, about 15 kHz, about 17.5 kHz, or about 20 kHz.

### Alternative Implementations

Droplet on Open Surface (Single Plate Configuration) or Sandwiched Between Two Plates (Two Plate Configuration)

For electrowetting droplet manipulation, a droplet may either be placed on an open surface (single plate) or sandwiched between two plates (double plate). In the double plate configuration, a droplet may be sandwiched between two plates, typically separated by 100 μm-500 μm. The two-plate configuration has electrodes for providing actuation voltages on one side while the other side may provide a reference electrode (e.g., a common ground signal). A droplet's constant contact to the reference electrode in a two-plate configuration provides stronger force from the electric field on the droplet and hence robust control over droplets. In the two plate configuration droplets may be split at a lower actuation voltage. In the single plate configuration the actuation electrodes and the reference electrode are on the same side.

Two-plate electrowetting systems may be improved by the surface treatments described above. In two-plate systems, a droplet is sandwiched between plates separated by a small distance. The space between the plates may be filled with another fluid or just air. Smoothing the liquid-facing surfaces of the two plates to 2 μm, 1 μm, or 500 nm, using the techniques described above, may allow two-plate systems to operate at lower voltages, with reduced droplet pinning, reduced leave-behind tracks, reduced cross-contamination, and reduced sample loss.

Some aspects of present disclosure provide for the reagent or droplet making contact with a surface only on one side. In some embodiments, the first reagent or droplet makes contact with a surface only on one side. In some embodiments, the second reagent or droplet makes contact with a surface only on one side. In some embodiments, the third reagent or droplet makes contact with a surface only on one side. In some embodiments, the merged reagent or droplet makes contact with a surface only on one side.

Optoelectrowetting and Photoelectrowetting

In some embodiments, applying electric potential directly to an array of electrodes is one way of actuating droplets using electrowetting; however, there are alternate electrowetting mechanisms that differ from this conventional electrowetting mechanism. Two notable mechanisms, both of which use light for actuating the droplets, are described herein-optoelectrowetting and photoelectrowetting. The general principles for manufacturing the electrowetting arrays, creating a smooth surface and slippery surface described above are applicable not only to conventional electrowetting described earlier, but is also applicable to optoelectrowetting, photoelectrowetting and other forms of electrowetting.

A liquid film may be laid on a grid of photoconductors, to yield “liquid-on-liquid optoelectrowetting.” Instead of having a grid of electrodes under the lubricating liquid layer, the grid may be formed of light active photoconductor, either in a grid of pads, or as a single photoconductive circuit. Light shone on the photoconductor may form patterns and provide electrowetting effect. The textured solid and oil may be chosen to be sufficiently transparent to light so that the underlying surface is exposed to light to create differential wetting.

Optoelectrowetting

In some embodiments, the optoelectrowetting mechanism may use a photoconductor underneath the conventional electrowetting circuit, with an AC power source attached. Under normal (dark) conditions, the majority of the system's impedance lies in the photoconducting region, and therefore the majority of the voltage drop may occur here. However, when light is shone on the system, carrier generation and recombination causes the conductivity of the photoconductor to spike and the voltage drop across the photoconductor reduces. As a result, a voltage drop occurs across the insulating layer, changing the contact angle as a function of the voltage.

Photoelectrowetting

In some embodiments, photoelectrowetting is a modification of the wetting properties of a surface (typically a hydrophobic surface) using incident light. Whereas ordinary electrowetting is observed in a droplet sitting on a dielectric coated conductor (liquid/insulator/conductor stack), photoelectrowetting may be observed by replacing the conductor with a semiconductor (liquid/insulator/semiconductor stack).

Incident light above the band gap of semiconductor can create photo-induced carriers via electron-hole pair generation in the depletion region of the underlying semiconductor. This leads to a modification of the capacitance of the insulator/semiconductor stack, resulting in a modification of the contact angle of a liquid droplet resting on the surface of the stack. The figure illustrates the principle of the photoelectrowetting effect. At zero bias (0V) the conducting droplet has a large contact angle (left image) if the insulator is hydrophobic. As the bias is increased (positive for a p-type semiconductor, negative for an n-type semiconductor) the droplet spreads out—i.e. the contact angle decreases (middle image). In the presence of light (having an energy superior to the band gap of the semiconductor) the droplet spreads out more due to the reduction of the thickness of the space charge region at the insulator/semiconductor interface.

Some aspects of present disclosure provide for subjecting the reagents to light. In some embodiments, the first droplet is subject to light. In some embodiments, the second droplet is subject to light. In some embodiments, the third droplet is subject to light. In some embodiments, the merged droplet is subject to light.

### Methods and Systems for Droplet Correction During Droplet Operations

In an aspect, the present disclosure provides a method for processing a plurality of biological samples. The method may comprise receiving, adjacent to an array, a plurality of droplets that may comprise the plurality of biological samples, and using at least the array to process the plurality of biological samples in the plurality of droplets or derivatives thereof at a coefficient of variation (CV) of at least one parameter of the plurality of droplets or derivatives thereof, or the array, of less than 20% at cross-talk between the plurality of droplets at less than 5%. This may be used to process the plurality of biological samples. The array may be an electrowetting device, as described elsewhere herein.

The at least one parameter may comprise one or more members selected from the group consisting of droplet size, droplet volume, droplet position, droplet speed, droplet wetting, droplet temperature, droplet pH, beads in droplets, number of cells in droplets, droplet color, concentration of chemical material, concentration of biological substance, or any combination thereof. The at least one parameter may be at least 1, 2, 3, 4, 5, 6, 7, 8, 9, 10, or more parameters. The at least one parameter may be a measurable property of a droplet.

In some embodiments, the concentration of a chemical material or biological substance within a droplet is monitored such that it does not exceed or fall below a predetermine threshold. In some embodiments, the predetermined threshold of a concentration of a chemical material or biological substance is 5%, 10%, 15%, 20%, 25%, 30%, 35%, 40%, 45%, 50%, 55%, 60%, 65%, 70% 75%, 80%, 85%, 90%, or 95%.

The location may be of a droplet, a reagent, a biological sample, a component of the array, a position of the array, an area of the array, an area adjacent to the array, a point of the array, or any combination thereof. The location may be corrected by at least 0.001%, 0.01%, 0.1%, 1%, 5%, 10%, 15%, 20%, 30%, 40%, 50%, 60%, 70%, 80%, 90%, 95%, 99%, or more. The location may be corrected by at most 99%, 95%, 90%, 80%, 70%, 60%, 50%, 40%, 30%, 20%, 15%, 10%, 5%, 1%, 0.1%, 0.01%, 0.001%, or less. The location may be corrected from 0.001% to 20%, 0.01% to 10%, 0.01% to 5%, or 0.1% to 1%.

The droplet volume may comprise a volume of at least 1 picoliter (μL), 10 μL, 100 μL, 1 nanoliter (nL), 10 nL, 100 nL, 1 μL, 10 μL, 100 μL, 1 milliliter (mL), 10 mL or more. The droplet volume may comprise a volume of at most 10 mL, 1 mL, 100 μL, 10 μL, 1 μL, 100 nL, 10 nL, 1 nL, 100 μL, 10 μL, 1 μL, or less. The droplet volume may be corrected by at least 0.001%, 0.01%, 0.1%, 1%, 5%, 10%, 15%, 20%, 30%, 40%, 50%, 60%, 70%, 80%, 90%, 95%, 99%, or more. The droplet volume may be corrected by at most 99%, 95%, 90%, 80%, 70%, 60%, 50%, 40%, 30%, 20%, 15%, 10%, 5%, 1%, 0.1%, 0.01%, 0.001%, or less. The droplet volume may be corrected from 0.001% to 20%, 0.01% to 10%, 0.01% to 5%, or 0.1% to 1%. In some embodiments, a droplet is replenished if the volume of the droplet falls below a predetermined threshold. In some embodiments, the predetermined threshold may be a volume of at least 1 picoliter (μL), 10 μL, 100 μL, 1 nanoliter (nL), 10 nL, 100 nL, 1 μL, 10 μL, 100 μL, 1 milliliter (mL), 10 mL or more. In some embodiments, the predetermined threshold may be a volume at most 10 mL, 1 mL, 100 μL, 10 μL, 1 μL, 100 nL, 10 nL, 1 nL, 100 μL, 10 μL, 1 μL, or less. In some embodiments, a droplet is reduced if the volume of the droplet exceeds a predetermined threshold. In some embodiments, the predetermined threshold may be a volume of at least 1 picoliter (μL), 10 μL, 100 μL, 1 nanoliter (nL), 10 nL, 100 nL, 1 μL, 10 μL, 100 μL, 1 milliliter (mL), 10 mL or more. In some embodiments, the predetermined threshold may be a volume at most 10 mL, 1 mL, 100 μL, 10 μL, 1 μL, 100 nL, 10 nL, 1 nL, 100 μL, 10 μL, 1 μL, or less.

A biological sample may comprise a nucleic acid, protein, cell, salt, buffer, or enzyme, wherein the droplet comprises one or more reagents for nucleic acid isolation, cell isolation, protein isolation, peptide purification, isolation or purification of a biopolymer, immunoprecipitation, in vitro diagnostics, exosome isolation, cell activation, cell expansion, or isolation of a specific biomolecule, and wherein the droplet is manipulated by the reagents to perform the nucleic acid isolation, cell isolation, protein isolation, peptide purification, isolation or purification of a biopolymer, immunoprecipitation, in vitro diagnostics, exosome isolation, cell activation, cell expansion, or isolation of a specific biomolecule. The presence of the biological sample may be corrected by an amount of at least 0.001%, 0.01%, 0.1%, 1%, 5%, 10%, 15%, 20%, 30%, 40%, 50%, 60%, 70%, 80%, 90%, 95%, 99%, or more. The presence of the biological sample may be corrected by an amount of at most 99%, 95%, 90%, 80%, 70%, 60%, 50%, 40%, 30%, 20%, 15%, 10%, 5%, 1%, 0.1%, 0.01%, 0.001%, or less. The presence of the biological sample may be corrected by an amount from 0.001% to 20%, 0.01% to 10%, 0.01% to 5%, or 0.1% to 1%.

The activity of biological material may comprise enzymatic activity, cellular activity, small-molecule activity, reagent activity, wherein the activity may be a measure of affinity, specificity, reactivity, rate, inhibition, toxicity (e.g., IC₅₀, LD₅₀, EC₅₀, ED₅₀, GI₅₀, etc.), or any combination thereof. The activity of the biological sample may be corrected by an amount of at least 0.001%, 0.01%, 0.1%, 1%, 5%, 10%, 15%, 20%, 30%, 40%, 50%, 60%, 70%, 80%, 90%, 95%, 99%, or more. The activity of the biological sample may be corrected by an amount of at most 99%, 95%, 90%, 80%, 70%, 60%, 50%, 40%, 30%, 20%, 15%, 10%, 5%, 1%, 0.1%, 0.01%, 0.001%, or less. The activity of the biological sample may be corrected by an amount from 0.001% to 20%, 0.01% to 10%, 0.01% to 5%, or 0.1% to 1%.

In some embodiments, the droplet has a viscosity of about 0% glycerol to about 60% glycerol at room temperature (˜25° C.). In some embodiments, the droplet has a viscosity of about 0% glycerol to about 10% glycerol, about 0% glycerol to about 15% glycerol, about 0% glycerol to about 20% glycerol, about 0% glycerol to about 25% glycerol, about 0% glycerol to about 30% glycerol, about 0% glycerol to about 35% glycerol, about 0% glycerol to about 40% glycerol, about 0% glycerol to about 45% glycerol, about 0% glycerol to about 50% glycerol, about 0% glycerol to about 55% glycerol, about 0% glycerol to about 60% glycerol, about 10% glycerol to about 15% glycerol, about 10% glycerol to about 20% glycerol, about 10% glycerol to about 25% glycerol, about 10% glycerol to about 30% glycerol, about 10% glycerol to about 35% glycerol, about 10% glycerol to about 40% glycerol, about 10% glycerol to about 45% glycerol, about 10% glycerol to about 50% glycerol, about 10% glycerol to about 55% glycerol, about 10% glycerol to about 60% glycerol, about 15% glycerol to about 20% glycerol, about 15% glycerol to about 25% glycerol, about 15% glycerol to about 30% glycerol, about 15% glycerol to about 35% glycerol, about 15% glycerol to about 40% glycerol, about 15% glycerol to about 45% glycerol, about 15% glycerol to about 50% glycerol, about 15% glycerol to about 55% glycerol, about 15% glycerol to about 60% glycerol, about 20% glycerol to about 25% glycerol, about 20% glycerol to about 30% glycerol, about 20% glycerol to about 35% glycerol, about 20% glycerol to about 40% glycerol, about 20% glycerol to about 45% glycerol, about 20% glycerol to about 50% glycerol, about 20% glycerol to about 55% glycerol, about 20% glycerol to about 60% glycerol, about 25% glycerol to about 30% glycerol, about 25% glycerol to about 35% glycerol, about 25% glycerol to about 40% glycerol, about 25% glycerol to about 45% glycerol, about 25% glycerol to about 50% glycerol, about 25% glycerol to about 55% glycerol, about 25% glycerol to about 60% glycerol, about 30% glycerol to about 35% glycerol, about 30% glycerol to about 40% glycerol, about 30% glycerol to about 45% glycerol, about 30% glycerol to about 50% glycerol, about 30% glycerol to about 55% glycerol, about 30% glycerol to about 60% glycerol, about 35% glycerol to about 40% glycerol, about 35% glycerol to about 45% glycerol, about 35% glycerol to about 50% glycerol, about 35% glycerol to about 55% glycerol, about 35% glycerol to about 60% glycerol, about 40% glycerol to about 45% glycerol, about 40% glycerol to about 50% glycerol, about 40% glycerol to about 55% glycerol, about 40% glycerol to about 60% glycerol, about 45% glycerol to about 50% glycerol, about 45% glycerol to about 55% glycerol, about 45% glycerol to about 60% glycerol, about 50% glycerol to about 55% glycerol, about 50% glycerol to about 60% glycerol, or about 55% glycerol to about 60% glycerol at room temperature (˜25° C.). In some embodiments, the droplet has a viscosity of about 0% glycerol, about 10% glycerol, about 15% glycerol, about 20% glycerol, about 25% glycerol, about 30% glycerol, about 35% glycerol, about 40% glycerol, about 45% glycerol, about 50% glycerol, about 55% glycerol, or about 60% glycerol. In some embodiments, the droplet has a viscosity of at least about 0% glycerol, about 10% glycerol, about 15% glycerol, about 20% glycerol, about 25% glycerol, about 30% glycerol, about 35% glycerol, about 40% glycerol, about 45% glycerol, about 50% glycerol, or about 55% glycerol at room temperature (˜25° C.). In some embodiments, the droplet has a viscosity of at most about 10% glycerol, about 15% glycerol, about 20% glycerol, about 25% glycerol, about 30% glycerol, about 35% glycerol, about 40% glycerol, about 45% glycerol, about 50% glycerol, about 55% glycerol, or about 60% glycerol. In some embodiments, the droplet has a viscosity of about 40% glycerol at room temperature (˜25° C.).

In some embodiments, the droplet has a viscosity of about 0.1 centipoise (cP) to about 200 cP at room temperature (˜25° C.). In some embodiments, the droplet has a viscosity of about 0.1 cP to about 1 cP, about 0.1 cP to about 2 cP, about 0.1 cP to about 5 cP, about 0.1 cP to about 10 cP, about 0.1 cP to about 30 cP, about 0.1 cP to about 50 cP, about 0.1 cP to about 70 cP, about 0.1 cP to about 100 cP, about 0.1 cP to about 150 cP, about 0.1 cP to about 200 cP, about 1 cP to about 2 cP, about 1 cP to about 5 cP, about 1 cP to about 10 cP, about 1 cP to about 30 cP, about 1 cP to about 50 cP, about 1 cP to about 70 cP, about 1 cP to about 100 cP, about 1 cP to about 150 cP, about 1 cP to about 200 cP, about 2 cP to about 5 cP, about 2 cP to about 10 cP, about 2 cP to about 30 cP, about 2 cP to about 50 cP, about 2 cP to about 70 cP, about 2 cP to about 100 cP, about 2 cP to about 150 cP, about 2 cP to about 200 cP, about 5 cP to about 10 cP, about 5 cP to about 30 cP, about 5 cP to about 50 cP, about 5 cP to about 70 cP, about 5 cP to about 100 cP, about 5 cP to about 150 cP, about 5 cP to about 200 cP, about 10 cP to about 30 cP, about 10 cP to about 50 cP, about 10 cP to about 70 cP, about 10 cP to about 100 cP, about 10 cP to about 150 cP, about 10 cP to about 200 cP, about 30 cP to about 50 cP, about 30 cP to about 70 cP, about 30 cP to about 100 cP, about 30 cP to about 150 cP, about 30 cP to about 200 cP, about 50 cP to about 70 cP, about 50 cP to about 100 cP, about 50 cP to about 150 cP, about 50 cP to about 200 cP, about 70 cP to about 100 cP, about 70 cP to about 150 cP, about 70 cP to about 200 cP, about 100 cP to about 150 cP, about 100 cP to about 200 cP, or about 150 cP to about 200 cP at room temperature (˜25° C.). In some embodiments, the droplet has a viscosity of about 0.1 cP, about 1 cP, about 2 cP, about 5 cP, about 10 cP, about 30 cP, about 50 cP, about 70 cP, about 100 cP, about 150 cP, or about 200 cP at room temperature (˜25° C.). In some embodiments, the droplet has a viscosity of at least about 0.1 cP, about 1 cP, about 2 cP, about 5 cP, about 10 cP, about 30 cP, about 50 cP, about 70 cP, about 100 cP, or about 150 cP at room temperature (˜25° C.). In some embodiments, the droplet has a viscosity of at most about 1 cP, about 2 cP, about 5 cP, about 10 cP, about 30 cP, about 50 cP, about 70 cP, about 100 cP, about 150 cP, or about 200 cP at room temperature (˜25° C.).

In some embodiments, the droplet has a viscosity of about 0% glycerol to about 30% glycerol at room temperature (˜25° C.). In some embodiments, the droplet has a viscosity of about 0% glycerol to about 5% glycerol, about 0% glycerol to about 7.5% glycerol, about 0% glycerol to about 10% glycerol, about 0% glycerol to about 12.5% glycerol, about 0% glycerol to about 15% glycerol, about 0% glycerol to about 17.5% glycerol, about 0% glycerol to about 20% glycerol, about 0% glycerol to about 22.5% glycerol, about 0% glycerol to about 25% glycerol, about 0% glycerol to about 27.5% glycerol, about 0% glycerol to about 30% glycerol, about 5% glycerol to about 7.5% glycerol, about 5% glycerol to about 10% glycerol, about 5% glycerol to about 12.5% glycerol, about 5% glycerol to about 15% glycerol, about 5% glycerol to about 17.5% glycerol, about 5% glycerol to about 20% glycerol, about 5% glycerol to about 22.5% glycerol, about 5% glycerol to about 25% glycerol, about 5% glycerol to about 27.5% glycerol, about 5% glycerol to about 30% glycerol, about 7.5% glycerol to about 10% glycerol, about 7.5% glycerol to about 12.5% glycerol, about 7.5% glycerol to about 15% glycerol, about 7.5% glycerol to about 17.5% glycerol, about 7.5% glycerol to about 20% glycerol, about 7.5% glycerol to about 22.5% glycerol, about 7.5% glycerol to about 25% glycerol, about 7.5% glycerol to about 27.5% glycerol, about 7.5% glycerol to about 30% glycerol, about 10% glycerol to about 12.5% glycerol, about 10% glycerol to about 15% glycerol, about 10% glycerol to about 17.5% glycerol, about 10% glycerol to about 20% glycerol, about 10% glycerol to about 22.5% glycerol, about 10% glycerol to about 25% glycerol, about 10% glycerol to about 27.5% glycerol, about 10% glycerol to about 30% glycerol, about 12.5% glycerol to about 15% glycerol, about 12.5% glycerol to about 17.5% glycerol, about 12.5% glycerol to about 20% glycerol, about 12.5% glycerol to about 22.5% glycerol, about 12.5% glycerol to about 25% glycerol, about 12.5% glycerol to about 27.5% glycerol, about 12.5% glycerol to about 30% glycerol, about 15% glycerol to about 17.5% glycerol, about 15% glycerol to about 20% glycerol, about 15% glycerol to about 22.5% glycerol, about 15% glycerol to about 25% glycerol, about 15% glycerol to about 27.5% glycerol, about 15% glycerol to about 30% glycerol, about 17.5% glycerol to about 20% glycerol, about 17.5% glycerol to about 22.5% glycerol, about 17.5% glycerol to about 25% glycerol, about 17.5% glycerol to about 27.5% glycerol, about 17.5% glycerol to about 30% glycerol, about 20% glycerol to about 22.5% glycerol, about 20% glycerol to about 25% glycerol, about 20% glycerol to about 27.5% glycerol, about 20% glycerol to about 30% glycerol, about 22.5% glycerol to about 25% glycerol, about 22.5% glycerol to about 27.5% glycerol, about 22.5% glycerol to about 30% glycerol, about 25% glycerol to about 27.5% glycerol, about 25% glycerol to about 30% glycerol, or about 27.5% glycerol to about 30% glycerol at room temperature (˜25° C.). In some embodiments, the droplet has a viscosity of about 0% glycerol, about 5% glycerol, about 7.5% glycerol, about 10% glycerol, about 12.5% glycerol, about 15% glycerol, about 17.5% glycerol, about 20% glycerol, about 22.5% glycerol, about 25% glycerol, about 27.5% glycerol, or about 30% glycerol at room temperature (˜25° C.). In some embodiments, the droplet has a viscosity of at least about 0% glycerol, about 5% glycerol, about 7.5% glycerol, about 10% glycerol, about 12.5% glycerol, about 15% glycerol, about 17.5% glycerol, about 20% glycerol, about 22.5% glycerol, about 25% glycerol, or about 27.5% glycerol at room temperature (˜25° C.). In some embodiments, the droplet has a viscosity of at most about 5% glycerol, about 7.5% glycerol, about 10% glycerol, about 12.5% glycerol, about 15% glycerol, about 17.5% glycerol, about 20% glycerol, about 22.5% glycerol, about 25% glycerol, about 27.5% glycerol, or about 30% glycerol at room temperature (˜25° C.).

In some embodiments, the droplet has a viscosity of about 0.5 cP to about 15 cP at room temperature (˜25° C.). In some embodiments, the droplet has a viscosity of about 0.5 cP to about 1 cP, about 0.5 cP to about 2 cP, about 0.5 cP to about 3 cP, about 0.5 cP to about 4 cP, about 0.5 cP to about 5 cP, about 0.5 cP to about 7 cP, about 0.5 cP to about 9 cP, about 0.5 cP to about 11 cP, about 0.5 cP to about 13 cP, about 0.5 cP to about 15 cP, about 1 cP to about 2 cP, about 1 cP to about 3 cP, about 1 cP to about 4 cP, about 1 cP to about 5 cP, about 1 cP to about 7 cP, about 1 cP to about 9 cP, about 1 cP to about 11 cP, about 1 cP to about 13 cP, about 1 cP to about 15 cP, about 2 cP to about 3 cP, about 2 cP to about 4 cP, about 2 cP to about 5 cP, about 2 cP to about 7 cP, about 2 cP to about 9 cP, about 2 cP to about 11 cP, about 2 cP to about 13 cP, about 2 cP to about 15 cP, about 3 cP to about 4 cP, about 3 cP to about 5 cP, about 3 cP to about 7 cP, about 3 cP to about 9 cP, about 3 cP to about 11 cP, about 3 cP to about 13 cP, about 3 cP to about 15 cP, about 4 cP to about 5 cP, about 4 cP to about 7 cP, about 4 cP to about 9 cP, about 4 cP to about 11 cP, about 4 cP to about 13 cP, about 4 cP to about 15 cP, about 5 cP to about 7 cP, about 5 cP to about 9 cP, about 5 cP to about 11 cP, about 5 cP to about 13 cP, about 5 cP to about 15 cP, about 7 cP to about 9 cP, about 7 cP to about 11 cP, about 7 cP to about 13 cP, about 7 cP to about 15 cP, about 9 cP to about 11 cP, about 9 cP to about 13 cP, about 9 cP to about 15 cP, about 11 cP to about 13 cP, about 11 cP to about 15 cP, or about 13 cP to about 15 cP at room temperature (˜25° C.). In some embodiments, the droplet has a viscosity of about 0.5 cP, about 1 cP, about 2 cP, about 3 cP, about 4 cP, about 5 cP, about 7 cP, about 9 cP, about 11 cP, about 13 cP, or about 15 cP at room temperature (˜25° C.). In some embodiments, the droplet has a viscosity of at least about 0.5 cP, about 1 cP, about 2 cP, about 3 cP, about 4 cP, about 5 cP, about 7 cP, about 9 cP, about 11 cP, or about 13 cP at room temperature (˜25° C.). In some embodiments, the droplet has a viscosity of at most about 1 cP, about 2 cP, about 3 cP, about 4 cP, about 5 cP, about 7 cP, about 9 cP, about 11 cP, about 13 cP, or about 15 cP at room temperature (˜25° C.).

The droplet radius may be at least 0.0001 μm, 0.001 μm, 0.01 μm, 0.1 μm, 1 μm, 5 μm, 10 μm, 20 μm, 30 μm, 40 μm, 50 μm, 60 μm, 70 μm, 80 μm, 90 μm, 100 μm, 500 μm, 1000 μm, 5000 μm, 10,000 μm, 50,000 μm, 100,000 μm, or more. The droplet radius may be at most 100,000 μm, 50,000 μm, 10,000 μm, 5000 μm, 1000 μm, 500 μm, 100 μm, 90 μm, 80 μm, 70 μm, 60 μm, 50 μm, 40 μm, 30 μm, 20 μm, 10 μm, 5 μm, 1 μm, 0.1 μm, 0.01 μm, 0.001 μm, 0.0001 μm, or less. The droplet radius may be from 1000 μm to 0.0001 μm, 500 μm to 0.01 μm, or 100 μm to 1 μm. The droplet radius may be corrected by an amount of at least 0.001%, 0.01%, 0.1%, 1%, 5%, 10%, 15%, 20%, 30%, 40%, 50%, 60%, 70%, 80%, 90%, 95%, 99%, or more. The droplet radius may be corrected by an amount of at most 99%, 95%, 90%, 80%, 70%, 60%, 50%, 40%, 30%, 20%, 15%, 10%, 5%, 1%, 0.1%, 0.01%, 0.001%, or less. The droplet radius may be corrected by an amount from 0.001% to 20%, 0.01% to 10%, 0.01% to 5%, or 0.1% to 1%.

In some embodiments, a droplet is replenished if the size of the droplet falls below a predetermined threshold. In some embodiments, a droplet is reduced if the size of the droplet exceeds a predetermined threshold. In some embodiments, the predetermined threshold may be a radius of at least 0.0001 μm, 0.001 μm, 0.01 μm, 0.1 μm, 1 μm, 5 μm, 10 μm, 20 μm, 30 μm, 40 μm, 50 μm, 60 μm, 70 μm, 80 μm, 90 μm, 100 μm, 500 μm, 1000 μm, 5000 μm, 10,000 μm, 50,000 μm, 100,000 μm, or more. In some embodiments, the predetermined threshold may be a volume at most 100,000 μm, 50,000 μm, 10,000 μm, 5000 μm, 1000 μm, 500 μm, 100 μm, 90 μm, 80 μm, 70 μm, 60 μm, 50 μm, 40 μm, 30 μm, 20 μm, 10 μm, 5 μm, 1 μm, 0.1 μm, 0.01 μm, 0.001 μm, 0.0001 μm, or less.

The droplet shape may be flat, round, spherical, oblong, oval, circular, or any combination thereof. The droplet shape may be corrected to be any shape. The droplet may be corrected to be flat, round, spherical, oblong, oval, circular, or any combination thereof.

The droplet height may be at least 0.0001 μm, 0.001 μm, 0.01 μm, 0.1 μm, 1 μm, 5 μm, 10 μm, 20 μm, 30 μm, 40 μm, 50 μm, 60 μm, 70 μm, 80 μm, 90 μm, 100 μm, 500 μm, 1000 μm, 5000 μm, 10,000 μm, 50,000 μm, 100,000 μm, or more. The droplet height may be at most 100,000 μm, 50,000 μm, 10,000 μm, 5,000 μm, 1000 μm, 500 μm, 100 μm, 90 μm, 80 μm, 70 μm, 60 μm, 50 μm, 40 μm, 30 μm, 20 μm, 10 μm, 5 μm, 1 μm, 0.1 μm, 0.01 μm, 0.001 μm, 0.0001 μm, or less. The droplet height may be from 1000 μm to 0.0001 μm, 500 μm to 0.01 μm, or 100 μm to 1 μm. The droplet height may be corrected by an amount of at least 0.001%, 0.01%, 0.1%, 1%, 5%, 10%, 15%, 20%, 30%, 40%, 50%, 60%, 70%, 80%, 90%, 95%, 99%, or more. The droplet height may be corrected by an amount of at most 99%, 95%, 90%, 80%, 70%, 60%, 50%, 40%, 30%, 20%, 15%, 10%, 5%, 1%, 0.1%, 0.01%, 0.001%, or less. The droplet height may be corrected by an amount from 0.001% to 20%, 0.01% to 10%, 0.01% to 5%, or 0.1% to 1%.

In some embodiments, a pH of a droplet is monitored by one or more methods as disclosed herein. In some embodiments, a pH of a droplet is maintained within a predetermined threshold. In some embodiments, pH of a droplet is maintained to not exceed 2, 3, 4, 5, 6, 7, 8, 9, 10, 11, 12, or 13. In some embodiments, a pH of droplet is maintained not to drop below 2, 3, 4, 5, 6, 7, 8, 9, 10, 11, 12, or 13.

In some embodiments, the relative humidity level achieved is about 50% to about 100%, about 60% to about 100%, about 70% to about 100%, about 80% to about 100%, or about 90% to about 100%. In some embodiments, the relative humidity level achieved is about 89% to about 100%. In some embodiments, the relative humidity level achieved is about 89% to about 90%, about 89% to about 91%, about 89% to about 92%, about 89% to about 93%, about 89% to about 94%, about 89% to about 95%, about 89% to about 96%, about 89% to about 97%, about 89% to about 98%, about 89% to about 99%, about 89% to about 100%, about 90% to about 91%, about 90% to about 92%, about 90% to about 93%, about 90% to about 94%, about 90% to about 95%, about 90% to about 96%, about 90% to about 97%, about 90% to about 98%, about 90% to about 99%, about 90% to about 100%, about 91% to about 92%, about 91% to about 93%, about 91% to about 94%, about 91% to about 95%, about 91% to about 96%, about 91% to about 97%, about 91% to about 98%, about 91% to about 99%, about 91% to about 100%, about 92% to about 93%, about 92% to about 94%, about 92% to about 95%, about 92% to about 96%, about 92% to about 97%, about 92% to about 98%, about 92% to about 99%, about 92% to about 100%, about 93% to about 94%, about 93% to about 95%, about 93% to about 96%, about 93% to about 97%, about 93% to about 98%, about 93% to about 99%, about 93% to about 100%, about 94% to about 95%, about 94% to about 96%, about 94% to about 97%, about 94% to about 98%, about 94% to about 99%, about 94% to about 100%, about 95% to about 96%, about 95% to about 97%, about 95% to about 98%, about 95% to about 99%, about 95% to about 100%, about 96% to about 97%, about 96% to about 98%, about 96% to about 99%, about 96% to about 100%, about 97% to about 98%, about 97% to about 99%, about 97% to about 100%, about 98% to about 99%, about 98% to about 100%, or about 99% to about 100%. In some embodiments, the relative humidity level achieved is about 89%, about 90%, about 91%, about 92%, about 93%, about 94%, about 95%, about 96%, about 97%, about 98%, about 99%, or about 100%. In some embodiments, the relative humidity level achieved is at least about 89%, about 90%, about 91%, about 92%, about 93%, about 94%, about 95%, about 96%, about 97%, about 98%, or about 99%. In some embodiments, the relative humidity level achieved is at most about 90%, about 91%, about 92%, about 93%, about 94%, about 95%, about 96%, about 97%, about 98%, about 99%, or about 100%.

Beyond controlling evaporation, heated arrays may also be used for precise control of the droplet temperature. Droplets may be heated on an open surface with heaters embedded on or below the array substrate. Without some form of environmental control, these substrate heaters may experience large temperature differences between the internal droplet temperature and the temperature on the surface of the heater. These large temperature differences may lead to imprecise droplet temperature control and may be subject to large temperature fluctuations based on, for example, surrounding air currents. Furthermore, without environmental temperature control, the difference between the heater temperature and the droplet temperature may be a function of parameters, including, for example, droplet surface area to volume ratio, droplet size, and temperature setpoint. These enclosures may be completely sealed to prevent the escape of heated humid air, but they may also be left partially open. For example, this design may allow control of condensation within a cooling temperature environment.

Examples of methods of creating smooth dielectric surfaces for EWOD droplet actuation can be found in WO2021041709, which is hereby incorporated by reference in its entirety.

### Arrays Without a Dedicated Reference Electrode

In some aspects of the present disclosure, the arrays described herein do not comprise one or more dedicated reference electrode(s). In these aspects, EWOD can be induced by using one or more neighboring electrode(s)/electrode(s) adjacent to the actuating electrode(s) as the current-return path.

These aspects of the present disclosure comprise a system for processing a droplet, the system comprising: an array comprising: a plurality of electrodes, wherein no electrode of the plurality of electrodes is permanently grounded; and a surface configured to support a droplet comprising the sample; a controller operatively coupled to the plurality of electrodes, wherein the controller is configured to: activate at least a subset of the plurality of electrodes with a time-varying voltage to alter a wetting characteristic of the surface. In some embodiments, the system does not comprise an overlying electrode. In some embodiments, the plurality of electrodes comprises at least one electrode comprising a cross-section or overlap with the droplet sufficient to generate a current-return path adjacent to the electrode and an adjacent electrode. In some embodiments, the plurality of electrodes is co-planar. In some embodiments, the time-varying voltage is bipolar. In some embodiments, the time-varying voltage is from about 1 Hz to about 20 kHz.

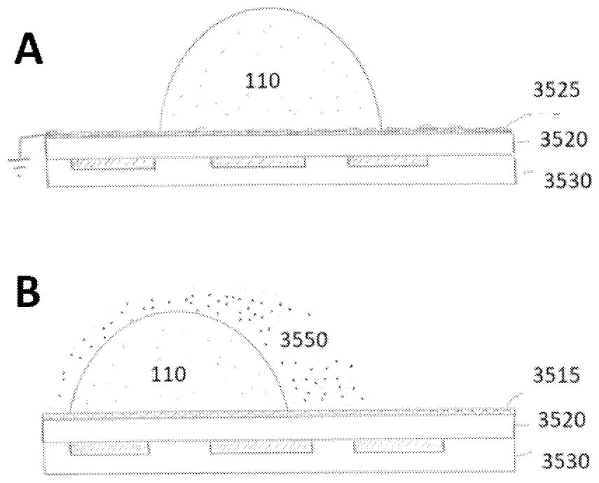

In some embodiments, a layer of oil, such as silicone oil, can serve as a hydrophobic coating as well as a reference electrode when grounded (FIG. 3A. The layer of oil may be slightly conductive or polar. The dielectric surface may contain microstructures introduced by the methods including, but not limited to, the methods described herein. These microstructures can wick oil and can be connected to a ground potential by, for example: a temporarily grounded actuation electrode, a dedicated ground electrode, a dedicated connection elsewhere on the array, or any combination thereof.

In some embodiments, ionized air may surround droplets in the array (FIG. 3B). Ionized air may be used for the array as a reference electrode for electrowetting actuation. Ionized air may be introduced through an ionized air blower and directed towards the droplet. A droplet may be permanently stuck to a location due to charging of the surface or the droplet (i.g., pinning). Droplet pinning may be mitigated by neutralizing the droplet with ions introduced through the blower.

In some embodiments, the time-varying voltage is from 1 Hertz (hz) to 20 kilohertz (khz). In some embodiments, the time-varying voltage is from 1 Hertz (hz) to 20 kilohertz (khz). In some embodiments, the time-varying voltage is from about 1 Hz to about 10 Hz. In some embodiments, the time-varying voltage is from about 1 Hz to about 2 Hz, about 1 Hz to about 3 Hz, about 1 Hz to about 4 Hz, about 1 Hz to about 5 Hz, about 1 Hz to about 6 Hz, about 1 Hz to about 7 Hz, about 1 Hz to about 8 Hz, about 1 Hz to about 9 Hz, about 1 Hz to about 10 Hz, about 2 Hz to about 3 Hz, about 2 Hz to about 4 Hz, about 2 Hz to about 5 Hz, about 2 Hz to about 6 Hz, about 2 Hz to about 7 Hz, about 2 Hz to about 8 Hz, about 2 Hz to about 9 Hz, about 2 Hz to about 10 Hz, about 3 Hz to about 4 Hz, about 3 Hz to about 5 Hz, about 3 Hz to about 6 Hz, about 3 Hz to about 7 Hz, about 3 Hz to about 8 Hz, about 3 Hz to about 9 Hz, about 3 Hz to about 10 Hz, about 4 Hz to about 5 Hz, about 4 Hz to about 6 Hz, about 4 Hz to about 7 Hz, about 4 Hz to about 8 Hz, about 4 Hz to about 9 Hz, about 4 Hz to about 10 Hz, about 5 Hz to about 6 Hz, about 5 Hz to about 7 Hz, about 5 Hz to about 8 Hz, about 5 Hz to about 9 Hz, about 5 Hz to about 10 Hz, about 6 Hz to about 7 Hz, about 6 Hz to about 8 Hz, about 6 Hz to about 9 Hz, about 6 Hz to about 10 Hz, about 7 Hz to about 8 Hz, about 7 Hz to about 9 Hz, about 7 Hz to about 10 Hz, about 8 Hz to about 9 Hz, about 8 Hz to about 10 Hz, or about 9 Hz to about 10 Hz. In some embodiments, the time-varying voltage is from about 1 Hz, about 2 Hz, about 3 Hz, about 4 Hz, about 5 Hz, about 6 Hz, about 7 Hz, about 8 Hz, about 9 Hz, or about 10 Hz. In some embodiments, the time-varying voltage is from at least about 1 Hz, about 2 Hz, about 3 Hz, about 4 Hz, about 5 Hz, about 6 Hz, about 7 Hz, about 8 Hz, or about 9 Hz. In some embodiments, the time-varying voltage is from at most about 2 Hz, about 3 Hz, about 4 Hz, about 5 Hz, about 6 Hz, about 7 Hz, about 8 Hz, about 9 Hz, or about 10 Hz. In some embodiments, the time-varying voltage is from about 10 Hz to about 1,000 Hz. In some embodiments, the time-varying voltage is from about 10 Hz to about 100 Hz, about 10 Hz to about 200 Hz, about 10 Hz to about 300 Hz, about 10 Hz to about 400 Hz, about 10 Hz to about 500 Hz, about 10 Hz to about 600 Hz, about 10 Hz to about 700 Hz, about 10 Hz to about 800 Hz, about 10 Hz to about 900 Hz, about 10 Hz to about 1,000 Hz, about 100 Hz to about 200 Hz, about 100 Hz to about 300 Hz, about 100 Hz to about 400 Hz, about 100 Hz to about 500 Hz, about 100 Hz to about 600 Hz, about 100 Hz to about 700 Hz, about 100 Hz to about 800 Hz, about 100 Hz to about 900 Hz, about 100 Hz to about 1,000 Hz, about 200 Hz to about 300 Hz, about 200 Hz to about 400 Hz, about 200 Hz to about 500 Hz, about 200 Hz to about 600 Hz, about 200 Hz to about 700 Hz, about 200 Hz to about 800 Hz, about 200 Hz to about 900 Hz, about 200 Hz to about 1,000 Hz, about 300 Hz to about 400 Hz, about 300 Hz to about 500 Hz, about 300 Hz to about 600 Hz, about 300 Hz to about 700 Hz, about 300 Hz to about 800 Hz, about 300 Hz to about 900 Hz, about 300 Hz to about 1,000 Hz, about 400 Hz to about 500 Hz, about 400 Hz to about 600 Hz, about 400 Hz to about 700 Hz, about 400 Hz to about 800 Hz, about 400 Hz to about 900 Hz, about 400 Hz to about 1,000 Hz, about 500 Hz to about 600 Hz, about 500 Hz to about 700 Hz, about 500 Hz to about 800 Hz, about 500 Hz to about 900 Hz, about 500 Hz to about 1,000 Hz, about 600 Hz to about 700 Hz, about 600 Hz to about 800 Hz, about 600 Hz to about 900 Hz, about 600 Hz to about 1,000 Hz, about 700 Hz to about 800 Hz, about 700 Hz to about 900 Hz, about 700 Hz to about 1,000 Hz, about 800 Hz to about 900 Hz, about 800 Hz to about 1,000 Hz, or about 900 Hz to about 1,000 Hz. In some embodiments, the time-varying voltage is from about 10 Hz, about 100 Hz, about 200 Hz, about 300 Hz, about 400 Hz, about 500 Hz, about 600 Hz, about 700 Hz, about 800 Hz, about 900 Hz, or about 1,000 Hz. In some embodiments, the time-varying voltage is from at least about 10 Hz, about 100 Hz, about 200 Hz, about 300 Hz, about 400 Hz, about 500 Hz, about 600 Hz, about 700 Hz, about 800 Hz, or about 900 Hz. In some embodiments, the time-varying voltage is from at most about 100 Hz, about 200 Hz, about 300 Hz, about 400 Hz, about 500 Hz, about 600 Hz, about 700 Hz, about 800 Hz, about 900 Hz, or about 1,000 Hz. In some embodiments, the time-varying voltage is from about 1 kHz to about 20 kHz. In some embodiments, the time-varying voltage is from about 1 kHz to about 2.5 kHz, about 1 kHz to about 5 kHz, about 1 kHz to about 7.5 kHz, about 1 kHz to about 10 kHz, about 1 kHz to about 12.5 kHz, about 1 kHz to about 15 kHz, about 1 kHz to about 17.5 kHz, about 1 kHz to about 20 kHz, about 2.5 kHz to about 5 kHz, about 2.5 kHz to about 7.5 kHz, about 2.5 kHz to about 10 kHz, about 2.5 kHz to about 12.5 kHz, about 2.5 kHz to about 15 kHz, about 2.5 kHz to about 17.5 kHz, about 2.5 kHz to about 20 kHz, about 5 kHz to about 7.5 kHz, about 5 kHz to about 10 kHz, about 5 kHz to about 12.5 kHz, about 5 kHz to about 15 kHz, about 5 kHz to about 17.5 kHz, about 5 kHz to about 20 kHz, about 7.5 kHz to about 10 kHz, about 7.5 kHz to about 12.5 kHz, about 7.5 kHz to about 15 kHz, about 7.5 kHz to about 17.5 kHz, about 7.5 kHz to about 20 kHz, about 10 kHz to about 12.5 kHz, about 10 kHz to about 15 kHz, about 10 kHz to about 17.5 kHz, about 10 kHz to about 20 kHz, about 12.5 kHz to about 15 kHz, about 12.5 kHz to about 17.5 kHz, about 12.5 kHz to about 20 kHz, about 15 kHz to about 17.5 kHz, about 15 kHz to about 20 kHz, or about 17.5 kHz to about 20 kHz. In some embodiments, the time-varying voltage is from about 1 kHz, about 2.5 kHz, about 5 kHz, about 7.5 kHz, about 10 kHz, about 12.5 kHz, about 15 kHz, about 17.5 kHz, or about 20 kHz. In some embodiments, the time-varying voltage is from at least about 1 kHz, about 2.5 kHz, about 5 kHz, about 7.5 kHz, about 10 kHz, about 12.5 kHz, about 15 kHz, or about 17.5 kHz. In some embodiments, the time-varying voltage is from at most about 2.5 kHz, about 5 kHz, about 7.5 kHz, about 10 kHz, about 12.5 kHz, about 15 kHz, about 17.5 kHz, or about 20 kHz.

In some embodiments, upon activation of at least the subset of the plurality of electrodes, the system further comprises a current-return path adjacent to the droplet and one or more inactive electrodes. In some embodiments, the activation of at least the subset of the plurality of electrodes generates an antagonistic current driving scheme in one or more adjacent electrodes. In some embodiments, the system further comprises a dielectric layer. In some embodiments, the dielectric layer comprises a thickness, wherein the thickness is sufficient to ground an electric current generated by the plurality of electrodes. In some embodiments, the thickness is 0.025 micrometer (μm) to 10,000 μm.

In some embodiments, the thickness of the dielectric layer is about 0.025 μm to about 10,000 μm. In some embodiments, the thickness of the dielectric layer is about 0.025 μm to about 0.05 μm, about 0.025 μm to about 0.1 μm, about 0.025 μm to about 1 μm, about 0.025 μm to about 10 μm, about 0.025 μm to about 50 μm, about 0.025 μm to about 100 μm, about 0.025 μm to about 200 μm, about 0.025 μm to about 500 μm, about 0.025 μm to about 1,000 μm, about 0.025 μm to about 5,000 μm, about 0.025 μm to about 10,000 μm, about 0.05 μm to about 0.1 μm, about 0.05 μm to about 1 μm, about 0.05 μm to about 10 μm, about 0.05 μm to about 50 μm, about 0.05 μm to about 100 μm, about 0.05 μm to about 200 μm, about 0.05 μm to about 500 μm, about 0.05 μm to about 1,000 μm, about 0.05 μm to about 5,000 μm, about 0.05 μm to about 10,000 μm, about 0.1 μm to about 1 μm, about 0.1 μm to about 10 μm, about 0.1 μm to about 50 μm, about 0.1 μm to about 100 μm, about 0.1 μm to about 200 μm, about 0.1 μm to about 500 μm, about 0.1 μm to about 1,000 μm, about 0.1 μm to about 5,000 μm, about 0.1 μm to about 10,000 μm, about 1 μm to about 10 μm, about 1 μm to about 50 μm, about 1 μm to about 100 μm, about 1 μm to about 200 μm, about 1 μm to about 500 μm, about 1 μm to about 1,000 μm, about 1 μm to about 5,000 μm, about 1 μm to about 10,000 μm, about 10 μm to about 50 μm, about 10 μm to about 100 μm, about 10 μm to about 200 μm, about 10 μm to about 500 μm, about 10 μm to about 1,000 μm, about 10 μm to about 5,000 μm, about 10 μm to about 10,000 μm, about 50 μm to about 100 μm, about 50 μm to about 200 μm, about 50 μm to about 500 μm, about 50 μm to about 1,000 μm, about 50 μm to about 5,000 μm, about 50 μm to about 10,000 μm, about 100 μm to about 200 μm, about 100 μm to about 500 μm, about 100 μm to about 1,000 μm, about 100 μm to about 5,000 μm, about 100 μm to about 10,000 μm, about 200 μm to about 500 μm, about 200 μm to about 1,000 μm, about 200 μm to about 5,000 μm, about 200 μm to about 10,000 μm, about 500 μm to about 1,000 μm, about 500 μm to about 5,000 μm, about 500 μm to about 10,000 μm, about 1,000 μm to about 5,000 μm, about 1,000 μm to about 10,000 μm, or about 5,000 μm to about 10,000 μm. In some embodiments, the thickness of the dielectric layer is about 0.025 μm, about 0.05 μm, about 0.1 μm, about 1 μm, about 10 μm, about 50 μm, about 100 μm, about 200 μm, about 500 μm, about 1,000 μm, about 5,000 μm, or about 10,000 μm. In some embodiments, the thickness of the dielectric layer is at least about 0.025 μm, about 0.05 μm, about 0.1 μm, about 1 μm, about 10 μm, about 50 μm, about 100 μm, about 200 μm, about 500 μm, about 1,000 μm, or about 5,000 μm. In some embodiments, the thickness of the dielectric layer is at most about 0.05 μm, about 0.1 μm, about 1 μm, about 10 μm, about 50 μm, about 100 μm, about 200 μm, about 500 μm, about 1,000 μm, about 5,000 μm, or about 10,000 μm.

In some embodiments, the dielectric layer comprises a natural polymeric material, a synthetic polymeric material, a fluorinated material, a surface modification, or any combination thereof. In some embodiments, the natural polymeric material comprises shellac, amber, wool, silk, natural rubber, cellulose, wax, chiton, or any combination thereof. In some embodiments, the synthetic polymeric material comprises polyethylene, polypropylene, polystyrene, polyetheretherketone (PEEK), polyimide, polyacetal, polysilfone, polyphenulene ether, polyphenylene Sulfide (PPS), polyvinyl chloride, synthetic rubber, neoprene, nylon, polyacrylonitrile, polyvinyl butyral, silicone, parafilm, polyethylene terephthalate, polybutylene terephthalate, polyamides, polyoxymethlyene, polycarbonate, polymethylpentene, polyphenylene oxide (Polyphenyl ether), polyphthalamide (PPA), polylactic acid, synthetic cellulose ethers (e.g., methyl cellulose, ethyl cellulose, propyl cellulose, hydroxyethyl cellulose, hydroxyethyl cellulose, hydroxypropyl cellulose (HPC), hydroxyethyl methyl cellulose, hydroxypropyl methyl cellulose (HPMC), ethyl hydroxyethyl cellulose), paraffins, microcrystalline wax, epoxy, or any combination thereof. In some embodiments, the fluorinated material comprises polytetrafluoroethylene (PTFE), tetrafluoroethylene (TFE), fluorinated ethylenepropylene copolymer (FEP), polyvinylidene fluoride (PVDF), perfluoroalkoxytetrafluoroethylene copolymer (PFA), perfluoromethyl vinylether copolymer (MFA), ethylenechlorotrifluoroethylene copolymer (ECTFE), ethylene-tetrafluoroethylene copolymer (ETFE), perfluoropolyether (PFPE), polychlorotetrafluoroethylene (PCTFE), or any combination thereof. In some embodiments, the surface modification comprises silicone, silane, fluoro-polymer treatment, parylene coating, any other suitable surface chemistry modification process, ceramic, clay minerals, bentonite, kaolinite, vermiculite, graphite, molybdenum disulfide, mica, boron nitride, sodium formate, sodium oleate, sodium palmitate, sodium sulfate, sodium alginate, or any combination thereof. In some embodiments, the surface comprises a liquid layer. In some embodiments, the liquid layer comprises silicone oils, fluorinated oils, ionic liquids, mineral oils, ferrofluids, polyphenyl ether, vegetable oil, esters of saturated fatty and dibasic acids, grease, fatty acids, triglycerides, polyalphaolefin, polyglycol hydrocarbons, other Non-hydrocarbon synthetic oils, or any combination thereof. In some embodiments, the liquid layer further comprises surfactants, electrolytes, rheology modifier, wax, graphite, graphene, molybdenum disulfide, PTFE particles, or any combination thereof. In some embodiments, the system further comprises a liquid disposed in an interspace adjacent to the dielectric layer and the plurality of electrodes. In some embodiments, the liquid generates adhesion between the plurality of electrodes and the dielectric layer. In some embodiments, the liquid comprises a dielectric material. In some embodiments, the liquid prevents or reduces electrical conductivity of air disposed in the interspace. In some embodiments, the liquid comprises silicone oils, fluorinated oils, ionic liquids, mineral oils, ferrofluids, polyphenyl ether, vegetable oil, esters of saturated fatty and dibasic acids, grease, fatty acids, triglycerides, polyalphaolefin, polyglycol hydrocarbons, other Non-hydrocarbon synthetic oils, or any combination thereof. In some embodiments, the liquid further comprises surfactants, electrolytes, rheology modifier, wax, graphite, graphene, molybdenum disulfide, PTFE particles, or any combination thereof.

Other embodiments of this aspect of the present disclosure comprise a system for processing a droplet, the system comprising: an array comprising: a plurality of electrodes, wherein no electrode of the plurality of electrodes is permanently grounded; and a surface configured to support a droplet comprising the sample; a controller operatively coupled to the plurality of electrodes, wherein the controller is configured to: activate at least a subset of the plurality of electrodes with a voltage to alter a wetting characteristic of the surface; wherein the array does not comprise a permanent reference electrode. In some embodiments, the voltage is a time-varying voltage. In some embodiments, the system does not comprise an overlying electrode. In some embodiments, the plurality of electrodes comprises at least one electrode comprising a cross-section or overlap with the droplet sufficient to generate a current-return path adjacent to the electrode and an adjacent electrode. In some embodiments, the plurality of electrodes are co-planar. In some embodiments, the time-varying voltage is bipolar. In some embodiments, the time-varying voltage is from about 1 Hz to about 20 kHz.

In some embodiments, the time-varying voltage is from 1 Hertz (hz) to 20 kilohertz (khz). In some embodiments, the time-varying voltage is from 1 Hertz (hz) to 20 kilohertz (khz). In some embodiments, the time-varying voltage is from about 1 Hz to about 10 Hz. In some embodiments, the time-varying voltage is from about 1 Hz to about 2 Hz, about 1 Hz to about 3 Hz, about 1 Hz to about 4 Hz, about 1 Hz to about 5 Hz, about 1 Hz to about 6 Hz, about 1 Hz to about 7 Hz, about 1 Hz to about 8 Hz, about 1 Hz to about 9 Hz, about 1 Hz to about 10 Hz, about 2 Hz to about 3 Hz, about 2 Hz to about 4 Hz, about 2 Hz to about 5 Hz, about 2 Hz to about 6 Hz, about 2 Hz to about 7 Hz, about 2 Hz to about 8 Hz, about 2 Hz to about 9 Hz, about 2 Hz to about 10 Hz, about 3 Hz to about 4 Hz, about 3 Hz to about 5 Hz, about 3 Hz to about 6 Hz, about 3 Hz to about 7 Hz, about 3 Hz to about 8 Hz, about 3 Hz to about 9 Hz, about 3 Hz to about 10 Hz, about 4 Hz to about 5 Hz, about 4 Hz to about 6 Hz, about 4 Hz to about 7 Hz, about 4 Hz to about 8 Hz, about 4 Hz to about 9 Hz, about 4 Hz to about 10 Hz, about 5 Hz to about 6 Hz, about 5 Hz to about 7 Hz, about 5 Hz to about 8 Hz, about 5 Hz to about 9 Hz, about 5 Hz to about 10 Hz, about 6 Hz to about 7 Hz, about 6 Hz to about 8 Hz, about 6 Hz to about 9 Hz, about 6 Hz to about 10 Hz, about 7 Hz to about 8 Hz, about 7 Hz to about 9 Hz, about 7 Hz to about 10 Hz, about 8 Hz to about 9 Hz, about 8 Hz to about 10 Hz, or about 9 Hz to about 10 Hz. In some embodiments, the time-varying voltage is from about 1 Hz, about 2 Hz, about 3 Hz, about 4 Hz, about 5 Hz, about 6 Hz, about 7 Hz, about 8 Hz, about 9 Hz, or about 10 Hz. In some embodiments, the time-varying voltage is from at least about 1 Hz, about 2 Hz, about 3 Hz, about 4 Hz, about 5 Hz, about 6 Hz, about 7 Hz, about 8 Hz, or about 9 Hz. In some embodiments, the time-varying voltage is from at most about 2 Hz, about 3 Hz, about 4 Hz, about 5 Hz, about 6 Hz, about 7 Hz, about 8 Hz, about 9 Hz, or about 10 Hz. In some embodiments, the time-varying voltage is from about 10 Hz to about 1,000 Hz. In some embodiments, the time-varying voltage is from about 10 Hz to about 100 Hz, about 10 Hz to about 200 Hz, about 10 Hz to about 300 Hz, about 10 Hz to about 400 Hz, about 10 Hz to about 500 Hz, about 10 Hz to about 600 Hz, about 10 Hz to about 700 Hz, about 10 Hz to about 800 Hz, about 10 Hz to about 900 Hz, about 10 Hz to about 1,000 Hz, about 100 Hz to about 200 Hz, about 100 Hz to about 300 Hz, about 100 Hz to about 400 Hz, about 100 Hz to about 500 Hz, about 100 Hz to about 600 Hz, about 100 Hz to about 700 Hz, about 100 Hz to about 800 Hz, about 100 Hz to about 900 Hz, about 100 Hz to about 1,000 Hz, about 200 Hz to about 300 Hz, about 200 Hz to about 400 Hz, about 200 Hz to about 500 Hz, about 200 Hz to about 600 Hz, about 200 Hz to about 700 Hz, about 200 Hz to about 800 Hz, about 200 Hz to about 900 Hz, about 200 Hz to about 1,000 Hz, about 300 Hz to about 400 Hz, about 300 Hz to about 500 Hz, about 300 Hz to about 600 Hz, about 300 Hz to about 700 Hz, about 300 Hz to about 800 Hz, about 300 Hz to about 900 Hz, about 300 Hz to about 1,000 Hz, about 400 Hz to about 500 Hz, about 400 Hz to about 600 Hz, about 400 Hz to about 700 Hz, about 400 Hz to about 800 Hz, about 400 Hz to about 900 Hz, about 400 Hz to about 1,000 Hz, about 500 Hz to about 600 Hz, about 500 Hz to about 700 Hz, about 500 Hz to about 800 Hz, about 500 Hz to about 900 Hz, about 500 Hz to about 1,000 Hz, about 600 Hz to about 700 Hz, about 600 Hz to about 800 Hz, about 600 Hz to about 900 Hz, about 600 Hz to about 1,000 Hz, about 700 Hz to about 800 Hz, about 700 Hz to about 900 Hz, about 700 Hz to about 1,000 Hz, about 800 Hz to about 900 Hz, about 800 Hz to about 1,000 Hz, or about 900 Hz to about 1,000 Hz. In some embodiments, the time-varying voltage is from about 10 Hz, about 100 Hz, about 200 Hz, about 300 Hz, about 400 Hz, about 500 Hz, about 600 Hz, about 700 Hz, about 800 Hz, about 900 Hz, or about 1,000 Hz. In some embodiments, the time-varying voltage is from at least about 10 Hz, about 100 Hz, about 200 Hz, about 300 Hz, about 400 Hz, about 500 Hz, about 600 Hz, about 700 Hz, about 800 Hz, or about 900 Hz. In some embodiments, the time-varying voltage is from at most about 100 Hz, about 200 Hz, about 300 Hz, about 400 Hz, about 500 Hz, about 600 Hz, about 700 Hz, about 800 Hz, about 900 Hz, or about 1,000 Hz. In some embodiments, the time-varying voltage is from about 1 kHz to about 20 kHz. In some embodiments, the time-varying voltage is from about 1 kHz to about 2.5 kHz, about 1 kHz to about 5 kHz, about 1 kHz to about 7.5 kHz, about 1 kHz to about 10 kHz, about 1 kHz to about 12.5 kHz, about 1 kHz to about 15 kHz, about 1 kHz to about 17.5 kHz, about 1 kHz to about 20 kHz, about 2.5 kHz to about 5 kHz, about 2.5 kHz to about 7.5 kHz, about 2.5 kHz to about 10 kHz, about 2.5 kHz to about 12.5 kHz, about 2.5 kHz to about 15 kHz, about 2.5 kHz to about 17.5 kHz, about 2.5 kHz to about 20 kHz, about 5 kHz to about 7.5 kHz, about 5 kHz to about 10 kHz, about 5 kHz to about 12.5 kHz, about 5 kHz to about 15 kHz, about 5 kHz to about 17.5 kHz, about 5 kHz to about 20 kHz, about 7.5 kHz to about 10 kHz, about 7.5 kHz to about 12.5 kHz, about 7.5 kHz to about 15 kHz, about 7.5 kHz to about 17.5 kHz, about 7.5 kHz to about 20 kHz, about 10 kHz to about 12.5 kHz, about 10 kHz to about 15 kHz, about 10 kHz to about 17.5 kHz, about 10 kHz to about 20 kHz, about 12.5 kHz to about 15 kHz, about 12.5 kHz to about 17.5 kHz, about 12.5 kHz to about 20 kHz, about 15 kHz to about 17.5 kHz, about 15 kHz to about 20 kHz, or about 17.5 kHz to about 20 kHz. In some embodiments, the time-varying voltage is from about 1 kHz, about 2.5 kHz, about 5 kHz, about 7.5 kHz, about 10 kHz, about 12.5 kHz, about 15 kHz, about 17.5 kHz, or about 20 kHz. In some embodiments, the time-varying voltage is from at least about 1 kHz, about 2.5 kHz, about 5 kHz, about 7.5 kHz, about 10 kHz, about 12.5 kHz, about 15 kHz, or about 17.5 kHz. In some embodiments, the time-varying voltage is from at most about 2.5 kHz, about 5 kHz, about 7.5 kHz, about 10 kHz, about 12.5 kHz, about 15 kHz, about 17.5 kHz, or about 20 kHz.

In some embodiments, upon activation of at least the subset of the plurality of electrodes, the system further comprises a current-return path adjacent to the droplet and one or more inactive electrodes. In some embodiments, the activation of at least the subset of the plurality of electrodes generates an antagonistic current driving scheme in one or more adjacent electrodes. In some embodiments, the system further comprises a dielectric layer. In some embodiments, the dielectric layer comprises a thickness, wherein the thickness is sufficient to ground an electric current generated by the plurality of electrodes. In some embodiments, the thickness is 0.025 micrometer (μm) to 10,000 μm.

In some embodiments, the thickness of the dielectric layer is about 0.025 μm to about 10,000 μm. In some embodiments, the thickness of the dielectric layer is about 0.025 μm to about 0.05 μm, about 0.025 μm to about 0.1 μm, about 0.025 μm to about 1 μm, about 0.025 μm to about 10 μm, about 0.025 μm to about 50 μm, about 0.025 μm to about 100 μm, about 0.025 μm to about 200 μm, about 0.025 μm to about 500 μm, about 0.025 μm to about 1,000 μm, about 0.025 μm to about 5,000 μm, about 0.025 μm to about 10,000 μm, about 0.05 μm to about 0.1 μm, about 0.05 μm to about 1 μm, about 0.05 μm to about 10 μm, about 0.05 μm to about 50 μm, about 0.05 μm to about 100 μm, about 0.05 μm to about 200 μm, about 0.05 μm to about 500 μm, about 0.05 μm to about 1,000 μm, about 0.05 μm to about 5,000 μm, about 0.05 μm to about 10,000 μm, about 0.1 μm to about 1 μm, about 0.1 μm to about 10 μm, about 0.1 μm to about 50 μm, about 0.1 μm to about 100 μm, about 0.1 μm to about 200 μm, about 0.1 μm to about 500 μm, about 0.1 μm to about 1,000 μm, about 0.1 μm to about 5,000 μm, about 0.1 μm to about 10,000 μm, about 1 μm to about 10 μm, about 1 μm to about 50 μm, about 1 μm to about 100 μm, about 1 μm to about 200 μm, about 1 μm to about 500 μm, about 1 μm to about 1,000 μm, about 1 μm to about 5,000 μm, about 1 μm to about 10,000 μm, about 10 μm to about 50 μm, about 10 μm to about 100 μm, about 10 μm to about 200 μm, about 10 μm to about 500 μm, about 10 μm to about 1,000 μm, about 10 μm to about 5,000 μm, about 10 μm to about 10,000 μm, about 50 μm to about 100 μm, about 50 μm to about 200 μm, about 50 μm to about 500 μm, about 50 μm to about 1,000 μm, about 50 μm to about 5,000 μm, about 50 μm to about 10,000 μm, about 100 μm to about 200 μm, about 100 μm to about 500 μm, about 100 μm to about 1,000 μm, about 100 μm to about 5,000 μm, about 100 μm to about 10,000 μm, about 200 μm to about 500 μm, about 200 μm to about 1,000 μm, about 200 μm to about 5,000 μm, about 200 μm to about 10,000 μm, about 500 μm to about 1,000 μm, about 500 μm to about 5,000 μm, about 500 μm to about 10,000 μm, about 1,000 μm to about 5,000 μm, about 1,000 μm to about 10,000 μm, or about 5,000 μm to about 10,000 μm. In some embodiments, the thickness of the dielectric layer is about 0.025 μm, about 0.05 μm, about 0.1 μm, about 1 μm, about 10 μm, about 50 μm, about 100 μm, about 200 μm, about 500 μm, about 1,000 μm, about 5,000 μm, or about 10,000 μm. In some embodiments, the thickness of the dielectric layer is at least about 0.025 μm, about 0.05 μm, about 0.1 μm, about 1 μm, about 10 μm, about 50 μm, about 100 μm, about 200 μm, about 500 μm, about 1,000 μm, or about 5,000 μm. In some embodiments, the thickness of the dielectric layer is at most about 0.05 μm, about 0.1 μm, about 1 μm, about 10 μm, about 50 μm, about 100 μm, about 200 μm, about 500 μm, about 1,000 μm, about 5,000 μm, or about 10,000 μm.

In some embodiments, the dielectric layer comprises a natural polymeric material, a synthetic polymeric material, a fluorinated material, a surface modification, or any combination thereof. In some embodiments, the natural polymeric material comprises shellac, amber, wool, silk, natural rubber, cellulose, wax, chiton, or any combination thereof. In some embodiments, the synthetic polymeric material comprises polyethylene, polypropylene, polystyrene, polyetheretherketone (PEEK), polyimide, polyacetal, polysilfone, polyphenulene ether, polyphenylene Sulfide (PPS), polyvinyl chloride, synthetic rubber, neoprene, nylon, polyacrylonitrile, polyvinyl butyral, silicone, parafilm, polyethylene terephthalate, polybutylene terephthalate, polyamides, polyoxymethlyene, polycarbonate, polymethylpentene, polyphenylene oxide (Polyphenyl ether), polyphthalamide (PPA), polylactic acid, synthetic cellulose ethers (e.g., methyl cellulose, ethyl cellulose, propyl cellulose, hydroxyethyl cellulose, hydroxyethyl cellulose, hydroxypropyl cellulose (HPC), hydroxyethyl methyl cellulose, hydroxypropyl methyl cellulose (HPMC), ethyl hydroxyethyl cellulose), paraffins, microcrystalline wax, epoxy, or any combination thereof. In some embodiments, the fluorinated material comprises polytetrafluoroethylene (PTFE), tetrafluoroethylene (TFE), fluorinated ethylenepropylene copolymer (FEP), polyvinylidene fluoride (PVDF), perfluoroalkoxytetrafluoroethylene copolymer (PFA), perfluoromethyl vinylether copolymer (MFA), ethylenechlorotrifluoroethylene copolymer (ECTFE), ethylene-tetrafluoroethylene copolymer (ETFE), perfluoropolyether (PFPE), polychlorotetrafluoroethylene (PCTFE), or any combination thereof. In some embodiments, the surface modification comprises silicone, silane, fluoro-polymer treatment, parylene coating, any other suitable surface chemistry modification process, ceramic, clay minerals, bentonite, kaolinite, vermiculite, graphite, molybdenum disulfide, mica, boron nitride, sodium formate, sodium oleate, sodium palmitate, sodium sulfate, sodium alginate, or any combination thereof. In some embodiments, the surface comprises a liquid layer. In some embodiments, the liquid layer comprises silicone oils, fluorinated oils, ionic liquids, mineral oils, ferrofluids, polyphenyl ether, vegetable oil, esters of saturated fatty and dibasic acids, grease, fatty acids, triglycerides, polyalphaolefin, polyglycol hydrocarbons, other Non-hydrocarbon synthetic oils, or any combination thereof. In some embodiments, the liquid layer further comprises surfactants, electrolytes, rheology modifier, wax, graphite, graphene, molybdenum disulfide, PTFE particles, or any combination thereof. In some embodiments, the system further comprises a liquid disposed in an interspace adjacent to the dielectric layer and the plurality of electrodes. In some embodiments, the liquid generates adhesion between the plurality of electrodes and the dielectric layer. In some embodiments, the liquid comprises a dielectric material. In some embodiments, the liquid prevents or reduces electrical conductivity of air disposed in the interspace. In some embodiments, the liquid comprises silicone oils, fluorinated oils, ionic liquids, mineral oils, ferrofluids, polyphenyl ether, vegetable oil, esters of saturated fatty and dibasic acids, grease, fatty acids, triglycerides, polyalphaolefin, polyglycol hydrocarbons, other Non-hydrocarbon synthetic oils, or any combination thereof. In some embodiments, the liquid further comprises surfactants, electrolytes, rheology modifier, wax, graphite, graphene, molybdenum disulfide, PTFE particles, or any combination thereof.

Another embodiment of this aspect of the disclosure comprises a method for motioning a droplet over an array, wherein the array comprises a plurality of electrodes, wherein no electrode of the plurality of electrodes is permanently grounded; and a surface configured to support the droplet comprising the sample, the method comprising: activating at least a subset of the plurality of electrodes with a time-varying voltage to alter a wetting characteristic of the surface; wherein the time-varying voltage generates a current-return path adjacent to the droplet and one or more inactive electrodes, thereby inducing motion of the droplet. In some embodiments, the plurality of electrodes are co-planar. In some embodiments, the time-varying voltage is bipolar. In some embodiments, the time-varying voltage is from about 1 Hz to about 20 kHz.

In some embodiments, the time-varying voltage is from 1 Hertz (hz) to 20 kilohertz (khz). In some embodiments, the time-varying voltage is from 1 Hertz (hz) to 20 kilohertz (khz). In some embodiments, the time-varying voltage is from about 1 Hz to about 10 Hz. In some embodiments, the time-varying voltage is from about 1 Hz to about 2 Hz, about 1 Hz to about 3 Hz, about 1 Hz to about 4 Hz, about 1 Hz to about 5 Hz, about 1 Hz to about 6 Hz, about 1 Hz to about 7 Hz, about 1 Hz to about 8 Hz, about 1 Hz to about 9 Hz, about 1 Hz to about 10 Hz, about 2 Hz to about 3 Hz, about 2 Hz to about 4 Hz, about 2 Hz to about 5 Hz, about 2 Hz to about 6 Hz, about 2 Hz to about 7 Hz, about 2 Hz to about 8 Hz, about 2 Hz to about 9 Hz, about 2 Hz to about 10 Hz, about 3 Hz to about 4 Hz, about 3 Hz to about 5 Hz, about 3 Hz to about 6 Hz, about 3 Hz to about 7 Hz, about 3 Hz to about 8 Hz, about 3 Hz to about 9 Hz, about 3 Hz to about 10 Hz, about 4 Hz to about 5 Hz, about 4 Hz to about 6 Hz, about 4 Hz to about 7 Hz, about 4 Hz to about 8 Hz, about 4 Hz to about 9 Hz, about 4 Hz to about 10 Hz, about 5 Hz to about 6 Hz, about 5 Hz to about 7 Hz, about 5 Hz to about 8 Hz, about 5 Hz to about 9 Hz, about 5 Hz to about 10 Hz, about 6 Hz to about 7 Hz, about 6 Hz to about 8 Hz, about 6 Hz to about 9 Hz, about 6 Hz to about 10 Hz, about 7 Hz to about 8 Hz, about 7 Hz to about 9 Hz, about 7 Hz to about 10 Hz, about 8 Hz to about 9 Hz, about 8 Hz to about 10 Hz, or about 9 Hz to about 10 Hz. In some embodiments, the time-varying voltage is from about 1 Hz, about 2 Hz, about 3 Hz, about 4 Hz, about 5 Hz, about 6 Hz, about 7 Hz, about 8 Hz, about 9 Hz, or about 10 Hz. In some embodiments, the time-varying voltage is from at least about 1 Hz, about 2 Hz, about 3 Hz, about 4 Hz, about 5 Hz, about 6 Hz, about 7 Hz, about 8 Hz, or about 9 Hz. In some embodiments, the time-varying voltage is from at most about 2 Hz, about 3 Hz, about 4 Hz, about 5 Hz, about 6 Hz, about 7 Hz, about 8 Hz, about 9 Hz, or about 10 Hz. In some embodiments, the time-varying voltage is from about 10 Hz to about 1,000 Hz. In some embodiments, the time-varying voltage is from about 10 Hz to about 100 Hz, about 10 Hz to about 200 Hz, about 10 Hz to about 300 Hz, about 10 Hz to about 400 Hz, about 10 Hz to about 500 Hz, about 10 Hz to about 600 Hz, about 10 Hz to about 700 Hz, about 10 Hz to about 800 Hz, about 10 Hz to about 900 Hz, about 10 Hz to about 1,000 Hz, about 100 Hz to about 200 Hz, about 100 Hz to about 300 Hz, about 100 Hz to about 400 Hz, about 100 Hz to about 500 Hz, about 100 Hz to about 600 Hz, about 100 Hz to about 700 Hz, about 100 Hz to about 800 Hz, about 100 Hz to about 900 Hz, about 100 Hz to about 1,000 Hz, about 200 Hz to about 300 Hz, about 200 Hz to about 400 Hz, about 200 Hz to about 500 Hz, about 200 Hz to about 600 Hz, about 200 Hz to about 700 Hz, about 200 Hz to about 800 Hz, about 200 Hz to about 900 Hz, about 200 Hz to about 1,000 Hz, about 300 Hz to about 400 Hz, about 300 Hz to about 500 Hz, about 300 Hz to about 600 Hz, about 300 Hz to about 700 Hz, about 300 Hz to about 800 Hz, about 300 Hz to about 900 Hz, about 300 Hz to about 1,000 Hz, about 400 Hz to about 500 Hz, about 400 Hz to about 600 Hz, about 400 Hz to about 700 Hz, about 400 Hz to about 800 Hz, about 400 Hz to about 900 Hz, about 400 Hz to about 1,000 Hz, about 500 Hz to about 600 Hz, about 500 Hz to about 700 Hz, about 500 Hz to about 800 Hz, about 500 Hz to about 900 Hz, about 500 Hz to about 1,000 Hz, about 600 Hz to about 700 Hz, about 600 Hz to about 800 Hz, about 600 Hz to about 900 Hz, about 600 Hz to about 1,000 Hz, about 700 Hz to about 800 Hz, about 700 Hz to about 900 Hz, about 700 Hz to about 1,000 Hz, about 800 Hz to about 900 Hz, about 800 Hz to about 1,000 Hz, or about 900 Hz to about 1,000 Hz. In some embodiments, the time-varying voltage is from about 10 Hz, about 100 Hz, about 200 Hz, about 300 Hz, about 400 Hz, about 500 Hz, about 600 Hz, about 700 Hz, about 800 Hz, about 900 Hz, or about 1,000 Hz. In some embodiments, the time-varying voltage is from at least about 10 Hz, about 100 Hz, about 200 Hz, about 300 Hz, about 400 Hz, about 500 Hz, about 600 Hz, about 700 Hz, about 800 Hz, or about 900 Hz. In some embodiments, the time-varying voltage is from at most about 100 Hz, about 200 Hz, about 300 Hz, about 400 Hz, about 500 Hz, about 600 Hz, about 700 Hz, about 800 Hz, about 900 Hz, or about 1,000 Hz. In some embodiments, the time-varying voltage is from about 1 kHz to about 20 kHz. In some embodiments, the time-varying voltage is from about 1 kHz to about 2.5 kHz, about 1 kHz to about 5 kHz, about 1 kHz to about 7.5 kHz, about 1 kHz to about 10 kHz, about 1 kHz to about 12.5 kHz, about 1 kHz to about 15 kHz, about 1 kHz to about 17.5 kHz, about 1 kHz to about 20 kHz, about 2.5 kHz to about 5 kHz, about 2.5 kHz to about 7.5 kHz, about 2.5 kHz to about 10 kHz, about 2.5 kHz to about 12.5 kHz, about 2.5 kHz to about 15 kHz, about 2.5 kHz to about 17.5 kHz, about 2.5 kHz to about 20 kHz, about 5 kHz to about 7.5 kHz, about 5 kHz to about 10 kHz, about 5 kHz to about 12.5 kHz, about 5 kHz to about 15 kHz, about 5 kHz to about 17.5 kHz, about 5 kHz to about 20 kHz, about 7.5 kHz to about 10 kHz, about 7.5 kHz to about 12.5 kHz, about 7.5 kHz to about 15 kHz, about 7.5 kHz to about 17.5 kHz, about 7.5 kHz to about 20 kHz, about 10 kHz to about 12.5 kHz, about 10 kHz to about 15 kHz, about 10 kHz to about 17.5 kHz, about 10 kHz to about 20 kHz, about 12.5 kHz to about 15 kHz, about 12.5 kHz to about 17.5 kHz, about 12.5 kHz to about 20 kHz, about 15 kHz to about 17.5 kHz, about 15 kHz to about 20 kHz, or about 17.5 kHz to about 20 kHz. In some embodiments, the time-varying voltage is from about 1 kHz, about 2.5 kHz, about 5 kHz, about 7.5 kHz, about 10 kHz, about 12.5 kHz, about 15 kHz, about 17.5 kHz, or about 20 kHz. In some embodiments, the time-varying voltage is from at least about 1 kHz, about 2.5 kHz, about 5 kHz, about 7.5 kHz, about 10 kHz, about 12.5 kHz, about 15 kHz, or about 17.5 kHz. In some embodiments, the time-varying voltage is from at most about 2.5 kHz, about 5 kHz, about 7.5 kHz, about 10 kHz, about 12.5 kHz, about 15 kHz, about 17.5 kHz, or about 20 kHz.

In some embodiments, upon activation of at least the subset of the plurality of electrodes, the system further comprises a current-return path adjacent to the droplet and one or more inactive electrodes. In some embodiments, the activation of at least the subset of the plurality of electrodes generates an antagonistic current driving scheme in one or more adjacent electrodes.

Another embodiment of this aspect of the disclosure comprises a method for motioning a droplet over an array, wherein the array comprises a plurality of electrodes, wherein no electrode of the plurality of electrodes is permanently grounded; and a surface configured to support the droplet comprising the sample, the method comprising: activating at least a subset of the plurality of electrodes with a voltage to alter a wetting characteristic of the surface; wherein the array does not comprise a permanent reference electrode. wherein the time-varying voltage generates a current-return path adjacent to the droplet and one or more inactive electrodes, thereby inducing motion of the droplet. In some embodiments, the plurality of electrodes are co-planar. In some embodiments, the time-varying voltage is bipolar. In some embodiments, the time-varying voltage is from about 1 Hz to about 20 kHz.

In some embodiments, the time-varying voltage is from 1 Hertz (hz) to 20 kilohertz (khz). In some embodiments, the time-varying voltage is from 1 Hertz (hz) to 20 kilohertz (khz). In some embodiments, the time-varying voltage is from about 1 Hz to about 10 Hz. In some embodiments, the time-varying voltage is from about 1 Hz to about 2 Hz, about 1 Hz to about 3 Hz, about 1 Hz to about 4 Hz, about 1 Hz to about 5 Hz, about 1 Hz to about 6 Hz, about 1 Hz to about 7 Hz, about 1 Hz to about 8 Hz, about 1 Hz to about 9 Hz, about 1 Hz to about 10 Hz, about 2 Hz to about 3 Hz, about 2 Hz to about 4 Hz, about 2 Hz to about 5 Hz, about 2 Hz to about 6 Hz, about 2 Hz to about 7 Hz, about 2 Hz to about 8 Hz, about 2 Hz to about 9 Hz, about 2 Hz to about 10 Hz, about 3 Hz to about 4 Hz, about 3 Hz to about 5 Hz, about 3 Hz to about 6 Hz, about 3 Hz to about 7 Hz, about 3 Hz to about 8 Hz, about 3 Hz to about 9 Hz, about 3 Hz to about 10 Hz, about 4 Hz to about 5 Hz, about 4 Hz to about 6 Hz, about 4 Hz to about 7 Hz, about 4 Hz to about 8 Hz, about 4 Hz to about 9 Hz, about 4 Hz to about 10 Hz, about 5 Hz to about 6 Hz, about 5 Hz to about 7 Hz, about 5 Hz to about 8 Hz, about 5 Hz to about 9 Hz, about 5 Hz to about 10 Hz, about 6 Hz to about 7 Hz, about 6 Hz to about 8 Hz, about 6 Hz to about 9 Hz, about 6 Hz to about 10 Hz, about 7 Hz to about 8 Hz, about 7 Hz to about 9 Hz, about 7 Hz to about 10 Hz, about 8 Hz to about 9 Hz, about 8 Hz to about 10 Hz, or about 9 Hz to about 10 Hz. In some embodiments, the time-varying voltage is from about 1 Hz, about 2 Hz, about 3 Hz, about 4 Hz, about 5 Hz, about 6 Hz, about 7 Hz, about 8 Hz, about 9 Hz, or about 10 Hz. In some embodiments, the time-varying voltage is from at least about 1 Hz, about 2 Hz, about 3 Hz, about 4 Hz, about 5 Hz, about 6 Hz, about 7 Hz, about 8 Hz, or about 9 Hz. In some embodiments, the time-varying voltage is from at most about 2 Hz, about 3 Hz, about 4 Hz, about 5 Hz, about 6 Hz, about 7 Hz, about 8 Hz, about 9 Hz, or about 10 Hz. In some embodiments, the time-varying voltage is from about 10 Hz to about 1,000 Hz. In some embodiments, the time-varying voltage is from about 10 Hz to about 100 Hz, about 10 Hz to about 200 Hz, about 10 Hz to about 300 Hz, about 10 Hz to about 400 Hz, about 10 Hz to about 500 Hz, about 10 Hz to about 600 Hz, about 10 Hz to about 700 Hz, about 10 Hz to about 800 Hz, about 10 Hz to about 900 Hz, about 10 Hz to about 1,000 Hz, about 100 Hz to about 200 Hz, about 100 Hz to about 300 Hz, about 100 Hz to about 400 Hz, about 100 Hz to about 500 Hz, about 100 Hz to about 600 Hz, about 100 Hz to about 700 Hz, about 100 Hz to about 800 Hz, about 100 Hz to about 900 Hz, about 100 Hz to about 1,000 Hz, about 200 Hz to about 300 Hz, about 200 Hz to about 400 Hz, about 200 Hz to about 500 Hz, about 200 Hz to about 600 Hz, about 200 Hz to about 700 Hz, about 200 Hz to about 800 Hz, about 200 Hz to about 900 Hz, about 200 Hz to about 1,000 Hz, about 300 Hz to about 400 Hz, about 300 Hz to about 500 Hz, about 300 Hz to about 600 Hz, about 300 Hz to about 700 Hz, about 300 Hz to about 800 Hz, about 300 Hz to about 900 Hz, about 300 Hz to about 1,000 Hz, about 400 Hz to about 500 Hz, about 400 Hz to about 600 Hz, about 400 Hz to about 700 Hz, about 400 Hz to about 800 Hz, about 400 Hz to about 900 Hz, about 400 Hz to about 1,000 Hz, about 500 Hz to about 600 Hz, about 500 Hz to about 700 Hz, about 500 Hz to about 800 Hz, about 500 Hz to about 900 Hz, about 500 Hz to about 1,000 Hz, about 600 Hz to about 700 Hz, about 600 Hz to about 800 Hz, about 600 Hz to about 900 Hz, about 600 Hz to about 1,000 Hz, about 700 Hz to about 800 Hz, about 700 Hz to about 900 Hz, about 700 Hz to about 1,000 Hz, about 800 Hz to about 900 Hz, about 800 Hz to about 1,000 Hz, or about 900 Hz to about 1,000 Hz. In some embodiments, the time-varying voltage is from about 10 Hz, about 100 Hz, about 200 Hz, about 300 Hz, about 400 Hz, about 500 Hz, about 600 Hz, about 700 Hz, about 800 Hz, about 900 Hz, or about 1,000 Hz. In some embodiments, the time-varying voltage is from at least about 10 Hz, about 100 Hz, about 200 Hz, about 300 Hz, about 400 Hz, about 500 Hz, about 600 Hz, about 700 Hz, about 800 Hz, or about 900 Hz. In some embodiments, the time-varying voltage is from at most about 100 Hz, about 200 Hz, about 300 Hz, about 400 Hz, about 500 Hz, about 600 Hz, about 700 Hz, about 800 Hz, about 900 Hz, or about 1,000 Hz. In some embodiments, the time-varying voltage is from about 1 kHz to about 20 kHz. In some embodiments, the time-varying voltage is from about 1 kHz to about 2.5 kHz, about 1 kHz to about 5 kHz, about 1 kHz to about 7.5 kHz, about 1 kHz to about 10 kHz, about 1 kHz to about 12.5 kHz, about 1 kHz to about 15 kHz, about 1 kHz to about 17.5 kHz, about 1 kHz to about 20 kHz, about 2.5 kHz to about 5 kHz, about 2.5 kHz to about 7.5 kHz, about 2.5 kHz to about 10 kHz, about 2.5 kHz to about 12.5 kHz, about 2.5 kHz to about 15 kHz, about 2.5 kHz to about 17.5 kHz, about 2.5 kHz to about 20 kHz, about 5 kHz to about 7.5 kHz, about 5 kHz to about 10 kHz, about 5 kHz to about 12.5 kHz, about 5 kHz to about 15 kHz, about 5 kHz to about 17.5 kHz, about 5 kHz to about 20 kHz, about 7.5 kHz to about 10 kHz, about 7.5 kHz to about 12.5 kHz, about 7.5 kHz to about 15 kHz, about 7.5 kHz to about 17.5 kHz, about 7.5 kHz to about 20 kHz, about 10 kHz to about 12.5 kHz, about 10 kHz to about 15 kHz, about 10 kHz to about 17.5 kHz, about 10 kHz to about 20 kHz, about 12.5 kHz to about 15 kHz, about 12.5 kHz to about 17.5 kHz, about 12.5 kHz to about 20 kHz, about 15 kHz to about 17.5 kHz, about 15 kHz to about 20 kHz, or about 17.5 kHz to about 20 kHz. In some embodiments, the time-varying voltage is from about 1 kHz, about 2.5 kHz, about 5 kHz, about 7.5 kHz, about 10 kHz, about 12.5 kHz, about 15 kHz, about 17.5 kHz, or about 20 kHz. In some embodiments, the time-varying voltage is from at least about 1 kHz, about 2.5 kHz, about 5 kHz, about 7.5 kHz, about 10 kHz, about 12.5 kHz, about 15 kHz, or about 17.5 kHz. In some embodiments, the time-varying voltage is from at most about 2.5 kHz, about 5 kHz, about 7.5 kHz, about 10 kHz, about 12.5 kHz, about 15 kHz, about 17.5 kHz, or about 20 kHz.

In some embodiments, upon activation of at least the subset of the plurality of electrodes, the system further comprises a current-return path adjacent to the droplet and one or more inactive electrodes. In some embodiments, the activation of at least the subset of the plurality of electrodes generates an antagonistic current driving scheme in one or more adjacent electrodes.

### Disposable Cartridge

Various methods through which the EWOD platform may be used along with a replaceable cartridge and/or upper-surface films are described herein. A replaceable, flexible, or a combination thereof construct, such as, for example, a film or membrane, allows for reuse of the actuation and/or reference electrodes. The replaceable cartridge may also eliminate cross-contamination between samples in separate experiments or the same experiments. The disposable cartridge construct may contain combinations of dielectric, hydrophobic layers, reference electrodes, inlets, outlets, or any combination thereof for the introduction and removal of fluids. The replaceable construct may be permanently bonded to the array. The construct can be bonded to the actuation electrodes using adhesives, heat, application of vacuum, strong static electric field, or any combination thereof. Examples of disposable cartridges for EWOD droplet actuation can be found in WO2021041709, which is hereby incorporated by reference in its entirety.

### Large Volume Sample Processing

In some embodiments, processing large volume samples (e.g., microliter-, centiliter-, or milliliter-scale) may be carried out by segmenting or fractionating the starting material (e.g., biological samples) into aliquots using a dispenser, and then introducing the aliquots to a processing area of the array. Input material can be processed on the array as droplets in parallel or sequentially. The input material may be, for example, biological samples (e.g., blood, tissue, or plasma) or environmental samples (e.g., water or soil). Sample processing on the array may involve, for example, extraction of nucleic acids (e.g., DNA, RNA), isolation of specific cell types (e.g., immune cell subtypes, circulating tumor cells, or cells isolated from tissue biopsies), or isolation of extracellular vesicles (e.g. exosomes).

### Array Scaling

### Multiplexing

In some embodiments, the number of drive signals can be reduced for scaling from a single array tile to a large number of array tiles (e.g., 10, 20, 30, 40, 50, 100, 500, or more array tiles) for parallel processing of samples (e.g. 96 samples processed simultaneously on 96 individual tiles). For example, a common drive signal can be used to actuate electrodes on multiple tiles simultaneously. Furthermore, the reference electrode(s) on each tile may be driven by separate signals. At any given time, activating the reference electrodes on particular tile may enable droplet mobility on that tile, while the droplets on other (e.g., inactive) tiles may not experience an electromotive forces.

A configuration comprising a number of reconfigurable array tiles stacked next to each other in a reconfigurable bay may provide customization for the number of tiles to be activated for a run. The assembly may allow for loading a single tile or a column of tiles in a reconfigurable tray. The reconfigurable bay, trays, and tiles can be of any arbitrary shape. Multiple trays can be loaded on to a reconfigurable bay to process, for example, 8, 96, 384, 1,536, 6,144, 24,576, or more samples in parallel. The bays, trays, and tiles can be stacked vertically, horizontally, or a combination thereof.

An aspect of the disclosure presents microfluidic dispense chips. Another aspect of the disclosure presents dispense accessories. In some embodiments, the dispense accessory is a chip tray. In some embodiments, the dispense accessory is a wash station. In some embodiments, the dispense accessory is a barcode dispense tip.

### Individual Control Vs Global Control of Evaporation

Regulating evaporation of one or more droplets (samples) on the arrays can be accomplished by processing multiple samples on an array or a plurality of arrays. Enclosing a single droplet on an array tile using methods described herein may accomplish large-scale processing. An entire array tile or a plurality of array tiles may be covered to enclose one or more droplets simultaneously. Enclosures can be lowered on to the array before, during, and/or after droplet processing.

### Common Reagent Dispenser

The same set of reagents (e.g., biological samples, chemical reagents, solutions, nucleic acids (e.g., DNA, RNA, PNA, etc.), optical reagents, etc.) may be introduced to one or more tiles of an array while processing samples. A shared dispenser that distributes reagents across tiles may accomplish the introduction of such reagents. These dispensers may include dispensing mechanisms described herein. The dispensers can comprise one or more distinct channels. Each channel of the distinct channels may be utilized to dispense a single reagent throughout a given process. The dispensers may only comprise a single channel. The single channel may be used to dispense various reagents in a single process. A washing solution may be used to wash a single channel between dispensing different reagents to prevent any possible cross-contamination. The dispensers described herein can also be used to aspirate samples/reagents from the array surface. Wash steps can be performed between consecutive aspiration steps.

An array or a plurality of arrays may be positioned inside a liquid handling automation instrument as described herein. Samples and reagents may be dispensed on to the array by the liquid handler. The array, or plurality thereof, can be removed from the liquid handler (e.g., manually or autonomously) and located adjacent to the liquid handler.

### Single Sample to Multiple Samples

A two-step approach to developing and deploying biological and chemical automation workflows on the arrays may be performed using methods and systems described herein. The workflow may be developed on a single array element and the reactions may be iterated (e.g., manually or autonomously). An optimized workflow can be deployed across multiple arrays. For example, next generation sequencing (NGS) sample preparation workflows on a single sample processing unit can be developed. The developed single NGS sample preparation workflow can then be deployed on an array capable of processing 96 samples in parallel, each of these 96 samples being processed according to the developed single NGS sample preparation workflow.

Film Composites

In order to prevent charge accumulation in a droplet, a patterned electrode may be used on the droplet-facing surface of the dielectric substrate. This patterned electrode may be made using a number of different fabrication methods including screen printing, flexographic printing, gravure printing, inkjet printing, sputtering, and vapor phase deposition techniques. The metallic inks used in the printing processes play an important role in determining the properties of the printed electrode. Silver-particle inks can regularly produce features sizes down to approximately 100 um and have a typical minimum thickness of deposition of approximately 1 um.

If a thin (typically <1 um) conformal hydrophobic coating is used to produce the hydrophobic layer of the coating stack, the thickness of the printed electrode is important in determining whether droplets will be able to move freely on the surface or be pinned in place. It is typically desirable for the trace-height of the printed features to be substantially smaller than the droplet itself. For 100 uL droplets or smaller, 1 um thick traces with a thin hydrophobic coating can greatly impede motion.

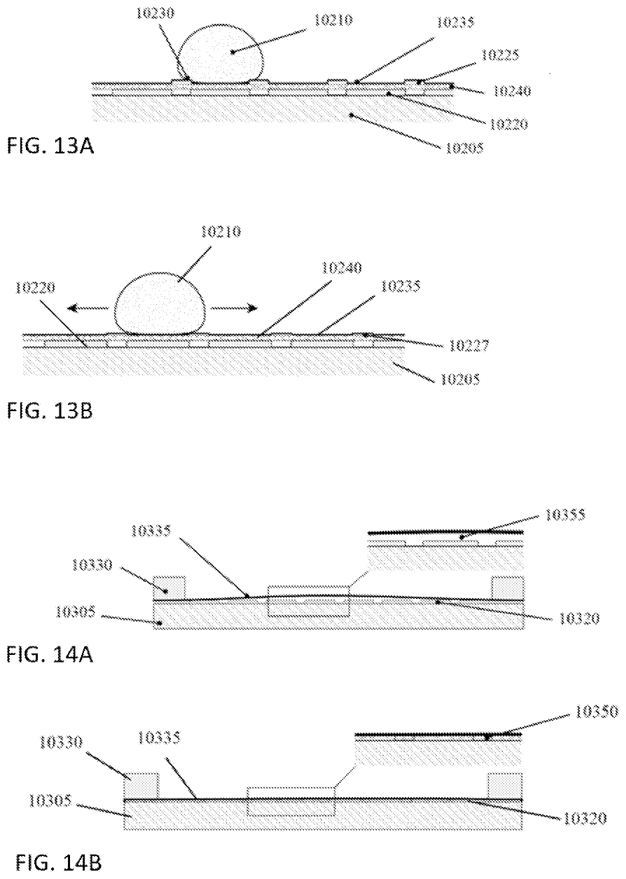

It is therefore desirable to pattern electrodes that are substantially thinner than 1 um, when using thin conformal hydrophobic coatings, as depicted in FIGS. 13A and 13B. Particle free ink formulations that exploit a chemical reaction that precipitate metallic particles are able to reach much smaller feature sizes (˜5 um) and produce much thinner traces (<100 nm). These inks can be patterned using conventional printing processes and are compatible with a variety of substrates including PET and PI dielectrics. FIG. 13A depicts a droplet 10210 to be transported across an array. In some embodiments, the array comprises a first layer of electrodes 10220 adjacent to a substrate 10205. A dielectric layer 10240 may be provided above the first layer of electrodes 10220. A second layer of electrodes 10225 may be provided above the dielectric layer 10240. A conformal coating 10235 may be provided on top of the second layer of electrodes 10225. In some embodiments, the conformal coating is hydrophobic. If the electrodes are too thick (e.g. produced by some screen-printing methods), they may create pinning features 10230 which impede movement of a droplet. Therefore, electrodes may be printed by methods disclosed herein to produce a layer of particle free electrodes 10227 which will not impede movement of a droplet, as depicted in FIG. 13B.

In some embodiments, the configurations described herein can be applied to the cartridges described several sections above.

Film Frame to Tile Application

In one embodiment of an electrowetting device, a thin (<5 um) porous film may be used to create a liquid infused surface on which droplets can freely move. This porous film may be attached to the dielectric film with the use of a film-frame that adheres the three layers (dielectric, porous, frame) at the periphery of the frame. This adhesion can be accomplished with a wet adhesive, dry adhesive, or through thermal lamination. These adhesive strategies can be selectively implemented in regions (e.g. along the periphery of the frame) or across the entire surface of the films.

Thermal lamination is possible when using certain combinations of materials. The dielectric film may be composed of PET, FEP, or PFA to allow for thermal lamination to a textured and porous membrane (Ex: PTFE porous membrane). This thermal lamination process results in a robust film that maintains a porous top surface that can be infused with a liquid to create a liquid infused surface, which enables high performance droplet mobility.

To achieve consistent droplet mobility across the entire electrode array, it is necessary for the film or coating stack-up to be in consistent and close contact with the electrode array and substrate. A variety of methods can be used to achieve this close contact. Tensioning devices can be used to stretch a film-based coating to ensure tight contact with the substrate. Alternatively, vacuum pressure can be used to pull the film tightly against the substrate through small holes or porous features in the substrate.

As depicted in FIGS. 14A and 14B, a substrate 10305 may be provided having comprising an electrodes 10320. In some embodiments, a film 10335 may be provided on over electrodes 10320 and held in place by a film frame 10330. Air bubbles 10355 may be trapped between the film 10335 and electrode array 10320 when the film is attached. These can be easily pushed to the edge of the film with the use of a squeegee or brush. In some embodiments, as depicted in FIG. 14B a filler-fluid 10350 is used to ensure good adhesion between the film-layer and the substrate. A thin layer of filler fluid 10350 may be placed between the electrode array 10320 and the bottom film-layer 10335 to smooth any wrinkling of the film and, through surface tension, removes any air gaps. Filler fluids may include a variety of insulating materials including silicone oil or fluorinated oils.

In some embodiments, the configurations described herein can be applied to the cartridges described several sections above.

### Use of Filler Fluid Adjacent to Electrodes in an Array

In addition to embodiments comprising removable arrays and/or cartridges, where the filler fluid can provide adhesive forces between electrodes and the remaining components of the arrays disclosed herein, filler fluids adjacent to the electrodes of an array described herein can provide additional advantages. For example, in filling the air gap(s) between any two neighboring electrodes the filler fluid acts as a high dielectric breakdown material and prevents air from breaking down. Air typically has a breakdown voltage of about 1 kilovolt per millimeter. So while reducing the gap between two neighboring electrodes is beneficial to allow for smooth transition of droplets, if the gap between two electrodes is reduced, at some point it will start conducting and rendering the electrowetting device non-functional. By adding a filler fluid to fill the gap between two electrodes the gap between the electrodes can be reduced while still maintaining the operability of the array at high voltage for reliable droplet motion. In some embodiments, the filler fluid is a liquid. Further, in some embodiments, particularly embodiments wherein the array comprises a liquid layer disposed on the surface of the dielectric, the filler fluid liquid can be a different composition than the liquid layer disposed on the surface of the dielectric.

An aspect of the present disclosure comprises a system for processing a sample, the system comprising: a plurality of electrodes; a dielectric layer disposed over the plurality of electrodes, wherein the dielectric layer comprises a surface configured to support a droplet comprising the sample; a liquid disposed in an interspace adjacent to the plurality of electrodes and the dielectric layer. In some embodiments, the liquid generates adhesion between the plurality of electrodes and the dielectric layer. In some embodiments, the liquid comprises a dielectric material. In some embodiments, the liquid prevents or reduces electrical conductivity of air disposed in the interspace. In some embodiments, the dielectric layer comprises a natural polymeric material, a synthetic polymeric material, a fluorinated material, a surface modification, or any combination thereof. In some embodiments, the natural polymeric material comprises shellac, amber, wool, silk, natural rubber, cellulose, wax, chiton, or any combination thereof. In some embodiments, the synthetic polymeric material comprises polyethylene, polypropylene, polystyrene, polyetheretherketone (PEEK), polyimide, polyacetal, polysilfone, polyphenulene ether, polyphenylene Sulfide (PPS), polyvinyl chloride, synthetic rubber, neoprene, nylon, polyacrylonitrile, polyvinyl butyral, silicone, parafilm, polyethylene terephthalate, polybutylene terephthalate, polyamides, polyoxymethlyene, polycarbonate, polymethylpentene, polyphenylene oxide (Polyphenyl ether), polyphthalamide (PPA), polylactic acid, synthetic cellulose ethers (e.g., methyl cellulose, ethyl cellulose, propyl cellulose, hydroxyethyl cellulose, hydroxyethyl cellulose, hydroxypropyl cellulose (HPC), hydroxyethyl methyl cellulose, hydroxypropyl methyl cellulose (HPMC), ethyl hydroxyethyl cellulose), paraffins, microcrystalline wax, epoxy, or any combination thereof. In some embodiments, the fluorinated material comprises polytetrafluoroethylene (PTFE), tetrafluoroethylene (TFE), fluorinated ethylenepropylene copolymer (FEP), polyvinylidene fluoride (PVDF), perfluoroalkoxytetrafluoroethylene copolymer (PFA), perfluoromethyl vinylether copolymer (MFA), ethylenechlorotrifluoroethylene copolymer (ECTFE), ethylene-tetrafluoroethylene copolymer (ETFE), perfluoropolyether (PFPE), polychlorotetrafluoroethylene (PCTFE), or any combination thereof. In some embodiments, the surface modification comprises silicone, silane, fluoro-polymer treatment, parylene coating, any other suitable surface chemistry modification process, ceramic, clay minerals, bentonite, kaolinite, vermiculite, graphite, molybdenum disulfide, mica, boron nitride, sodium formate, sodium oleate, sodium palmitate, sodium sulfate, sodium alginate, or any combination thereof. In some embodiments, the liquid comprises silicone oils, fluorinated oils, ionic liquids, mineral oils, ferrofluids, polyphenyl ether, vegetable oil, esters of saturated fatty and dibasic acids, grease, fatty acids, triglycerides, polyalphaolefin, polyglycol hydrocarbons, other Non-hydrocarbon synthetic oils, or any combination thereof. In some embodiments, the liquid further comprises surfactants, electrolytes, rheology modifier, wax, graphite, graphene, molybdenum disulfide, PTFE particles, or any combination thereof. In some embodiments, the surface comprises a liquid layer. In some embodiments, the liquid layer comprises silicone oils, fluorinated oils, ionic liquids, mineral oils, ferrofluids, polyphenyl ether, vegetable oil, esters of saturated fatty and dibasic acids, grease, fatty acids, triglycerides, polyalphaolefin, polyglycol hydrocarbons, other Non-hydrocarbon synthetic oils, or any combination thereof. In some embodiments, the liquid layer further comprises surfactants, electrolytes, rheology modifier, wax, graphite, graphene, molybdenum disulfide, PTFE particles, or any combination thereof. In some embodiments, the dielectric layer is removable. In some embodiments, said dielectric layer comprises a natural polymeric material, a synthetic polymeric material, a fluorinated material, a surface modification, or any combination thereof. In some embodiments, said natural polymeric material comprises shellac, amber, wool, silk, natural rubber, cellulose, wax, chiton, or any combination thereof. In some embodiments, said synthetic polymeric material comprises polyethylene, polypropylene, polystyrene, polyetheretherketone (PEEK), polyimide, polyacetal, polysilfone, polyphenulene ether, polyphenylene Sulfide (PPS), polyvinyl chloride, synthetic rubber, neoprene, nylon, polyacrylonitrile, polyvinyl butyral, silicone, parafilm, polyethylene terephthalate, polybutylene terephthalate, polyamides, polyoxymethlyene, polycarbonate, polymethylpentene, polyphenylene oxide (Polyphenyl ether), polyphthalamide (PPA), polylactic acid, synthetic cellulose ethers (e.g., methyl cellulose, ethyl cellulose, propyl cellulose, hydroxyethyl cellulose, hydroxyethyl cellulose, hydroxypropyl cellulose (HPC), hydroxyethyl methyl cellulose, hydroxypropyl methyl cellulose (HPMC), ethyl hydroxyethyl cellulose), paraffins, microcrystalline wax, epoxy, or any combination thereof. In some embodiments, said fluorinated material comprises polytetrafluoroethylene (PTFE), tetrafluoroethylene (TFE), fluorinated ethylenepropylene copolymer (FEP), polyvinylidene fluoride (PVDF), perfluoroalkoxytetrafluoroethylene copolymer (PFA), perfluoromethyl vinylether copolymer (MFA), ethylenechlorotrifluoroethylene copolymer (ECTFE), ethylene-tetrafluoroethylene copolymer (ETFE), perfluoropolyether (PFPE), polychlorotetrafluoroethylene (PCTFE), or any combination thereof. In some embodiments, said surface modification comprises silicone, silane, fluoro-polymer treatment, parylene coating, any other suitable surface chemistry modification process, ceramic, clay minerals, bentonite, kaolinite, vermiculite, graphite, molybdenum disulfide, mica, boron nitride, sodium formate, sodium oleate, sodium palmitate, sodium sulfate, sodium alginate, or any combination thereof. In some embodiments, said liquid comprises silicone oils, fluorinated oils, ionic liquids, mineral oils, ferrofluids, polyphenyl ether, vegetable oil, esters of saturated fatty and dibasic acids, grease, fatty acids, triglycerides, polyalphaolefin, polyglycol hydrocarbons, other Non-hydrocarbon synthetic oils, or any combination thereof. In some embodiments, said liquid further comprises surfactants, electrolytes, rheology modifier, wax, graphite, graphene, molybdenum disulfide, PTFE particles, or any combination thereof. In some embodiments, said surface comprises a liquid layer. In some embodiments, said liquid layer comprises silicone oils, fluorinated oils, ionic liquids, mineral oils, ferrofluids, polyphenyl ether, vegetable oil, esters of saturated fatty and dibasic acids, grease, fatty acids, triglycerides, polyalphaolefin, polyglycol hydrocarbons, other Non-hydrocarbon synthetic oils, or any combination thereof. In some embodiments, said liquid layer further comprises surfactants, electrolytes, rheology modifier, wax, graphite, graphene, molybdenum disulfide, PTFE particles, or any combination thereof. In some embodiments, said dielectric layer is removable. In some embodiments, said adhesion is sufficient to immobilize said liquid onto said surface and wherein said liquid is resistant to gravity. In some embodiments, said liquid is selected to preferentially wet said surface to facilitate a motion of said droplet on said surface.

### Monitoring Droplets

The present disclosure provides methods for monitoring at least one droplet on an electrowetting array. The droplet may be in operation on the electrowetting array. The droplet may be in a reaction state.

Examples of methods for monitoring droplets on surfaces for EWOD droplet actuation can be found in WO2021041709, which is hereby incorporated by reference in its entirety.

### Methods for Analysis of Nucleic Acids

### High Molecular Weight (HMW) Nucleic Acid Isolation and Transfer

Intact genomic DNA can be greater than about 100 megabases (Mb) in length, but isolation protocols may fragment the genomic DNA to fragments of 10-200 kilobases (Kb) in length. However, as sequencing technologies are capable of processing longer read lengths (e.g., greater than about 1 Mb), the low yield of intact genomic DNA molecules (e.g., >100 kb) is an unresolved limitation of DNA isolation technologies.

HMW nucleic acid may be extracted from, for example, whole blood, serum, or saliva. HMW nucleic acid can be extracted from whole cells. The nucleic acid may be DNA. The nucleic acid may be RNA. The nucleic acid may be extracted for various applications including, for example, library preparation, amplification, sequencing, polymerase chain reaction (PCR), gel electrophoresis, and other processes.

Described herein are systems and methods that minimize mechanical fragmentation (e.g., due to shear forces of air-displacement pipetting) of nucleic acids (e.g., DNA). Described herein are systems and methods that reduce sample loss due to, for example, dead volumes of traditional handling devices. The systems and methods described herein may be capable of automating high throughput and high molecular weight (HMW) DNA isolation, wherein the median DNA fragment size is at least about 1 Kb, 10 Kb, 100 Kb, 1,000 Kb, 10,000 Kb, 100,000 Kb, 1,000,000 Kb, or more. The systems and methods described herein may be capable of automating high throughput and high molecular weight DNA isolation, wherein the median DNA fragment size is at most about 1,000,000 Kb, 100,000 Kb, 10,000 Kb, 1,000 Kb, 100 Kb, 10 Kb, 1 Kb, or less. The systems and methods described herein may be capable of automating high throughput and high molecular weight DNA isolation, wherein the median DNA fragment size is from about 1 Kb to about 1,000,000 Kb, 100 Kb to about 500,000 Kb, or about 1,000 Kb to about 100,000 Kb.

Described herein are systems and methods for whole blood extraction on an electrowetting array. In some embodiments, at least about 10 μL to at least about 500 μL of whole blood is used for extraction. In some embodiments, at least about 100 μL of whole blood is used for extraction. In some embodiments, about 10 μL to about 250 μL. In some embodiments, at least about 10, about 20, about 30, about 40, about 50, about 60, about 70, about 80, about 90, about 100, or about 150. In some embodiments, at most about 20, about 30, about 40, about 50, about 60, about 70, about 80, about 90, about 100, about 150, about 200, about 250, about 300, about 350, about 500, about 450, about 500, or more μL of whole blood is used for extraction. In some embodiments, at least about 0.2 to at most about 5 μg of DNA is extracted from 100 μL of blood. In some embodiments, at least about 0.2, 0.3, 0.4, 0.5, 0.6, 0.7, 0.8, 0.9, 1, 2, 3, 4, or 5 μg of DNA is extracted from 100 μL of blood.

Described herein are universal open electrowetting-on-dielectric (EWOD) systems and methods that can manipulate reaction volumes suitable for HMW DNA isolation. By integrating capabilities such as, for example, magnetic bead separation and heater/cooler in the same system, the system and methods described herein may not comprise custom instrumentation. The systems and methods described herein can provide straightforward reprogramming to expand the number of executable workflows, enabling new recipes with, for example, variable input, reagents, incubations, wash steps, and thousands of droplets controlled in a programmable manner on a single device.

The system described herein can manipulate droplets on a 2D or 3D grid of electrodes in at least two configurations (e.g., droplets sandwiched between two plates separated by a small gap or on an open surface). For example, on a two plate PDM system (e.g., with electrodes dimension of 25 μm) a droplet of 5 μL can be aliquoted, transported, and mix with another droplet. On an open surface, EWOD device (e.g., with electrodes dimension of 2 mm) droplets (e.g., about 200 μL) can be manipulated. The systems described herein can handle volumes suitable for, for example, bulk DNA extraction (e.g., 100 μl to 1 ml) as well as droplets small enough to encapsulate single cells and individual nuclei (e.g., 50 nL).

Additionally, in order to increase the yield of HMW DNA from cell samples, enhanced agitation techniques may be performed on the array. Agitation techniques may include methods such as, for example, mechanical buzzers, shakers, vortexers, sonication, or any combination thereof. Magnetic micro-stirrers may be introduced into the samples to enhance mixing. These stirrers may be coupled with different magnet configurations described herein. Magnets of different shapes may be used to alter the shape and spread of the magnetic beads on the array. Magnets with tunable strength may be used to accommodate the magnetic beads being manipulated on the array.

DNA extracted from cells in a stabilization buffer can produce intact HMW DNA. For example, alginate hydrogels can be used as a scaffold material to stabilize HMW DNA. Alginate can form stable gels in the presence of cations, gelling conditions may be mild, and the gelation process can be reversed by, for example, extracting calcium ions (e.g., by adding citrate or EDTA). Extracted DNA can be stabilized in high viscosity/low-shear solutions (e.g., alginate droplets) formed on-chip. This stabilization method may allow the transfer (e.g. within a lab or by shipping between sites) of HMW genomic DNA without substantial degradation. HMW DNA can be stored in reagents to prevent shearing (e.g. alginate hydrogels). Extracted HMW DNA can be, for example, transferred to a tube after extraction or stored on the EWOD array. To prevent DNA shearing prior to sequencing, sequencing libraries can be assembled on the same device used for HMW DNA extraction. Similarly, nanopores may be integrated to the array for direct sequencing without sample transfer.

### Sample Preparation

Circular Sample Preparation

The present disclosure provides methods of sample preparation for sequencing. The method may comprise conducting high molecular weight (HMW) nucleic acid extraction. The method may comprise further comprise sample preparation. The method of sample preparation may result in a circular nucleic acid. In an example, the nucleic acid may be deoxyribonucleic acid (DNA). The method of sample preparation may be circularization or cyclization. In an example., addition of polymerase and/or ligase may result in conversion of a double-stranded nucleic acid to circular form. The nucleic acid may be circularized by covalent closure of DNA “sticky” ends. The nucleic acid may be circularized by recombination between redundant terminal sequences. The nucleic acid may be circularized via the binding of a protein at viral DNA extremities. The nucleic acid may be circularized on an electrowetting array.

The present disclosure provides methods of sample preparation on an electrowetting array. The method of sample preparation may comprise preparing a sample for sequencing. The method of sample preparation may comprise preparing a sample for circular consensus sequencing (CCS). The method of sample preparation may be circularization. The method of sample preparation may comprise preparing a sample for rolling circle amplification (RCA). The nucleic acid may be circularized by providing a droplet adjacent to an electrowetting array, wherein the droplet comprises the nucleic acid. The nucleic acid may be circularized by combining the droplet with one or more reagent droplets. The one or more reagents may be reagents to circularize a nucleic acid sample. The one or more reagents may be enzymes. Examples of enzymes may include polymerase and ligase. In an example, a single polymerizing enzyme may be used to subject the nucleic acid sample to a sequencing reaction.

The nucleic acid may be circularized by using the electrowetting array to process the droplet to circularize the nucleic acid. The circularized nucleic acid sample may be separated from the one or more reagent droplets. The sample droplet may be combined with the one or more reagent droplets and subsequently separated from the one or more reagent droplets. The one or more reagent droplets may then be combined with a second droplet. The method may further comprise performing one or more droplet operations on the electrowetting array to process the droplet, wherein the one or more droplet operations comprise contacting the one or more reagent droplets with the droplet. Examples of nucleic acid circularization may also be provided in Pacific Biosciences of California's “Template Preparation and Sequencing Guide” (see https://www.pacb.com/wp-content/uploads/2015/09/Guide-Pacific-Biosciences-Template-Preparation-and-Sequencing.pdf) and WO2009120374, which are incorporated herein by reference in its entirety.

Methods of nucleic acid sample preparation may yield a high sequencing read. Methods of nucleic acid sample preparation may yield a sequencing read having a length of at least about 70 kilobase (kb). Methods of nucleic acid sample preparation may yield a sequencing read having a length of at least about 80 kb. Methods of nucleic acid sample preparation may yield a sequencing read having a length of at least about 200 kb. Methods of nucleic acid sample preparation may yield a sequencing read having a length of at least about 70 kb, at least about 80 kb, at least about 90 kb, at least about 100 kb, at least about 110 kb, at least about 120 kb, at least about 130 kb, at least about 140 kb, at least about 150 kb, at least about 160 kb, at least about 170 kb, at least about 180 kb, at least about 190 kb, at least about 200 kb, or more. In an example, methods of nucleic acid sample preparation may be conducted on an electrowetting array. In another example, the nucleic acid may be deoxyribonucleic acid (DNA).

Methods of nucleic acid sample preparation may yield at least about 100 Gigabytes (Gb) of sequencing data. Methods of nucleic acid sample preparation may yield at least about 10 Gb of data. Methods of nucleic acid sample preparation may yield at least about 30 Gb of sequencing data. Methods of nucleic acid sample preparation may yield at least about 500 Gb of sequencing data. Methods of nucleic acid sample preparation may yield at least about 512 Gb of sequencing data. Methods of nucleic acid sample preparation may yield at least about 1, at least about 2, at least about 3, at least about 4, at least about 5, at least about 6, at least about 7, at least about 8, at least about 9, at least about 10, at least about 20, at least about 30, at least about 40, at least about 50, at least about 60, at least about 70, at least about 80, at least about 90, at least about 100, at least about 150, at least about 200, at least about 250, at least about 300, at least about 350, at least about 400, at least about 450, at least about 500 Gb or more of sequencing data.

The circularized nucleic acid sample may comprise a plurality of sequences comprising a target sequence. At least about 80% of the plurality of sequences may comprise the target sequence. At least about 10%, at least about 20%, at least about 30%, at least about 40%, at least about 50%, at least about 60%, at least about 70%, at least about 80%, at least about 90% or more of the plurality of sequences may comprise the target sequence.

Methods of preparing a nucleic acid sample as disclosed herein may provide a nucleic acid sample with high purity. The purity of a nucleic acid sample may be evaluated by measuring absorbance. Absorbance reads may be taken on a spectrophotometer. The method of measuring purity may comprise providing a sample comprising at least one molecule; measuring the absorbance of said sample at 260 nanometers (nm); measuring the absorbance of said sample at 280 nm; and providing a ratio of absorbance at 260 nm over 280 nm (A260/A280 ratio). Methods of preparing a nucleic acid sample as disclosed herein may provide at least one sequencing read with an A260/A280 ratio of at most about 1.93. Methods of preparing a nucleic acid sample as disclosed herein may provide at least one sequencing read with A260/A280 ratio of at most about 1.84. Methods of preparing a nucleic acid sample as disclosed herein may provide at least one sequencing read with a A260/A280 ratio of at most about 5, at most 4.5, at most about 4, at most about 3.5, at most about 3, at most about 2.5, at most about 2, at most about 1.9, at most about 1.8. at most about 1.7, at most about 1.6, at most about 1.5, at most about 1.4, at most about 1.3, at most about 1.2, at most about 1.1, or less.

The circularized nucleic acid sample may comprise a plurality of sequences comprising a target sequence. At least about 80% of the plurality of sequences may comprise the target sequence. At least about 10%, at least about 20%, at least about 30%, at least about 40%, at least about 50%, at least about 60%, at least about 70%, at least about 80%, at least about 90% or more of the plurality of sequences may comprise the target sequence.

The present disclosure provides a method of producing a circularized nucleic acid sample with a longer insert size, comprising (a) providing a droplet adjacent to an electrowetting array, which droplet comprises the nucleic acid sample, (b) using the electrowetting array to process the droplet to circularize the nucleic acid sample, and (c) using a single polymerizing enzyme to subject the circularized nucleic acid sample to a sequencing reaction. The electrowetting array may further comprise one or more reagent droplets. The one or more reagent droplets may comprise one or more reagents for circularizing the nucleic acid sample. The method may further comprise combining the sample droplet with the one or more reagent droplets; separating the sample droplet from the one or more reagent droplets; and combining the one or more reagent droplets with a second droplet. The method may further comprise performing one or more droplet operations on the electrowetting array to process the droplet, wherein the one or more droplet operations comprise contacting the one or more reagent droplets with the droplet.

Methods of producing a circularized nucleic acid sample with a longer insert size may yield a high sequencing read. Methods of producing a circularized nucleic acid sample with a longer insert size may yield a sequencing read having a length of at least about 70 kilobase (kb). Methods of producing a circularized nucleic acid sample with a longer insert size may yield a sequencing read having a length of at least about 80 kb. Methods of producing a circularized nucleic acid sample with a longer insert size may yield a sequencing read having a length of at least about 200 kb.

Methods of producing a circularized nucleic acid sample with a longer insert size may yield a sequencing read having a length of at least about 70 kb, at least about 80 kb, at least about 90 kb, at least about 100 kb, at least about 110 kb, at least about 120 kb, at least about 130 kb, at least about 140 kb, at least about 150 kb, at least about 160 kb, at least about 170 kb, at least about 180 kb, at least about 190 kb, at least about 200 kb, or more.

In an example, methods of producing a circularized nucleic acid sample with a longer insert size may be conducted on an electrowetting array. The nucleic acid may be deoxyribonucleic acid (DNA).

Methods of producing a circularized nucleic acid sample with a longer insert size may yield at least about 100 Gigabytes (Gb) of sequencing data. Methods of producing a circularized nucleic acid sample with a longer insert size may yield at least about 10 Gb of data. Methods of producing a circularized nucleic acid sample with a longer insert size may yield at least about 30 Gb of sequencing data. Methods of producing a circularized nucleic acid sample with a longer insert size may yield at least about 500 Gb of sequencing data. Methods of producing a circularized nucleic acid sample with a longer insert size may yield at least about 512 Gb of sequencing data. Methods of producing a circularized nucleic acid sample with a longer insert size may yield at least about 1, at least about 2, at least about 3, at least about 4, at least about 5, at least about 6, at least about 7, at least about 8, at least about 9, at least about 10, at least about 20, at least about 30, at least about 40, at least about 50, at least about 60, at least about 70, at least about 80, at least about 90, at least about 100, at least about 150, at least about 200, at least about 250, at least about 300, at least about 350, at least about 400, at least about 450, at least about 500 Gb or more of sequencing data.

The circularized nucleic acid sample may comprise a plurality of sequences comprising a target sequence. At least about 80% of the plurality of sequences may comprise the target sequence. At least about 10%, at least about 20%, at least about 30%, at least about 40%, at least about 50%, at least about 60%, at least about 70%, at least about 80%, at least about 90% or more of the plurality of sequences may comprise the target sequence.

Methods of producing a circularized nucleic acid sample with a longer insert size may provide a nucleic acid sample with high purity. The purity of a nucleic acid sample may be evaluated by measuring absorbance. Absorbance reads may be taken on a spectrophotometer. The method of measuring purity may comprise providing a sample comprising at least one molecule; measuring the absorbance of said sample at 260 nanometers (nm); measuring the absorbance of said sample at 280 nm; and providing a ratio of absorbance at 260 nm over 280 nm (A260/A280 ratio). Methods of producing a circularized nucleic acid sample with a longer insert size may provide at least one sequencing read with an A260/A280 ratio of at most about 1.93. Methods of producing a circularized nucleic acid sample with a longer insert size may provide at least one sequencing read with A260/A280 ratio of at most about 1.84. Methods of producing a circularized nucleic acid sample with a longer insert size may provide at least one sequencing read with a A260/A280 ratio of at most about 5, at most 4.5, at most about 4, at most about 3.5, at most about 3, at most about 2.5, at most about 2, at most about 1.9, at most about 1.8. at most about 1.7, at most about 1.6, at most about 1.5, at most about 1.4, at most about 1.3, at most about 1.2, at most about 1.1, or less.

The circularized nucleic acid sample may comprise a plurality of sequences comprising a target sequence. At least about 80% of the plurality of sequences may comprise the target sequence. At least about 10%, at least about 20%, at least about 30%, at least about 40%, at least about 50%, at least about 60%, at least about 70%, at least about 80%, at least about 90% or more of the plurality of sequences may comprise the target sequence.

The present disclosure provides a method for circularizing a nucleic acid sample, comprising: providing a droplet adjacent to an electrowetting array, wherein the droplet comprises the nucleic acid sample; combining the droplet with one or more reagent droplets; using the electrowetting array to process the droplet to circularize the nucleic acid sample; separating the droplet from the one or more reagent droplets; and combining the one or more reagent droplets with the sample droplet to yield a circularized nucleic acid sample. The electrowetting array may further comprise one or more reagent droplets. The one or more reagent droplets may comprise one or more reagents for circularizing the nucleic acid sample. The method may further comprise combining the sample droplet with the one or more reagent droplets; separating the sample droplet from the one or more reagent droplets; and combining the one or more reagent droplets with a second droplet. The method may further comprise performing one or more droplet operations on the electrowetting array to process the droplet, wherein the one or more droplet operations comprise contacting the one or more reagent droplets with the droplet.

Methods of circularizing a nucleic acid sample may yield a sequencing read having a length of at least about 70 kb, at least about 80 kb, at least about 90 kb, at least about 100 kb, at least about 110 kb, at least about 120 kb, at least about 130 kb, at least about 140 kb, at least about 150 kb, at least about 160 kb, at least about 170 kb, at least about 180 kb, at least about 190 kb, at least about 200 kb, or more.

In an example, methods of circularizing a nucleic acid sample may be conducted on an electrowetting array. The nucleic acid may be deoxyribonucleic acid (DNA).

Methods of circularizing a nucleic acid sample may yield at least about 100 Gigabytes (Gb) of sequencing data. Methods of circularizing a nucleic acid sample may yield at least about 10 Gb of data. Methods of circularizing a nucleic acid sample may yield at least about 30 Gb of sequencing data. Methods of circularizing a nucleic acid sample may yield at least about 500 Gb of sequencing data. Methods of circularizing a nucleic acid sample may yield at least about 512 Gb of sequencing data. Methods of circularizing a nucleic acid sample may yield at least about 1, at least about 2, at least about 3, at least about 4, at least about 5, at least about 6, at least about 7, at least about 8, at least about 9, at least about 10, at least about 20, at least about 30, at least about 40, at least about 50, at least about 60, at least about 70, at least about 80, at least about 90, at least about 100, at least about 150, at least about 200, at least about 250, at least about 300, at least about 350, at least about 400, at least about 450, at least about 500 Gb or more of sequencing data.

The circularized nucleic acid sample may comprise a plurality of sequences comprising a target sequence. At least about 80% of the plurality of sequences may comprise the target sequence. At least about 10%, at least about 20%, at least about 30%, at least about 40%, at least about 50%, at least about 60%, at least about 70%, at least about 80%, at least about 90% or more of the plurality of sequences may comprise the target sequence.

Methods of circularizing a nucleic acid sample may provide a nucleic acid sample with high purity. The purity of a nucleic acid sample may be evaluated by measuring absorbance. Absorbance reads may be taken on a spectrophotometer. The method of measuring purity may comprise providing a sample comprising at least one molecule; measuring the absorbance of said sample at 260 nanometers (nm); measuring the absorbance of said sample at 280 nm; and providing a ratio of absorbance at 260 nm over 280 nm (A260/A280 ratio). Methods of circularizing a nucleic acid sample may provide at least one sequencing read with an A260/A280 ratio of at most about 1.9. Methods of circularizing a nucleic acid sample may provide at least one sequencing read with A260/A280 ratio of at most about 1.84. Methods of circularizing a nucleic acid sample may provide at least one sequencing read with a A260/A280 ratio of at most about 5, at most 4.5, at most about 4, at most about 3.5, at most about 3, at most about 2.5, at most about 2, at most about 1.93, at most about 1.8. at most about 1.7, at most about 1.6, at most about 1.5, at most about 1.4, at most about 1.3, at most about 1.2, at most about 1.1, or less.

The circularized nucleic acid sample may comprise a plurality of sequences comprising a target sequence. At least about 80% of the plurality of sequences may comprise the target sequence. At least about 10%, at least about 20%, at least about 30%, at least about 40%, at least about 50%, at least about 60%, at least about 70%, at least about 80%, at least about 90% or more of the plurality of sequences may comprise the target sequence.

The present disclosure provides a method for generating a sequencing library, comprising (a) providing a nucleic acid sample comprising a plurality of nucleic acid molecules comprising a plurality of sequences, (b) using the nucleic acid sample to generate the sequencing library, wherein the sequencing library comprises at least 80% of the plurality of sequences of complements thereof; (c) using said electrowetting array to process said droplet to circularize said nucleic acid sample; (d) separating said droplet from said one or more reagent droplets; and; (e) combining said one or more reagent droplets with said sample droplet to yield a circularized nucleic acid sample. The electrowetting array may further comprise one or more reagent droplets. The one or more reagent droplets may comprise one or more reagents for circularizing the nucleic acid sample. The method may further comprise combining the sample droplet with the one or more reagent droplets; separating the sample droplet from the one or more reagent droplets; and combining the one or more reagent droplets with a second droplet. The method may further comprise performing one or more droplet operations on the electrowetting array to process the droplet, wherein the one or more droplet operations comprise contacting the one or more reagent droplets with the droplet.

Methods of generating a sequencing library may yield a sequencing read having a length of at least about 70 kb, at least about 80 kb, at least about 90 kb, at least about 100 kb, at least about 110 kb, at least about 120 kb, at least about 130 kb, at least about 140 kb, at least about 150 kb, at least about 160 kb, at least about 170 kb, at least about 180 kb, at least about 190 kb, at least about 200 kb, or more.

In an example, methods of generating a sequencing library size may be conducted on an electrowetting array. The nucleic acid may be deoxyribonucleic acid (DNA).

Methods of generating a sequencing library may yield at least about 100 Gigabytes (Gb) of sequencing data. Methods of generating a sequencing library may yield at least about 10 Gb of data. Methods of generating a sequencing library may yield at least about 30 Gb of sequencing data. Methods of generating a sequencing library may yield at least about 500 Gb of sequencing data. Methods of generating a sequencing library may yield at least about 512 Gb of sequencing data. Methods of generating a sequencing library may yield at least about 1, at least about 2, at least about 3, at least about 4, at least about 5, at least about 6, at least about 7, at least about 8, at least about 9, at least about 10, at least about 20, at least about 30, at least about 40, at least about 50, at least about 60, at least about 70, at least about 80, at least about 90, at least about 100, at least about 150, at least about 200, at least about 250, at least about 300, at least about 350, at least about 400, at least about 450, at least about 500 Gb or more of sequencing data.

The circularized nucleic acid sample may comprise a plurality of sequences comprising a target sequence. At least about 80% of the plurality of sequences may comprise the target sequence. At least about 10%, at least about 20%, at least about 30%, at least about 40%, at least about 50%, at least about 60%, at least about 70%, at least about 80%, at least about 90% or more of the plurality of sequences may comprise the target sequence.

Methods of generating a sequencing library size may provide a nucleic acid sample with high purity. The purity of a nucleic acid sample may be evaluated by measuring absorbance. Absorbance reads may be taken on a spectrophotometer. The method of measuring purity may comprise providing a sample comprising at least one molecule; measuring the absorbance of said sample at 260 nanometers (n); measuring the absorbance of said sample at 280 n; and providing a ratio of absorbance at 260 nm over 280 nm (A260/A280 ratio). Methods of generating a sequencing library may provide at least one sequencing read with an A260/A280 ratio of at most about 1.93. Methods of generating a sequencing library may provide at least one sequencing read with A260/A280 ratio of at most about 1.84. Methods of generating a sequencing library may provide at least one sequencing read with a A260/A280 ratio of at most about 5, at most 4.5, at most about 4, at most about 3.5, at most about 3, at most about 2.5, at most about 2, at most about 1.9, at most about 1.8. at most about 1.7, at most about 1.6, at most about 1.5, at most about 1.4, at most about 1.3, at most about 1.2, at most about 1.1, or less.

The circularized nucleic acid sample may comprise a plurality of sequences comprising a target sequence. At least about 80% of the plurality of sequences may comprise the target sequence. At least about 10%, at least about 20%, at least about 30%, at least about 40%, at least about 50%, at least about 60%, at least about 70%, at least about 80%, at least about 90% or more of the plurality of sequences may comprise the target sequence.

Next Generation Sequencing (NGS) Sample Preparation

The systems and methods described herein may accomplish fully digital for high-throughput automation of NGS sample preparation. Whole genome sequencing (WSG) libraries can be prepared starting from purified DNA using systems and methods described herein. For example, DNA can be fragmented enzymatically on an array described herein, end-repaired, and A-overhangs added. Dual indexed barcodes can be ligated onto the DNA fragments and the final ligation product can be purified and size-selected by magnetic-bead based purification. The method may be performed on a single device described herein. In an example, library preparation comprises attaching adapters on both ends of a nucleic acid. Examples of sample preparation for NGS include, but are not limited to Illumina, Inc.'s Cluster Generation technology (see “Technology Spotlight: Illumina Sequencing Technology,” which is incorporated herein by reference in its entirety.) The method of sample preparation for NGS may be conducive to whole genome sequencing (WGS). The method of sample preparation for NGS may comprise employing hybridization capture. The method of sample preparation for NGS may comprise employing unique molecular identifiers (UMIs) for improved sensitivity and sequencing.

The present disclosure provides methods of sample preparation on an electrowetting array. The method of sample preparation may comprise preparing a sample for sequencing. The method of sample preparation may comprise preparing a sample for NGS. The method may comprise providing a sample droplet and at least one reagent droplet. The method may further comprise using droplet operations on an electrowetting array to bring the sample droplet in contact with the at least one reagent droplet. The one or more reagent droplets may comprise one or more reagents for performing sample preparation for NGS. The one or more reagents may be, for example, a part of Illumina's NovaSeq 6000 kit (see “NovaSeq 6000 Reagent Kit,” which is incorporated herein by reference in its entirety.) The bridge amplification may be conducted in preparation for sequencing. Sample preparation for NGS may be performed on an electrowetting array with a single-tube protocol. Sample preparation for NGS may be performed on an electrowetting array through automated methods. Sample preparation for NGS may be performed on an electrowetting array between at least about 1 to at least about 14 days. Sample preparation for NGS may be performed on an electrowetting array within at least about 1, 2, 3, 4, 5, 6, 7, 8, 9, 10, 11, 12, 13, 14, or more days. Sample preparation for NGS may yield at least about 1 to at least about 10 μg of DNA. Sample preparation for NGS may yield at least about 1, 2, 3, 4, 5, 6, 7, 8, 9, 10, or more gg of DNA. Sample preparation for NGS may yield read lengths of at least about 10 to at least about 500 kb. Sample preparation for NGS may yield read lengths of at least about 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500 kb or more. Sample preparation for NGS may comprise using various insert sizes. Sample preparation for NGS may comprise using about 100 bp to about 600 bp for insert size. Sample preparation for NGS may comprise using about 100, 110, 120, 130, 140, 150, 160, 170, 180, 190, 200, 210, 220, 23, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600 bp or more for insert size. Sample preparation for NGS may comprise using about 300 to about 450 bp for insert size. Sample preparation for NGS may yield various library sizes. Sample preparation for NGS may yield about 100 bp to about 600 bp of library size. Sample preparation for NGS may yield about 100, 110, 120, 130, 140, 150, 160, 170, 180, 190, 200, 210, 220, 23, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600 bp or more for library size. Sample preparation for NGS may comprise using about 400 to about 600 bp for library size.

The present disclosure provides systems and methods for sample preparation for nanopore sequencing. In some embodiments, nanopore sequencing may comprise subjecting a nucleic acid to a one-step real-time PCR (RT-PCR), and subsequently sequencing the nucleic acid on a nanopore device. Sample preparation may be conducted on an electrowetting array by bringing at least one sample droplet in contact with at least one reagent droplet using droplet operations on the electrowetting array. Sample preparation for nanopore sequencing on an electrowetting array may yield a high N50, or the sequence length of the shortest contig at 50% of the total genome length. Sample preparation for nanopore sequencing on an electrowetting array may yield an N50 of at least about 10 kb to at least about 50 kb. Sample preparation for nanopore sequencing on an electrowetting array may yield an N50 of at least about 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50 kb or more. Sample preparation for nanopore sequencing on an electrowetting array may yield nucleic acid with higher pore occupancy. Sample preparation for nanopore sequencing on an electrowetting array may yield nucleic acid with at least about 24 hours of high pore occupancy. Sample preparation for nanopore sequencing on an electrowetting array may yield nucleic acid with at least about 6, 7, 8, 9, 10, 11, 12, 13, 14, 15, 16, 17, 18, 19, 20, 21, 22, 23, 24, 25, 26, 27, 28, 29, 30, or more hours of high pore occupancy. Examples of nanopore sequencing include, but are not limited to Nanopore's MinION sequencing (see Product Description for MinION; Lu, Hengyun, Francesca Giordano, and Zemin Ning. “Oxford Nanopore MinION sequencing and genome assembly.” Genomics, proteomics & bioinformatics 14.5 (2016): 265-279; which are incorporated herein by reference in their entirety).

Amplification

The present disclosure provides a method of sample preparation for amplification. The amplification may be in preparation for sequencing. The amplification may involve an initiator protein binding to a double-stranded nucleic acid. The initiator protein may bind to the 5′ end of one strand of the double-stranded nucleic acid. The initiator protein may cause at least one nick in the double-stranded nucleic acid. A helicase-dependent amplification may occur wherein a helicase unwinds the double-stranded nucleic acid, and at least one single-stranded binding protein coats at least one strand of the double-stranded nucleic acid. The process may be single-stranded binding (SSB) protein-dependent amplification. The nucleic acid may be deoxyribonucleic acid (DNA). The method of amplification may comprise rolling circle amplification (RCA). The method of amplification may comprise bridge amplification.

### RCA

The present disclosure provides a method of conducting amplification. The amplification may be in preparation for sequencing. The amplification may involve an initiator protein binding to a double-stranded nucleic acid. The initiator protein may bind to the 5′ end of one strand of the double-stranded nucleic acid. The initiator protein may cause at least one nick in the double-stranded nucleic acid. A helicase-dependent amplification may occur wherein a helicase unwinds the double-stranded nucleic acid, and at least one single-stranded binding protein coats at least one strand of the double-stranded nucleic acid. The process may be single-stranded binding (SSB) protein-dependent amplification. The nucleic acid may be deoxyribonucleic acid (DNA). The method of amplification may comprise rolling circle amplification (RCA).

Examples of rolling circle amplification, but are not limited to, protocols of circularizing nucleic acid as described in U.S. Pat. Nos. 9,290,800; 11,067,562; U.S.

Publication Number US20190360997; PacBio SMRT Sequencing (see https://www.pacb.com/smrt-science/smrt-sequencing/); Wenger, Aaron M., et al. “Accurate circular consensus long-read sequencing improves variant detection and assembly of a human genome.” Nature biotechnology 37.10 (2019): 1155-1162; Illumina's CirSeq (see https://www.illumina.com/science/sequencing-method-explorer/kits-and-arrays/cirseq.html); Acevedo, A., Andino R., “Library preparation for highly accurate population sequencing of RNA viruses.” Nat Protoc. 2014 July;9(7):1760-9); Hunt, M., Silva, N.D., Otto, T. D. et al. “Circlator: automated circularization of genome assemblies using long sequencing reads.” Genome Biol 16, 294 (2015); and Wilson, Brandon D et al. “High-Fidelity Nanopore Sequencing of Ultra-Short DNA Targets.” Analytical chemistry vol. 91,10 (2019): 6783-6789, all of which are incorporated herein by reference in their entirety).

The method of amplification may be conducted on an electrowetting array. The electrowetting array may further comprise one or more reagent droplets. The one or more reagent droplets may comprise one or more reagents for circularizing the nucleic acid sample. The one or more reagent droplets may comprise one or more reagents performing rolling circle amplification (RCA). The one or more reagents may be enzymes. Examples of enzymes may include a nicking enzyme, a DNA polymerase, and an RCR protein. The one or more reagents may be control nucleic acids, buffer solutions and/or salt solutions, including, for example, divalent metal ions, i.e., Mg²⁺, Mn²⁺, Ca²⁺ and/or Fe²⁺. The one or more reagents may prepare single-stranded nucleic acids.

Methods of conducting amplification may yield a sequencing read having a length of at least about 70 kb, at least about 80 kb, at least about 90 kb, at least about 100 kb, at least about 110 kb, at least about 120 kb, at least about 130 kb, at least about 140 kb, at least about 150 kb, at least about 160 kb, at least about 170 kb, at least about 180 kb, at least about 190 kb, at least about 200 kb, or more.

In an example, methods of conducting amplification may be conducted on an electrowetting array. The nucleic acid may be deoxyribonucleic acid (DNA).

Methods of conducting amplification may yield at least about 100 Gigabytes (Gb) of sequencing data. Methods of conducting amplification may yield at least about 10 Gb of data. Methods of conducting amplification may yield at least about 30 Gb of sequencing data. Methods of conducting RCA may yield at least about 500 Gb of sequencing data. Methods of conducting amplification may yield at least about 512 Gb of sequencing data. Methods of conducting amplification may yield at least about 1, at least about 2, at least about 3, at least about 4, at least about 5, at least about 6, at least about 7, at least about 8, at least about 9, at least about 10, at least about 20, at least about 30, at least about 40, at least about 50, at least about 60, at least about 70, at least about 80, at least about 90, at least about 100, at least about 150, at least about 200, at least about 250, at least about 300, at least about 350, at least about 400, at least about 450, at least about 500 Gb or more of sequencing data.

The circularized nucleic acid sample may comprise a plurality of sequences comprising a target sequence. At least about 80% of the plurality of sequences may comprise the target sequence. At least about 10%, at least about 20%, at least about 30%, at least about 40%, at least about 50%, at least about 60%, at least about 70%, at least about 80%, at least about 90% or more of the plurality of sequences may comprise the target amplification.

Methods of conducting amplification may provide a nucleic acid sample with high purity. The purity of a nucleic acid sample may be evaluated by measuring absorbance. Absorbance reads may be taken on a spectrophotometer. The method of measuring purity may comprise providing a sample comprising at least one molecule; measuring the absorbance of said sample at 260 nanometers (n); measuring the absorbance of said sample at 280 n; and providing a ratio of absorbance at 260 nm over 280 nm (A260/A280 ratio). Methods of conducting amplification may provide at least one sequencing read with an A260/A280 ratio of at most about 1.93. Methods of conducting amplification may provide at least one sequencing read with A260/A280 ratio of at most about 1.84. Methods of conducting amplification may provide at least one sequencing read with a A260/A280 ratio of at most about 5, at most 4.5, at most about 4, at most about 3.5, at most about 3, at most about 2.5, at most about 2, at most about 1.9, at most about 1.8. at most about 1.7, at most about 1.6, at most about 1.5, at most about 1.4, at most about 1.3, at most about 1.2, at most about 1.1, or less.

The circularized nucleic acid sample may comprise a plurality of sequences comprising a target sequence. At least about 80% of the plurality of sequences may comprise the target sequence. At least about 10%, at least about 20%, at least about 30%, at least about 40%, at least about 50%, at least about 60%, at least about 70%, at least about 80%, at least about 90% or more of the plurality of sequences may comprise the target sequence.

Bridge Amplification

The present disclosure provides a method of conducting amplification. The amplification may be in preparation for sequencing. The amplification may comprise bridge amplification. A double-stranded nucleic acid molecule may be provided and denatured. The original template of the denature double-stranded nucleic acid molecule may be washed away, thereby yielding a single-stranded nucleic acid molecule. The single-stranded nucleic acid molecule may be covalently attached to a flow cell surface. The single-stranded molecule may form a bridge. A second strand that is complementary to the bridge may be formed, thereby yielding a double-stranded nucleic acid bridge. The second strand may be formed via polymerases. The two strands may then separate, yielding two separate single-stranded nucleic acid molecules. This process may be repeated. Some of the strands may be removed from the flow cell surface. The 3′ ends of the remaining strands may be blocked. A sequencing primer may hybridize with the adapter sequence of each remaining strand. Examples of bridge amplification include, but are not limited to Illumina, Inc.'s Cluster Generation technology (see “Technology Spotlight: Illumina Sequencing Technology,” which is incorporated herein by reference in its entirety.)

The present disclosure provides a method of conducting bridge amplification on an electrowetting array. The electrowetting array may further comprise one or more reagent droplets. The one or more reagent droplets may comprise one or more reagents for performing bridge amplification. The one or more reagents may be, for example, a part of Illumina's NovaSeq 6000 kit (see “NovaSeq 6000 Reagent Kit,” which is incorporated herein by reference in its entirety.) The bridge amplification may be conducted in preparation for sequencing.

### Sequencing

Circular Consensus Sequencing

The present disclosure provides methods of sequencing a nucleic acid sample. The method of sequencing may generate long reads. The method of sequencing may generate long high-fidelity (HiFi) reads. The method of sequencing may comprise multiple passes of a single template molecule. In an example, the method of sequencing may be circular consensus sequencing. The sequencing reaction may comprise multiple passes. Each pass may produce at least one sequencing read. One or more subreads of the sequencing read may be produced. A consensus sequence may be produced from the subreads of the sequencing reads.

Examples of circular consensus sequencing are provided in Wenger, Aaron M., et al. “Accurate circular consensus long-read sequencing improves variant detection and assembly of a human genome.” Nature biotechnology 37.10 (2019): 1155-1162; Illumina's CirSeq (see https://www.illumina.com/science/sequencing-method-explorer/kits-and-arrays/cirseq.html); Acevedo, A., Andino R., “Library preparation for highly accurate population sequencing of RNA viruses.” Nat Protoc. 2014 July;9(7):1760-9); Hunt, M., Silva, N.D., Otto, T. D. et al. “Circlator: automated circularization of genome assemblies using long sequencing reads.” Genome Biol 16, 294 (2015); and Wilson, Brandon D et al. “High-Fidelity Nanopore Sequencing of Ultra-Short DNA Targets.” Analytical chemistry vol. 91,10 (2019): 6783-6789; U.S. Pat. Nos. 7,906,284; 10,563,255; and WO2009120374, all of which are incorporated herein by reference in their entirety.

The present disclosure provides a method of sequencing a nucleic acid sample, comprising (a) providing a droplet adjacent to an electrowetting array, which droplet comprises the nucleic acid sample, (b) using the electrowetting array to process the droplet to circularize the nucleic acid sample, and (c) using a single polymerizing enzyme to subject the circularized nucleic acid sample to a sequencing reaction.

The method of sequencing may further comprise combining the sample droplet with the one or more reagent droplets; separating the sample droplet from the one or more reagent droplets; and combining the one or more reagent droplets with a second droplet. The method may further comprise performing one or more droplet operations on the electrowetting array to process the droplet, wherein the one or more droplet operations comprise contacting the one or more reagent droplets with the droplet.

The electrowetting array may further comprise one or more reagent droplets. The one or more reagent droplets may comprise one or more reagents for circularizing the nucleic acid sample. The one or more reagent droplets may comprise one or more reagents performing rolling circle amplification (RCA). The one or more reagents may be enzymes. Examples of enzymes may include a nicking enzyme, a DNA polymerase, and an RCR protein. The one or more reagents may be control nucleic acids, buffer solutions and/or salt solutions, including, for example, divalent metal ions, i.e., Mg2+, Mn2+, Ca2+ and/or Fe2+. The one or more reagents may prepare single-stranded nucleic acids.

Methods of sequencing may yield a sequencing read having a length of at least about 70 kb, at least about 80 kb, at least about 90 kb, at least about 100 kb, at least about 110 kb, at least about 120 kb, at least about 130 kb, at least about 140 kb, at least about 150 kb, at least about 160 kb, at least about 170 kb, at least about 180 kb, at least about 190 kb, at least about 200 kb, or more.

In an example, methods of sequencing may be conducted on an electrowetting array. The nucleic acid may be deoxyribonucleic acid (DNA).

Methods of sequencing may yield at least about 100 Gigabytes (Gb) of sequencing data. Methods of sequencing may yield at least about 10 Gb of data. Methods of sequencing may yield at least about 30 Gb of sequencing data. Methods of sequencing may yield at least about 500 Gb of sequencing data. Methods of sequencing may yield at least about 512 Gb of sequencing data. Methods of sequencing may yield at least about 1, at least about 2, at least about 3, at least about 4, at least about 5, at least about 6, at least about 7, at least about 8, at least about 9, at least about 10, at least about 20, at least about 30, at least about 40, at least about 50, at least about 60, at least about 70, at least about 80, at least about 90, at least about 100, at least about 150, at least about 200, at least about 250, at least about 300, at least about 350, at least about 400, at least about 450, at least about 500 Gb or more of sequencing data.

The circularized nucleic acid sample may comprise a plurality of sequences comprising a target sequence. At least about 80% of the plurality of sequences may comprise the target sequence. At least about 10%, at least about 20%, at least about 30%, at least about 40%, at least about 50%, at least about 60%, at least about 70%, at least about 80%, at least about 90% or more of the plurality of sequences may comprise the target amplification.

Methods of sequencing may provide a nucleic acid sample with high purity. The purity of a nucleic acid sample may be evaluated by measuring absorbance. Absorbance reads may be taken on a spectrophotometer. The method of measuring purity may comprise providing a sample comprising at least one molecule; measuring the absorbance of said sample at 260 nanometers (nm); measuring the absorbance of said sample at 280 nm; and providing a ratio of absorbance at 260 nm over 280 nm (A260/A280 ratio). Methods of sequencing may provide at least one sequencing read with an A260/A280 ratio of at most about 1.93. Methods of sequencing may provide at least one sequencing read with A260/A280 ratio of at most about 1.84. Methods of sequencing may provide at least one sequencing read with a A260/A280 ratio of at most about 5, at most 4.5, at most about 4, at most about 3.5, at most about 3, at most about 2.5, at most about 2, at most about 1.9, at most about 1.8. at most about 1.7, at most about 1.6, at most about 1.5, at most about 1.4, at most about 1.3, at most about 1.2, at most about 1.1, or less.

Next Generation Sequencing

The present disclosure provides methods of sequencing a nucleic acid sample. The method of sequencing may comprise sequencing by synthesis (SBS). Four different fluorescently labelled deoxynucleotide triphosphates (dNTPs) and at least one polymerase may be provided. At least one fluorescent dNTP may be incorporated with at least one nucleic acid molecule to be sequenced, and subsequently imaged. A fluorescent dye and terminator may be cleared from the dNTP. The at least one nucleic acid can continue to be sequenced in this manner. In an example, four images during each cycle, and each dNTP may emit a different intensity. In another example, two images may be taken during each cycle, and the clyster intensities may be plotted. Examples of SBS include, but are not limited to Illumina, Inc.'s Cluster Generation technology (see “Technology Spotlight: Illumina Sequencing Technology,” which is incorporated herein by reference in its entirety.)

The present disclosure provides methods of sequencing a nucleic acid sample on an electrowetting array. The method may comprise combining a sample droplet with one or more reagent droplets; separating the sample droplet from the one or more reagent droplets; and combining the one or more reagent droplets with a second droplet. In an aspect, the one or more reagent droplets contain at least one reagent for NGS. In an aspect, the one or more reagent droplets contains at least one reagent for SBS. The reagent may be, for example, a part of Illumina's NovaSeq 6000 kit (see “NovaSeq 6000 Reagent Kit,” which is incorporated herein by reference in its entirety.)

Next-Generation Sequencing (NGS) Library Preparation and Evaporation Compensation

Evaporation compensation techniques described herein may not affect the reaction kinetics of NGS library preparation, providing applicability to a broad range of biological and chemical workflows described herein. Furthermore, a number of experiments can be run, and datasets can be built, from the same array for the evaporative loss for each of such chemical/biological reaction. For example, the datasets can be used to calculate compensation volumes required to keep a reaction volumes within a margin of error of, for example, 20%, 10%, 5%, 1%, or less. In reactions where there is volume loss, the compensation volume can be introduced in a timed fashion (e.g., in an open loop with no sensing and feedback). Alternatively, the dataset can be fed through a machine learning model to develop algorithms to learn how to estimate compensation volumes based on characteristics of the reactions. The datasets for feeding into the machine learning model can be generated from sensors adjacent to the arrays or can be generated from sensors external to the arrays. Similarly, datasets for improved ligation from simultaneous mixing and heating or improved fragmentation in response to active mixing on the array can be used to optimize performance of NGS sample preparation workflows using machine learning algorithms.

Polymerase Chain Reaction (PCR), Clean-Up, and Quantitative PCR (qPCR)

Nucleic acid molecules may be amplified by thermocycling-based Polymerase Chain Reaction (PCR) on an array described herein. A fixed region of the array may be heated or cooled. Alternatively, different regions on the array can be heated or cooled to different temperatures or temperature ranges. For example, one or more droplets containing PCR reagents and samples, can be shuttled back and forth between different zones of the array to perform PCR. A sensor (e.g., a fluorescent camera) can be used to illuminate and record a signal (e.g., fluorescence) of the droplet on the array. The detection may be carried out in real-time, providing qPCR functionality. For example, during qPCR operation, the signal may be read by monitoring a dsDNA binding dye (e.g., SYBR) or a fluorogenic probe (e.g., TaqMan). The signal may increase with accumulation of newly generated PCR products during each PCR cycle. To perform qPCR, an aliquot from a droplet (e.g., droplet volume can be on the pL-mL scale) can be used. By monitoring qPCR in this aliquot in real-time, the performance of the main sample can be inferred, and the amount of amplification required can be adjusted in response. PCR and qPCR operations on an array or a plurality of arrays may be multiplexed to track various amplicons in parallel (e.g., genes, markers of interest, NGS libraries, etc.). PCR and qPCR may be used for quantification of, for example, NGS libraries, gene expression, or target detection (e.g. diagnostics).

Nanoliter NGS

Input material and reagent quantity can be scaled-down to nanoliter-size or picoliter-size reaction volumes (e.g., droplets) on the arrays described herein. Reagent concentration may remain constant (e.g., for accurate reaction stoichiometry). Reagent starting and final concentration may not remain constant (e.g., increased or decreased), for example, optimizing reaction efficiency in a nanoliter—or picoliter-sized reaction volume.

Nanoliter- or picoliter-sized droplets on the open surface of an array (e.g., EWOD array or DEP array) or solid support (e.g., glass) may contact a much smaller area of an array compared to droplets sandwiched between two plates. The smaller areal occupancy may allow a large number of droplets (e.g., thousands of nanoliter droplets and millions of picoliter droplets) to be packed in a small footprint of the array (e.g., size of a standard SBS well plate). On an open array, for example, with a smooth slippery surface and with no interfacial forces from a second surface (e.g., from a second plate), nanoliter-sized droplets can be transported and mixed with forces (e.g., electromotive force from EWOD). Furthermore, the droplets can be, for example, heated, cooled, subjected to magnetic fields, or any combination thereof. Actuation of nanoliter- or picoliter-sized droplets may be accomplished on electrodes of dimensions comparable to the droplet contact area (e.g., 0.00001 millimeters (mm), 0.0001 mm, 0.001 mm, 0.01 mm, 0.1 mm, 1 mm, 10 mm, 100 mm, 1,000 mm, or more). Alternatively, a continuous set of electrodes surrounding the nanoliter- or picoliter-sized droplet can be activated simultaneously to generate sufficient electromotive force for transportation of the droplet(s). Reaction volumes and electrode sizes at this scale may provide at least about 1, 10, 100, 1,000, 10,000, 100,000, 1,000,000, or more reactions to proceed in parallel (e.g., making possible high-throughput applications in a the nanoliter- or picoliter-level. The process of scaling-down reactions, electrodes, input material, reagents, or any combination thereof may be automated using software simulation.

### Enzymatic Biopolymer Synthesis

Biopolymers (e.g., polynucleotides and polypeptides) may be synthesized on an array by dispensing and moving reagents sequentially, in parallel, or a combination thereof. The reagents may include, for example, nucleoside triphosphates, nucleotides, enzymes, buffers, beads, deblocking agents, water, salts, or any combination thereof. For example, polynucleotide (e.g. DNA) synthesis may occur directly on the surface of the array by functionalizing specific locations on the array. For example, the functionalized locations may act as reaction sites. DNA synthesis may also be performed on beads contained in droplets manipulated by the array (e.g., EWOD). DNA synthesis may be performed on the array at volumes on the scale of milliliters, microliters, nanoliters, picoliters, or femtoliters. DNA fragments may be assembled into longer fragments directly on the array by processes such as, for example, Gibson assembly. Combinatorial merging of droplets may be used to, for example, create a diversity of DNA fragments. The quality of the assembled DNA fragments can be assessed by sequencing library preparation on the array for downstream sequencing, such as, for example, Illumina—or Oxford Nanopore Technologies-based sequencing.

The present disclosure provides systems and methods for synthesizing at least one biopolymer on an array. In some embodiments, a sample droplet is brought into contact with at least one reagent droplet. In some embodiments the sample droplet and at least one reagent droplet are brought into contact by droplet operations. In some embodiments, the at least one reagent comprises a reagent for enzymatic biopolymer synthesis. Reagents and materials as used in the methods as disclosed herein can be found in, for example, U.S. Pat. No. 10,870,872 which is incorporated herein by reference in its entirety. In some embodiments, a biological product of synthesis is produced. In some aspects of the disclosure, the biological product of the synthesis is a polynucleotide. In other aspects of the disclosure, the biological product of the synthesis is a polypeptide.

Another aspect of the present disclosure is a method of generating a biopolymer, comprising: providing a plurality of droplets adjacent to a surface, wherein said plurality of droplets comprises a first droplet comprising a first reagent and a second droplet comprising a second reagent; subjecting said first droplet and said second droplet to motion relative to one another to (i) bring said first droplet in contact with said second droplet and (ii) form a merged droplet comprising said first reagent and said second reagent; and in said merged droplet, using at least (i) said first reagent and (ii) said second reagent to form at least a portion of said biopolymer, wherein (b)-(c) are performed in a time period of 10 minutes or less. In some embodiments, said biopolymer is a polynucleotide. In some embodiments, said biopolymer is a polypeptide. In some embodiments, where said polynucleotide comprises about 10 to about 250 bases. In some embodiments, where said polynucleotide comprises about 260 to about 1 kb. In some embodiments, said polynucleotide comprises about 1 kb to about 10,000 kb. In some embodiments, a vibration is applied to said synthesis droplet during (b), (c), or both. In some embodiments, the method further comprises, one or more washing steps comprising subjecting a wash droplet to motion to contact said merged droplet. In some embodiments, a vibration is applied to said one or more washing steps. In some embodiments, said surface is dielectric. In some embodiments, said surface comprises a dielectric layer disposed over one or more electrodes. In some embodiments, said surface is the surface of a polymeric film. In some embodiments, the surface comprises one or more oligonucleotides bound to the surface. In some embodiments, said surface is the surface of a lubricating liquid layer. In some embodiments, said plurality of droplets comprises a third droplet comprising a third reagent. In some embodiments, said first reagent, said second reagent, said third reagent, or any combination thereof, comprises one or more functionalized beads. In some embodiments, said functional beads comprise one or more oligonucleotides immobilized thereto. In some embodiments, a vibration is applied to either said first droplet, said second droplet, said third droplet, a wash droplet, or the mixtures thereof. In some embodiments, said first reagent, said second reagent, said third reagent or any combination thereof comprises a polymerase. In some embodiments, said first reagent, said second reagent, said third reagent or any combination thereof comprises a bio-monomer. In some embodiments, said bio-monomer is an amino acid. In some embodiments, said bio-monomer is a nucleic acid molecule. In some embodiments, said nucleic acid molecule comprises of adenine, cytosine, guanine, thymine, or uracil. In some embodiments, said first reagent, said second reagent, said third reagent, or any combination thereof, comprises one or more functionalized discs. In some embodiments, said functionalized disc comprise one or more oligonucleotides immobilized thereto. In some embodiments, said first reagent, said second reagent, said third reagent, or any combination thereof comprises an enzyme that mediates synthesis or polymerization. In some embodiments, said enzyme is from the group consisting of Polynucleotide Phosphorylase (PNPase), Terminal Denucleotidyl Transferas (TdT), DNA polymerase Beta, DNA polymerase lambda, DNA polymerase mu and other enzymes from X family of DNA polymerases. In some embodiments, at least one nucleic acid molecule of said polynucleotide is generated in 20 minutes or less within said merged droplet. In some embodiments, at least one nucleic acid molecule of said polynucleotide is generated in 15 minutes or less within said merged droplet. In some embodiments, at least one nucleic acid molecule of said polynucleotide is generated in 10 minutes or less within said merged droplet. In some embodiments, at least one nucleic acid molecule of said polynucleotide is generated in 1 minute or less within said merged droplet. In some embodiments, said merged droplet is temperature-controlled. In some embodiments, said first droplet, said second droplet, said third droplet, or said merged droplet is subjected to a magnetic field. In some embodiments, said first droplet, said second droplet, said third droplet, or said merged droplet is subjected to light. “In some embodiments, said first droplet, said second droplet, said third droplet, or said merged droplet is subjected to pH change. In some embodiments, said first droplet, said second droplet, said third droplet, or said merged droplet comprises of deoxynucleoside triphosphate (dNTP). In some embodiments, said deoxynucleoside triphosphate may have a protective group. In some embodiments, said protective group can be removed during the reaction. In some embodiments, said first droplet, said second droplet, said third droplet, or said merged droplet make contact with a surface only on one side. In some embodiments, volumes of said first droplet, said second droplet, said third droplet, or said merged droplet is between 1 nanoliter (1 nl) and 500 microliters (500 μl). In some embodiments, volumes of said first droplet, said second droplet, said third droplet, or said merged droplet is between 1 microliter (1 μl) and 500 microliters (500 μl). In some embodiments, volumes of said first droplet, said second droplet, said third droplet, or said merged droplet is between 1 microliter (1 μl) and 200 microliters (200 μl). In some embodiments, the method further comprises ligating said biopolymer to a second biopolymer. In some embodiments, said second biopolymer was generated using any method as disclosed herein.

Another aspect of the present disclosure provides a method of generating a biopolymer, comprising: providing a plurality of droplets adjacent to a surface, wherein said plurality of droplets comprises a first droplet comprising a first reagent and a second droplet comprising a second reagent; subjecting said first droplet and said second droplet to motion relative to one another to (i) bring said first droplet in contact with said second droplet and (ii) form a merged droplet comprising said first reagent and said second reagent; and in said merged droplet, using at least (i) said first reagent and (ii) said second reagent to form at least a portion of said biopolymer, wherein a vibration is applied to (b), (c), or both. In some embodiments, said biopolymer is a polynucleotide. In some embodiments, said biopolymer is a polypeptide. In some embodiments, said polynucleotide comprises 2 to 10,000,000 nucleic acid molecules. In some embodiments, the method further comprises, one or more washing steps comprising subjecting a wash droplet to motion to contact said merged droplet. In some embodiments, a vibration is applied to said one or more washing steps. In some embodiments, at least one nucleic acid molecule of said polynucleotide is generated in 30 minutes or less within said merged droplet. In some embodiments, said surface is dielectric. In some embodiments, said surface comprises a dielectric layer disposed over one or more electrodes. In some embodiments, said surface is the surface of a polymeric film. In some embodiments, the surface comprises one or more oligonucleotides bound to the surface. In some embodiments, said surface is the surface of a lubricating liquid layer. In some embodiments, said plurality of droplets comprises a third droplet comprising a third reagent. In some embodiments, said first reagent, said second reagent, said third reagent, or any combination thereof comprises one or more functionalized beads. In some embodiments, said functional beads comprise one or more oligonucleotides immobilized thereto. In some embodiments, said first reagent, said second reagent, said third reagent, or any combination thereof comprises a polymerase. In some embodiments, said first reagent, said second reagent, said third reagent or any combination thereof comprises a bio-monomer. In some embodiments, said bio-monomer is an amino acid. In some embodiments, said bio-monomer is a nucleic acid molecule. In some embodiments, said nucleic acid molecule is adenine, cytosine, guanine, thymine, or uracil. In some embodiments, said first reagent comprises one or more functionalized discs. In some embodiments, said functionalized disc comprise one or more oligonucleotides immobilized thereto. In some embodiments, said first droplet, second droplet, third droplet, or both comprises an enzyme that mediate synthesis or polymerization. In some embodiments, said enzyme is from the group consisting of Polynucleotide Phosphorylase (PNPase), Terminal Denucleotidyl Transferas (TdT), DNA polymerase Beta, DNA polymerase lambda, DNA polymerase mu and other enzymes from X family of DNA polymerases.

In some embodiments, said merged droplet is heated. In some embodiments, said first droplet, said second droplet, said third droplet, or said merged droplet is subjected to magnetic field. In some embodiments, said first droplet, said second droplet, said third droplet, or said merged droplet is subjected to light. In some embodiments, said first droplet, said second droplet, said third droplet, or said merged droplet is subjected to pH change. In some embodiments, said first droplet, said second droplet, said third droplet, or said merged droplet comprises of deoxynucleoside triphosphate (dNTP). In some embodiments, said deoxynucleoside triphosphate may have a protective group. In some embodiments, said protective group can be removed during the reaction. In some embodiments, said first droplet, said second droplet, said third droplet, or said merged droplet make contact with a surface only on one side. In some embodiments, volumes of said first droplet, said second droplet, said third droplet, or said merged droplet is between 1 nanoliter (1 nl) and 500 microliters (500 μl). In some embodiments, volumes of said first droplet, said second droplet, said third droplet, or said merged droplet is between 1 microliter (1 μl) and 500 microliters (500 μl). In some embodiments, volumes of said first droplet, said second droplet, said third droplet, or said merged droplet is between 1 microliter (1 μl) and 200 microliters (200 μl).

Another aspect of the present disclosure comprises a method for processing a nucleic acid sample, comprising: providing a biological sample adjacent to an electrowetting array, wherein said sample droplet comprises said nucleic acid sample; and extracting said nucleic acid sample from said biological sample adjacent to said electrowetting array wherein said nucleic acid sample comprises a sequencing read having a length of at least about 70 kilobases (kb) In some embodiments, said length is at least about 80 kilobases (kb). In some embodiments, said length is at least about 200 kilobases (kb). In some embodiments, said sequencing read comprises an A260/A280 ratio of less than about 1.84.

In some aspects of the disclosure, a biopolymer is ligated to a second biopolymer. In some embodiments, the second biopolymer is generated using any one of the methods described in this disclosure. The reservoirs for storing reagents (e.g., nucleoside triphosphates, magnetic beads, enzymes, salts, water, cleaving agents, or deblocking reagents) may be integrated on the surface of the array, integrated above the array, or dispensed from an external reservoir using a dispensing method described herein.

The biopolymer may be a polynucleotide. The polynucleotide may be at least 10, 100, 1,000, 10,000, 100,000, 1,000,000, or more base pairs long. In some embodiments, the length of the polynucleotide is about 1 base. In some embodiments, the length of the polynucleotide is about 1 base to about 750 bases. In some embodiments, the length of the polynucleotide is about 1 base to about 10 bases, about 1 base to about 20 bases, about 1 base to about 50 bases, about 1 base to about 100 bases, about 1 base to about 150 bases, about 1 base to about 200 bases, about 1 base to about 250 bases, about 1 base to about 500 bases, about 1 base to about 750 bases, about 1 base to about 1 base, about 10 bases to about 20 bases, about 10 bases to about 50 bases, about 10 bases to about 100 bases, about 10 bases to about 150 bases, about 10 bases to about 200 bases, about 10 bases to about 250 bases, about 10 bases to about 500 bases, about 10 bases to about 750 bases, about 10 bases to about 1 base, about 20 bases to about 50 bases, about 20 bases to about 100 bases, about 20 bases to about 150 bases, about 20 bases to about 200 bases, about 20 bases to about 250 bases, about 20 bases to about 500 bases, about 20 bases to about 750 bases, about 20 bases to about 1 base, about 50 bases to about 100 bases, about 50 bases to about 150 bases, about 50 bases to about 200 bases, about 50 bases to about 250 bases, about 50 bases to about 500 bases, about 50 bases to about 750 bases, about 50 bases to about 1 base, about 100 bases to about 150 bases, about 100 bases to about 200 bases, about 100 bases to about 250 bases, about 100 bases to about 500 bases, about 100 bases to about 750 bases, about 100 bases to about 1 base, about 150 bases to about 200 bases, about 150 bases to about 250 bases, about 150 bases to about 500 bases, about 150 bases to about 750 bases, about 150 bases to about 1 base, about 200 bases to about 250 bases, about 200 bases to about 500 bases, about 200 bases to about 750 bases, about 200 bases to about 1 base, about 250 bases to about 500 bases, about 250 bases to about 750 bases, about 250 bases to about 1 base, about 500 bases to about 750 bases, about 500 bases to about 1 base, or about 750 bases to about 1 base. In some embodiments, the length of the polynucleotide is about 1 base, about 10 bases, about 20 bases, about 50 bases, about 100 bases, about 150 bases, about 200 bases, about 250 bases, about 500 bases, about 750 bases, or about 1 base. In some embodiments, the length of the polynucleotide is at least about 1 base, about 10 bases, about 20 bases, about 50 bases, about 100 bases, about 150 bases, about 200 bases, about 250 bases, about 500 bases, or about 750 bases. In some embodiments, the length of the polynucleotide is at most about 10 bases, about 20 bases, about 50 bases, about 100 bases, about 150 bases, about 200 bases, about 250 bases, about 500 bases, about 750 bases, or about 1 base.

In some embodiments, the length of the polynucleotide is about 1 kilobase (kb) to about 250 kilobases (kbs). In some embodiments, the length of the polynucleotide is about 1 kilobase (kb) to about 2 kilobases (kbs), about 1 kilobase (kb) to about 3 kilobases (kbs), about 1 kilobase (kb) to about 4 kilobases (kbs), about 1 kilobase (kb) to about 5 kilobases (kbs), about 1 kilobase (kb) to about 10 kilobases (kbs), about 1 kilobase (kb) to about 20 kilobases (kbs), about 1 kilobase (kb) to about 50 kilobases (kbs), about 1 kilobase (kb) to about 100 kilobases (kbs), about 1 kilobase (kb) to about 150 kilobases (kbs), about 1 kilobase (kb) to about 200 kilobases (kbs), about 1 kilobase (kb) to about 250 kilobases (kbs), about 2 kilobases (kbs) to about 3 kilobases (kbs), about 2 kilobases (kbs) to about 4 kilobases (kbs), about 2 kilobases (kbs) to about 5 kilobases (kbs), about 2 kilobases (kbs) to about 10 kilobases (kbs), about 2 kilobases (kbs) to about 20 kilobases (kbs), about 2 kilobases (kbs) to about 50 kilobases (kbs), about 2 kilobases (kbs) to about 100 kilobases (kbs), about 2 kilobases (kbs) to about 150 kilobases (kbs), about 2 kilobases (kbs) to about 200 kilobases (kbs), about 2 kilobases (kbs) to about 250 kilobases (kbs), about 3 kilobases (kbs) to about 4 kilobases (kbs), about 3 kilobases (kbs) to about 5 kilobases (kbs), about 3 kilobases (kbs) to about 10 kilobases (kbs), about 3 kilobases (kbs) to about 20 kilobases (kbs), about 3 kilobases (kbs) to about 50 kilobases (kbs), about 3 kilobases (kbs) to about 100 kilobases (kbs), about 3 kilobases (kbs) to about 150 kilobases (kbs), about 3 kilobases (kbs) to about 200 kilobases (kbs), about 3 kilobases (kbs) to about 250 kilobases (kbs), about 4 kilobases (kbs) to about 5 kilobases (kbs), about 4 kilobases (kbs) to about 10 kilobases (kbs), about 4 kilobases (kbs) to about 20 kilobases (kbs), about 4 kilobases (kbs) to about 50 kilobases (kbs), about 4 kilobases (kbs) to about 100 kilobases (kbs), about 4 kilobases (kbs) to about 150 kilobases (kbs), about 4 kilobases (kbs) to about 200 kilobases (kbs), about 4 kilobases (kbs) to about 250 kilobases (kbs), about 5 kilobases (kbs) to about 10 kilobases (kbs), about 5 kilobases (kbs) to about 20 kilobases (kbs), about 5 kilobases (kbs) to about 50 kilobases (kbs), about 5 kilobases (kbs) to about 100 kilobases (kbs), about 5 kilobases (kbs) to about 150 kilobases (kbs), about 5 kilobases (kbs) to about 200 kilobases (kbs), about 5 kilobases (kbs) to about 250 kilobases (kbs), about 10 kilobases (kbs) to about 20 kilobases (kbs), about 10 kilobases (kbs) to about 50 kilobases (kbs), about 10 kilobases (kbs) to about 100 kilobases (kbs), about 10 kilobases (kbs) to about 150 kilobases (kbs), about 10 kilobases (kbs) to about 200 kilobases (kbs), about 10 kilobases (kbs) to about 250 kilobases (kbs), about 20 kilobases (kbs) to about 50 kilobases (kbs), about 20 kilobases (kbs) to about 100 kilobases (kbs), about 20 kilobases (kbs) to about 150 kilobases (kbs), about 20 kilobases (kbs) to about 200 kilobases (kbs), about 20 kilobases (kbs) to about 250 kilobases (kbs), about 50 kilobases (kbs) to about 100 kilobases (kbs), about 50 kilobases (kbs) to about 150 kilobases (kbs), about 50 kilobases (kbs) to about 200 kilobases (kbs), about 50 kilobases (kbs) to about 250 kilobases (kbs), about 100 kilobases (kbs) to about 150 kilobases (kbs), about 100 kilobases (kbs) to about 200 kilobases (kbs), about 100 kilobases (kbs) to about 250 kilobases (kbs), about 150 kilobases (kbs) to about 200 kilobases (kbs), about 150 kilobases (kbs) to about 250 kilobases (kbs), or about 200 kilobases (kbs) to about 250 kilobases (kbs). In some embodiments, the length of the polynucleotide is about 1 kilobase (kb), about 2 kilobases (kbs), about 3 kilobases (kbs), about 4 kilobases (kbs), about 5 kilobases (kbs), about 10 kilobases (kbs), about 20 kilobases (kbs), about 50 kilobases (kbs), about 100 kilobases (kbs), about 150 kilobases (kbs), about 200 kilobases (kbs), or about 250 kilobases (kbs). In some embodiments, the length of the polynucleotide is at least about 1 kilobase (kb), about 2 kilobases (kbs), about 3 kilobases (kbs), about 4 kilobases (kbs), about 5 kilobases (kbs), about 10 kilobases (kbs), about 20 kilobases (kbs), about 50 kilobases (kbs), about 100 kilobases (kbs), about 150 kilobases (kbs), or about 200 kilobases (kbs). In some embodiments, the length of the polynucleotide is at most about 2 kilobases (kbs), about 3 kilobases (kbs), about 4 kilobases (kbs), about 5 kilobases (kbs), about 10 kilobases (kbs), about 20 kilobases (kbs), about 50 kilobases (kbs), about 100 kilobases (kbs), about 150 kilobases (kbs), about 200 kilobases (kbs), or about 250 kilobases (kbs).

In some embodiments, the length of the polynucleotide is about 250 kilobases (kbs) to about 10,000 kilobases (kbs). In some embodiments, the length of the polynucleotide is about 250 kilobases (kbs) to about 500 kilobases (kbs), about 250 kilobases (kbs) to about 750 kilobases (kbs), about 250 kilobases (kbs) to about 1,000 kilobases (kbs), about 250 kilobases (kbs) to about 2,000 kilobases (kbs), about 250 kilobases (kbs) to about 3,000 kilobases (kbs), about 250 kilobases (kbs) to about 4,000 kilobases (kbs), about 250 kilobases (kbs) to about 5,000 kilobases (kbs), about 250 kilobases (kbs) to about 10,000 kilobases (kbs), about 500 kilobases (kbs) to about 750 kilobases (kbs), about 500 kilobases (kbs) to about 1,000 kilobases (kbs), about 500 kilobases (kbs) to about 2,000 kilobases (kbs), about 500 kilobases (kbs) to about 3,000 kilobases (kbs), about 500 kilobases (kbs) to about 4,000 kilobases (kbs), about 500 kilobases (kbs) to about 5,000 kilobases (kbs), about 500 kilobases (kbs) to about 10,000 kilobases (kbs), about 750 kilobases (kbs) to about 1,000 kilobases (kbs), about 750 kilobases (kbs) to about 2,000 kilobases (kbs), about 750 kilobases (kbs) to about 3,000 kilobases (kbs), about 750 kilobases (kbs) to about 4,000 kilobases (kbs), about 750 kilobases (kbs) to about 5,000 kilobases (kbs), about 750 kilobases (kbs) to about 10,000 kilobases (kbs), about 1,000 kilobases (kbs) to about 2,000 kilobases (kbs), about 1,000 kilobases (kbs) to about 3,000 kilobases (kbs), about 1,000 kilobases (kbs) to about 4,000 kilobases (kbs), about 1,000 kilobases (kbs) to about 5,000 kilobases (kbs), about 1,000 kilobases (kbs) to about 10,000 kilobases (kbs), about 2,000 kilobases (kbs) to about 3,000 kilobases (kbs), about 2,000 kilobases (kbs) to about 4,000 kilobases (kbs), about 2,000 kilobases (kbs) to about 5,000 kilobases (kbs), about 2,000 kilobases (kbs) to about 10,000 kilobases (kbs), about 3,000 kilobases (kbs) to about 4,000 kilobases (kbs), about 3,000 kilobases (kbs) to about 5,000 kilobases (kbs), about 3,000 kilobases (kbs) to about 10,000 kilobases (kbs), about 4,000 kilobases (kbs) to about 5,000 kilobases (kbs), about 4,000 kilobases (kbs) to about 10,000 kilobases (kbs), or about 5,000 kilobases (kbs) to about 10,000 kilobases (kbs). In some embodiments, the length of the polynucleotide is about 250 kilobases (kbs), about 500 kilobases (kbs), about 750 kilobases (kbs), about 1,000 kilobases (kbs), about 2,000 kilobases (kbs), about 3,000 kilobases (kbs), about 4,000 kilobases (kbs), about 5,000 kilobases (kbs), or about 10,000 kilobases (kbs). In some embodiments, the length of the polynucleotide is at least about 250 kilobases (kbs), about 500 kilobases (kbs), about 750 kilobases (kbs), about 1,000 kilobases (kbs), about 2,000 kilobases (kbs), about 3,000 kilobases (kbs), about 4,000 kilobases (kbs), or about 5,000 kilobases (kbs). In some embodiments, the length of the polynucleotide is at most about 500 kilobases (kbs), about 750 kilobases (kbs), about 1,000 kilobases (kbs), about 2,000 kilobases (kbs), about 3,000 kilobases (kbs), about 4,000 kilobases (kbs), about 5,000 kilobases (kbs), or about 10,000 kilobases (kbs).

In some aspects of present disclosure, the polynucleotide comprises about 2 to about 10,000,000 nucleic acid molecules. In some embodiments, the polynucleotide comprises about 1 nucleic acid to about 1,000 nucleic acids. In some embodiments, the polynucleotide comprises about 1 nucleic acid to about 2 nucleic acids, about 1 nucleic acid to about 5 nucleic acids, about 1 nucleic acid to about 10 nucleic acids, about 1 nucleic acid to about 25 nucleic acids, about 1 nucleic acid to about 50 nucleic acids, about 1 nucleic acid to about 100 nucleic acids, about 1 nucleic acid to about 250 nucleic acids, about 1 nucleic acid to about 500 nucleic acids, about 1 nucleic acid to about 750 nucleic acids, about 1 nucleic acid to about 1,000 nucleic acids, about 2 nucleic acids to about 5 nucleic acids, about 2 nucleic acids to about 10 nucleic acids, about 2 nucleic acids to about 25 nucleic acids, about 2 nucleic acids to about 50 nucleic acids, about 2 nucleic acids to about 100 nucleic acids, about 2 nucleic acids to about 250 nucleic acids, about 2 nucleic acids to about 500 nucleic acids, about 2 nucleic acids to about 750 nucleic acids, about 2 nucleic acids to about 1,000 nucleic acids, about 5 nucleic acids to about 10 nucleic acids, about 5 nucleic acids to about 25 nucleic acids, about 5 nucleic acids to about 50 nucleic acids, about 5 nucleic acids to about 100 nucleic acids, about 5 nucleic acids to about 250 nucleic acids, about 5 nucleic acids to about 500 nucleic acids, about 5 nucleic acids to about 750 nucleic acids, about 5 nucleic acids to about 1,000 nucleic acids, about 10 nucleic acids to about 25 nucleic acids, about 10 nucleic acids to about 50 nucleic acids, about 10 nucleic acids to about 100 nucleic acids, about 10 nucleic acids to about 250 nucleic acids, about 10 nucleic acids to about 500 nucleic acids, about 10 nucleic acids to about 750 nucleic acids, about 10 nucleic acids to about 1,000 nucleic acids, about 25 nucleic acids to about 50 nucleic acids, about 25 nucleic acids to about 100 nucleic acids, about 25 nucleic acids to about 250 nucleic acids, about 25 nucleic acids to about 500 nucleic acids, about 25 nucleic acids to about 750 nucleic acids, about 25 nucleic acids to about 1,000 nucleic acids, about 50 nucleic acids to about 100 nucleic acids, about 50 nucleic acids to about 250 nucleic acids, about 50 nucleic acids to about 500 nucleic acids, about 50 nucleic acids to about 750 nucleic acids, about 50 nucleic acids to about 1,000 nucleic acids, about 100 nucleic acids to about 250 nucleic acids, about 100 nucleic acids to about 500 nucleic acids, about 100 nucleic acids to about 750 nucleic acids, about 100 nucleic acids to about 1,000 nucleic acids, about 250 nucleic acids to about 500 nucleic acids, about 250 nucleic acids to about 750 nucleic acids, about 250 nucleic acids to about 1,000 nucleic acids, about 500 nucleic acids to about 750 nucleic acids, about 500 nucleic acids to about 1,000 nucleic acids, or about 750 nucleic acids to about 1,000 nucleic acids. In some embodiments, the polynucleotide comprises about 1 nucleic acid, about 2 nucleic acids, about 5 nucleic acids, about 10 nucleic acids, about 25 nucleic acids, about 50 nucleic acids, about 100 nucleic acids, about 250 nucleic acids, about 500 nucleic acids, about 750 nucleic acids, or about 1,000 nucleic acids. In some embodiments, the polynucleotide comprises at least about 1 nucleic acid, about 2 nucleic acids, about 5 nucleic acids, about 10 nucleic acids, about 25 nucleic acids, about 50 nucleic acids, about 100 nucleic acids, about 250 nucleic acids, about 500 nucleic acids, or about 750 nucleic acids. In some embodiments, the polynucleotide comprises at most about 2 nucleic acids, about 5 nucleic acids, about 10 nucleic acids, about 25 nucleic acids, about 50 nucleic acids, about 100 nucleic acids, about 250 nucleic acids, about 500 nucleic acids, about 750 nucleic acids, or about 1,000 nucleic acids.

In some embodiments, the polynucleotide comprises about 1,000 nucleic acids to about 100,000 nucleic acids. In some embodiments, the polynucleotide comprises about 1,000 nucleic acids to about 2,000 nucleic acids, about 1,000 nucleic acids to about 5,000 nucleic acids, about 1,000 nucleic acids to about 7,500 nucleic acids, about 1,000 nucleic acids to about 10,000 nucleic acids, about 1,000 nucleic acids to about 20,000 nucleic acids, about 1,000 nucleic acids to about 50,000 nucleic acids, about 1,000 nucleic acids to about 75,000 nucleic acids, about 1,000 nucleic acids to about 100,000 nucleic acids, about 2,000 nucleic acids to about 5,000 nucleic acids, about 2,000 nucleic acids to about 7,500 nucleic acids, about 2,000 nucleic acids to about 10,000 nucleic acids, about 2,000 nucleic acids to about 20,000 nucleic acids, about 2,000 nucleic acids to about 50,000 nucleic acids, about 2,000 nucleic acids to about 75,000 nucleic acids, about 2,000 nucleic acids to about 100,000 nucleic acids, about 5,000 nucleic acids to about 7,500 nucleic acids, about 5,000 nucleic acids to about 10,000 nucleic acids, about 5,000 nucleic acids to about 20,000 nucleic acids, about 5,000 nucleic acids to about 50,000 nucleic acids, about 5,000 nucleic acids to about 75,000 nucleic acids, about 5,000 nucleic acids to about 100,000 nucleic acids, about 7,500 nucleic acids to about 10,000 nucleic acids, about 7,500 nucleic acids to about 20,000 nucleic acids, about 7,500 nucleic acids to about 50,000 nucleic acids, about 7,500 nucleic acids to about 75,000 nucleic acids, about 7,500 nucleic acids to about 100,000 nucleic acids, about 10,000 nucleic acids to about 20,000 nucleic acids, about 10,000 nucleic acids to about 50,000 nucleic acids, about 10,000 nucleic acids to about 75,000 nucleic acids, about 10,000 nucleic acids to about 100,000 nucleic acids, about 20,000 nucleic acids to about 50,000 nucleic acids, about 20,000 nucleic acids to about 75,000 nucleic acids, about 20,000 nucleic acids to about 100,000 nucleic acids, about 50,000 nucleic acids to about 75,000 nucleic acids, about 50,000 nucleic acids to about 100,000 nucleic acids, or about 75,000 nucleic acids to about 100,000 nucleic acids. In some embodiments, the polynucleotide comprises about 1,000 nucleic acids, about 2,000 nucleic acids, about 5,000 nucleic acids, about 7,500 nucleic acids, about 10,000 nucleic acids, about 20,000 nucleic acids, about 50,000 nucleic acids, about 75,000 nucleic acids, or about 100,000 nucleic acids. In some embodiments, the polynucleotide comprises at least about 1,000 nucleic acids, about 2,000 nucleic acids, about 5,000 nucleic acids, about 7,500 nucleic acids, about 10,000 nucleic acids, about 20,000 nucleic acids, about 50,000 nucleic acids, or about 75,000 nucleic acids. In some embodiments, the polynucleotide comprises at most about 2,000 nucleic acids, about 5,000 nucleic acids, about 7,500 nucleic acids, about 10,000 nucleic acids, about 20,000 nucleic acids, about 50,000 nucleic acids, about 75,000 nucleic acids, or about 100,000 nucleic acids.

In some embodiments, the polynucleotide comprises about 100,000 nucleic acids to about 10,000,000 nucleic acids. In some embodiments, the polynucleotide comprises about 100,000 nucleic acids to about 200,000 nucleic acids, about 100,000 nucleic acids to about 750,000 nucleic acids, about 100,000 nucleic acids to about 1,000,000 nucleic acids, about 100,000 nucleic acids to about 2,000,000 nucleic acids, about 100,000 nucleic acids to about 5,000,000 nucleic acids, about 100,000 nucleic acids to about 7,500,000 nucleic acids, about 100,000 nucleic acids to about 10,000,000 nucleic acids, about 200,000 nucleic acids to about 750,000 nucleic acids, about 200,000 nucleic acids to about 1,000,000 nucleic acids, about 200,000 nucleic acids to about 2,000,000 nucleic acids, about 200,000 nucleic acids to about 5,000,000 nucleic acids, about 200,000 nucleic acids to about 7,500,000 nucleic acids, about 200,000 nucleic acids to about 10,000,000 nucleic acids, about 750,000 nucleic acids to about 1,000,000 nucleic acids, about 750,000 nucleic acids to about 2,000,000 nucleic acids, about 750,000 nucleic acids to about 5,000,000 nucleic acids, about 750,000 nucleic acids to about 7,500,000 nucleic acids, about 750,000 nucleic acids to about 10,000,000 nucleic acids, about 1,000,000 nucleic acids to about 2,000,000 nucleic acids, about 1,000,000 nucleic acids to about 5,000,000 nucleic acids, about 1,000,000 nucleic acids to about 7,500,000 nucleic acids, about 1,000,000 nucleic acids to about 10,000,000 nucleic acids, about 2,000,000 nucleic acids to about 5,000,000 nucleic acids, about 2,000,000 nucleic acids to about 7,500,000 nucleic acids, about 2,000,000 nucleic acids to about 10,000,000 nucleic acids, about 5,000,000 nucleic acids to about 7,500,000 nucleic acids, about 5,000,000 nucleic acids to about 10,000,000 nucleic acids, or about 7,500,000 nucleic acids to about 10,000,000 nucleic acids. In some embodiments, the polynucleotide comprises about 100,000 nucleic acids, about 200,000 nucleic acids, about 750,000 nucleic acids, about 1,000,000 nucleic acids, about 2,000,000 nucleic acids, about 5,000,000 nucleic acids, about 7,500,000 nucleic acids, or about 10,000,000 nucleic acids. In some embodiments, the polynucleotide comprises at least about 100,000 nucleic acids, about 200,000 nucleic acids, about 750,000 nucleic acids, about 1,000,000 nucleic acids, about 2,000,000 nucleic acids, about 5,000,000 nucleic acids, or about 7,500,000 nucleic acids. In some embodiments, the polynucleotide comprises at most about 200,000 nucleic acids, about 750,000 nucleic acids, about 1,000,000 nucleic acids, about 2,000,000 nucleic acids, about 5,000,000 nucleic acids, about 7,500,000 nucleic acids, or about 10,000,000 nucleic acids.

In some embodiments, at least one nucleic acid molecule of said polynucleotide is generated in 20 minutes or less within said merged droplet. In some embodiments, at least one nucleic acid molecule of said polynucleotide is generated in 15 minutes or less within said merged droplet. In some embodiments, at least one nucleic acid molecule of said polynucleotide is generated in 10 minutes or less within said merged droplet. In some embodiments, at least one nucleic acid molecule of said polynucleotide is generated in about 5 minutes or less to about 20 minutes or less. In some embodiments, at least one nucleic acid molecule of said polynucleotide is generated in about 20 minutes or less to about 19 minutes or less, about 20 minutes or less to about 18 minutes or less, about 20 minutes or less to about 17 minutes or less, about 20 minutes or less to about 16 minutes or less, about 20 minutes or less to about 15 minutes or less, about 20 minutes or less to about 14 minutes or less, about 20 minutes or less to about 13 minutes or less, about 20 minutes or less to about 12 minutes or less, about 20 minutes or less to about 11 minutes or less, about 20 minutes or less to about 10 minutes or less, about 20 minutes or less to about 5 minutes or less, about 19 minutes or less to about 18 minutes or less, about 19 minutes or less to about 17 minutes or less, about 19 minutes or less to about 16 minutes or less, about 19 minutes or less to about 15 minutes or less, about 19 minutes or less to about 14 minutes or less, about 19 minutes or less to about 13 minutes or less, about 19 minutes or less to about 12 minutes or less, about 19 minutes or less to about 11 minutes or less, about 19 minutes or less to about 10 minutes or less, about 19 minutes or less to about 5 minutes or less, about 18 minutes or less to about 17 minutes or less, about 18 minutes or less to about 16 minutes or less, about 18 minutes or less to about 15 minutes or less, about 18 minutes or less to about 14 minutes or less, about 18 minutes or less to about 13 minutes or less, about 18 minutes or less to about 12 minutes or less, about 18 minutes or less to about 11 minutes or less, about 18 minutes or less to about 10 minutes or less, about 18 minutes or less to about 5 minutes or less, about 17 minutes or less to about 16 minutes or less, about 17 minutes or less to about 15 minutes or less, about 17 minutes or less to about 14 minutes or less, about 17 minutes or less to about 13 minutes or less, about 17 minutes or less to about 12 minutes or less, about 17 minutes or less to about 11 minutes or less, about 17 minutes or less to about 10 minutes or less, about 17 minutes or less to about 5 minutes or less, about 16 minutes or less to about 15 minutes or less, about 16 minutes or less to about 14 minutes or less, about 16 minutes or less to about 13 minutes or less, about 16 minutes or less to about 12 minutes or less, about 16 minutes or less to about 11 minutes or less, about 16 minutes or less to about 10 minutes or less, about 16 minutes or less to about 5 minutes or less, about 15 minutes or less to about 14 minutes or less, about 15 minutes or less to about 13 minutes or less, about 15 minutes or less to about 12 minutes or less, about 15 minutes or less to about 11 minutes or less, about 15 minutes or less to about 10 minutes or less, about 15 minutes or less to about 5 minutes or less, about 14 minutes or less to about 13 minutes or less, about 14 minutes or less to about 12 minutes or less, about 14 minutes or less to about 11 minutes or less, about 14 minutes or less to about 10 minutes or less, about 14 minutes or less to about 5 minutes or less, about 13 minutes or less to about 12 minutes or less, about 13 minutes or less to about 11 minutes or less, about 13 minutes or less to about 10 minutes or less, about 13 minutes or less to about 5 minutes or less, about 12 minutes or less to about 11 minutes or less, about 12 minutes or less to about 10 minutes or less, about 12 minutes or less to about 5 minutes or less, about 11 minutes or less to about 10 minutes or less, about 11 minutes or less to about 5 minutes or less, or about 10 minutes or less to about 5 minutes or less. In some embodiments, at least one nucleic acid molecule of said polynucleotide is generated in about 20 minutes or less, about 19 minutes or less, about 18 minutes or less, about 17 minutes or less, about 16 minutes or less, about 15 minutes or less, about 14 minutes or less, about 13 minutes or less, about 12 minutes or less, about 11 minutes or less, about 10 minutes or less, or about 5 minutes or less. In some embodiments, at least one nucleic acid molecule of said polynucleotide is generated in at least about 20 minutes or less, about 19 minutes or less, about 18 minutes or less, about 17 minutes or less, about 16 minutes or less, about 15 minutes or less, about 14 minutes or less, about 13 minutes or less, about 12 minutes or less, about 11 minutes or less, or about 10 minutes or less. In some embodiments, at least one nucleic acid molecule of said polynucleotide is generated in at most about 19 minutes or less, about 18 minutes or less, about 17 minutes or less, about 16 minutes or less, about 15 minutes or less, about 14 minutes or less, about 13 minutes or less, about 12 minutes or less, about 11 minutes or less, about 10 minutes or less, or about 5 minutes or less.

1, 10, 100, 1,000, 10,000, 100,000, 1,000,000, or more reactions may be performed in parallel on a single array or on multiple arrays. Droplets with polynucleotide (e.g., DNA) sequences can be purified and size-selected using magnetic bead. The purified DNA sequences can be merged and assembled in a combinatorial fashion using DNA assembly techniques, such as, for example, Gibson assembly. The assembled polynucleotides (e.g., DNA) may contain errors. To correct for the errors, the assembled polynucleotide (e.g., DNA) may be treated with mismatch binding or mismatch cleaving proteins (e.g., MutS, T4 endonuclease VII, or T7 endonuclease I).

The arrays for synthesizing DNA using enzymatic processes can be stacked vertically or horizontally as described herein. The stacks may be connected to a cloud server infrastructure. For example, when a user procures a sequence of, for example, DNA, gene pools, RNA, guide RNA, or other biopolymers, the user can interact with a dashboard on a computer that connects directly to the cloud infrastructure. Upon submitting an input sequence for synthesis, a finite set of arrays may be instantiated on demand. The number of arrays can be from, for example, one to several billion. Once the arrays are instantiated, the entire synthesis process may be run autonomously.

### Sample Quantification

Optical-based (e.g., fluorescence) detection of nucleic acids (e.g., DNA) on the array may be implemented by using, for example, intercalating fluorescent dyes (e.g., SYBR Green). For making fluorescence-based measurements, a sample can be positioned in the sample detection zone (5710) from another portion of the array. The sample detection zone may be an optically clear path (e.g., transparent or a hole in the surface). The excitation source, an excitation filter, a mirror, an emission filter, the detection sensor, or any combination thereof may be positioned below the sample to allow light to excite and travel back through the optically clear path.

For example, A size selection unit may precede the fluorescence-based detection zone. The size-based separation unit may employ electrophoresis or capillary electrophoresis to separate nucleic acid fragments based on their size. The size-separated sample can be passed through a detection zone where the fluorescence signal distribution of the sample may be indicative of the sample's size distribution. The total fluorescence of the sample may be used to quantify the concentration of total nucleic acid in the sample.

The nucleic acid sequencer may be a Maxam-Gilbert sequencer or a Sanger sequencer. The biological protein channel may be a biological nanopore. The biological protein channel may be a hemolysin or an MspA porin. The solid state nanopore may be silicon nitride or graphene. The protein sequencer may be a mass spectrometer, a single molecule sequencer, or an Edman degradation sequencer. The nucleic acid sequencing may comprise sequencing by synthesis, pyrosequencing, sequencing by hybridization, sequencing by ligation, sequencing by detection of ions released during polymerization of DNA, single-molecule sequencing, or any combination thereof. The single molecule sequencing may be nanopore sequencing. The single molecule sequencing may be single molecule real time (SMRT) sequencing.

In some embodiments, the nucleic acid and/or protein sequencing/identification assay(s) can be integrated into the EWOD systems, devices, and/or arrays described herein. In some embodiments, a nanopore sensor may be integrated into an EWOD systems, devices, and/or arrays described herein to perform biomolecular sensing and sequencing. In some embodiments, the EWOD systems, devices, and/or arrays described herein and the Nanopore sensor can all be fabricated in a monolithic silicon. In some embodiments, the EWOD systems, devices, and/or arrays described herein can be fabricated with standard electronics fabrication practices (e.g. as described herein) and nanopore sensor can be fabricated with silicon process and mounted adjacent to, or coupled to the EWOD systems, devices, and/or arrays described herein. In some embodiments, the nanopore sensor may contain a protein-based pore for sensing or it can be entirely solid state.

In some embodiments, the nanopore may be integrated into the EWOD systems, devices, and/or arrays described herein on the same plane as the droplets. In some embodiments, the nanopore may be situated above, below, or to the side of the electrowetting surface/array. The EWOD systems, devices, and/or arrays described herein may have a hole through which the droplet containing biological samples is transferred to the nanopore sensor.

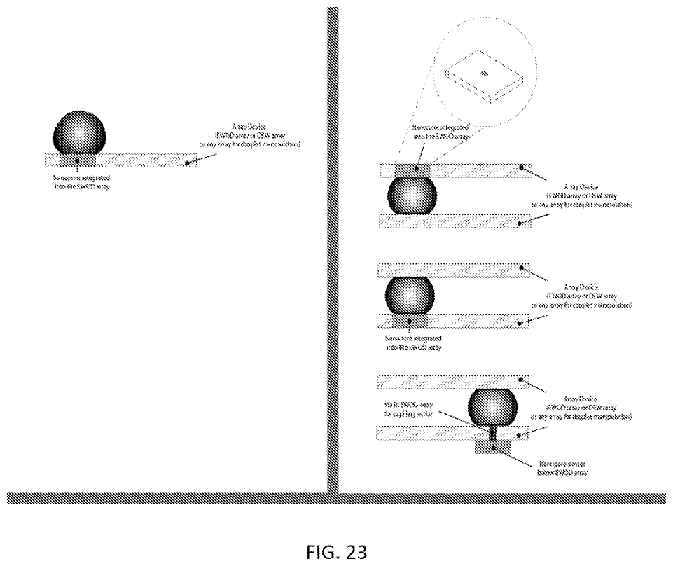

An embodiment of this aspect of the present disclosure is exemplified in FIG. 23.

The acoustic transducer may be subsonic, ultrasonic, or a combination thereof. The acoustic transducer may be coupled to the array by an acoustic coupling medium. The acoustic coupling medium may be a solid or a liquid. The MEMS transducer may measure force, pressure, or temperature. The capillary tubes as liquid dispensers may be about 2 millimeters (mm) in diameter, 1.5 mm in diameter, 1 mm in diameter, 0.5 mm in diameter, 0.25 mm in diameter, or smaller. There may be 1, 2, 3, 4, 5, 10, 50, 100, or more capillary tubes in the array. The holes for dispensing or transferring liquids using gravity may be treated with different materials to increase or decrease the hydrophobicity of the hole. There may be 1, 2, 3, 4, 5, 10, 50, 100, or more holes in the array. The holes may be at least about 100 μm, 200 μm, 300 μm, 400 μm, 500 μm, 600 μm, 700 μm, 800 μm, 900 μm, 1,000 μm, 1,100 μm, 1,200 μm, 1,300 μm, 1,400 μm, 1,500 μm, 1,600 μm, 1,700 μm, 1,800 μm, 1,900 μm, 2,000 μm, or more in diameter. The holes may be at most about 2,000 μm, 1,900 μm, 1,800 μm, 1,700 μm, 1,600 μm, 1,500 μm, 1,400 μm, 1,300 μm, 1,200 μm, 1,000 μm, 900 μm, 800 μm, 700 μm, 600 μm, 500 μm, 400 μm, 300 μm, 200 μm, 100 μm, or less in diameter. The holes may be from 100 μm to 500 μm in diameter. The electrode in a hole to dispense or transfer liquid may use the electrowetting effect. The holes may be for optical inspection. The holes may be of a size described herein. The holes for liquids to interact through a membrane may have a membrane of a material described herein. The holes may be used for any combination of dispensing or transferring liquids using electric field, pneumatic forces, optical inspection, allowing liquids to interact through membranes.

The array may interface with a liquid handling unit, which the liquid handling unit may direct the plurality of droplets adjacent to the array. The liquid handling unit may be selected from the group consisting of robotic liquid handling systems, acoustic liquid dispensers, syringe pumps, inkjet nozzles, microfluidic devices, needles, diaphragm based pump dispensers, piezoelectric pumps, and other liquid dispensers. The robotic liquid handling systems may be stationary liquid dispensing platforms or be motorized for mapped liquid dispensing. The robotic liquid handling systems may have one or more tips for dispensing liquid. The acoustic liquid dispensers may dispense liquid volumes from less than 1 nanoliter (nL). The acoustic liquid dispensers may have from about 1 to 1600 wells for liquid storage. The syringe pumps may be configured to handle from 1 to 10 or more syringes in parallel. The syringe pumps may use syringes from less than 1 mL in volume to 50 mL or more. The inkjet nozzles may be fixed head or disposable head nozzles. The inkjet nozzles may comprise an array of nozzles from about 1 nozzle to 10 nozzles or more. The inkjet nozzles may be driven by piezoelectric actuators or by thermal drop creation. The microfluidic devices may comprise arrays of microfluidic channels ranging from 1 channel to 1000 or more. The microfluidic devices may be used to start a reaction before the liquid is dispensed into the droplet. The needles may range in size from less than 7 gauge to 24 gauge or more. The needles may comprise an array with a number of needles from 1 needle to 100 needles or more. The diaphragm pump may have a diaphragm made from rubber, thermoplastic, fluorinated polymer, another plastic, or any combination thereof.

The array may be coupled to a reagent storage unit, a sample storage unit, a plurality of reagent storage units, a plurality of sample storage units, or any combination thereof. The reagent storage unit, sample storage unit, plurality of reagent storage units, plurality of sample storage units, or any combination thereof may comprise at least one multi-well plate, tubes, bottles, reservoirs, inkjet cartridges, plates, petri dishes, or any combination thereof. A multi-well plate may include at least about 2, 6, 12, 24, 48, 96, 384, 1536, 3456, 9600, or more wells. The tubes may be selected from Eppendorf tubes or falcon tubes. The bottles may be made of glass, polycarbonate, polyethylene, or another material compatible with what may be stored in the bottle. The bottles may have a capacity of greater than about 10 mL, 20 mL, 30 mL, 40 mL, 50 mL, 60 mL, 70 mL, 80 mL, 90 mL, 100 mL, 200 mL, 300 mL, 400 mL, 500 mL, 600 mL, 700 mL, 800 mL, 900 mL, 1 L, 2 L, 3 L, 4 L, 5 L, or more. The bottles may be replicable. The reservoir may be a high-performance liquid chromatography (HPLC) solvent reservoir. The reservoir may be made of glass, polycarbonate, polyethylene, or another material compatible with what may be stored in the reservoir. The reservoir may have a capacity of greater than about 10 mL, 20 mL, 30 mL, 40 mL, 50 mL, 60 mL, 70 mL, 80 mL, 90 mL, 100 mL, 200 mL, 300 mL, 400 mL, 500 mL, 600 mL, 700 mL, 800 mL, 900 mL, 1 L, 2 L, 3 L, 4 L, 5 L, 6 L, 7 L, 8 L, 9 L, 10 L, 15 L, 20 L, 25 L, 30 L, 35 L, 40 L, 45 L, 50 L, or more. The inkjet cartridge may be commercially available, made specifically for the array, or a combination thereof. The inkjet cartridge may dispense liquid by thermal methods, piezoelectric methods, or a combination thereof. The inkjet cartridge may be refillable, disposable, or have both refillable and disposable components. The inkjet cartridge may contain at least 1, 2, 3, 4, 5, 6, 7, 8, 9, 10, or more different liquids. The plate may be a medium for cell growth. The medium for cell growth may be agar. The agar may have nutrients for the promotion of cell growth. The nutrients for the promotion of cell growth may be blood, derived from blood, sugars, other essential nutrients, or any combination thereof. The petri dishes may incorporate plates. The petri dishes may be bare. The petri dishes may be made of glass, plastic, or a combination thereof. The petri dish may be a replicate organism detection and counting (RODAC) plate. The plurality of wells of the multi-well plate may be thermally conductive, electronically receptive, or a combination thereof. The reagent or sample may be manipulated in or out of the well by an electric field, a magnetic field, an acoustic wave, heat, pressure, vibration, a liquid handling unit, or a combination thereof.

The array may comprise a coating. The coating may be a hydrophobic coating. The coating may be a hydrophilic coating. The coating may comprise both hydrophobic and hydrophilic coatings. The coating may be cleaned by washing. The coating may reduce evaporation. The coating may reduce evaporation by 10% to 100%. The coating may reduce evaporation by 50% to 100%. The coating may reduce biofouling. The coating may reduce biofouling by 10% to 100%. The coating may be resistant to biofouling. The coating may be antibiofouling. The hydrophobic coating may be a fluoropolymer, a polyethylene, or a polystyrene. The hydrophobic coating may also be a modification of the surface with molecules, such as fatty acids, polyaromatic compounds, or the like. For example, oleic acid may be bound to the surface, presenting a carbon chain that would increase the hydrophobicity of the surface. The hydrophilic coating may be a hydrophilic polymer such as poly-vinyl alcohol, poly-ethylene glycol, or the like. The coating comprising both hydrophobic and hydrophilic coatings may be combination of the hydrophilic and hydrophobic polymers above, or it may be a polymer that has both hydrophilic and hydrophobic properties, for example, a copolymer.

The coating may be easily cleaned by washing. Such a coating can be slippery to the samples placed on it to facilitate easy removal of those samples. The droplet may include a coating to prevent or reduce evaporation of material from within the droplet to an environment external to the droplet, from the environment to within the droplet, or any combination thereof. Such coating may reduce evaporation of content from within the droplet by at least 10%, 20%, 30%, 40%, 50%, 60%, 70%, 80%, 90% or more. The coating may be a polymeric coating (e.g., polyethylene glycol). The coating may be formed as a skin around the droplet. The coating may be generated, for example, by bringing the droplet in contact with a fluid comprising a polymeric material (e.g., a polymer or polymer precursor). When the polymeric material comes in contact with the water droplet, diffusion of the fluid into water induces polymerization or cross-linking.

The coating may reduce biofouling, or the accumulation of undesired biological species, by being biocidal or non-toxic. Examples of biocidal coating may be coatings containing a moiety toxic to biological systems, such as tributyl tin or other biocides. Examples of non-toxic coating may include coating with decreased attachment of biological species, such as fluoropolymers or polydimethylsiloxane. Such a coating may be antibiofouling.

The coefficient of variation may be less than 15%, 10%, 5%, or 1%. For example, a coefficient of variation of 1% in droplet size means that for the same series of processes performed on a number of droplets, the standard deviation of the change in droplet size divided by the mean decrease in droplet size would be 1%.

The processing of the plurality of biological samples may comprise nucleic acid sequencing. The nucleic acid sequencing may comprise polymerase chain reaction (PCR). The PCR may comprise highly multiplexed PCR, quantitative PCR, droplet digital PCR, reverse transcriptase PCR, or any combination thereof. The highly multiplexed PCR may be a single or multiple template PCR reaction. The quantitative PCR may use a variety of makers to show the PCR products in real time, such as Sybr green or the TaqMan probe. The droplet digital PCR may use initial droplets from less than 1 microliter to more than 50 microliters, and may separate those droplets into more than 10,000 droplets via an oil water emulsion technique. The reverse transcriptase PCR may be one step or two steps, (i.e., it may require only one droplet or multiple droplets to be completed). The reverse transcriptase PCR may utilize endpoint or real time quantification of the products, which can be done using fluorescence measurements.

The processing of the plurality of biological samples may comprise sample preparation for genomic sequencing. The preparation for genomic sequencing may involve removing DNA from a host cell, cell-free DNA, or any combination thereof. The preparation for genomic sequencing may involve amplification to provide enough DNA for sequencing. The preparation for genomic sequencing may utilize enzymatic fragmentation of the DNA, mechanical fragmentation of the DNA, or any combination thereof.

The processing of the plurality of biological samples may comprise a combinatorial assembly of genes. The combinatorial assembly of genes may comprise a Gibson Assembly, restriction enzyme cloning, gBlocks fragments assembly (IDT), BioBricks assembly, NEBuilder HiFi DNA assembly, Golden Gate assembly, site-directed mutagenesis, sequence and ligase independent cloning (SLIC), circular polymerase extension cloning (CPEC), and seamless ligation cloning extract (SLiCE), topoisomerase mediated ligation, homologous recombination, Gateway cloning, GeneArt gene synthesis, or any combination thereof.

The processing of the plurality of biological samples may comprise cell-free protein expression. The cell-free protein expression may be used to express toxic proteins. The cell-free protein expression may be used to incorporate non-natural amino acids. The cell-free protein expression may utilize phosphoenol pyruvate, acetyl phosphate, creatine phosphate, or any combination thereof as an energy source. The cell-free protein expression may be done at ambient temperatures, temperatures below ambient temperature (e.g., 0° C.), temperatures above ambient temperature (e.g., 60° C.), or any combination thereof.

The processing of the plurality of biological samples may comprise preparation for plasmid DNA extraction. The preparation for plasmid DNA extraction may comprise precipitating the DNA from a lysed cell solution. The preparation for plasmid DNA extraction may comprise using a spin-column based separation technique. The preparation for plasmid DNA extraction may comprise a phenol-chloroform extraction.

The processing of the plurality of biological samples may comprise extracting ribosomes, mitochondria, endoplasmic reticulum, golgi apparatus, lysosomes, peroxisomes, centrioles, or any combination thereof. The ribosomes, mitochondria, endoplasmic reticulum, golgi apparatus, lysosomes, peroxisomes, centrioles, or any combination thereof may remain intact.

The processing of the plurality of biological samples may comprise extraction of nucleic acids from cells. The extraction of nucleic acids from cells may further comprise extracting long strands of nucleic acid, where the long strands of nucleic acid remain completely intact. The long strands of nucleic acid may also be at least 10, 100, 1,000, 10,000, 100,000, 1,000,000, or more base pairs long. The extraction of nucleic acid may involve the lysing of cells via the addition of surfactants and detergents such as octyl glucoside, sodium dodecyl sulfate, or octyl phenol ethoxylate. The extraction of nucleic acids may involve centrifugation, including ultracentrifugation.

The processing of the plurality of biological samples may comprise sample preparation for mass spectrometry. Sample preparation for mass spectrometry may involve cell lysis, digestion, protein amplification, DNA amplification, or other standard sample preparations. Sample preparation for mass spectrometry may include application of a sample to an electrospray ionization (ESI) substrate, incorporation into a matrix-assisted laser desorption ionization (MALDI) matrix, or other preparation for ionization. Mass spectrometry may include ion trap, quadrupole, and other detection methods. The inlet of the mass spectrometer may be directly coupled to at least one droplet. The inlet of the mass spectrometer may be adjacent to one or more droplets. The sample for mass spectrometry may be transferred to the inlet of the mass spectrometer by pipetting.

The processing of the plurality of biological samples may comprise sample extraction and library preparation for nucleic acid sequencing. The nucleic acid sequencing may comprise sequencing by synthesis, pyrosequencing, sequencing by hybridization, sequencing by ligation, sequencing by detection of ions released during polymerization of DNA, single-molecule sequencing, or any combination thereof. The single molecule sequencing may be nanopore sequencing. The single molecule sequencing may be single molecule real time (SMRT) sequencing.

The processing of the plurality of biological samples may comprise DNA synthesis using oligonucleotide synthesis, enzymatic synthesis, or any combination thereof. The oligonucleotide synthesis may be solid state, liquid phase, performed in solution, or any combination thereof. The oligonucleotide synthesis may produce oligonucleotides that may be at least 2, 5, 10, 20, 30, 40, 50, 100, 200, 300, 400, 500, or more nucleotides. The enzymatic synthesis may use polymerases, transferases, other enzymes, or any combination thereof.

The processing of the plurality of biological samples may comprise DNA data storage, random-access of stored DNA and DNA data retrieval through DNA sequencing. DNA data storage may utilize strands of DNA having greater than about 10, 50, 100, 150, 200, 250, 500, 1,000, 5,000, 10,000, 100,000, 1,000,000, or more base pairs. DNA sequencing may include at least one PCR reaction, a Maxam-Gilbert sequencer, a Sanger sequencer, or any combination thereof. The nucleic acid sequencing may comprise sequencing by synthesis, pyrosequencing, sequencing by hybridization, sequencing by ligation, sequencing by detection of ions released during polymerization of DNA, single-molecule sequencing, or any combination thereof. The single molecule sequencing may be nanopore sequencing. The single molecule sequencing may be single molecule real time (SMRT) sequencing.

The processing of the plurality of biological samples may comprise nucleic acid extraction and sample preparation integrated directly into a sequencer. The nucleic acid extraction and sample preparation may be performed directly on the array. The nucleic acid extraction and sample preparation may be performed adjacent to the array. The sequencer may be adjacent to the array. The sequencer may be coupled to the array. The sequencer may be directly on the array.

The processing of the plurality of biological samples may comprise CRISPR genome editing. The editing may comprise Cas9 protein, Cpfl endonuclease, crRNA, tracrRNA, or any combination thereof. A repair DNA template may be used during the editing process. The repair DNA template may be a single-stranded oligonucleotide, double-stranded oligonucleotide, or a double-stranded DNA plasmid.

The processing of the plurality of biological samples may comprise transcription activator-like effector nucleases (TALENs) genome editing. The processing of the plurality of biological samples may comprise zinc fingers nuclease gene editing.

The processing of the plurality of biological samples may comprise at least one high-throughput process. The high-throughput process may be automated to not require input. The high-throughput process may comprise at least one of the assays or characterization methods applied to at least one of the sample types that are described herein.

The processing of the plurality of biological samples may comprise the screening of a plurality of chemical compounds against a plurality of cells. The chemical compound may be one or more chemical compounds. The chemical compound may show a biological effect. A biological effect may be the promotion or inhibition of cellular growth, the signaling of a cellular process to begin or end, the induction of cell division, or the like.

The chemical compounds may be antibacterial. Antibacterial chemicals may inhibit the growth of bacteria from at least 5% to greater than 99%. Antibacterial chemicals may kill bacteria.

The chemical compound may be screened for biological activity. The chemical compound may use the sensors of the array to determine biological activity. For example, an array of fluorescence detectors may be used to determine the relative amount of a fluorescent protein in a biological sample exposed to a chemical compound of interest. Similarly, for example, a microscope may be used to assay the total number of a cell species after exposure to a chemical compound. The chemical compound may be isolated. The isolation may involve centrifugation, transfer via pipetting or another liquid transfer technique, precipitation, a chromatographic technique (e.g., column chromatography, thin layer chromatography, etc.), distillation, lyophilization, or recrystallization. The screen for biological activity may involve mixing at least one biological sample in at least one droplet with at least one chemical.

The cells may be bacterial cells. The bacterial cells may be disease causing. The bacterial cells may be resistant to antibiotics. The bacterial cells may be genetically modified.

The cells may be eukaryotic cells. The eukaryotic cells may be single celled organisms (e.g. protozoans, algae), diatoms, fungal cells, insect cells, animal cells, mammalian cells, or human cells. The eukaryotic cells may be derived from single celled organisms (e.g. protozoans, algae), diatoms, fungi, insects, animals, mammalians, or humans. The eukaryotic cells may be derived from a larger tissue or organ. The eukaryotic cells may be genetically modified. The eukaryotic cells may be suspected of having or carrying a disease.

The cells may be prokaryotic cells. The prokaryotic cells may be genetically modified.

The processing of the plurality of biological samples may comprise culturing cells, thereby producing cultured cells. The culturing of the cells may occur in discrete droplets. The culturing of the cells may occur in discrete physical compartments. The culturing of cells may be done autonomously (with no input required). The culturing of cells may be performed on solid, liquid or semi-solid media.

The culturing of cells may occur in 2 or 3 dimensions. The culturing of cells may be done under ambient or non-ambient conditions (e.g., elevated temperature, low pressure, etc.). The discrete physical compartments may be discrete electrowetting chips.

The interactions between the cultured cells or between cultured cells and at least one biological sample may be determined. The interaction of two or more samples of cultured cells may be determined by mixing. The interaction of at least one biological sample and the cultured cells may be determined by mixing, applying the cultured cells directly onto the biological sample, or applying the biological sample directly onto the cultured cells. Applying the cultured cells may involve transferring liquid cell culture or placing a solid cell culture onto the sample of interest.

The cultured cells may be assayed on the array, or the plurality of arrays as described herein.

The cultured cells may be isolated from culture. The isolation may involve centrifugation, transfer via pipetting or another liquid transfer technique, precipitation, scraping the cells off of the culture, or a chromatographic technique (e.g., cellular chromatography). The isolated cells may be transferred to an external container. The external container may be a society for biomolecular screening (SBS) format plate, a petri dish, a bottle, a box, another culture medium, or the like.

The isolated cells may be prepared for nucleic acid sequencing.

The isolated cells may be prepared for protein analysis. The protein analysis may be an amino acid analysis, size analysis, absorption analysis, the Kjeldahl method, the Dumas method, western blot analysis, high-performance liquid chromatography (HPLC) analysis, liquid chromatography-mass spectrometry (LC/MS) analysis, or enzyme-linked immunosorbent assay (ELISA) analysis.

The isolated cells may be prepared for metabolomic analysis. The metabolomic analysis may be aqueous metabolite profiling, lipid metabolite profiling, nuclear magnetic resonance spectroscopy (NMR) analysis, or a mass spectrometry analysis.

The array may comprise a plurality of lyophilized reagents, dry reagents, stored beads, or any combination thereof. The plurality of lyophilized reagents, dry reagents, stored beads, or any combination thereof may be reconstituted. The lyophilized reagents may include proteins, bacteria, microorganisms, vaccines, pharmaceuticals, molecular barcodes, oligonucleotides, primers, DNA sequences for hybridization, enzymes (e.g., glucosidase, alcohol dehydrogenase, a DNA polymerase, etc.) and dehydrated chemicals. The dry reagents may include chemical powders (e.g. salts, metal oxides, etc.), biologically derived chemicals, dry buffer chemicals, other bioactive chemicals, and the like. The stored beads may be magnetic beads, beads for the storage of bacteria, enzymes, oligonucleotides, or molecular sieves. The molecular barcodes may be DNA fragments with at least 5, 10, 20, 30, 40, 50, 60, or more base pairs. The oligonucleotides may be at least 2, 5, 10, 20, 30, 40, 50, 100, 200, 300, 400, 500, or more nucleotides. The primer may be DNA or RNA. The DNA sequences for hybridization may be used to detect small differences in nucleotide order. The DNA sequence may be used in conjunction with mismatch detection proteins.

The droplet, a plurality of droplets, derivatives thereof, or any combination thereof may be used to reconstitute the lyophilized reagents, dry reagents, stored beads, or any combination thereof. The reconstitution may solubilize, suspend, or form colloids of the lyophilized reagents, dry reagents, stored beads, or any combination thereof. The reagents may be prefabricated into a component of the array.

In some embodiments, the plurality of droplets comprises a third droplet comprising a third reagent.

The array may store a plurality of reagents as a solid, liquid, gas, or any combination thereof. The array may condense, sublime, thaw, evaporate, or any combination thereof, the stored reagent. The reagent may be a compressed gas (e.g., air, argon, nitrogen, oxygen, carbon dioxide, etc.), a solvent (e.g., water, dimethyl sulfoxide, acetone, ethanol, etc.), a cleaner (e.g., ethanol, SDS, liquid soap, etc.), or a solution (e.g., a buffer, a chemical dissolved in a liquid, etc.). For an example of the array performing a physical state transformation of a stored reagent, solid carbon dioxide (dry ice) may be sublimed to provide cold carbon dioxide gas to a droplet. Another example may be for the array to boil water to introduce steam into a droplet or to clean the array.

The array may dispense a plurality of liquids. The array may use a variety of methods to dispense the plurality of liquids, such as, for example by pipetting, condensing, decanting, or any combination thereof, employing devices such as: microfluidic device, diaphragm pump, nozzle, piezoelectric pump, needle, tube, acoustic dispenser, capillary, or any combination thereof. The plurality of liquids may be from at least 2, 3, 4, 5, 6, 7, 8, 9, 10, 20, 30, 40, 50, 60, 70, 80, 90, 100, 500, 1,000, or more liquids.

The array may mix a plurality of liquids. The mixing may be performed by stirring, sonication, vibration, gas flow, bubbling, shaking, swirling, and electrowetting forces. The plurality of liquids may be from at least 2, 3, 4, 5, 6, 7, 8, 9, 10, 20, 30, 40, 50, 60, 70, 80, 90, 100, 500, 1,000, or more liquids. The liquids may be in the form of at least one droplet. The at least one droplet may be on an electrowetting array.

The processing of the plurality of biological samples may be automated (e.g., made able to be run without user input). The automation may use a program to run. The program may be a machine learning algorithm. The program may utilize a neural network. The automation may be controlled by a device. The device may be a computer, a tablet, a smartphone, or any other device capable of executing the code. The automation may interface with one or more components of the array (e.g., sensors, liquid handling devices, etc.) to perform the processing. In some embodiments, the automation may use a camera that tracks the size of a droplet on the array. When the droplet has lost sufficient volume due to evaporation, as determined by a computer vision program, the automation would instruct the liquid handling unit to dispense a precise amount of liquid to the droplet to maintain a pre-programmed volume. In this embodiment, an open configuration may allow for easier observation of the droplets. The automation program may also be self-diagnosing, using machine learning classifiers to monitor assays for atypical events that may indicate errors. The machine learning algorithms may also be used to improve the performance of the automated assays. The machine learning data may be compiled and analyzed to suggest changes in the control algorithms that may improve assay development.

The array may be reusable. The array may have a replaceable surface. The array may have a replaceable film. The array may have a replaceable cartridge. The replaceable cartridge may comprise a film. The film may be attached to the array. The film may be fastened to the array using vacuum. The film may be coupled to the array using an adhesive. The adhesive may be non-reactive, pressure-sensitive, contact reactive, heat reactive (e.g., anaerobic, multi-part (e.g., polyester, polyols, acrylic, etc.), pre-mixed, frozen, one-part), natural, synthetic, or any combination thereof. The adhesive may be applied by spraying, brushing, rolling, or by a film or applicator. The adhesive may be, but is not limited to, silicone, acrylic, epoxy, polyurethane, starch, cyanoacrylate, polyimide, or any combination thereof. The array may be reused from at least 2, 3, 4, 5, 6, 7, 8, 9, 10, 20, 30, 40, 50, 60, 70, 80, 90, 100, 500, 1,000, or more times. The replaceable surface may be easy to remove and reattach to the array. The replicable surface may be a layer of a liquid. The liquid may be an oil. The replaceable film may be a polymer (e.g., polyethylene, polytetrafluoroethylene, polydimethylsiloxane, etc.). The replaceable film may be from 1 nanometer to 1 millimeter thick. The replaceable cartridge may comprise a new electrowetting chip. The replaceable cartridge may comprise a new surface to be placed on the electrodes of an electrowetting chip.

The array may be washed. The array may be washed entirely. The array may be washed partially. The array may be washed using a material stored in the reagent dispensing array. The array may be washed using a solid cleaner (e.g., powdered soap, a solid antimicrobial, etc.), a liquid cleaner (e.g., liquid soap, ethanol, etc.), or a gaseous cleaner (e.g., steam). About 1% to 100% of the array may be washable.

The array may be disposable. The disposable array may comprise the entire sample assembly. The disposable array may comprise the surface of an electrowetting chip. The disposable array may be easily removed.

The volume of biomolecules of the array may be manipulated as a mixture. The volume of biomolecules may comprise a plurality of nucleic acids, protein sequences, or a combination thereof. The plurality of nucleic acid, protein sequences, or a combination thereof may be manipulated by modulation of local surface charge without physical contact on the mixture by another component of the array. For example, an electrowetting chip may be used to move a droplet containing a number of nucleic acids by changing the surface wetting properties of the droplet. This would allow the droplet to move without contact from another component of the array. The mixture may be within a droplet. The droplet may comprise a volume of at least 1 picoliter (μL), 10 μL, 100 μL, 1 nanoliter (nL), 10 nL, 100 nL, 1 μL, 10 μL, 100 μL, 1 milliliter (mL), 10 mL or more. The mixture may comprise a protein with DNA ligase activity. The mixture may comprise a protein with DNA transposase activity. The protein with DNA ligase activity may be derived from a virus (e.g., T4), a bacteria (e.g., E. coli), or a mammal (e.g., human DNA ligase 1). The protein with DNA transposase activity may be derived from a bacteria (e.g., Tn5) or a mammal (e.g., sleeping beauty (SB) transposase). The volume of biomolecules of the assay may be manipulated with lateral geospatial movement of the mixture of at least 1 mm. The volume of biomolecules of the assay may be manipulated by a predetermined or preprogrammed set of commands. The commands may be associated with a particular location of the array.

The array may comprise reagents for conducting a strand displacement amplification reaction, a self-sustained sequence replication and amplification reaction or a Q3 replicase amplification reaction. The reagent for conducting a strand displacement amplification reaction may be Bst DNA polymerase, cas9, or another hemiphosphorothioate form nicking protein. A self-sustained sequence replication and amplification reaction reagents may be avian myeloblastosis virus (AMV) reverse transcriptase (RT), Escherichia coli RNase H, T7 RNA polymerase, or any combination thereof. The reagents for the Q3 replicase amplification reaction may be derived from the Q3 bacteriophage, E. coli, or any combination thereof.

The array may comprise reagents including a DNA ligase, a nuclease or a restriction endonuclease. The DNA ligase may be derived from a virus (e.g., T4), a bacteria (e.g., E. coli), or a mammal (e.g., human DNA ligase 1). The nuclease may be an exonuclease (starting digestion from the end of a molecule) or an endonuclease (digesting from somewhere other than the end of a molecule). The nuclease may be a deoxyribonuclease (operating on DNA) or a ribonuclease (operating on RNA). The restriction endonuclease may be a type I, II, III, IV, or V restriction endonuclease. An example of a restriction endonuclease may be cas9 or a zinc finger nuclease.

The array may comprise reagents for the preparation of an amplified nucleic acid product. The reagents for the preparation of an amplified nucleic acid product may be Bst DNA polymerase, deoxyribonucleotide triphosphate, fragments of E. coli DNA polymerase 1, avian myeloblastosis virus reverse transcriptase, RNase H, T7 DNA dependent RNA polymerase, Taq polymerase, other DNA polymerases/transcriptases, or any combination thereof.

The array may be a component in the manufacture of a kit or system for the diagnosis or prognosis of a disease. The kit may process a biological sample. The biological sample may be a sample derived from a patient. In some embodiments, the array may be used to process a sample derived from a patient suspected of having a disease. The disease may be a disease classified by the Centers for Disease Control and Prevention (CDC). The array may mix the sample with a reagent. The array may mix the sample with a reagent for separating cells from serum. The array may process the cells, or derivatives thereof. The array may transfer cells, or derivatives thereof, to an optical device coupled to the array. The cells, or derivatives thereof, may be processed according to methods described herein.

The array may include a protein with nucleic acid cleavage activity. The array may include a biomolecule with RNA cleavage activity. The protein with nucleic acid cleavage activity may be a ribonuclease, a deoxyribonuclease, or any combination thereof. The biomolecule with RNA cleavage activity may be a small ribonucleolytic ribozyme, a large ribonucleolytic ribozyme, or any combination thereof.

An interchangeable set of reagents may be introduced by at least one solid phase support. The solid phase support may be a paper strip. The solid phase support may be a microbead. The solid phase support may be a pillar. The pillar may be attached to the base of the support or integral to the support. The solid phase support may be a strip of microwells. The solid phase support may be a glass slide, a scoop, or a plastic film. The solid phase support may be a bead. The bead may be magnetic. The interchangeable set of reagents may be chemical reagents (e.g., small molecules, metals, etc.), biological species (e.g., proteins, DNA, RNA, etc.), processing reagents (e.g., PCR reagents, etc.).

The interchangeable set of reagents may be introduced by at least one secondary support. The secondary support may be a strip of microwells. The secondary support may be a SBS plate, petri dish, bottle, slide, or another container. The interchangeable set of reagents may be chemical reagents (e.g., small molecules, metals, etc.), biological species (e.g., proteins, DNA, RNA, etc.), processing reagents (e.g., PCR reagents, etc.).

The array may contain a template independent polymerase. The template independent polymerase may be a terminal deoxynucleotidyl transferase (TdT). The array may include an enzyme that limits nucleic acid polymerization. The enzyme that limits nucleic acid polymerization may be an apyrase. The array may have sensors to detect the presence of at least one terminal ‘C’ tail in a nucleic acid molecule. The at least one terminal ‘C’ tail may be isolated. The apyrase may be derived from E. coli, S. tuberosum, or an arthropod.

The plurality of biological samples of the array may be stored by drying. The drying may be performed by heating, vacuum, flowing gas, lyophilization, or any combination thereof. The samples may be stored on the array or in another container. The other container may be a glass slide, petri dish, media bottle, tube, or (micro) well array.

The plurality of biological samples of the array may be retrieved by rehydration. The rehydration may be performed by adding liquid to or blowing a gas containing liquid over the dried plurality of biological samples. The rehydrated plurality of biological samples may be manipulated with any of the liquid handling mechanisms stated above.

The plurality of biological samples may be deposited onto the plurality of arrays in SBS format or on any random location of the plurality of arrays, thereby producing at least one deposited biological sample. The SBS format may be the dimensions of a 96 well plate. The deposited biological sample may be a solid or a liquid.

The plurality of biological samples may be deposited using commercial acoustic liquid handlers in preparation for manipulating samples on the chip. The acoustic liquid handlers may be an Echo® or an ATS Gen5®. The at least one deposited biological sample may be used for cell-free synthesis. The at least one deposited biological sample may be used for combinatorially assembling large DNA constructs. The combinatorially assembling large DNA constructs may be a Gibson assembly, circular polymerase extension cloning, and DNA Assembler method.

The processing of the plurality of biological samples may comprise at least one of the following assays, or any combination thereof: digital PCR, isothermal amplification of nucleic acids, antibody mediated detection, enzyme linked immunoassay (ELISA), electrochemical detection, colorimetric assay, fluorometric assay, and micronucleus assay.

The digital PCR assay may process droplets from at most about 1,000 microliters, 900 microliters, 800 microliters, 700 microliters, 600 microliters, 500 microliters, 400 microliters, 300 microliters, 200 microliters, 100 microliters, 50 microliters, 10 microliters, 1 microliter, 0.1 microliters, 0.01 microliters, 0.001 microliters, 0.0001 microliters, or less. The digital PCR may use initial droplets from at least about 0.0001 microliters, 0.001 microliters, 0.01 microliters, 0.1 microliters, 1 microliter, 10 microliters, 50 microliters, 100 microliters, 200 microliters, 300 microliters, 400 microliters, 500 microliters, 600 microliter, 700 microliters, 800 microliters, 900 microliters, 1,000 microliters, or more. The digital PCR may use initial droplets from about 100 microliters to about 1 microliter. The digital PCR may use initial droplets from about 50 microliters to about 1 microliter. In some embodiments, the digital PCR assay may separate a droplet, or a plurality thereof, into at least about 1, 2, 5, 10, 20, 30, 40, 50, 60, 70, 80, 90, 100, 200, 300, 400, 500, 600, 700, 800, 900, 1,000, 2,000, 3,000, 4,000, 5,000, 6,000, 7,000, 8,000, 9,000, 10,000, or more, droplets. The droplet, or plurality thereof, may be separated by an oil water emulsion technique.

The isothermal amplification of nucleic acids may be PCR, strand-displacement amplification (SDA), rolling circle amplification (RCA), loop-mediated isothermal amplification (LAMP), nucleic acid sequence based amplification (NASBA), helicase-dependent amplification (HDA), recombinase polymerase amplification (RPA), cross-priming amplification (CPA), or any combination thereof.

The antibody mediated detection may be used to detect cells, proteins, nucleic acid molecules (e.g., DNA, RNA, PNA, etc.), hormones, antibodies, small molecules, or any combination thereof. The antibody mediated detection may comprise antibodies that comprise antigen-binding sites specific to detect a cell, protein, nucleic acid, or any combination thereof. The antibody may be naturally-derived. The antibody may be a synthetic antibody. The synthetic antibody may be a recombinant antibody, a nucleic acid aptamer, a non-immunoglobulin protein scaffold, or any combination thereof.

The enzyme linked immunoassay (ELISA) may be direct, sandwich, competitive, reverse type, or any combination thereof. The ELISA may detect, quantify, or a combination thereof, substances, such as, for example, peptides, proteins, antibodies, hormones, small-molecules, or any combination thereof.

The electrochemical detection may be an oxidation- or reduction-based electrochemical detection. The oxidation- or reduction-based electrochemical detection may be conductometric, potentiometric, voltammetric, amperometric, coulometric, impedimetric, or any combination thereof. The electrochemical detection may be used to detect a cell, proteins, nucleic acids, hormones, small-molecules, antibodies, or any combination thereof. The electrochemical detection may detect electric currents generated from oxidative or reductive reactions of biological samples. The electrochemical detection may detect electric currents generated from oxidative or reductive reactions of biological samples.

The colorimetric assay may be used to detect cells, nucleic acids, proteins, small-molecules, antibodies, hormones, or any combination thereof. The colorimetric assay may be used to assay an absorption of a wavelength of at least 240 nm, 280 nm, 300 nm, 350 nm, 400 nm, 450 nm, 500 n, 550 n, 600 nm, 650 nm, 700 nm, 750 nm, 800 nm, 850 nm, 900 nm, 950 nm, 1000 nm, 1250 nm, 1500 nm, 1750 nm, 2000 nm, 2400 nm, or more. The colorimetric assay may be used to assay an absorption of a wavelength of at most 2400 nm, 2000 nm, 1750 nm, 1500 nm, 1250 nm, 1000 nm, 950 nm, 900 nm, 850 n, 800 nm, 750 nm, 700 nm, 650 nm, 600 nm, 550 nm, 500 nm, 450 nm, 400 nm, 350 nm, 300 nm, 280 nm, 240 nm, or less. The colorimetric assay may be used to assay an absorption of a wavelength from about 2400 nm to about 240 nm. The colorimetric assay may be used to assay an absorption of a wavelength from about 1000 nm to about 100 nm. The colorimetric assay may be used to assay an absorption of a wavelength from about 900 nm to about 400 nm. The colorimetric assay may be performed on solid, liquid, or gaseous samples. The colorimetric assay may use a broadband light source (e.g., an incandescent source, an LED, etc.), a laser source, or a combination thereof. The light source may be passed through a variety of optical elements (e.g., lenses, filters, mirrors, etc.) before and after it interacts with the sample. The transmitted or reflected light may be detected (e.g., by a mirror, a fiber optic, etc.) via a charge-coupled device (CCD), a photomultiplier tube, an avalanche photodiode, or any combination thereof. The detector may be coupled to a wavelength selecting device, such as, for example, a monochrometer or a filter or set of filters.

The fluorometric assay may be used to detect cells, nucleic acids, proteins, small-molecules, antibodies, hormones, or any combination thereof. The fluorometric assay may be used to assay an absorption of a wavelength of at least 240 nm, 280 nm, 300 nm, 350 nm, 400 n, 450 nm, 500 n, 550 n, 600 nm, 650 nm, 700 nm, 750 nm, 800 nm, 850 nm, 900 nm, 950 nm, 1000 nm, 1250 nm, 1500 n, 1750 n, 2000 nm, 2400 nm, or more. The fluorometric assay may be used to assay an absorption of a wavelength of at most 2400 nm, 2000 nm, 1750 nm, 1500 nm, 1250 nm, 1000 nm, 950 nm, 900 nm, 850 nm, 800 n, 750 nm, 700 nm, 650 nm, 600 n, 550 n, 500 nm, 450 nm, 400 nm, 350 nm, 300 n, 280 nm, 240 nm, or less. The fluorometric assay may be used to assay an emission of a wavelength from about 2400 nm to about 240 nm. The fluorometric assay may be used to assay an emission of a wavelength from about 1000 nm to about 100 nm. The fluorometric assay may be used to assay an emission of a wavelength from about 900 nm to about 400 nm. The fluorometric assay may use a broadband light source (e.g., an incandescent source, an LED, etc.), a laser source, or a combination thereof. The light source may be passed through a variety of optical elements (e.g., lenses, filters, mirrors, etc.) before and after it interacts with the sample. The fluoresced light may be detected via a CCD, a photomultiplier tube, an avalanche photodiode, or any combination thereof. The detector may be coupled to a wavelength selecting device, such as a monochrometer or a filter or set of filters. For example, a fluorometric assay may be used to determine the concentration of reduced NADPH, as it fluoresces in its reduced form but not in its oxidized form. In this example, the intensity of the observed fluorescence over time would correspond linearly with the amount of reduced NADPH in the sample.

The micronucleus assay may evaluate the presence of micronuclei in a biological sample. The micronuclei may contain chromosome fragments produced from DNA breakage (clastogens) or whole chromosomes produced by disruption of the mitotic apparatus (aneugens). The micronucleus assay may be used to identify genotoxic compound. The genotoxic compound may be a carcinogen. The micronucleus assay may be performed in vivo or in vitro. The in vivo micronucleus assay may utilize bone marrow or peripheral blood from a biological sample. The in vitro micronucleus assay may utilize cells or tissues derived from a plurality of biological samples.

The processing of the plurality of biological samples may comprise isothermal amplification of at least one selected nucleic acid or polynucleotide, which may comprise: providing at least one sample that may comprise at least one nucleic acid by merging droplets containing a plurality of reagents effective to permit at least one isothermal amplification reaction of the sample without mechanical manipulation; and conducting at least one isothermal amplification reaction to amplify the nucleic acid.

The at least one isothermal amplification of at least one selected nucleic acid may be PCR, strand-displacement amplification (SDA), rolling circle amplification (RCA), loop-mediated isothermal amplification (LAMP), nucleic acid sequence based amplification (NASBA), helicase-dependent amplification (HDA), recombinase polymerase amplification (RPA), cross-priming amplification (CPA), or any combination thereof. The at least one isothermal amplification may be at least 2, 3, 4, 5, 6, 7, 8, 9, 10 or more isothermal amplifications.

The merging droplets may be at least 2, 3, 4, 5, 6, 7, 8, 9, 10 or more droplets. The plurality of reagents may be any of the isothermal amplification reagents described herein.

The processing of the plurality of biological samples may comprise a device to detect a polymerase chain reaction (PCR) product on at least one droplet. The droplet may be an aqueous droplet. The device may: create at least one droplet containing a plurality of nucleic acid and protein molecules on an electrowetting array; perform the PCR reaction while the aqueous droplets are present on the array surface; and interrogate the droplet with a detector. The PCR product may be DNA or RNA. The protein molecules may be enzymes, utilized in the PCR reaction, or used to report the progress of a reaction (e.g., luminescent). The performance of the PCR reaction may include agitating the sample (e.g., stirring, vibration, electrowetting based movement, etc.), heating or cooling the sample (using the aforementioned heater and cooler arrays), and controlling the droplet size. The detector may be any detector described herein.

The device may comprise a plurality of reporter molecules. The reporter molecules may be fluorescent reporter molecules. The plurality of fluorescent reporter molecules may be separated by at least one enzyme from at least one quencher molecule during the PCR reaction. The at least one enzyme may comprise a polymerase, oxidoreductase, transferase, hydrolase, lyase, isomerase, or ligase. The plurality of fluorescent reporter molecules may be a protein, a luminescent small molecule, a luminescent nucleic acid, or a nanoparticle.

The nucleic acid may be detected by a sensor. The sensor may detect a radiolabel. The sensor may detect a fluorescent label. The sensor may detect a chromophore. The sensor may detect a redox label. The sensor may be a p-n-type diffusion diode. The nucleic acid may be detected by a smartphone.

The processing of the plurality of biological samples may include binding at least one biomolecule on the array. The at least one biomolecule may be immobilized on a surface. The at least one biomolecule may be immobilized on a diffusible matrix. The at least one biomolecule may be immobilized on a diffusible bead. The at least one biomolecule may be a protein, a compound derived from a biological system (e.g., a signaling molecule, a cofactor, etc.), a pharmaceutical, a molecule exhibiting or suspected of exhibiting biological activity, a carbohydrate, lipid, a nucleic acid, a natural product, or a nutrient. The immobilization may be by adsorption, ionic interaction, covalent bonding, or intercalation. The surface may be an electrowetting chip, a polymer, a dielectric, a metal, a fiber based sheet (e.g., a paper strip), or a stationary phase (e.g., silica gel). The diffusible matrix may be a polymer, a tissue (e.g., collegian), or an aerogel. The diffusible bead may be a polymer bead, a molecular sieve, or a bead formed of biological materials (e.g., a beaded protein or nucleic acid). The location of the biomolecule may be identified by a coding scheme. The coding scheme may be a preprogrammed method to determine the location of the biomolecule. The coding scheme may be based on a moiety to which it is immobilized.

In some embodiments, detectable labels may fluorescent labels for emitting a specific wavelength. In some embodiments, the fluorescent labels emit light upon excitation by a light source. In some embodiments, the detectable labels emit light at a wavelength of 380-450 nm. In some embodiments, the detectable labels emit light at a wavelength of 450-495 nm. In some embodiments, the detectable labels emit light at a wavelength of 495-570 nm. In some embodiments, the detectable labels emit light at a wavelength of 570-590 nm. In some embodiments, the detectable labels emit light at a wavelength of 590-620 nm. In some embodiments, the detectable labels emit light at a wavelength of 620-750 nm. In some embodiments, interchangeable optical filters are utilized by a computer-vision system. In some embodiments, optical filters are used in combination with one or more optical sensors or image sensors of the computer-vision system. In some embodiments, the optical filters are provided to filter wavelengths produced by detectable labels, such that only one or more labels corresponding to samples of a particular type are to be detected or monitored by the system. In some embodiments, the system may comprise one or more optical sensors, wherein each optical sensor is provided with a specific filter to monitor a specified label corresponding to samples of a particular type, as described herein.

In some embodiments, the array may induce an interaction of the plurality of biomolecules from two or more non-continuous liquid volumes without mechanical manipulations. The interaction may be mixing, a chemical reaction, adsorption, or an enzymatic reaction. Without mechanical manipulations may mean that the moving part of the interaction may be the two or more non-continuous liquid volumes. The plurality of biomolecules may be at least one of a protein, a compound derived from a biological system (e.g., a signaling molecule, a cofactor, etc.), a pharmaceutical, a molecule exhibiting or suspected of exhibiting biological activity, a carbohydrate, lipid, a nucleic acid, a natural product, or a nutrient.

The array may prepare an amplified nucleic acid product without mechanical manipulations. The array may conduct a diagnostic test on a nucleic acid sample without mechanical manipulations. The array may conduct a diagnostic or prognostic test on a biological sample without mechanical manipulations. The plurality of biological samples may be suspected of containing a nucleic acid biomarker.

The array may comprise a gas source that contacts and may be absorbed by at least one droplet. The at least one droplet may be manipulated on the device. The gas may be air, nitrogen, argon, carbon dioxide, hydrogen, or water vapor. The at least one droplet may absorb at least 0%, 10%, 20%, 30%, 40%, 50%, 60%, 70%, 80%, 90%, 99%, or more of the gas. The manipulation may be due to the pressure the gas exerts on the at least one droplet.

The plurality of biological samples may include reagents for conducting a strand displacement amplification reaction, a self-sustained sequence replication, an amplification reaction, or a Q3 replicase amplification reaction. The reagent for conducting a strand displacement amplification reaction may be Bst DNA polymerase, cas9, or another hemiphosphorothioate form nicking protein. A self-sustained sequence replication and amplification reaction reagents may be avian myeloblastosis virus (AMV) reverse transcriptase (RT), Escherichia coli RNase H, T7 RNA polymerase, or any combination thereof. The reagents for the Q3 replicase amplification reaction may be derived from the Q3 bacteriophage, E. coli, or any combination thereof i.

The array may receive at least one instruction from a remote computer to process the array of biological samples. The at least one instruction may be at least 1, 2, 3, 4, 5, 6, 7, 8, 9, 10, 20, 30, 40, 50, 60, 70, 80, 90, 100, 200, 300, 400, 500, 600, 700, 800, 900, 1,000 or more instructions. The remote computer may be any system capable of sending instructions (e.g., a desktop computer, a laptop computer, a tablet, a smartphone, an application-specific integrated circuit, etc.). The remote computer may not require user input to send the at least one instruction.

The array may be preprogrammed to perform the process on the array of biological samples. The preprogramming may be for at least 1, 2, 3, 4, 5, 6, 7, 8, 9, 10, 20, 30, 40, 50, 60, 70, 80, 90, 100, 200, 300, 400, 500, 600, 700, 800, 900, 1,000 or more steps of the process. The preprogramming may be stored in the array (e.g., on a hard drive, on a flash memory unit, on erasable programmable read-only memory (EPROM), on a tape cassette, etc.) or stored on an attached system capable of sending instructions (e.g., a desktop computer, a laptop computer, a tablet, a smartphone, an application-specific integrated circuit, etc.).

The array may receive information related to a DNA sequence. The information related to a DNA sequence may include the length of the DNA sequence, the composition of the DNA sequence (e.g., the total number of a given base, the sequence of the bases, etc.), or the presence of a particular DNA sequence. The DNA sequence may trigger an automated process. The information related to the DNA sequence may trigger an automated process. The automated process may include conversion of the DNA sequence into at least one constituent oligonucleotide sequence. The at least one constituent oligonucleotide sequence may be assembled, error corrected, reassembled, or any combination thereof, into DNA amplicons. The DNA amplicons may direct production of RNA, proteins, biological particles, or any combination thereof. The biological particles may be derived from a virus.

The array may produce at least one peptide or antibody from a DNA template. The array may produce using in vivo methods (e.g., using cells to produce) or cell-free production (e.g., not requiring a living organism to produce). The peptide may be at least 1, 2, 3, 4, 5, 6, 7, 8, 9, 10, 15, 20, 25, 30, 35, 40, 45, 50, or more amino acids. The amino acids may be naturally occurring or non-naturally occurring. The antibody may be surface bound or free. The antibody may be derived from any of the plurality of biological samples.

The array may partition at least one droplet into a plurality of droplets by: electromotive force, electrowetting force, dielectrowetting force, dielectrophoretic effect, acoustic force, hydrophobic knife, or any combination thereof. The electrowetting force may be induced by a configuration of the array mentioned above. The dielectrophoretic effect may be photoinduced (electromagnetic radiation may be used to induce the effect). The dielectrophoretic effect may be induced by wires, sheets, electrodes, or any combination thereof created by photolithography, laser ablation, electron beam patterning, or any combination thereof. The wires, sheets, and electrodes may be made of metals (e.g., gold, copper, silver, titanium, etc.), alloys of metals, semiconductors (e.g., silicon, gallium nitride), or conductive oxides (e.g., indium tin oxide). The acoustic force may be ultrasonic. The acoustic force may be generated by a transducer. The hydrophobic knife may be a hydrophobic microtome or a hydrophobic razor blade.

The partitioning may dispense reagents. The reagents may be any of the reagents as described herein.

The partitioning may dispense samples. The samples may be a plurality of biological samples. The samples may be non-biological samples (e.g., chemicals).

The partitioned droplets may be mixed to execute a reaction. The reaction may be an amplification reaction, a chemical transformation, a binding reaction, the reaction of an antimicrobial agent with a microbe, or a reaction mentioned above.

The partitioned droplets may be analyzed using the sensors. The sensors may be any of the sensors from the array of sensors mentioned above.

The partitioned droplets may be mixed with at least one target droplet to maintain a constant volume on the at least one target droplet. The constant volume may be determined by computer vision (coupled cameras and an algorithm), mass, or optical spectroscopy (e.g., absorption spectroscopy).

The array may process multiphase fluids. The fluids may have at least 2, 3, 4, 5, 6, or more phases. For example, a droplet of water containing a colloid that is itself surrounded by a droplet of oil would have 3 phases.

The array may use dielectrophoretic forces (DEP) for cell sorting, cell separation, manipulating at least one bead, or any combination thereof. The DEP may be photoinduced (electromagnetic radiation may be used to induce the effect). The DEP may be induced by wires, sheets, electrodes, or any combination thereof created by photolithography, laser ablation, electron beam patterning, or any combination thereof. The wires, sheets, and electrodes may be made of metals (e.g., gold, copper, silver, titanium, etc.), alloys of metals, semiconductors (e.g., silicon, gallium nitride), or conductive oxides (e.g., indium tin oxide). The bead may comprise a magnetic bead, a bead for the storage of bacteria, an enzyme, an oligonucleotide, a nucleic acid, an antibody, a PCR primer, a ligand, a molecular sieve, or any combination thereof. The sorting and separation may be used for pre-concentrating at least one cell in raw clinical samples. The raw clinical samples may be derived from the plurality of biological samples. The raw clinical samples may be from a subject having or suspected of having a disease.

A biological sample, or a plurality thereof, may be deposited on an array or a plurality of arrays. The plurality of array may comprise at least two arrays. An array of the plurality of arrays may comprise a surface. The surface may comprise glass, a polymer, ceramic, metal, or any combination thereof. The surface may comprise a EWOD array, a DEW array, a DEP array, a microfluidic array, or any combination thereof. The plurality of arrays may comprise at least 2, 3, 4, 5, 6, 7, 8, 9, 10, 20, 30, 40, 50, 60, 70, 80, 90, 100, 200, 300, 400, 500, 600, 700, 800, 900, 1,000, or more arrays. The plurality of arrays may comprise most 1,000, 900, 800, 700, 600, 500, 400, 300, 200, 100, 90, 80, 70, 60, 50, 40, 30, 20, 10, 9, 8, 7, 6, 5, 4, 3, or 2 arrays. The plurality of arrays may comprise from 1,000 to 2 arrays, 500 to 2 arrays, 500 to 100 arrays, 100 to 2 arrays, 100 to 50 arrays, 50 to 2 arrays, 50 to 10 arrays, or 10 to 2 arrays. An array of the at plurality of arrays may be adjacent to another array of the plurality of arrays. The arrays may be horizontally, vertically, or diagonally adjacent.

The surface may have a thickness of at most 1,000 μm, 500 μm, 100 μm, 90 μm, 80 μm, 70 μm, 60 μm, 50 μm, 40 μm, 30 μm, 20 μm, 10 μm, 5 μm, 1 μm, 0.1 μm, 0.01 μm, or less. The surface may have a thickness of at least 0.01 μm, 0.1 μm, 1 μm, 5 μm, 10 μm, 20 μm, 30 μm, 40 μm, 50 μm, 60 μm, 70 μm, 80 μm, 90 μm, 100 μm, 500 μm, 1,000 μm, or more. The surface may have a thickness from 1,000 μm to 0.01 μm, 500 μm to 1 μm, 100 μm to 1 μm, or 50 μm to 1 μm.

The surface may have a roughness of at most 1,000 μm, 500 μm, 100 μm, 90 μm, 80 μm, 70 μm, 60 μm, 50 μm, 40 μm, 30 μm, 20 μm, 10 μm, 5 μm, 1 μm, 0.1 μm, 0.01 μm, 0.001 μm, or less. The surface may have a roughness of at least 0.001 μm, 0.01 μm, 0.1 μm, 1 μm, 5 μm, 10 μm, 20 μm, 30 μm, 40 μm, 50 μm, 60 μm, 70 μm, 80 μm, 90 μm, 100 μm, 500 μm, 1,000 μm, or more. The surface may have a roughness from 1,000 μm to 0.001 μm, 500 μm to 0.01 μm, 100 μm to 0.1 μm, or 50 μm to 0.1 μm.

The surface may comprise a layer of a liquid that has a wetting affinity characteristic for the surface. The liquid may be immiscible with a droplet or a plurality thereof. The liquid may be dispensed on the surface. An upper surface of the liquid may reduce friction between a droplet, or a plurality thereof, and the surface as compared to the droplet directly contacting the surface.

The plurality of arrays may contain a channel, a hole, or any combination thereof. The plurality of arrays may contain a plurality of channels, a plurality of holes, or any combination thereof. The channel, or plurality thereof, may traverse between at least one surface. A gas, liquid, solid, or any combination thereof may be transferred through a channel or a hole. A gas, liquid, solid, or any combination thereof may be transferred through a plurality of channels or a plurality of holes. The gas, liquid, solid, or any combination thereof may be transferred from one array to another array. The arrays may be adjacent to each other. The gas, liquid, solid, or any combination thereof may be transferred from one array to at least one other array. The gas, liquid, solid, or any combination thereof may be transferred from one array to at least two, three, four, five, six, seven, eight, nine, ten, or more arrays.

At least two droplets of the plurality of droplets may be separated by at least one membrane. The membrane may comprise metal, ceramic (e.g., aluminum oxide, silicon carbide, zirconium oxide, etc.), homogeneous films (e.g., polymers (e.g., cellulose acetate, nitrocellulose, cellulose esters, polysulfone, polyether sulfone, polyacrilonitrile, polyamide, polyimide, polyethylene, polypropylene, polytetrafluoroethylene, polyvinylidene fluoride, polyvinylchloride, etc.)), heterogeneous solids (e.g., polymeric mixes, mixed glasses, etc.), a liquid (e.g., emulsion liquid membranes, immobilized (supported), liquid membranes, molten salts, hollow-fiber contained liquid membranes, etc.), or any combination thereof. The membrane may allow passage of molecules, ions, or a combination thereof from one side of the membrane to the other. The membrane may be impermeable, semi-permeable, permeable, or a combination thereof. The permeability may separate according to size, solubility, charge, affinity, or a combination thereof. The membrane may be porous or semi-porous. The membrane may be biological, synthetic, or a combination thereof. The membrane may facilitate exchange of constituents of one droplet to another droplet. The may membrane facilitate passive diffusion, active diffusion, passive transport, active transport, or any combination thereof. The membrane may be a cation exchange membrane, a charge mosaic membrane, a bipolar membrane, an anion exchange membrane, an alkali anion exchange membrane, a proton exchange membrane, or a combination thereof. The membrane may be permanently or temporarily attached to the array, or plurality thereof.

Reaction Time

Aspects of the present disclosure provide for a method of generating a biopolymer wherein the reaction time is 30 minutes or less. In some embodiments, the reaction time is about 1 minute to about 30 minutes. In some embodiments, the reaction time is about 1 minute to about 2 minutes, about 1 minute to about 3 minutes, about 1 minute to about 4 minutes, about 1 minute to about 5 minutes, about 1 minute to about 10 minutes, about 1 minute to about 15 minutes, about 1 minute to about 20 minutes, about 1 minute to about 25 minutes, about 1 minute to about 30 minutes, about 2 minutes to about 3 minutes, about 2 minutes to about 4 minutes, about 2 minutes to about 5 minutes, about 2 minutes to about 10 minutes, about 2 minutes to about 15 minutes, about 2 minutes to about 20 minutes, about 2 minutes to about 25 minutes, about 2 minutes to about 30 minutes, about 3 minutes to about 4 minutes, about 3 minutes to about 5 minutes, about 3 minutes to about 10 minutes, about 3 minutes to about 15 minutes, about 3 minutes to about 20 minutes, about 3 minutes to about 25 minutes, about 3 minutes to about 30 minutes, about 4 minutes to about 5 minutes, about 4 minutes to about 10 minutes, about 4 minutes to about 15 minutes, about 4 minutes to about 20 minutes, about 4 minutes to about 25 minutes, about 4 minutes to about 30 minutes, about 5 minutes to about 10 minutes, about 5 minutes to about 15 minutes, about 5 minutes to about 20 minutes, about 5 minutes to about 25 minutes, about 5 minutes to about 30 minutes, about 10 minutes to about 15 minutes, about 10 minutes to about 20 minutes, about 10 minutes to about 25 minutes, about 10 minutes to about 30 minutes, about 15 minutes to about 20 minutes, about 15 minutes to about 25 minutes, about 15 minutes to about 30 minutes, about 20 minutes to about 25 minutes, about 20 minutes to about 30 minutes, or about 25 minutes to about 30 minutes. In some embodiments, the reaction time is about 1 minute, about 2 minutes, about 3 minutes, about 4 minutes, about 5 minutes, about 10 minutes, about 15 minutes, about 20 minutes, about 25 minutes, or about 30 minutes. In some embodiments, the reaction time is at least about 1 minute, about 2 minutes, about 3 minutes, about 4 minutes, about 5 minutes, about 10 minutes, about 15 minutes, about 20 minutes, or about 25 minutes. In some embodiments, the reaction time is at most about 2 minutes, about 3 minutes, about 4 minutes, about 5 minutes, about 10 minutes, about 15 minutes, about 20 minutes, about 25 minutes, or about 30 minutes. In some embodiments, the reaction time is about 10 minutes.

Aspects of the present disclosure provide that at least one nucleic acid molecule of a polynucleotide is generated in 30 minutes or less within a merged droplet. In some embodiments, the reaction time within the merged droplet is about 1 minute to about 30 minutes. In some embodiments, the reaction time within the merged droplet is about 1 minute to about 2 minutes, about 1 minute to about 3 minutes, about 1 minute to about 4 minutes, about 1 minute to about 5 minutes, about 1 minute to about 10 minutes, about 1 minute to about 15 minutes, about 1 minute to about 20 minutes, about 1 minute to about 25 minutes, about 1 minute to about 30 minutes, about 2 minutes to about 3 minutes, about 2 minutes to about 4 minutes, about 2 minutes to about 5 minutes, about 2 minutes to about 10 minutes, about 2 minutes to about 15 minutes, about 2 minutes to about 20 minutes, about 2 minutes to about 25 minutes, about 2 minutes to about 30 minutes, about 3 minutes to about 4 minutes, about 3 minutes to about 5 minutes, about 3 minutes to about 10 minutes, about 3 minutes to about 15 minutes, about 3 minutes to about 20 minutes, about 3 minutes to about 25 minutes, about 3 minutes to about 30 minutes, about 4 minutes to about 5 minutes, about 4 minutes to about 10 minutes, about 4 minutes to about 15 minutes, about 4 minutes to about 20 minutes, about 4 minutes to about 25 minutes, about 4 minutes to about 30 minutes, about 5 minutes to about 10 minutes, about 5 minutes to about 15 minutes, about 5 minutes to about 20 minutes, about 5 minutes to about 25 minutes, about 5 minutes to about 30 minutes, about 10 minutes to about 15 minutes, about 10 minutes to about 20 minutes, about 10 minutes to about 25 minutes, about 10 minutes to about 30 minutes, about 15 minutes to about 20 minutes, about 15 minutes to about 25 minutes, about 15 minutes to about 30 minutes, about 20 minutes to about 25 minutes, about 20 minutes to about 30 minutes, or about 25 minutes to about 30 minutes. In some embodiments, the reaction time is about 1 minute, about 2 minutes, about 3 minutes, about 4 minutes, about 5 minutes, about 10 minutes, about 15 minutes, about 20 minutes, about 25 minutes, or about 30 minutes. In some embodiments, the reaction time is at least about 1 minute, about 2 minutes, about 3 minutes, about 4 minutes, about 5 minutes, about 10 minutes, about 15 minutes, about 20 minutes, or about 25 minutes. In some embodiments, reaction time within the merged droplet is at most about 2 minutes, about 3 minutes, about 4 minutes, about 5 minutes, about 10 minutes, about 15 minutes, about 20 minutes, about 25 minutes, or about 30 minutes. In some embodiments, the reaction time within the merged droplet is about 10 minutes.

### Washing Steps

Some aspects of present disclosure provide for one or more washing steps. In some embodiments, one or more washing steps comprise subjecting a wash droplet to motion to contact a merged droplet. In some embodiments, a vibration is applied to said one or more washing steps.

dNTPs

Some aspects of present disclosure provide for droplets or reagents comprising deoxynucleoside triphosphate (dNTP). In some embodiments, the first droplet or reagent comprising deoxynucleoside triphosphate (dNTP). In some embodiments, the second droplet or reagent comprising deoxynucleoside triphosphate (dNTP). In some embodiments, the third droplet or reagent comprising deoxynucleoside triphosphate (dNTP). In some embodiments, the merged droplet or reagent comprising deoxynucleoside triphosphate (dNTP). In some embodiments, the deoxynucleoside triphosphate (dNTP) may have a protective group. In some embodiments, said protective group can be removed during the reaction.

### User Experience

In some aspects of present disclosure, the user experience may comprise certain workflow steps. In some embodiments, the user loads a proprietary Volta consumable cartridge for each batch of runs. In some embodiments, the user loads reagents into the dispenser for each batch of runs. In some embodiments, the user loads samples for each batch of runs. In some embodiments, the sample is loaded via a pipette. In some embodiments, the user engages with the touch screen interface to select their workflow and any other parameters at the start of the run. In some embodiments, the user unloads samples at the completion of a batch, or mid-batch if offline processing is required. In some embodiments, the sample is unloaded via a pipette.

### Subsystems

Some aspects of present disclosure provide for subsystems. In some embodiments, the subsystem may manipulate four reactions simultaneously. In some embodiments, the reactions comprise electrowetting, magnetic, other mechanical degrees of freedom, or a combination thereof. In some embodiments, the instrument may contain one subsystem. In some embodiments, the instrument may contain two subsystems. In some embodiments, the instrument may manipulate four reactions simultaneously. In some embodiments, the instrument may manipulate eight reactions simultaneously.

### Computer Hardware

Some aspects of the present disclosure provide for the use of computer hardware. In some embodiments the hardware comprises one or more of the processors described herein. In some embodiments, the one or more processors described herein are integrated modules. In some embodiments, the one or more processors described herein are NVIDIA® Jetson Nano™ Developer Kit processors.

### Computer Systems

Various processes described herein may be implemented by appropriately programmed general purpose computers, special purpose computers, and computing devices. Typically, a processor (e.g., one or more microprocessors, one or more microcontrollers, one or more digital signal processors) will receive instructions (e.g., from a memory or like device), and execute those instructions, thereby performing one or more processes defined by those instructions. Instructions may be embodied in one or more computer programs, one or more 10 scripts, or in other forms. The processing may be performed on one or more microprocessors, central processing units (CPUs), computing devices, microcontrollers, digital signal processors, or like devices or any combination thereof. Programs that implement the processing and the data operated on, may be stored and transmitted using a variety of media. In some cases, hardwired circuitry or custom hardware may be used in place of, or in combination with, some or all 15 of the software instructions that can implement the processes. Algorithms other than those described may be used.

Programs and data may be stored in various media appropriate to the purpose, or a combination of heterogenous media that may be read and/or written by a computer, a processor or a like device. The media may include non-volatile media, volatile media, optical or magnetic 20 media, dynamic random access memory (DRAM), static ram, a floppy disk, a flexible disk, hard disk, magnetic tape, any other magnetic medium, a CD-ROM, DVD, any other optical medium, punch cards, paper tape, any other physical medium with patterns of holes, a RAM, a PROM, an EPROM, a FLASH-EEPROM, any other memory chip or cartridge or other memory technologies. Transmission media include coaxial cables, copper wire and fiber optics, including 25 the wires that comprise a system bus coupled to the processor.

Databases may be implemented using database management systems or ad hoc memory organization schemes. Alternative database structures to those described may be readily employed. Databases may be stored locally or remotely from a device which accesses data in such a database.

In some cases, the processing may be performed in a network environment including a computer that is in communication (e.g., via a communications network) with one or more devices. The computer may communicate with the devices directly or indirectly, via any wired or wireless medium (e.g. the Internet, LAN, WAN or Ethernet, Token Ring, a telephone line, a cable line, a radio channel, an optical communications line, commercial on-line service providers, bulletin board systems, a satellite communications link, or a combination thereof). Each of the devices may themselves comprise computers or other computing devices, such as those based on the Intel® Pentium® or Centrino™ processor, that are adapted to communicate with the computer. Any number and type of devices may be in communication with the computer.

A server computer or centralized authority may or may not be necessary or desirable. In various cases, the network may or may not include a central authority device. Various processing functions may be performed on a central authority server, one of several distributed servers, or other distributed devices

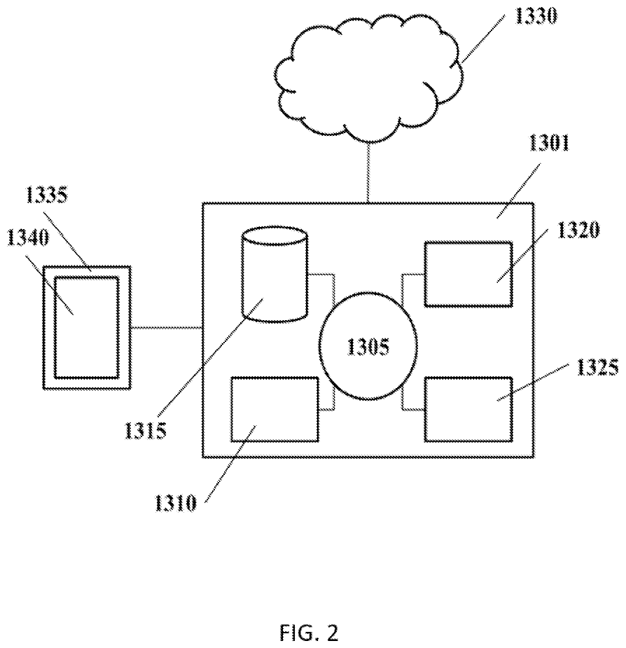

The present disclosure provides computer systems that are programmed to implement methods of the disclosure. FIG. 2 shows a computer system 1301 that is programmed or otherwise configured to manipulate a droplet, or a plurality thereof, on a system described herein. The computer system 1301 can regulate various aspects of sample manipulation of the present disclosure, such as, for example, droplet size, droplet volume, droplet position, droplet speed, droplet wetting, droplet temperature, droplet pH, beads in droplets, number of cells in droplets, droplet color, concentration of chemical material, concentration of biological substance, or any combination thereof. The computer system 1101 can be an electronic device of a user or a computer system that is remotely located with respect to the electronic device. The electronic device can be a mobile electronic device.

The computer system 1301 includes a central processing unit (CPU, also “processor” and “computer processor” herein) 1305, which can be a single core or multi core processor, or a plurality of processors for parallel processing. The computer system 1301 also includes memory or memory location 1310 (e.g., random-access memory, read-only memory, flash memory), electronic storage unit 1315 (e.g., hard disk), communication interface 1320 (e.g., network adapter) for communicating with one or more other systems, and peripheral devices 1325, such as cache, other memory, data storage, electronic display adapters, or any combination thereof. The memory 1310, storage unit 1315, interface 1320 and peripheral devices 1325 are in communication with the CPU 1305 through a communication bus (solid lines), such as a motherboard. The storage unit 1315 can be a data storage unit (or data repository) for storing data. The computer system 1301 can be operatively coupled to a computer network (“network”) 1330 with the aid of the communication interface 1320. The network 1330 can be the Internet, an internet, extranet, or any combination thereof, or an intranet, extranet, or any combination thereof that is in communication with the Internet. The network 1330 in some cases is a telecommunication, data network, or any combination thereof. The network 1330 can include one or more computer servers, which can enable distributed computing, such as cloud computing. The network 1330, in some cases with the aid of the computer system 1301, can implement a peer-to-peer network, which may enable devices coupled to the computer system 1301 to behave as a client or a server.

The CPU 1305 can execute a sequence of machine-readable instructions, which can be embodied in a program or software. The instructions may be stored in a memory location, such as the memory 1310. The instructions can be directed to the CPU 1305, which can subsequently program or otherwise configure the CPU 1305 to implement methods of the present disclosure. Examples of operations performed by the CPU 1305 can include fetch, decode, execute, and writeback.

The CPU 1305 can be part of a circuit, such as an integrated circuit. One or more other components of the system 1101 can be included in the circuit. In some cases, the circuit is an application specific integrated circuit (ASIC).

The storage unit 1315 can store files, such as drivers, libraries and saved programs. The storage unit 1315 can store user data, e.g., user preferences and user programs. The computer system 1301 in some cases can include one or more additional data storage units that are external to the computer system 1301, such as located on a remote server that is in communication with the computer system 1301 through an intranet or the Internet.

The computer system 1301 can communicate with one or more remote computer systems through the network 1330. For instance, the computer system 1301 can communicate with a remote computer system of a user (e.g., mobile electronic device). Examples of remote computer systems include personal computers (e.g., portable PC), slate or tablet PC's (e.g., Apple® iPad, Samsung® Galaxy Tab), telephones, Smart phones (e.g., Apple® iPhone, Android-enabled device, Blackberry®), or personal digital assistants. The user can access the computer system 1301 via the network 1330.

Methods as described herein can be implemented by way of machine (e.g., computer processor) executable code stored on an electronic storage location of the computer system 1301, such as, for example, on the memory 1310 or electronic storage unit 1315. The machine executable or machine readable code can be provided in the form of software. During use, the code can be executed by the processor 1305. In some cases, the code can be retrieved from the storage unit 1315 and stored on the memory 1310 for ready access by the processor 1305. In some situations, the electronic storage unit 1315 can be precluded, and machine-executable instructions are stored on memory 1310.

The code can be pre-compiled and configured for use with a machine having a processer adapted to execute the code, or can be compiled during runtime. The code can be supplied in a programming language that can be selected to enable the code to execute in a pre-compiled or as-compiled fashion.

Aspects of the systems and methods provided herein, such as the computer system 1301, can be embodied in programming. Various aspects of the technology may be thought of as “products” or “articles of manufacture” typically in the form of machine (or processor) executable code, associated data, or any combination thereof that is carried on or embodied in a type of machine readable medium. Machine-executable code can be stored on an electronic storage unit, such as memory (e.g., read-only memory, random-access memory, flash memory) or a hard disk. “Storage” type media can include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements includes optical, electrical and electromagnetic waves, such as used across physical interfaces between local devices, through wired and optical landline networks and over various air-links. The physical elements that carry such waves, such as wired or wireless links, optical links or the like, also may be considered as media bearing the software. As used herein, unless restricted to non-transitory, tangible “storage” media, terms such as computer or machine “readable medium” refer to any medium that participates in providing instructions to a processor for execution.

Hence, a machine readable medium, such as computer-executable code, may take many forms, including but not limited to, a tangible storage medium, a carrier wave medium or physical transmission medium. Non-volatile storage media include, for example, optical or magnetic disks, such as any of the storage devices in any computer(s) or the like, such as may be used to implement the databases, etc. shown in the drawings. Volatile storage media include dynamic memory, such as main memory of such a computer platform. Tangible transmission media include coaxial cables; copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code, data, or any combination thereof. Many of these forms of computer readable media may be involved in carrying one or more sequences of one or more instructions to a processor for execution.

The computer system 1301 can include or be in communication with an electronic display 1335 that comprises a user interface (UI) 1340 for providing, for example, information related to droplet manipulation, sample manipulation, or a combination thereof. Examples of UI's include, without limitation, a graphical user interface (GUI) and web-based user interface.

Methods and systems of the present disclosure can be implemented by way of one or more algorithms. An algorithm can be implemented by way of software upon execution by the central processing unit 1105. The algorithm can, for example, provide additional liquid to a droplet, replace evaporated solvent of a droplet, map out a path for a droplet, or any combination thereof.

Video, input, and control of the system may be accessed through a web-based software application. User inputs through software may include, for example, droplet motion, droplet sizes, and images of the array, and user inputs may be recorded and stored in a cloud-based computing system. Stored user inputs may be accessed and retrieved in subsets or in entirety to inform machine-learning based algorithms. Droplet movement patterns may be recorded and analyzed for use in training navigation algorithms. Trained algorithms may be used for automation of droplet movement. Spatial fluid properties may be recorded and analyzed for use in training protocol optimization and generation algorithms. Trained algorithms may be used for optimizing biological and droplet movement protocols or in the generation of new biological and droplet movement protocols. Biological quality control techniques (e.g., amplification-based quantification methods, fluorescence-based, absorbance-based quantification, surface plasmon resonance methods, and capillary-electrophoretic methods to analyze nucleic acid fragment size) may be used to analyze the effectiveness of the workflows performed on the array. The data from these techniques may then be used as an input into machine learning algorithms to improve output. The process may be automated so that the system can iteratively improve the output.

### EXAMPLES

### Example 1: Electrowetting Without a Dedicated Reference Electrode

In single sided electrowetting systems (e.g. the devices and systems described herein) a conductive top plate is not used as a current return path for the droplet. Instead, it's common for a dedicated coplanar (or nearly coplanar) electrode to be used in conjunction with actively driven electrode pads. This is to provide a low impedance discharge path for accumulated charge in the droplet. These electrodes often take the shape of a grid mesh with the same spacing as the active electrode grid below.

The fabrication and implementation of these coplanar (or nearly coplanar) reference electrodes can greatly complicate electrowetting systems and methods for utilizing such systems. Achieving a low impedance connection to the droplet without disrupting the fluidic mobility of the droplet on the surface can be a significant challenge. Instead, the systems and methods presented here remove the need for a dedicated reference electrode by using neighboring electrodes as the current-return path. These systems and methods provide comparable electrowetting performance and completely eliminate fabrication challenges related to the integration of a dedicated reference electrode.

The circuit with a conventional dedicated reference electrode includes a resistive return path which acts to ground the droplet. Without a dedicated reference electrode, the return path includes a capacitive element formed between the inactive electrode(s) and the droplet across the dielectric membrane (FIG. 15). For this reason, activating the electrodes with a time-varying voltage is necessary in order for this current-return path to be effective. This time varying voltage may be bipolar in which case the high voltage signal is both positive and negative relative to the “0V” inactive electrodes. In another embodiment, the time varying voltage may be unipolar in which case the high voltage signal is only positive and neighboring electrodes are driven antagonistically such that the electric field across the droplet flips direction periodically.

The circuit may be driven at a wide range of frequencies. The lower limit is determined by the droplet's hydrodynamic response to the excitation and for droplets in the range of ˜100 nL to 100 uL (but can include the volumes described herein); this is commonly at most ˜100 Hz. The upper limit of the frequency range is determined by the RC time constant of the circuit and, practically, is limited to ˜1 kHz as a result of current limiting resistors in the conductive path. This frequency range could be extended through the use of dedicated high voltage circuits that support higher currents (e.g. up to 20 kHz).

### Example 2: Electrowetting Array Comprising a Lubricating Fluid Disposed on the Surface of the Array

A smooth dielectric film (textureless) with lubricating oil film together providing a low friction surface can be used for efficient manipulation of droplets using electrowetting or other digital microfluidic or droplet manipulation approaches. In prior disclosures (US20190262829A1), the lubricating oil film is formed on a textured surface. However, an alternate approach that does not require a porous dielectric film to hold the lubricating oil but instead relies on chemical affinity between the surface of the film and the lubricating oil is proposed herein.

Similarly to the devices and systems described herein wherein the droplet motions across a liquid surface disposed on a textured surface, in the case of a textureless surface, the droplet is again above the surface of a lubricating film. The lubricating film comprises a lubricating liquid immiscible with the droplet. The lubricant film is thermodynamically stable such that it preferentially wets the surface of the dielectric and droplets sit on top of the lubricant film. Achieving this stability is important and is governed by the affinity of the lubricant liquid to the surface of the dielectric. For fluorinated surfaces of dielectric, it may be advantageous to use fluorinated lubricant liquids. The similar chemical structure leads to a greater affinity and therefore the lubricant is more likely to wet the surface in a stable way. On the other hand, when using dielectrics with hydrocarbon-based surfaces, or siliconized surfaces, (such as silicones and untreated polymer plastics), it may be advantageous to use a hydrocarbon based lubricant liquid (such as silicone oil).

The lubricating film can include, but is not limited to:

Silicone oils: polydimethylsiloxanes, polymethyl hydrogen siloxane/hydrogen silicone oil, amino silicone oil, phenyl methyl silicone oil, Dipheny silicone oil, vinyl silicone oil, hydroxy silicone oil, cyclosiloxanes, polyalkylene oxide silicones.

Fluorinated oils: perfluoropolyether (PFPE), perfluoroalkanes, fluorinated ionic fluid, fluorinated silicone oils, perfluoroalkylether, perfluoro tri-n-butylamine (FC-40), hydrofluoroether (HFE) liquids.

Other lubricants: ionic liquids, mineral oils, ferrofluids, polyphenyl ether, vegetable oil, esters of saturated fatty and dibasic acids, grease, fatty acids, triglycerides, polyalphaolefin, polyglycol hydrocarbons, other Non-hydrocarbon synthetic oils.

Lubricant liquid may contain other functional additives, including surfactants, electrolytes, rheology modifier, wax, graphite, graphene, molybdenum disulfide, PTFE particles.

### Example 3: Electrowetting Array Comprising a Filler Fluid Below the Dielectric

The devices and systems described herein generally include a dielectric layer disposed on a layer of co-planar (or nearly co-planar) electrodes. Described herein, are further embodiments wherein the devices and systems include a filler fluid disposed below the dielectric layer.

The filler fluid below the film serves to keep the dielectric film in close contact with the underlying PCB substrate and electrode grid through surface tension. When a dielectric film is applied to the surface of an electrode array, a layer of oil fills the air gap between the film and the electrodes and the air gap between the electrodes. So, while filling the air gap between the electrodes and the film, the oil layer keeps the film adhered to the surface via surface tension. Further, filling the airgap between any two neighboring electrodes the oil acts as a high dielectric breakdown material and prevents air from breaking down.

Air typically has a breakdown voltage of about 1 kilovolt per millimeter. So while reducing the gap between two neighboring electrodes is beneficial to allow for smooth transition of droplets, if the gap between two electrodes is reduced at some point, then it will start conducting and rendering the electrowetting device non-functional. By adding an oil to fill the gap between two electrodes, the gap between the electrodes will be reduced and high voltages can be utilized for reliable droplet motion.

A layer of oil below the dielectric film also has the benefit of smoothening the surface of the film. It allows for the dielectric film to easily stretch and unstretch on a lubricated layer. This easy stretching-unstretching allows for the film settle with no wrinkles in the film. Wrinkles in the film can prevent droplets from being mobile and/or provide further hinderances to droplet motion. Finally, having a layer of oil below the dielectric film provides a way to easily attach and detach the film from an electrode array. The filler oil layer here acts as a semi-permanent adhesive and keeps the dielectric layer on the electrode array when the device is in use. However, a user can easily remove the dielectric layer from the surface since it is not permanently fixed to the surface of the electrode array. The alternative to not using oil would mean that the dielectric film/layer is permanently attached to the electrode array or the user would need more complex instrumentation to pull the film down to the surface evenly across the entire surface. In these embodiments, the use of the filler fluid on the electrode array can enable devices and systems with removable “cartridges” wherein the cartridges can include the surface which supports the droplet.

### Example 4: Enzymatic DNA Synthesis

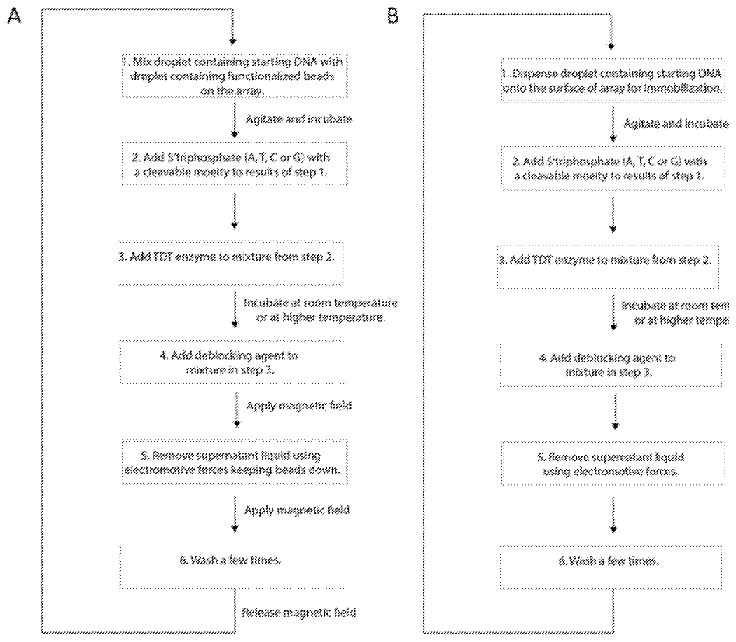

A method to synthesize polynucleotides (e.g., DNA) using an enzyme catalyzed process in an aqueous medium on arrays described herein was performed. Terminal Deoxynucleotidyl Transferase (TDT) is a template independent polymerase that catalyzes the formation of phosphodiester bonds between the 3′ and 5′ end of DNA. FIG. 5A and FIG. 5B show example workflows to afford the synthesis of DNA. FIG. 5C shows a schematic diagram for a single reaction site that performs step by step addition of nucleotides to synthesize a long molecule of DNA.

Aspects of the present disclosure provide that a reagent comprises of an enzyme that mediates synthesis or polymerization. In some embodiments, the first reagent, second reagent, third reagent, or any combination thereof comprises an enzyme that mediates synthesis or polymerization. In some embodiments, the enzyme is from the group consisting of Polynucleotide Phosphorylase (PNPase), Terminal Denucleotidyl Transferas (TdT), DNA polymerase Beta, DNA polymerase lambda, DNA polymerase mu and other enzymes from X family of DNA polymerases.

A droplet containing a starting DNA material with an unprotected 3′-hydroxyl group is mixed with a droplet containing functionalized magnetic beads. After a brief period of agitation, the DNA molecules are bound to the magnetic beads. Alternatively, a droplet containing starting DNA material is dispensed onto a location of the array that is functionalized to immobilize the DNA to a solid support. A droplet containing a nucleoside 5′-triphosphate with a cleavable/removable moiety is mixed with the droplet containing immobilized starting DNA. TDT enzyme, which catalyzes the 5′ to 3′ phosphodiester linkage between the unprotected 3′-hydroxyl end of the starting DNA and the 5′-phosphate end of the nucleoside triphosphate, in a droplet is then merged and mixed with the droplet containing immobilized DNA. The reaction is incubated for at room temperature or higher temperature for 5-30 minutes.

A droplet containing a deblocking agent is then mixed with the subsequent reaction mixture, producing the nucleotide with a free 3′-hydroxyl. In the case of using magnetic beads for immobilization, a magnetic field is then applied to pull the beads down to the surface of the array and the excess liquid is removed. The beads are then washed multiple times (e.g., 2-4) by flowing a washing buffer over the beads. The washed liquid is then discarded to the waste area of the array. Additional nucleotides are added to the DNA by repeating the method described above. During each addition of a nucleoside triphosphate, a controller instructs the array to dispense one of the nucleoside triphosphates from respective reservoirs. After multiple iterations, a polynucleotide of known sequence is produced, staying immobilized either to the beads or on the functional surface of the array. The final DNA product is cleaved and released from the surface (e.g., the beads or the surface of the array) by bringing a droplet containing a cleaving agent. The final product is then suspended in a droplet and recovered from the array.

Errors in DNA synthesis can be corrected with mismatch binding and mismatch cleaving proteins. A mismatch binding protein (e.g., MutS) is bound to a magnetic bead and mixed with a droplet containing assembled DNA comprising at least one error (e.g., identified as distortion in the double helix). For example, DNA molecules comprising an error are bound to the magnetic beads and the DNA without errors are not attached to the beads. The beads are then moved to another area of the array using a magnetic field, removing the DNA comprising at least one error. The excess liquid containing DNA with no errors is separated from the beads using electromotive force (e.g. EWOD).

Alternately, errors are corrected using mismatch cleaving enzymes, such as, for example, T4 endonuclease VII or T7 endonuclease I. A droplet comprising a cleaving enzyme is mixed with a droplet containing assembled DNA. The mismatch cleaving enzymes target the regions at or near the errors. The error-free fragments are then retrieved using magnetic bead-based separation. Alternately, exonucleases are used to remove additional errors on fragments left over by the mismatch cleaving enzymes. These trimmed fragments are assembled correctly using PCR assembly in a droplet.

The assembled and error corrected DNA is amplified using PCR in a droplet. The final product from PCR is then prepared into libraries for sequencing on the array using methods described herein. The libraries are sequenced using any of the sequencing techniques described herein for final sequence verification of the synthesized DNA.

The protocols described herein were performed using the platform using a 10 base initiator DNA. Two reactions were performed to synthesize the addition of 5 and 10 thymine bases to the base DNA. The results were analyzed using denaturing polyacrylamide gel electrophoresis on an Azure 600 with blue light illumination. The DNA was conjugated to fluorescein dye for visualization. The synthesis was confirmed by Agarose gel assay.

The protocol described herein has also been used to prepare function DNA primers for use in PCR amplification. A forward and reverse primer were synthesized at a 200 pmole scale and required manual post synthesis processing. Functionality of the primers was assayed by performing a 40 cycle PCR protocol and having the results analyzed using 2% Agarose gel (e-gel EX). The results, confirmed by gel assays, suggest that the primers synthesized using the systems and methods described herein have comparable functionality as IDT DNA primers based on the endpoint PCR analysis.

### Example 5: Extraction of high molecular weight (HMW) nucleic acid

Cells from various sources (e.g., mammalian, bacterial, plants) are lysed directly on the array by merging a droplet containing cells with another droplet containing lysis agent (e.g., detergent or enzymatic). This mixture is heated and mixed (e.g., separately or simultaneously) on the EWOD array to promote lysis of cells and, if applicable, lysis of the nucleus. Enzymatic digestion of proteins, RNA, or a combination thereof are performed to improve the purity of the sample. While cells are lysed, the progress of lysis reaction and lysis efficiency is monitored via DNA-specific fluorescent stains. DNA is purified directly on the array by solid phase (e.g., bead-based capture or by precipitation (e.g. salt and ethanol or phenol-chloroform extraction)). Recovered DNA is manipulated and transferred to different locations of the array by EWOD with minimal shearing. DNA purity, critical for high quality long-read sequencing, is improved by increasing the number of washing cycles performed on the array. Small DNA fragments are removed using silica-nanostructured magnetic disks. The yield of recovered DNA is increased by performing additional successive elution in buffers.

After DNA extraction, samples are analyzed by Pulse Field Gel Electrophoresis (PFGE), quantifying size distribution for each sample relative to one another and analyzing commercially available ladders (BioRad) and ImageJ (NIH) profile analysis tools. For smaller inputs, (e.g., cell input) recovery/size distribution is measured by Femto Pulse (Agilent) and qPCR for lower input amounts. Genomic intactness is assessed by additional complementary methods, e.g. the BioNano Genomics Saphyr System, allowing rapid and cost-effective prototyping at a macro scale as well as independent comparability of data using the Saphyr System.

The passivation of the EWOD surface is determined by testing DNA deposition and retention in the presence of solution- and surface-deposited PEG200 or BlockAid (Invitrogen) passivated devices. Measurements are obtained i) by staining the surfaces after use with Hoechst 33342, ii) calculating surface retention of a commercial preparation of Lambda DNA (New England Biolabs, linearized 48.5 Kb), and/or iii) measuring % loss by qPCR of sample pre- and post-manipulation and input quantities from 109 to 10² copies of DNA.

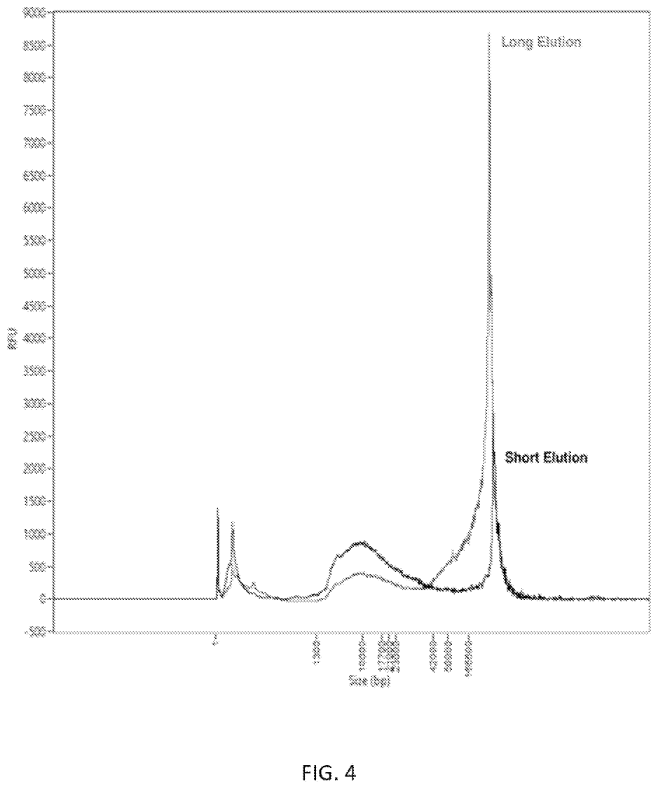

Mammalian cell lysis, RNA, and protein digestion followed by HMW DNA isolation was performed on the EWOD array. The distribution of the high molecular weight DNA fragments are seen in FIG. 4 with DNA fragments larger than 165,000 bp being isolated from the sample. The longer duration of elution recovers more DNA (e.g., indicated by the taller peak) and is a way to obtain higher DNA yield.

These techniques were utilized to prepare DNA libraries for sequencing as described in the following examples.

### Example 6: Next-Generation Sequencing (NGS) Library Preparation

224 nanograms (ng) of purified genomic DNA was used as starting material and Genome In A Bottle NA12878 was used as the DNA source. Final libraries were amplified by two cycles of PCR, which was performed on a thermal cycler in a separate post-PCR area. A control library was performed off-chip manually for data comparison. Libraries were quantified by Qubit and fragment size distribution was assessed by BioAnalyzer. Libraries were normalized accordingly and sequenced on a NextSeq500 (e.g., shallow sequencing with initial mid-output run at 2×75 cycles and 2×8 cycles for the indexes followed by additional coverage generation with a high-output 2×150 cycles run). Sequencing data was demultiplexed using Illumina's bc12fastq v2.20 without adapter trimming. Bioinformatics analysis was performed using well-established algorithms (e.g., FASTQC, BWA-MEM, SAMtools, Picard and GATK).

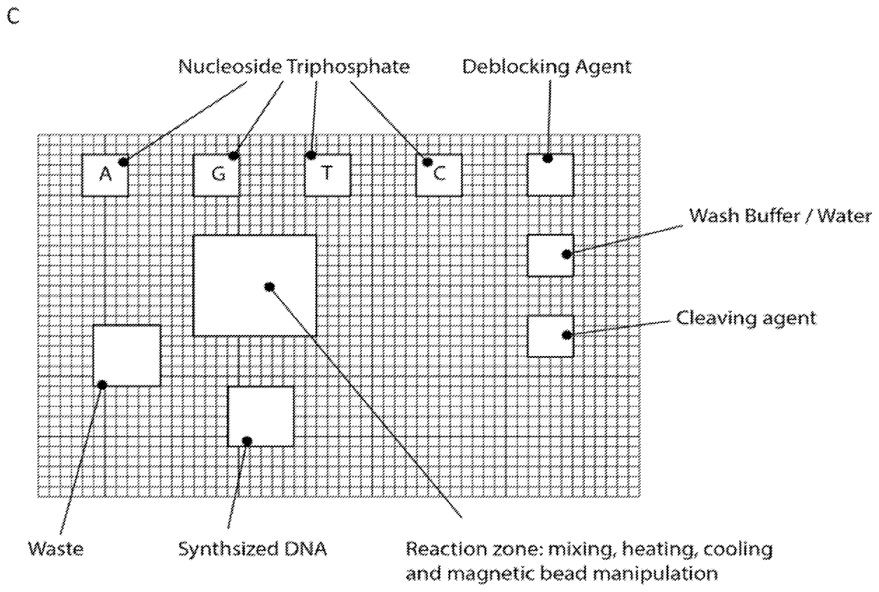

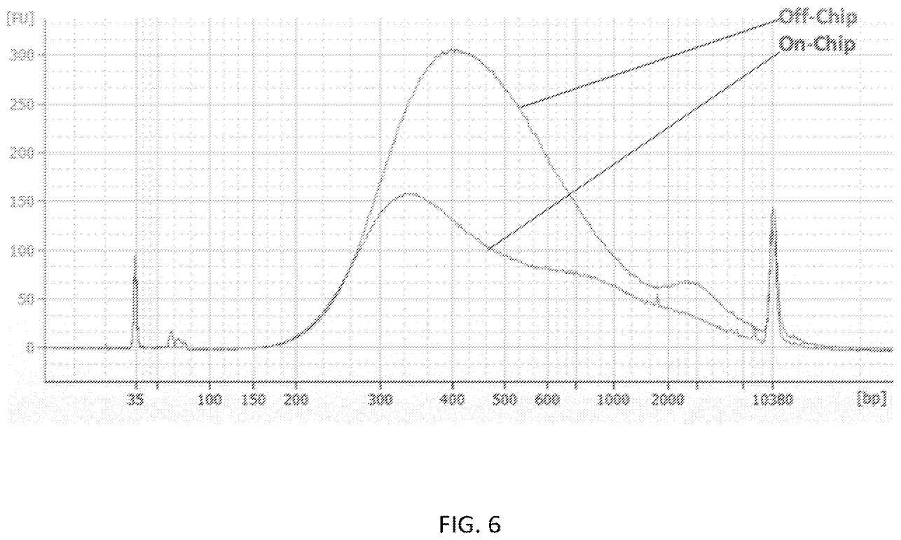

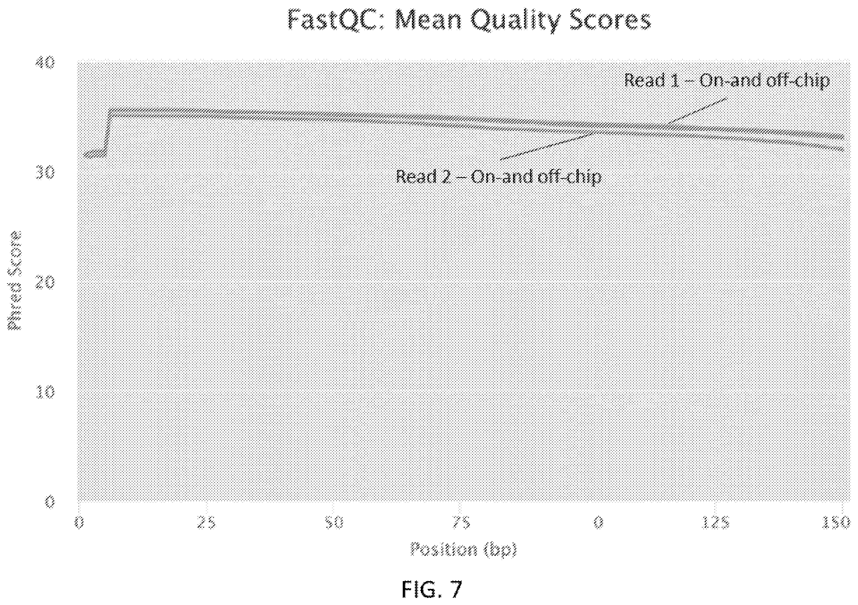

The library prepared on chip generated enough material for sequencing (Table 1). The off-chip control generated ˜2.3× more DNA material than the on-chip experiment; however, the average fragment size was higher than described previously for both on-chip and off-chip libraries (Table 1 & FIG. 6). All sequencing and mapping QC data demonstrated that high quality sequencing libraries were generated (Table 1), with Q30 >90% (FIG. 7), % PF reads >90%, using systems and methods described herein.

TABLE 1

| Metrics | Target Value | | On Chip | Manual - Off-chip |
|---|---|---|---|---|
| DNA Yield | 125-625 | ng | 294.32 | 712.4 |
| Average fragment Size | 250 | bp | 450 | 467 |

| Number of Reads | — | 158693266 | 106693500 |
|---|---|---|---|
| Q30 | >75% | 93.35% | |
| PF Reads | >80% | 92.90% | |
| % Duplicates | — | 9.35% | 8% |
| % PF reads aligned (hg19) | >0.9 | 0.994232 | 0.993074 |
| Median Coverage | — | 9 | 7 |
| HET SNP Sensitivity | >0.8 | 0.836866 | 0.875379 |

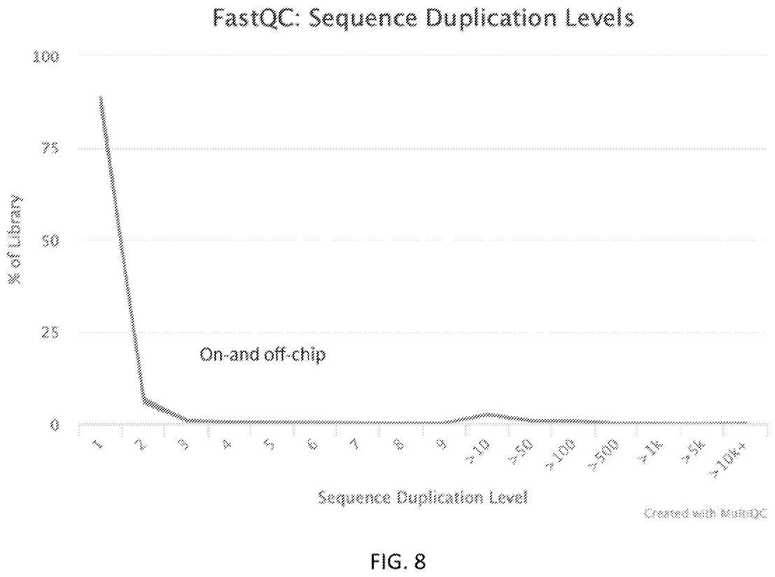

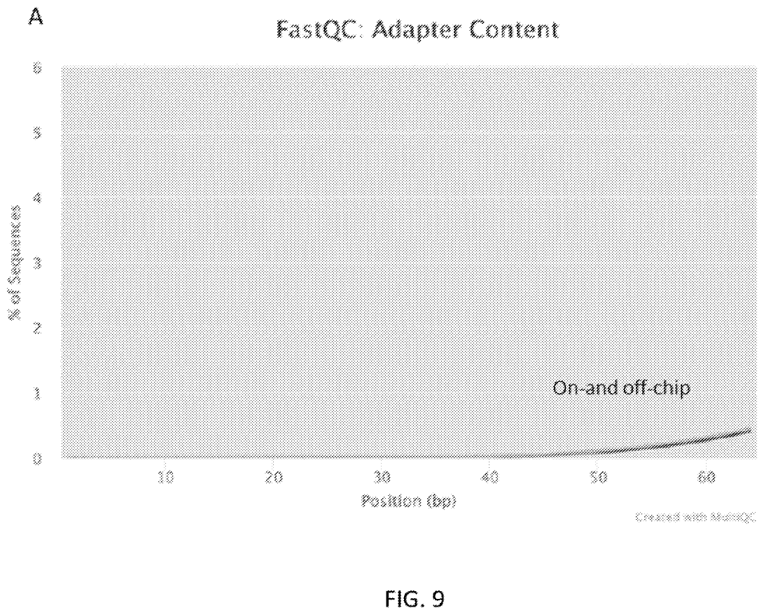

The level of duplicates for both on and off-chip libraries was low (FIG. 8) and <0% overall (Table 1). The low level of duplicated reads was also reflected in the limited content in adapters. Our initial shallow sequencing (2×75) indicated <1% adapter contamination (FIG. 9A) while up to 15% and 10% adapters for on-chip and off-chip libraries, respectively, were detected when increasing sequencing depth and read length to 2×150 (FIG. 9B). The difference between on and off-chip can be due to the higher number of reads generated for the on-chip library compared to off-chip control.

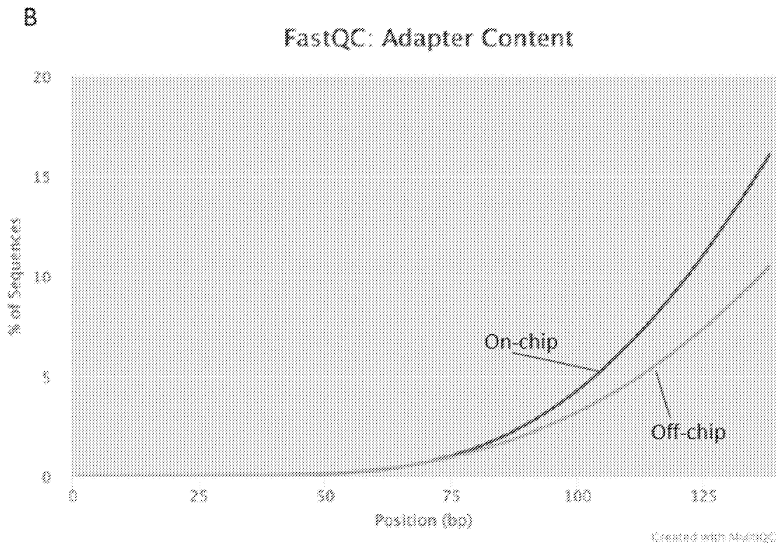

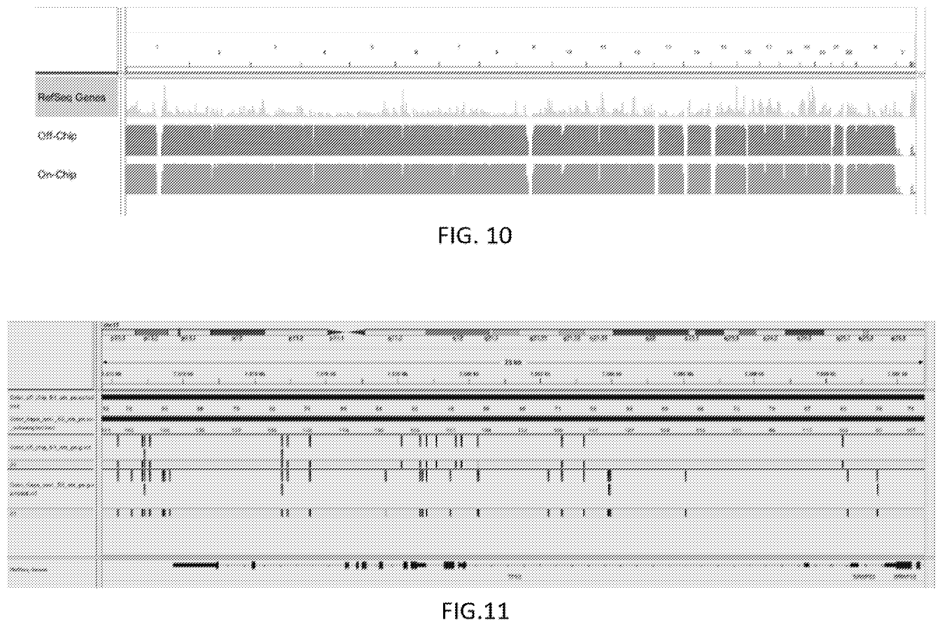

Mapping rate of passed-filter reads was high (>99%) and coverage across the genome was comparable between both libraries (FIG. 10), at a median coverage of 9× and 7× for the on-chip and off-chip libraries, respectively. Variants and the ability to call single nucleotide polymorphisms (SNPs) was determined. The heterozygous (HET) single nucleotide polymorphism (SNP) sensitivity was comparable at similar coverage between on- and off-chip (Table 1). This was confirmed by looking specifically at SNPs on the TP53 locus where identical genotypic variants were detected for both libraries in intergenic regions (FIG. 11).

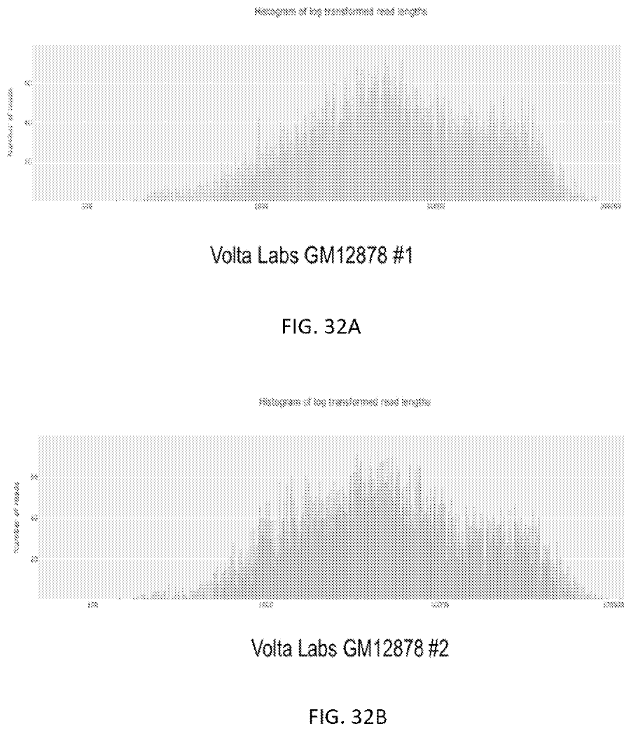

The LSK-110 ligated-based library preparation was performed on the platform to generate a library for analysis on the Oxford Nanopore MinION. Kit consumables were prepared and loaded onto the platform along with the Film Consumable for the platform itself. 1 μL of HMW gDNA derived from GM12878 cells were then loaded onto the platform. The systems automated preparation protocol was loaded and launched. At the end of the automated protocol the library was attached to beads and was manually eluted using a 37° C. incubation for 10 minutes followed by a magnetic rack. 12 μL of the supernatant containing the prepared library were transferred to an Oxford Nanopore system for analysis. The results from the experiment are displayed in Table 2 and histograms showing the read lengths for two different experimental runs are shown in FIG. 32A and FIG. 32B.

TABLE 2
Feature

| General summary | Volta Labs GM12878 #1 | Volta Labs GM12878 #2 |
|---|---|---|
| Active channels | 480.0 | 510.0 |
| Mean read length | 10,917.9 | 10,066.3 |
| Mean read quality | 12.7 | 12.5 |
| Median read length | 5,544.0 | 4,793.0 |
| Median read quality | 13.5 | 13.2 |
| Number of reads | 1,361,151.0 | 1,546,672.0 |
| Read length N50 | 23,628.0 | 23,263.0 |
| STDEV read length | 12,862.8 | 12,655.0 |
| Total basses | 14,860,952,865.0 | 15,569,239,873.0 |

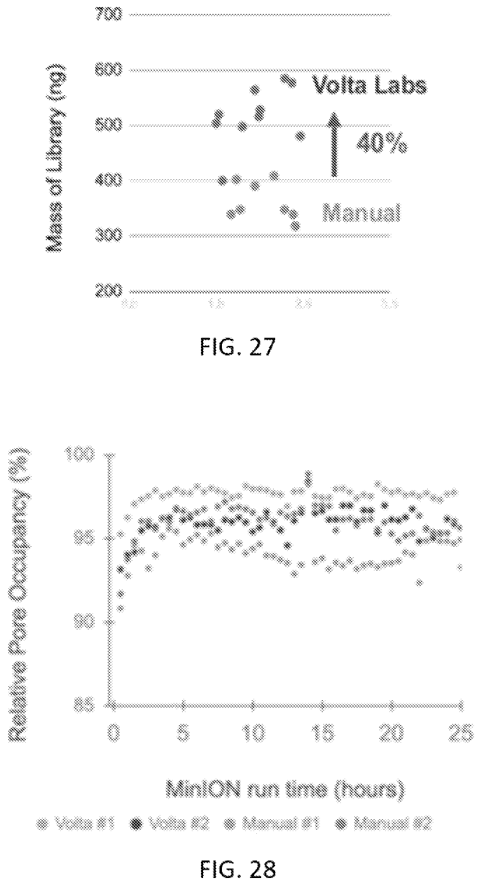

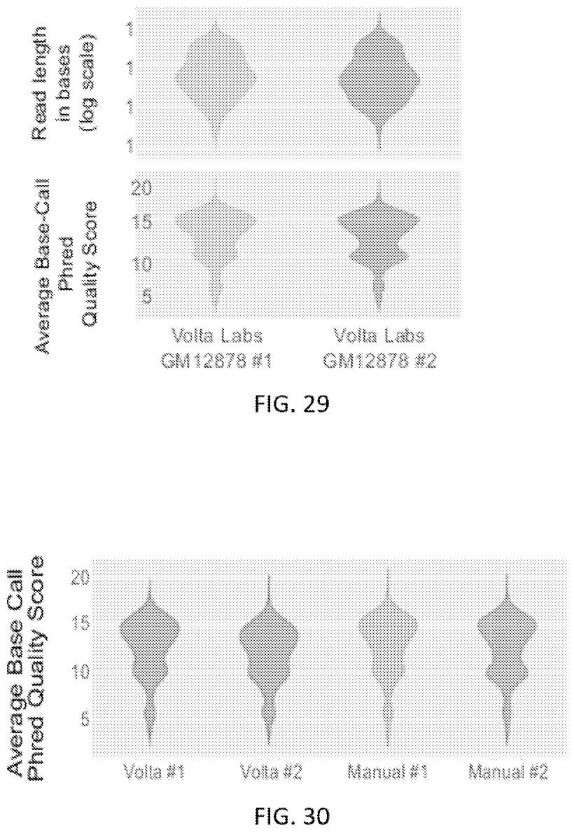

The preparation on the platform resulted in a library that contained a 40% higher yield when compared to manual preparation as seen in the results in FIG. 27. The library also showed a high level of compatibility with the MinION sequencing chemistry through high relative pore occupancy percentage when compared to manually prepared samples as shown in FIG. 28. The base call quality scores were also highly similar to those derived from manually prepared libraries as seen in FIG. 30. The sequencing data also demonstrated that the gDNA from the platform resulted in N50 of 23 kb as shown in FIG. 29.

### Example 7: Workflow Sample Preparation for DNA Samples for Sequencing on an Array

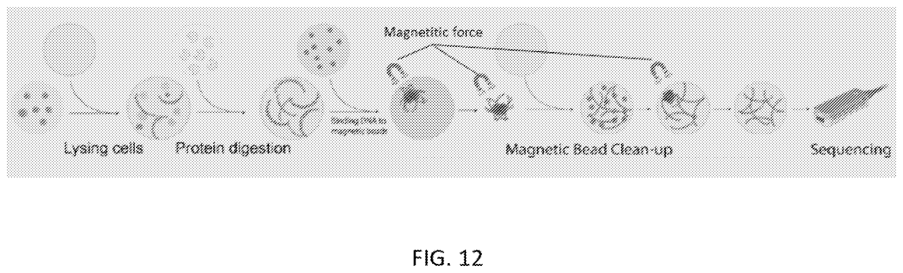

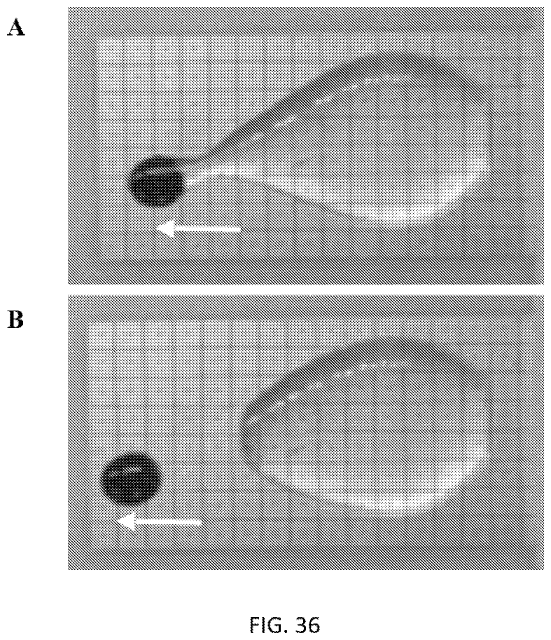

An example of a workflow for NGS on an array described herein is shown in FIG. 12. Cells in a droplet on an array are lysed on the array by introducing another droplet comprising chemical or enzymatic cellular lysis reagents. The proteins contained in the droplet are degraded by introducing degradation enzymes contained in another droplet of the array, and magnetic particles specific for DNA molecules are introduced to the droplet containing the DNA molecules. The magnetic beads are attached to the surface of the array or the magnetic beads are suspended in a droplet. The DNA molecules are separated and isolated from the cellular debris and degraded proteins using magnetic fields of the array (e.g. the movable magnets as described herein). The isolated DNA, attached to magnetic particles suspended in solution, is separated from the droplet by translating the movable magnet across a plane parallel to the surface of the substrate as depicted in FIG. 36A-36B. The isolated, DNA-coated beads undergo a magnetic bead washing process. The DNA is introduced to a DNA sequencer on, adjacent to, or separate from the array. The DNA is sequenced.

### Example 8: High-Molecular Weight DNA Extraction with Vibration Assisted Mixing

### Aims

In this experiment the intended goal is to extract long and clean DNA from biological samples such as blood, mammalian cells and cultured microbes. Typically, the aim is to isolate DNA of length greater than 50 kb (50 kilobases) and isolate enough nucleic acid material for downstream applications such as DNA sequencing and optical mapping.

The workflow starts with lysing the cells in biological samples (e.g. cells, blood). The lysis is carried out on an electrowetting array by merging two droplets-one containing the cells that need to be lysed and the other containing the lysis reagents. The lysed cells release everything from within including long pieces of DNA, proteins and other cell debri (collectively called “cell lysate”). This mixture of cell lysate is generally quite viscous. Viscous fluids do not move in response to electrowetting forces or experience severely impaired movement (e.g. more energy is needed to induce motion).

A typical method to isolate nucleic acid molecules (e.g. DNA, RNA) from this type of cell lysate mixture is by using magnetically responsive functionalized substrates (e.g. beads, discs) that have the affinity to bind to nucleic acid molecules. So even if magnetic beads are added to the mixture containing the cell lysate, due to the viscosity of the cell lysate and its non-responsiveness to electrowetting, it is difficult to mix the substrate(s) with the lysate. The substrate(s) will remain stationary within the fluid and not sufficiently bind to the nucleic acid molecules. And most often, it might be difficult to proceed to next steps in the workflow to complete the isolation of nucleic acid molecules. This is because it might be difficult to remove the excess fluid from the mixture-which is essential to separate everything from the nucleic acid molecules that are bound to the substrate(s). Even if the excess fluid was separated, the amount of nucleic acid molecules bound to the substrate(s) is typically very low. As a result, most of the nucleic acid molecules are lost and % isolated from the sample of interest is very low.

Introducing vibration/acoustic forces into this system allows for mixing viscous cell lysate with magnetic substrates (e.g. DNA with beads). This encourages most of the DNA in the lysate to bind to the beads. Once the DNA is bound to the beads, the excess fluid then becomes less viscous. The excess, less viscous fluid can then be easily separated from the beads using more standard electrowetting as described herein.

In addition to enabling efficient DNA binding onto the beads from the cell lysate, vibration assisted mixing allows for highly efficient elution of DNA from the beads (removal of DNA from beads). This typically happens when the beads with DNA are suspended in the solution in which the DNA is released. In this case, efficiency is measured by how quickly the DNA is eluted and how much of the DNA bound to the beads is eluted.

### Methods

DNA was extracted from 750,000 human cells (GM12878) on an electrowetting array with vibration assisted mixing and without. These cells are estimated to contain about 4500 ng of total genomic DNA. The results of the respective assays are exemplified in Table 3. With vibration assisted mixing, the total DNA recovered is about 2830 ng or about 63%. Whereas without vibration, the total DNA recovered is 480 ng or just about 11%. Vibration assisted mixing allows for much higher binding of DNA and isolation in comparison to if we relied only on electrowetting for mixing.

TABLE 3

| | DNA Yield | | DNA Yield with vibration | |
|---|---|---|---|---|
| | with no vibration | | assisted mixing | |
| DNA Concentration | 4.8 | ng/μL | 28.3 | ng/μL |
| (nanograms of DNA per | | | | |
| microliter) | | | | |
| Total DNA Extracted | 480 | ng | 2830 | ng |
| (in nanograms) in 100 μL | | | | |

| | | |
|---|---|---|
| % DNA isolated from | 11% | 63% |
| sample | | |

### Example 9: DNA Clean Up Using Magnetically Responsive Beads with Vibration Assisted Mixing

In applications where nucleic acid molecules (DNA or RNA) are the molecules of interest (e.g. as next-generation DNA sequencing (NGS), protein sequencing, quantitative PCR (qPCR), droplet digital PCR (ddPCR) and other molecular biology applications to immobilize DNA, RNA, proteins and other bio-molecules), the functionalized substrates can be used for: binding to a molecule of known size, binding to a molecule of known type and generally for isolation and cleanups from other contaminants. A typical usage of the beads and in particular for cleaning up nucleic acids from contaminants looks as shown in the diagram below.

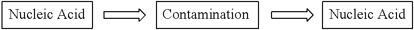

When this cleanup workflow is performed on an electrowetting device, this is all done in droplets as follows:

First, a droplet consisting of the nucleic acid(s) of interest is merged with another droplet consisting of the functionalized substrates. During this step, the nucleic acid selectively binds to the beads and leaves all the contaminants in the solution.

Then, the substrates are then pulled down to the surface of the electrowetting array by applying a strong local magnetic field. Once the substrates are pelletized, the liquid consisting of contaminants is pulled away from the substrates using electrowetting forces. Additionally, the substrate pellet on the surface of the electrowetting array can be washed with a washing liquid such as ethanol one or more times.

Finally, the substrates are suspended by adding a droplet of water, or other elution buffer, with the magnetic field lowered. The nucleic acids bound to the beads then gets released into the solution under aqueous condition.

In the above three step process, the quality of mixing has a direct impact on the quantity of nucleic acids that are recovered at the end of the workflow. In particular, during step the first step of nucleic acid immobilization, it is important that the substrates in the liquid are mixed well to bind to most of the nucleic acid in the solution. Similarly, the amount of nucleic acid eluted in the last step is directly proportional to the quality of mixing.

On an electrowetting device (e.g. the devices described herein), mixing using electrowetting motion alone may not be good enough to achieve sufficient binding in the mixing step described immediately above and sufficient elution as described above. This results in unbound nucleic acids lost during the clean-up process. Whereas with vibration assisted mixing on an electrowetting device, the quantity of nucleic acid binding to the substrates is high. Similarly, with vibration assisted mixing, nucleic acids eluting from the substrates results in most of the nucleic acids eluted. As a result, with vibration assisted mixing, most of the DNA is recovered.

### Methods

To demonstrate this, a cleanup reaction of DNA with contaminants using SPRI (Solid Phase Reversible Immobilization) magnetic beads was carried out.

For this workflow, about 2900 ng of DNA was used as input and clean-up using three steps shown above was implemented. The workflow was carried without vibration assisted mixing and with vibration assisted mixing. Both reactions were carried out for about 5 minutes. As shown in Table 4 below, with vibration assisted mixing DNA cleanup, 2444 ng of DNA was recovered. Whereas when there is no vibration, only about 931 ng of DNA was recovered. Vibration assisted mixing recovers 2.5 times higher amount of DNA from the same input material in a cleanup process performed on an electrowetting device.

TABLE 4

| | DNA recovered | DNA recovered with |
|---|---|---|
| | with no vibration | vibration assisted mixing |
| Mass of DNA recovered | 930.6 ng | 2444 ng |
| (in nanograms) | | |
| % Yield as a function of | 32% | 84% |
| input DNA | | |

### Vibration Assisted Mixing Generally

In general, vibration assisted mixing aides in achieving high quality results that are relevant biologically and chemically, that is difficult to achieve with pure electrowetting based mixing alone. The examples above illustrate improvements in
1. % recovery of certain molecules;
2. faster processing of samples; and
3. high efficiency in isolating DNA from challenging samples such as cell lysates.

Generally, vibration assisted mixing can improve the performance of several other biological processes. Some additional process that could benefit from this include any and all enzymatic and bead based reactions in next-generation sequencing, protein sequencing, PCR, nucleic acid restriction, nucleic acid digestion, nucleic acid amplification, gene editing, molecular cloning, biopolymer synthesis, biopolymer assembly, DNA repair, RNA repair, DNA ligation, DNA error detection and DNA replication. In particular, in these reactions, vibration assisted mixing provides the benefit of:
1. speeding up these processes and hence reducing the time for completion of reaction;
2. helps utilize these enzymes efficiently;
3. help reduce the usage of the input samples; and
4. carryout reactions with minimal errors.

Furthermore, this technique can be expanded to general biological and chemical processing with liquids containing nucleic acids, proteins, salts, surfactants, beads, cells, metabolites, organic molecules and inorganic molecules.

### Example 10: Extraction of High Molecular Weight (HMW) Genomic DNA (gDNA) from GM12878 Cells and Whole Human Blood

Cells from various sources (e.g., mammalian, bacterial, plants) are lysed directly on the array by merging a droplet containing cells with another droplet containing lysis agent (e.g., detergent or enzymatic). This mixture is heated and mixed (e.g., separately or simultaneously) on the EWOD array to promote lysis of cells and, if applicable, lysis of the nucleus. Enzymatic digestion of proteins, RNA, or a combination thereof are performed to improve the purity of the sample. While cells are lysed, the progress of lysis reaction and lysis efficiency is monitored via DNA-specific fluorescent stains. DNA is purified directly on the array by solid phase (e.g., bead-based capture or by precipitation (e.g. salt and ethanol or phenol-chloroform extraction)). Recovered DNA is manipulated and transferred to different locations of the array by EWOD with minimal shearing. DNA purity, critical for high quality long-read sequencing, is improved by increasing the number of washing cycles performed on the array. Small DNA fragments are removed using silica-nanostructured magnetic disks. The yield of recovered DNA is increased by performing additional successive elution in buffers.

After DNA extraction, samples are analyzed by Pulse Field Gel Electrophoresis (PFGE), quantifying size distribution for each sample relative to one another and analyzing commercially available ladders (BioRad) and ImageJ (NIH) profile analysis tools. For smaller inputs, (e.g., cell input) recovery/size distribution is measured by Femto Pulse (Agilent) and qPCR for lower input amounts. Genomic intactness is assessed by additional complementary methods, e.g. the BioNano Genomics Saphyr System, allowing rapid and cost-effective prototyping at a macro scale as well as independent comparability of data using the Saphyr System.

The passivation of the EWOD surface is determined by testing DNA deposition and retention in the presence of solution- and surface-deposited PEG200 or BlockAid (Invitrogen) passivated devices. Measurements are obtained i) by staining the surfaces after use with Hoechst 33342, ii) calculating surface retention of a commercial preparation of Lambda DNA (New England Biolabs, linearized 48.5 Kb), and/or iii) measuring % loss by qPCR of sample pre- and post-manipulation and input quantities from 10⁹ to 10² copies of DNA.

Mammalian cell lysis, RNA, and protein digestion followed by HMW DNA isolation was performed on the EWOD array. The distribution of the high molecular weight DNA fragments are seen in FIG. 4 with DNA fragments larger than 165,000 bp being isolated from the sample. The longer duration of elution recovers more DNA (e.g., indicated by the taller peak) and is a way to obtain higher DNA yield.

Automated extraction procedures referenced herein were performed on the EWOD array in order to derive isolated HMW DNA from GM12878 cells. The platform performed automated DNA binding, bead pelleting, washing and cleanup and finally elution without requiring manual interactions with the sample. More than 5 μg of DNA were extracted in under an hour and were of a high purity with lengths exceeding 100 kb as demonstrated in the results in FIG. 24 and in Table 5.

TABLE 5

| | GM12878 |
|---|---|
| Yield (μg) | 6.4 ± 0.9 |
| A260/A280 | 1.84 ± 0.02 |
| A260/A280 | 1.93 ± 0.09 |

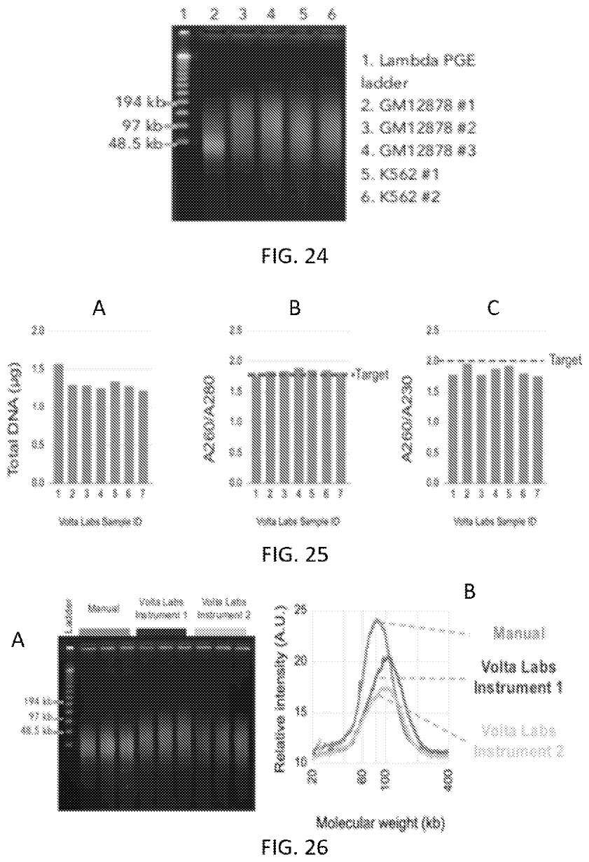

The same system was also used to extract HMW gDNA from whole human blood samples. An average of approx. 1.2 μg of DNA were derived per 100 μL of whole blood per lane in under an hour as seen in FIG. 25A-25C. Multiple lanes of extraction were run simultaneously resulting in rapid high yield isolation of gDNA. PFGE gel analysis was performed to assess the extracted fragment length compared to a manually performed extraction. The results in FIGS. 26A and 26B demonstrate that the extraction performed on the platform had similar or greater average gDNA fragment lengths.

Assay using similar techniques have been performed successfully on multiple mammalian cell lines including GM06852, GM09237, GM20241, GM07537, and K562.

### Example 11: Whole Genome Sequencing on Cellular Nucleic Acids

Genomic intactness is demonstrated by long read sequencing through an Oxford Nanopore device. DNA can be extracted using protocols described herein alongside Qiagen HMW kit and Loman protocols. Libraries are prepared according to an optimized protocol for keeping strands in >1 Mb lengths. The repeatability of the extractions is evaluated by sequencing a minimum of 3 each of Qiagen and Loman libraries and 7 Flexomics libraries to ensure robustness of evaluation of size performance. Regular input and low input (e.g., 1000 cells) libraries are assessed. At low input, ˜24 subsets are barcoded of 1000 cells each to provide enough material for downstream sequencing (˜150 ng theoretical).

Cell HMW DNA input is titrated down, for example, i) by supplementation with carrier DNA, e.g. Lambda DNA, to ensure balanced library preparation or ii) dilution of an absolute number of cells and scaling of library preparation and analysis reagents for subsequent reactions. Lambda DNA is biotinylated (e.g. with Pierce 3′ biotinylation kit, Thermo Fisher) to allow depletion to concentrate on-target library prior to sequencing. Performance of the ONT transposase library preparation is assessed on-device, e.g., without moving the sample to a separate tube.

Preparation of samples for whole genome sequencing across a variety of platforms has been demonstrated. Using the HMW gDNA extracted from GM 12878 and whole human blood using the extraction protocols described herein, libraries were prepared for the Illumnina NovaSeq 6000. The sequencing data was then compared to gDNA derived from manual extractions. The results in Table 6 and Table 7 demonstrate that the sample derived from the automated platform are of similar quality as to the samples prepared manually.

TABLE 6

| | GM12878 gDNA | GM12878 gDNA |
|---|---|---|
| Sequencing Metrics | (Manual) | (Volta Labs) |
| Total Reads (in billions) | 1.545 | 1.106 |
| % Aligned | 94.6 | 95 |
| Mappable Mean Coverage | 74.8x | 53.7x |
| % Genome Covered ≥ 10x | 97.3 | 97.1 |
| % Genome Covered ≥ 20x | 96.7 | 96.3 |
| % Genome Covered ≥ 30x | 96.1 | 94.8 |
| Median Insert Size | 281 bp | 322 bp |
| % Bases Q ≥ 30 | 89.95 | 89.39 |
| (Illumina_{cut-off} = 85%) | | |

TABLE 7

| | Whole Blood gDNA | Whole Blood gDNA |
|---|---|---|
| Sequencing Metrics | (Manual) | (Volta Labs) |
| Total Reads (in billions) | 1.009 | 1.671 |
| % Aligned | 94.7 | 94.2 |
| Mappable Mean Coverage | 48.9x | 80.5x |
| % Genome Covered ≥ 10x | 97.5 | 97.9 |
| % Genome Covered ≥ 20x | 95 | 97.3 |
| % Genome Covered ≥ 30x | 88.9 | 95.8 |
| Median Insert Size | 265 bp | 275 bp |
| % Bases Q ≥ 30 | 90.80 | 90.13 |
| (Illumina_{cut-off} = 85%) | | |

These preparations and assays were also repeated using the same inputs and protocols to demonstrate that the library preparations were both consistent and reliable. The results in Table 8 show that the libraries prepared on the EWOD platform for the Illumnina sequencer reliably produced high quality scores across gDNA sources.

TABLE 8

| | | | Whole | Whole |
|---|---|---|---|---|
| | GM12878 | GM12878 | Blood | Blood |
| Sequencing | gDNA | gDNA | gDNA | gDNA |
| Metrics | #1 | #2 | #1 | #2 |
| Total Reads (in billions) | 0.718 | 0.510 | 0.883 | 1.075 |
| % Aligned | 95.1 | 95.5 | 94.6 | 94.7 |
| Mappable Mean | 34.9 | 24.9 | 40.3 | 52.1 |
| Coverage | | | | |
| % Genome Covered ≥ 10x | 96.6 | 95.8 | 97.3 | 97.6 |
| % Genome Covered ≥ 20x | 92.7 | 70.6 | 93.1 | 95.7 |
| % Genome Covered ≥ 30x | 61.6 | 17.2 | 79.9 | 90.9 |
| % Bases Q ≥ 30 | 90.2 | 90.5 | 90.3 | 89.9 |
| (Illumina_{cut-off} = 85%) | | | | |

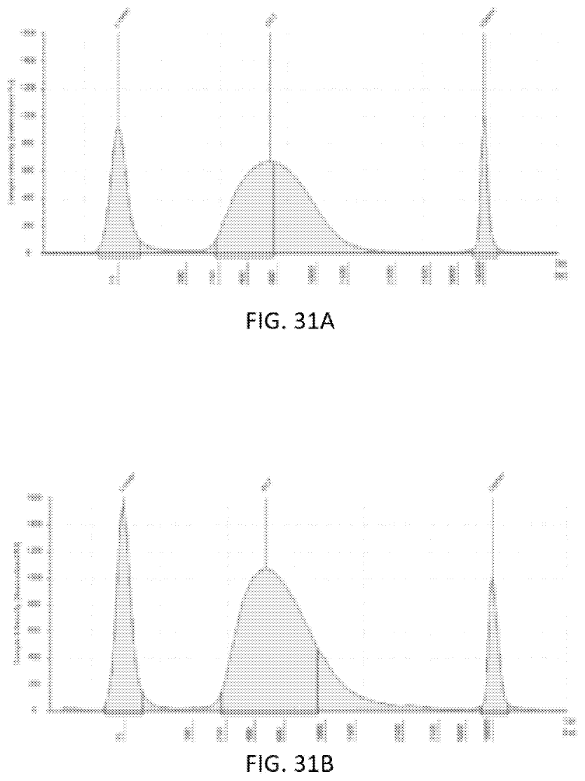

The library preparation procedure was repeated for the Illumina sequencing system to demonstrate the consistency of the streamlined, single droplet reaction automated workflow. Using an Agilent TapeStation the size of the inserts and library fragments were analyzed and the results are displayed in Table 9. The distribution of the library fragment size for one GM 12878 and one whole blood sample are displayed in FIG. 31A and FIG. 31B respectively.

TABLE 9

| | | Insert Size | Library Size |
|---|---|---|---|
| Sample | gDNA | Desired target size: | Desired target size: |
| Number | Source | 300-450 bp | 400-600 bp |
| 1 | GM12878 | 366 | 543 |
| 2 | GM12878 | 382 | 531 |
| 3 | GM12878 | 369 | 532 |
| 4 | Whole Blood | 234 | 467 |
| 5 | Whole Blood | 342 | 511 |
| 6 | Whole Blood | 224 | 451 |

The functionality of the fragments produced in these workflows was also analyzed to ensure functionality using qPCR-based library quantification. The data from these experiments are displayed in

### Table 10.

TABLE 10

| Sample | gDNA | qPCR-based | Qubit-based |
|---|---|---|---|
| Number | Source | concentration (nM) | concentration (nM) |
| 1 | GM12878 | 25.2 | 58 |
| 2 | GM12878 | 28.7 | 58.9 |
| 3 | Whole Blood | 17.5 | 33.1 |
| 4 | Whole Blood | 27.4 | 34.1 |

Libraries were similarly prepared for the PacBio Sequel II sequencer from gDNA extracted from a SMRTcell of the GM07537 cell line using the automatic platform. Preparation of the library for sequencing on the PacBio equipment was accomplished using the PacBio SMRTbell v3.0 kit. A fresh 80% ethanol in nuclease free water solution and a 35% AMPure PB Bead solution in Elution buffer were prepared. These two solutions along with nuclease free water were placed into the Dispenser Deck. The rest of the reagents had the volume required calculated based on the amount required per sample and were pipetted onto the reagent cartridge in the volumes listed in Table 11.

TABLE 11

| Reagent | Volume per sample (μL) |
|---|---|
| Repair Buffer | 8 |
| End Repair Mix | 4 |
| DNA Repair Mix | 2 |
| SMRTbell Adapter | 4 |
| Ligation Mix | 30 |
| Ligation Enhancer | 1 |
| Nuclease Buffer | 5 |
| Nuclease Mix | 5 |
| SMRTbell Cleanup Beads | 95 |
| Elution Buffer | 60 |

The Volta consumable was then loaded into the platform by pressing the “Load Consumable” button on the user interface. When prompted by the system a Film Consumable was loaded. 46 μL of HMW gDNA was then pipetted using wide-bore tips onto the platform. The mass of HWM gDNA was between 300 ng and 5 μg and were between 15 k and 18 k base pairs in length. The A260/280 was 1.8 and the A260/230 was between 2.0 and 2.2.

After the platform was entirely prepared, the proper library preparation protocol was loaded by the user. Once engaged the instrument performed the entire automated workflow without intervention by the users. Once the protocol was finished running, a wide-bore pipette tip was used to aspirate 1.5 μL of the supernatant containing the purified library and dispensed into a 1.5 ml LoBind tube. 1 μL of the library was then dilute with 9 μL of elution buffer and analyzed using a Qubit and FemtoPulse to determine the quality of the purified library. Upon confirmation that the sample has successfully passed the quality control check, it was then used to sequence the gDNA using the PacBio sequencing equipment. These methods were conducted in droplet form on an array using methods as described in the present disclosure.

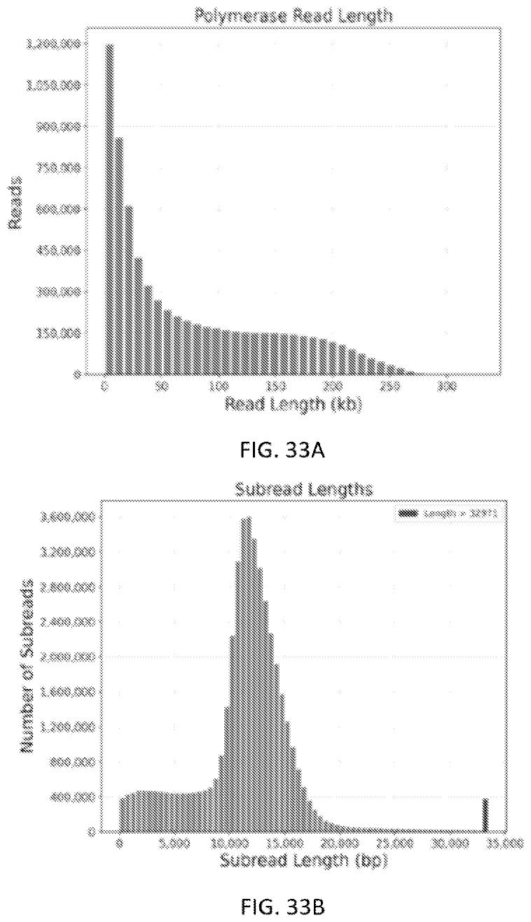

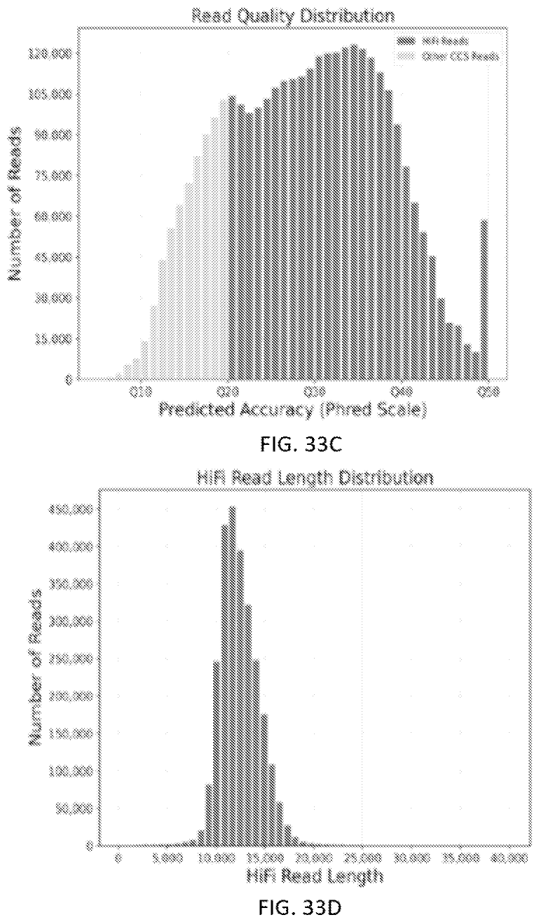

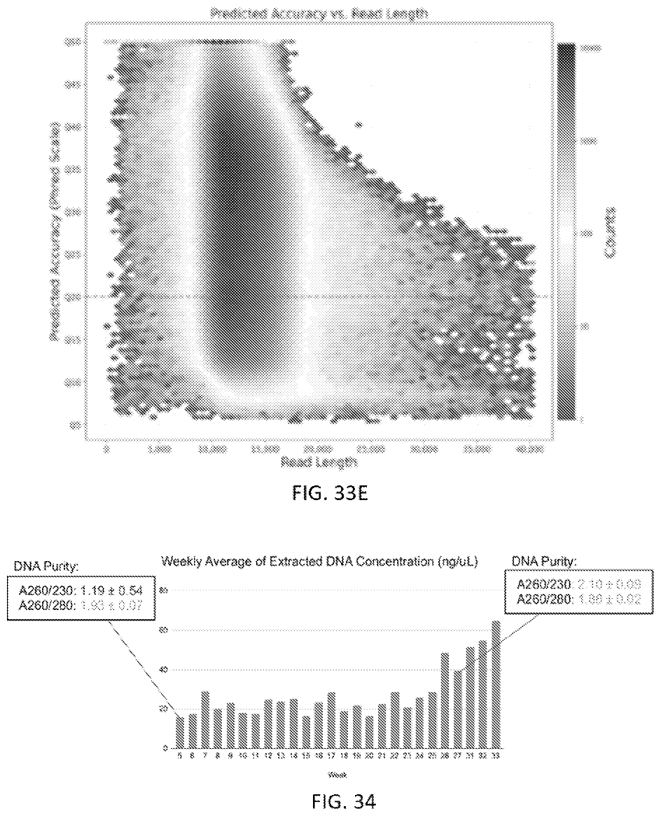

The results from the experiment are displayed in FIG. 33A-E. The raw sequence output resulted in 512 Gb of data which is above the average output of approx. 300-400 Gb experienced by the lab performing the assay. Circular consensus sequencing was performed on the sample as well and resulted in 32.5 Gb of HiFi compared to the expected average of 16-20 Gb of HiFi data. There results demonstrate 10× coverage of the genome of the single SMRTcell confirming both the high quality extraction in terms of intactness and purity as well as high compatibility with the PacBio sequencing chemistry.

### Example 12: Next Gen. Library Preparation Using the QIAseq FX DNA Kit

Preparation of the library for sequencing on the QIAseq equipment was accomplished using the QIAseq FX DNA library kit. A fresh 80% ethanol in nuclease free water solution was prepared. It, along with nuclease free water and AMPure XP beads were placed into the Dispenser Deck. The rest of the reagents had the volume required calculated based on the amount required per sample and were pipetted onto the reagent cartridge in the volumes listed in Table 12.

TABLE 12

| Reagent | Volume per sample (μL) |
|---|---|
| FX Buffer, 10x | 5 |
| Nuclease-free water | 70 |
| FX Enzyme Mix | 10 |
| DNA ligase | 10 |
| Ligation Buffer, 5x | 20 |
| Buffer EB | 140 |
| Barcodes | 5 |

The Volta consumable was then loaded into the platform by pressing the “Load Consumable” button on the user interface. When prompted by the system, a Film Consumable was loaded. 35 μL of HMW gDNA was then pipetted using 200 μL tips onto the platform. The mass of HWM gDNA was between 100 ng and 1 μg and A260/280 was 1.8 and A260/230 was between 2.0 and 2.2.

After the platform was entirely prepared, the proper library preparation protocol was loaded by the user and the desired fragmentation time was selected. Once engaged the instrument performed the entire automated workflow without intervention by the users. Once the protocol was finished running, a pipette tip was used to aspirate 30 μL of the supernatant containing the purified library and dispensed into a 1.5 ml LoBind tube. l pL of the library was then analyzed using a Qubit to determine the quality of the purified library. Upon confirmation that the sample has successfully passed the quality control check, it was then used to sequence the gDNA using the PacBio sequencing equipment. These methods were conducted in droplet form on an array using methods as described in the present disclosure.

### Example 13: Increased Efficiency and Decreased Costs Resulting from Continuing Use of the Platform

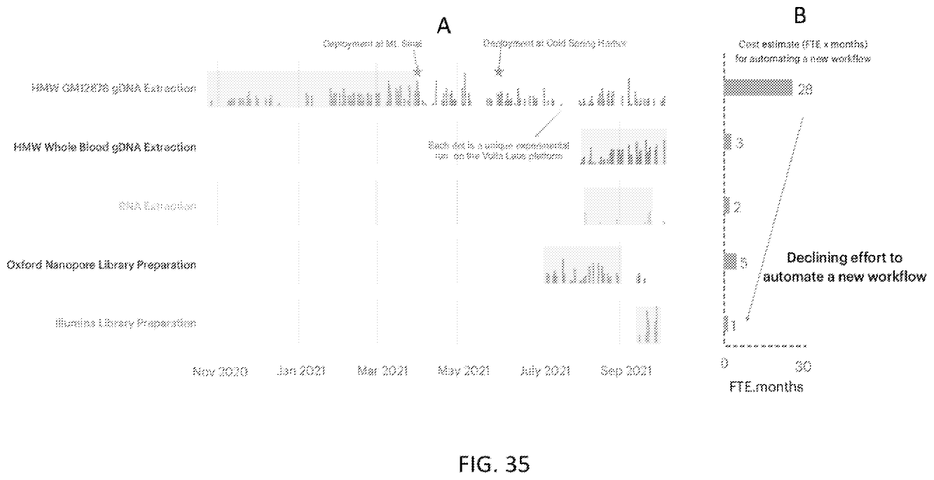

The reliable instrumentation has enabled increasing workflow experimentation capacity. This has resulted in faster experimentation and increasing workflow robustness. The cumulative experiments run on the platform are displayed in FIG. 35A. The dots within the grey boxes represent unique experiments for automating an application with acceptable biological outputs while the dots outside are either internally validated or external deployment runs. This has demonstrated that over time the investment required to develop new automated workflows, as measured in FTE months, is decreased as experience with the platform has increased as shown in FIG. 35B. Further efficiency will be demonstrated as a greater number of assays are developed.

The quality of the biological outputs from the platform will also increase as user experience with the platform increases. FIG. 34 demonstrates that over time the average concentration of DNA extracted using the platform has increased. The purity of the extracted DNA has also been shown to increase over the same time period. Further increases in the quality of the DNA from extractions on the platform as well as outputs for other applications will be demonstrated as the time the platform is in use and the number of people using the platform increases.

The design of the user assay creation software will contribute to future increases in assay development speed and efficiency. Users will be able to easily make changes to protocols and customize each program for their individual applications with little required knowledge of programing, software, or hardware operation. There will also be a database available to users which will store created protocols and facilitating collaborative development further increasing development speed. This will become clear from the results more widespread distribution of the platform to more users and development of protocols in the platform for a greater number of applications.

The use of machine learning algorithms derived from analysis of platform operation will also, over time, create greater efficiencies and cost savings from use of the platform. These algorithms will assist with the detection and isolation of errors that occur during assay performance. The algorithms will then allow for all other users of the same protocols to benefit from the machine learning based on all users running the protocol. This will allow for significantly greater protocol development and refinement when compared to instrument that do not allow for this type of cumulative learning and modification.

### Example 14: Software Architecture

A user interface that allows a user to configure the actuation of droplets (actuation means to subject a droplet to motion, mixing, heating or other operations) on an array device can be applied to a computer processor configured to directed the methods and systems described herein. On the user interface, one can define a biological or chemical protocol to be performed on the array device. Through this interface, information about liquids (such as prescribing volumes) to be used in a protocol may be entered manually by a user or automatically populated using natural language processing algorithms. The prescribed volumes may be translated into compatible volumes for the array device (volumes that are appropriate on the array device). This translation can be achieved by normalizing maximum and minimum values and then calculating the relative intermediate volumes. It may be possible that liquids with different chemical properties spread differently on the array device and hence occupy different number of actuation electrodes on the array device. These droplet volumes may be adjusted to improve mobility on the array device within the normalized range.

The software interface stores a set of values referred to as “droplet interaction properties”. These could include but are not limited to reagent compatibility (the ability for reagents to come into contact without affecting biological properties), history of its temperature over time, history of it's volume or reagent concentrations. Droplet interaction properties may be entered manually by the user or automatically recorded by the software using sensors such as temperature probes and optical sensors. These properties may be used to dictate which droplets may contact the same regions on the array device. These interaction properties may also be used to determine the ability and order for droplets to come into contact with each other (mixing or traversing same paths). Droplets may be grouped by common properties in software in order to generate a user interface and automated droplet pathways. Protocols may be generated by adding droplets to the array device. Droplet footprints on the grid area may be determined using the automatically calculated volumes. These footprints may be used to determine the area contaminated by a droplet. Contaminated areas may be stored and displayed to the user for the purpose of determining droplet placement and clean usable area on the array device. Throughout these reactions, the software can direct the evaporation and humidity control methods and systems to the array to maintain constant physical properties of the droplet(s), the array itself, and the area adjacent to the array and/or the droplet(s).

While a protocol is being executed on the device, “droplet interaction properties” may be recorded. These properties include but are not limited to constituent reagents, temperatures, presence of sample and errors during execution of a protocol. These properties may be displayed over a live video feed of droplets on an array device or accessed through a simulation of the protocol during execution. Areas previously covered by a selected droplet may be highlighted over the video feed, in the simulated grid area or projected (via a projector mounted above the array device) onto the physical grid area.

Data on the operation and performance of the device (array device or an instrument using the array device) may be collected by various sensors and software components. These sensors may include but are not limited to optical, capacitive, temperature and humidity sensors. The software components may include but are not limited to wireless communications, wired communications, device connections and user interactions. The data collected may be logged in order to diagnose device operations and malfunctions. This data may also be used to detect errors in real time. These detections may be used to notify users in real time when a user's intervention is required. This intervention may be administered locally by controls available on the device (for example a physical button or a software UI element) or remotely by the user or support team. The collected data may also be used to optimize the user interface.

A digital projector may be mounted over the grid area. This projector may be used to aid the user in manual pipetting of liquids onto the grid area. This may be accomplished by projecting lines or other patterns to guide the user to the desired position or region. Information about droplet positions, volumes and other droplet properties may be projected onto the grid area during operation to aid the user in monitoring of protocols. User aids such as progress to desired volume during pipetting may also be displayed when interacting with the device. Colors may be projected onto droplets in order to highlight positions, contaminated areas (areas already traversed by another droplet) on the array and future paths in order to correlate the physical grid area to the software simulation.

Neural networks may be trained to test for the presence or absence of droplets in an image. These machine learning models may be trained for various fields of view over the grid area of the arrays device. The models may then be used to determine which electrode areas are in contact with droplets. Using algorithms such as the sliding window method, confidence in droplet positions may be assigned and then correlated to expected droplet positions based on those prescribed by the planned protocol. This data may be used to adjust electrode states and to flag potential errors in operation. Neural networks may also be trained to correlate images of droplets to their volumes. These models may be created for various types of liquids in order to accurately predict droplet volumes with different properties. This data may be used to control feedback for use in applications such as droplet evaporation. These models may be used on live video feeds of droplets during device operation. These models may also be used to rapidly increase improvement of assays by compiling the learning images in a database for neural network analysis.

Biological protocol documents define the physical operations such as liquid mixing and heating as well as required reagents and liquid volumes. These protocols are broken down into step by step instructions which contain parameters to define these reagents and operations such as reagent concentrations and mixing speed. The feasibility of these operations and liquids on the array device can be determined by examining the parameters and comparing known limitations of the array device to deduce compatibility. The compatibility of these properties including but not limited to reagents, physical operations, droplet volumes and chemical reactions may be determined experimentally. These properties may then be used to develop a filter which is employed to determine whether a standard protocol is compatible or incompatible with the array device. A list of descriptors for these compatible and incompatible properties may then be compiled and used to create a natural language processing model. This model may be trained to extract the overall structure as well as the aforementioned compatibility properties from a standard protocol document. The extracted information may be passed through the filter to determine whether the standard protocol is compatible with the device. Once compatibility is determined, the key information may then be used to inform the translation of the standard protocol operations to device specific operations. These operations may be compiled and used to generate a device compatible protocol. Furthermore, web scraping algorithms may be developed which can locate biological protocol documents and compile them into a database. The data in the database may then be fed as inputs to the natural language processing model which determines compatibility and translates to device protocols. These protocols may then be collected and added to a library of protocols.

While preferred embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the invention described herein may be employed in practicing the invention. It is therefore contemplated that the invention shall also cover any such alternatives, modifications, variations or equivalents. It is intended that the following claims define the scope of the invention and that methods and structures within the scope of these claims and their equivalents be covered thereby.

### CURRENTLY PREFERRED EMBODIMENTS

### Embodiment 1

1. A system for processing a sample, comprising:
   a. an array comprising:
      i. a plurality of electrodes; and
      ii. a surface configured to support said sample;
         b. an electro-mechanical actuator coupled to said array, wherein said actuator is configured to vibrate said array; and
         c. a controller operatively coupled to said plurality of electrodes, or said electromechanical actuator, wherein said controller is configured to:
      i. direct at least a subset of said plurality of electrodes to supply an electric field to alter a wetting characteristic of said surface; or
      ii. direct said electro-mechanical actuator to apply a frequency of vibration to said array.
2. The system of any one of the preceding paragraphs, wherein said controller is configured to perform (i) and (ii).
3. The system of any one of the preceding paragraphs, wherein said controller is coupled to said plurality of electrodes and said electro-mechanical actuator.
4. The system of any one of the preceding paragraphs, wherein said sample is a droplet.
5. The system of any one of the preceding paragraphs, wherein said droplet comprises about 1 nanoliter to 1 milliliter.
6. The system of any one of the preceding paragraphs, wherein said droplet comprises a biological material.
7. The system of any one of the preceding paragraphs, wherein said biological sample comprises one or more bio-molecules.
8. The system of any one of the preceding paragraphs, wherein said bio-molecules comprise nucleic acid molecules, proteins, polypeptides, or any combination thereof.
9. The system of any one of the preceding paragraphs, wherein said electroactuator comprises a cantilever.
10. The system of any one of the preceding paragraphs, wherein said electroactuator comprises one or more coupling members coupled to said array.
11. The system of any one of the preceding paragraphs, wherein said one or more coupling members comprise electromagnetic actuators, piezoelectric actuators, ultrasonic transducers, rotating eccentric masses, one or more motors with oscillating linkage mechanisms, or any combination thereof.
12. The system of any one of the preceding paragraphs, wherein said one or more motors are brushed, brushless, stepper, or any combination thereof.
13. The system of any one of the preceding paragraphs, wherein said electromagnetic actuators comprise electromagnetic voice coil actuators.
14. The system of any one of the preceding paragraphs, wherein said frequency of vibration comprises a gradient.
15. The system of any one of the preceding paragraphs, wherein said gradient ascends from near a site wherein said cantilever is coupled to said array.
16. The system of any one of the preceding paragraphs, wherein said vibration comprises a pattern.
17. The system of any one of the preceding paragraphs, wherein said pattern is sinusoidal.
18. The system of any one of the preceding paragraphs, wherein said pattern is square.
19. The system of any one of the preceding paragraphs, wherein said surface is a top surface of a dielectric wherein said dielectric is disposed over said plurality of electrodes.
20. The system of any one of the preceding paragraphs, wherein said top surface comprises a layer.
21. The system of any one of the preceding paragraphs, wherein said layer comprises a liquid.
22. The system of any one of the preceding paragraphs, wherein said layer comprises a coating.
23. The system of any one of the preceding paragraphs, wherein said coating is hydrophobic.
24. The system of any one of the preceding paragraphs, wherein said layer comprises a film.
25. The system of any one of the preceding paragraphs, wherein said film is a dielectric film.
26. The system of any one of the preceding paragraphs, wherein said dielectric film comprises a natural polymeric material, a synthetic polymeric material, a fluorinated material, a surface modification, or any combination thereof.
27. The system of any one of the preceding paragraphs, wherein said natural polymeric material comprises shellac, amber, wool, silk, natural rubber, cellulose, wax, chiton, or any combination thereof.
28. The system of any one of the preceding paragraphs, wherein said synthetic polymeric material comprises polyethylene, polypropylene, polystyrene, polyetheretherketone (PEEK), polyimide, polyacetal, polysilfone, polyphenulene ether, polyphenylene Sulfide (PPS), polyvinyl chloride, synthetic rubber, neoprene, nylon, polyacrylonitrile, polyvinyl butyral, silicone, parafilm, polyethylene terephthalate, polybutylene terephthalate, polyamides, polyoxymethlyene, polycarbonate, polymethylpentene, polyphenylene oxide (Polyphenyl ether), polyphthalamide (PPA), polylactic acid, synthetic cellulose ethers (e.g., methyl cellulose, ethyl cellulose, propyl cellulose, hydroxyethyl cellulose, hydroxyethyl cellulose, hydroxypropyl cellulose (HPC), hydroxyethyl methyl cellulose, hydroxypropyl methyl cellulose (HPMC), ethyl hydroxyethyl cellulose), paraffins, microcrystalline wax, epoxy, or any combination thereof.
29. The system of any one of the preceding paragraphs, wherein said fluorinated material comprises polytetrafluoroethylene (PTFE), tetrafluoroethylene (TFE), fluorinated ethylenepropylene copolymer (FEP), polyvinylidene fluoride (PVDF), perfluoroalkoxytetrafluoroethylene copolymer (PFA), perfluoromethyl vinylether copolymer (MFA), ethylenechlorotrifluoroethylene copolymer (ECTFE), ethylene-tetrafluoroethylene copolymer (ETFE), perfluoropolyether (PFPE), polychlorotetrafluoroethylene (PCTFE), or any combination thereof.
30. The system of any one of the preceding paragraphs, wherein said surface modification comprises silicone, silane, fluoro-polymer treatment, parylene coating, any other suitable surface chemistry modification process, ceramic, clay minerals, bentonite, kaolinite, vermiculite, graphite, molybdenum disulfide, mica, boron nitride, sodium formate, sodium oleate, sodium palmitate, sodium sulfate, sodium alginate, or any combination thereof.
31. The system of any one of the preceding paragraphs, wherein said liquid comprises silicone oils, fluorinated oils, ionic liquids, mineral oils, ferrofluids, polyphenyl ether, vegetable oil, esters of saturated fatty and dibasic acids, grease, fatty acids, triglycerides, polyalphaolefin, polyglycol hydrocarbons, other Non-hydrocarbon synthetic oils, or any combination thereof.
32. The system of any one of the preceding paragraphs, wherein said liquid further comprises surfactants, electrolytes, rheology modifier, wax, graphite, graphene, molybdenum disulfide, PTFE particles, or any combination thereof.
33. The system of any one of the preceding paragraphs, wherein said first plurality of electrodes, said dielectric, said surface configured to support said droplet comprising said sample, or any combination thereof is removable from said array.
34. The method of any one of the preceding paragraphs, wherein said electro-mechanical actuator is configured to displace said surface or a portion of said surface from 0.05 millimeters (mm) to 10 mm.
35. The system of any one of the preceding paragraphs, wherein said frequency of said vibration is from 1 Hertz (hz) to 20 kilohertz (khz).
36. A method for processing a sample comprising:
   a. providing an array comprising:
      i. a plurality of electrodes; and
      ii. a surface configured to support said sample;
   wherein said array is coupled to an electro-mechanical actuator and said electromechanical actuator is configured to vibrate said array;
      b. introducing said droplet to said surface; and
      c. directing said electro-mechanical actuator to apply a frequency of vibration to said array.
37. The method of any one of the preceding paragraphs, wherein said sample is a droplet.
38. The method of any one of the preceding paragraphs, wherein said droplet comprises about 1 nanoliter to 1 milliliter.
39. The method of any one of the preceding paragraphs, wherein said droplet comprises a biological material.
40. The method of any one of the preceding paragraphs, wherein said biological sample comprises one or more bio-molecules.
41. The method of any one of the preceding paragraphs, wherein said bio-molecules comprise nucleic acid molecules, proteins, polypeptides, or any combination thereof. The method of any one of the preceding paragraphs, wherein said droplet comprises about 1 nanoliter to 1 milliliter.
42. The method of any one of the preceding paragraphs, further comprising directing at least a subset of said plurality of electrodes to supply an electric field to alter a wetting characteristic of said surface.
43. The method of any one of the preceding paragraphs, wherein said electro-mechanical actuator comprises a cantilever.
44. The method of any one of the preceding paragraphs, wherein said electro-mechanical actuator comprises one or more coupling members coupled to said array.
45. The method of any one of the preceding paragraphs, wherein said one or more coupling members comprise electromagnetic actuators, piezoelectric actuators, ultrasonic transducers, rotating eccentric masses, one or more motors with oscillating linkage mechanisms, or any combination thereof
46. The method of any one of the preceding paragraphs, wherein said one or more motors are brushed, brushless, stepper, or any combination thereof.
47. The method of any one of the preceding paragraphs, wherein said electromagnetic actuators comprise electromagnetic voice coil actuators.
48. The method of any one of the preceding paragraphs, wherein said frequency of vibration comprises a gradient.
49. The method of any one of the preceding paragraphs, wherein said gradient ascends from near a site wherein said cantilever is coupled to said array.
50. The method of any one of the preceding paragraphs, wherein said vibration comprises a pattern.
51. The method of any one of the preceding paragraphs, wherein said pattern is sinusoidal.
52. The method of any one of the preceding paragraphs, wherein said pattern is square.
53. The method of any one of the preceding paragraphs, wherein said surface is a top surface of a dielectric wherein said dielectric is disposed over said plurality of electrodes.
54. The method of any one of the preceding paragraphs, wherein said surface comprises a layer disposed over a dielectric wherein said dielectric is disposed over said plurality of electrodes.
55. The method of any one of the preceding paragraphs, wherein said layer comprises a liquid.
56. The method of any one of the preceding paragraphs, wherein said layer comprises a coating.
57. The method of any one of the preceding paragraphs, wherein said coating is hydrophobic.
58. The method of any one of the preceding paragraphs, wherein said layer comprises a film.
59. The method of any one of the preceding paragraphs, wherein said film is a dielectric film.
60. The method of any one of the preceding paragraphs, wherein said dielectric film comprises a natural polymeric material, a synthetic polymeric material, a fluorinated material, a surface modification, or any combination thereof.
61. The method of any one of the preceding paragraphs, wherein said natural polymeric material comprises shellac, amber, wool, silk, natural rubber, cellulose, wax, chiton, or any combination thereof.
62. The method of any one of the preceding paragraphs, wherein said synthetic polymeric material comprises polyethylene, polypropylene, polystyrene, polyetheretherketone (PEEK), polyimide, polyacetal, polysilfone, polyphenulene ether, polyphenylene Sulfide (PPS), polyvinyl chloride, synthetic rubber, neoprene, nylon, polyacrylonitrile, polyvinyl butyral, silicone, parafilm, polyethylene terephthalate, polybutylene terephthalate, polyamides, polyoxymethlyene, polycarbonate, polymethylpentene, polyphenylene oxide (Polyphenyl ether), polyphthalamide (PPA), polylactic acid, synthetic cellulose ethers (e.g., methyl cellulose, ethyl cellulose, propyl cellulose, hydroxyethyl cellulose, hydroxyethyl cellulose, hydroxypropyl cellulose (HPC), hydroxyethyl methyl cellulose, hydroxypropyl methyl cellulose (HPMC), ethyl hydroxyethyl cellulose), paraffins, microcrystalline wax, epoxy, or any combination thereof.
63. The method of any one of the preceding paragraphs, wherein said fluorinated material comprises polytetrafluoroethylene (PTFE), tetrafluoroethylene (TFE), fluorinated ethylenepropylene copolymer (FEP), polyvinylidene fluoride (PVDF), perfluoroalkoxytetrafluoroethylene copolymer (PFA), perfluoromethyl vinylether copolymer (MFA), ethylenechlorotrifluoroethylene copolymer (ECTFE), ethylene-tetrafluoroethylene copolymer (ETFE), perfluoropolyether (PFPE), polychlorotetrafluoroethylene (PCTFE), or any combination thereof.
64. The method of any one of the preceding paragraphs, wherein said surface modification comprises silicone, silane, fluoro-polymer treatment, parylene coating, any other suitable surface chemistry modification process, ceramic, clay minerals, bentonite, kaolinite, vermiculite, graphite, molybdenum disulfide, mica, boron nitride, sodium formate, sodium oleate, sodium palmitate, sodium sulfate, sodium alginate, or any combination thereof.
65. The method of any one of the preceding paragraphs, wherein said liquid comprises silicone oils, fluorinated oils, ionic liquids, mineral oils, ferrofluids, polyphenyl ether, vegetable oil, esters of saturated fatty and dibasic acids, grease, fatty acids, triglycerides, polyalphaolefin, polyglycol hydrocarbons, other Non-hydrocarbon synthetic oils, or any combination thereof.
66. The method of any one of the preceding paragraphs, wherein said liquid further comprises surfactants, electrolytes, rheology modifier, wax, graphite, graphene, molybdenum disulfide, PTFE particles, or any combination thereof.
67. The method of any one of the preceding paragraphs, wherein said first plurality of electrodes, said dielectric, said surface configured to support said droplet comprising said sample, or any combination thereof is removable from said array.
68. The method of any one of the preceding paragraphs, wherein said frequency of said vibration displaces said surface or a portion of said surface from 0.05 millimeters (mm) to 10 mm.
69. The method of any one of the preceding paragraphs, wherein said frequency of said vibration is from 1 Hertz (hz) to 20 kilohertz (khz).
70. A method of contacting a first sample with a second sample, wherein said first sample is contained in a first droplet and said second sample is contained in a second droplet, the method comprising:
   a. providing an array comprising:
      i. a plurality of electrodes; and
      ii. a surface configured to support said first droplet and said second droplet;
   wherein said array is coupled to an electro-mechanicalactuator and said electro-mechanicalactuator is configured to vibrate said array;
   b. introducing said first droplet and said second droplet to said surface;
   c. directing at least a subset of said plurality of electrodes to supply an electric field to alter a wetting characteristic of said surface thereby inducing a motion in said first droplet and said second droplet wherein said motion of said first droplet and said second droplet comprise said first droplet and said second droplet to converge to generate a mixed droplet; and
   d. directing said electro-mechanicalactuator to apply a frequency of vibration to said surface; thereby contacting said first sample with said second sample.
71. The method of any one of the preceding paragraphs, wherein said first sample, said second sample, or both comprise a viscous fluid.
72. The method of any one of the preceding paragraphs, wherein said first sample, said second sample, or both comprise a biological sample.
73. The method of any one of the preceding paragraphs, wherein said biological sample comprises one or more bio-molecules.
74. The method of any one of the preceding paragraphs, wherein said bio-molecules comprise nucleic acid molecules, proteins, polypeptides, or any combination thereof.
75. The method of any one of the preceding paragraphs, wherein said first sample, said second sample, or both comprise reagents for a biological assay.
76. The method of any one of the preceding paragraphs, wherein said first sample, said second sample, or both comprise one or more cell lysis reagents.
77. The method of any one of the preceding paragraphs, wherein said one or more cell lysis reagents comprise a substrate configured to bind to a said biological sample or a subset of said biological sample.
78. The method of any one of the preceding paragraphs, wherein said nucleic acid molecule comprises more than 100 bases, 1 kilobase (kb), 20 kb, 30 kb, 40 kb, or 50 kb.
79. The method of any one of the preceding paragraphs, wherein greater than 10%, 20%, 30%, 40%, 50%, 60%, 70%, 80%, or 90% of the biological sample binds to said substrate.
80. The method of any one of the preceding paragraphs, wherein said substrate is a functionalized bead.
81. The method of any one of the preceding paragraphs, wherein said substrate is a functionalized disc.
82. The method of any one of the preceding paragraphs, wherein the method further comprises, subsequent to (d):
   e. removing at least a portion of said mixed droplet by directing at least a subset of said plurality of electrodes to supply an electric field to alter a wetting characteristic of said surface thereby inducing a motion in said at least said portion of said mixed droplet.
83. The method of any one of the preceding paragraphs, wherein said at least said portion of said mixed droplet does not comprise said biological sample.
84. The method of any one of the preceding paragraphs, wherein the method further comprises, prior to or contemporaneously with (e) applying a magnetic field to said surface.
85. The method of any one of the preceding paragraphs, wherein said magnetic field immobilizes said substrate.
86. The method of any one of the preceding paragraphs, wherein said electro-mechanical actuator comprises a cantilever.
87. The method of any one of the preceding paragraphs, wherein said electro-mechanical actuator comprises one or more coupling members coupled to said array.
89. The method of any one of the preceding paragraphs, wherein said one or more coupling members comprise electromagnetic actuators, piezoelectric actuators, ultrasonic transducers, rotating eccentric masses, one or more motors with oscillating linkage mechanisms, or any combination thereof
90. The method of any one of the preceding paragraphs, wherein said one or more motors are brushed, brushless, stepper, or any combination thereof.
91. The method of any one of the preceding paragraphs, wherein said electromagnetic actuators comprise electromagnetic voice coil actuators.
92. The method of any one of the preceding paragraphs, wherein said frequency of vibration comprises a gradient.
93. The method of any one of the preceding paragraphs, wherein said gradient ascends from near a site wherein said cantilever is coupled to said array.
94. The method of any one of the preceding paragraphs, wherein said vibration comprises a pattern.
95. The method of any one of the preceding paragraphs, wherein said pattern is sinusoidal.
96. The method of any one of the preceding paragraphs, wherein said pattern is square.
97. The method of any one of the preceding paragraphs, wherein said surface is a top surface of a dielectric wherein said dielectric is disposed over said plurality of electrodes.
98. The method of any one of the preceding paragraphs, wherein said surface comprises a layer disposed over a dielectric wherein said dielectric is disposed over said plurality of electrodes.
99. The method of any one of the preceding paragraphs, wherein said layer comprises a liquid.
100. The method of any one of the preceding paragraphs, wherein said layer comprises a coating.
101. The method of any one of the preceding paragraphs, wherein said coating is hydrophobic.
102. The method of any one of the preceding paragraphs, wherein said layer comprises a film.
103. The method of any one of the preceding paragraphs, wherein said film is a dielectric film.
104. The method of any one of the preceding paragraphs, wherein said dielectric film comprises a natural polymeric material, a synthetic polymeric material, a fluorinated material, a surface modification, or any combination thereof.
105. The method of any one of the preceding paragraphs, wherein said natural polymeric material comprises shellac, amber, wool, silk, natural rubber, cellulose, wax, chiton, or any combination thereof.
106. The method of any one of the preceding paragraphs, wherein said synthetic polymeric material comprises polyethylene, polypropylene, polystyrene, polyetheretherketone (PEEK), polyimide, polyacetal, polysilfone, polyphenulene ether, polyphenylene Sulfide (PPS), polyvinyl chloride, synthetic rubber, neoprene, nylon, polyacrylonitrile, polyvinyl butyral, silicone, parafilm, polyethylene terephthalate, polybutylene terephthalate, polyamides, polyoxymethlyene, polycarbonate, polymethylpentene, polyphenylene oxide (Polyphenyl ether), polyphthalamide (PPA), polylactic acid, synthetic cellulose ethers (e.g., methyl cellulose, ethyl cellulose, propyl cellulose, hydroxyethyl cellulose, hydroxyethyl cellulose, hydroxypropyl cellulose (HPC), hydroxyethyl methyl cellulose, hydroxypropyl methyl cellulose (HPMC), ethyl hydroxyethyl cellulose), paraffins, microcrystalline wax, epoxy, or any combination thereof.
107. The method of any one of the preceding paragraphs, wherein said fluorinated material comprises polytetrafluoroethylene (PTFE), tetrafluoroethylene (TFE), fluorinated ethylenepropylene copolymer (FEP), polyvinylidene fluoride (PVDF), perfluoroalkoxytetrafluoroethylene copolymer (PFA), perfluoromethyl vinylether copolymer (MFA), ethylenechlorotrifluoroethylene copolymer (ECTFE), ethylene-tetrafluoroethylene copolymer (ETFE), perfluoropolyether (PFPE), polychlorotetrafluoroethylene (PCTFE), or any combination thereof.
108. The method of any one of the preceding paragraphs, wherein said surface modification comprises silicone, silane, fluoro-polymer treatment, parylene coating, any other suitable surface chemistry modification process, ceramic, clay minerals, bentonite, kaolinite, vermiculite, graphite, molybdenum disulfide, mica, boron nitride, sodium formate, sodium oleate, sodium palmitate, sodium sulfate, sodium alginate, or any combination thereof.
109. The method of any one of the preceding paragraphs, wherein said liquid comprises silicone oils, fluorinated oils, ionic liquids, mineral oils, ferrofluids, polyphenyl ether, vegetable oil, esters of saturated fatty and dibasic acids, grease, fatty acids, triglycerides, polyalphaolefin, polyglycol hydrocarbons, other Non-hydrocarbon synthetic oils, or any combination thereof.
110. The method of any one of the preceding paragraphs, wherein said liquid further comprises surfactants, electrolytes, rheology modifier, wax, graphite, graphene, molybdenum disulfide, PTFE particles, or any combination thereof.
111. The method of any one of the preceding paragraphs, wherein said first plurality of electrodes, said dielectric, said surface configured to support said droplet comprising said sample, or any combination thereof is removable from said array.
112. The method of any one of the preceding paragraphs, wherein said frequency of said vibration displaces said surface or a portion of said surface from 0.05 millimeters (mm) to 10 mm.
113. The method of any one of the preceding paragraphs, wherein said frequency of said vibration is from 1 Hertz (hz) to 20 kilohertz (khz).
114. A system for processing a droplet, comprising:
   a. an array comprising:
      i. a plurality of electrodes, wherein no electrode of said plurality of electrodes is permanently grounded; and
      ii. a surface configured to support a droplet comprising said sample;
   b. a controller operatively coupled to said plurality of electrodes, wherein said controller is configured to:
      i. activate at least a subset of said plurality of electrodes with a time-varying voltage to alter a wetting characteristic of said surface.
115. The system of any one of the preceding paragraphs, wherein said system does not comprise an overlying electrode.
116. The system of any one of the preceding paragraphs, wherein said plurality of electrodes comprises at least one electrode comprising a cross-section or overlap with said droplet sufficient to generate a current-return path adjacent to said electrode and an adjacent electrode.
117. The system of any one of the preceding paragraphs, wherein said overlap of said droplet with at least one electrode is sufficient to generate a minimized energy state or state of equilibrium in said droplet.
118. The system of any one of the preceding paragraphs, wherein said plurality of electrodes are co-planar.
119. The system of any one of the preceding paragraphs, wherein said time-varying voltage is bipolar.
120. The system of any one of the preceding paragraphs, wherein said time-varying voltage is from about 1 Hz to about 20 kHz.
121. The system of any one of the preceding paragraphs, wherein, upon activation of at least said subset of said plurality of electrodes, the system further comprises a current-return path adjacent to said droplet and one or more inactive electrodes.
122. The system of any one of the preceding paragraphs, wherein said activation of at least said subset of said plurality of electrodes generates an antagonistic current driving scheme in one or more adjacent electrodes.
123. The system of any one of the preceding paragraphs, further comprising a dielectric layer.
124. The system of any one of the preceding paragraphs, wherein said dielectric layer comprises a thickness, wherein said thickness is sufficient to ground an electric current generated by said plurality of electrodes.
125. The system of any one of the preceding paragraphs, wherein said dielectric layer comprises a thickness, wherein said thickness is sufficient to act as at least a partial electrical barrier.
126. The system of any one of the preceding paragraphs, wherein said thickness is 0.025 micrometer (μm) to 10,000 μm.
127. The system of any one of the preceding paragraphs, wherein said dielectric layer comprises a natural polymeric material, a synthetic polymeric material, a fluorinated material, a surface modification, or any combination thereof.
128. The system of any one of the preceding paragraphs, wherein said natural polymeric material comprises shellac, amber, wool, silk, natural rubber, cellulose, wax, chiton, or any combination thereof.
129. The system of any one of the preceding paragraphs, wherein said synthetic polymeric material comprises polyethylene, polypropylene, polystyrene, polyetheretherketone (PEEK), polyimide, polyacetal, polysilfone, polyphenulene ether, polyphenylene Sulfide (PPS), polyvinyl chloride, synthetic rubber, neoprene, nylon, polyacrylonitrile, polyvinyl butyral, silicone, parafilm, polyethylene terephthalate, polybutylene terephthalate, polyamides, polyoxymethlyene, polycarbonate, polymethylpentene, polyphenylene oxide (Polyphenyl ether), polyphthalamide (PPA), polylactic acid, synthetic cellulose ethers (e.g., methyl cellulose, ethyl cellulose, propyl cellulose, hydroxyethyl cellulose, hydroxyethyl cellulose, hydroxypropyl cellulose (HPC), hydroxyethyl methyl cellulose, hydroxypropyl methyl cellulose (HPMC), ethyl hydroxyethyl cellulose), paraffins, microcrystalline wax, epoxy, or any combination thereof.
130. The system of any one of the preceding paragraphs, wherein said fluorinated material comprises polytetrafluoroethylene (PTFE), tetrafluoroethylene (TFE), fluorinated ethylenepropylene copolymer (FEP), polyvinylidene fluoride (PVDF), perfluoroalkoxytetrafluoroethylene copolymer (PFA), perfluoromethyl vinylether copolymer (MFA), ethylenechlorotrifluoroethylene copolymer (ECTFE), ethylene-tetrafluoroethylene copolymer (ETFE), perfluoropolyether (PFPE), polychlorotetrafluoroethylene (PCTFE), or any combination thereof.
131. The system of any one of the preceding paragraphs, wherein said surface modification comprises silicone, silane, fluoro-polymer treatment, parylene coating, any other suitable surface chemistry modification process, ceramic, clay minerals, bentonite, kaolinite, vermiculite, graphite, molybdenum disulfide, mica, boron nitride, sodium formate, sodium oleate, sodium palmitate, sodium sulfate, sodium alginate, or any combination thereof.
132. The system of any one of the preceding paragraphs, wherein said surface comprises a liquid layer.
133. The system of any one of the preceding paragraphs, wherein said liquid layer comprises silicone oils, fluorinated oils, ionic liquids, mineral oils, ferrofluids, polyphenyl ether, vegetable oil, esters of saturated fatty and dibasic acids, grease, fatty acids, triglycerides, polyalphaolefin, polyglycol hydrocarbons, other Non-hydrocarbon synthetic oils, or any combination thereof.
134. The system of any one of the preceding paragraphs, wherein said liquid layer further comprises surfactants, electrolytes, rheology modifier, wax, graphite, graphene, molybdenum disulfide, PTFE particles, or any combination thereof.
135. The system of any one of the preceding paragraphs, further comprising a liquid disposed in an interspace adjacent to said dielectric layer and said plurality of electrodes.
136. The system of any one of the preceding paragraphs, wherein said liquid generates adhesion between said plurality of electrodes and said dielectric layer.
137. The system of any one of the preceding paragraphs, wherein said liquid comprises a dielectric material.
138. The system of any one of the preceding paragraphs, wherein said liquid prevents or reduces electrical conductivity of air disposed in said interspace.
139. The system of any one of the preceding paragraphs, wherein said liquid comprises silicone oils, fluorinated oils, ionic liquids, mineral oils, ferrofluids, polyphenyl ether, vegetable oil, esters of saturated fatty and dibasic acids, grease, fatty acids, triglycerides, polyalphaolefin, polyglycol hydrocarbons, other Non-hydrocarbon synthetic oils, or any combination thereof.
140. The system of any one of the preceding paragraphs, wherein said liquid further comprises surfactants, electrolytes, rheology modifier, wax, graphite, graphene, molybdenum disulfide, PTFE particles, or any combination thereof.
141. A system for processing a droplet, comprising:
   a. an array comprising:
      i. a plurality of electrodes, wherein no electrode of said plurality of electrodes is permanently grounded; and
      ii. a surface configured to support a droplet comprising said sample;
   b. a controller operatively coupled to said plurality of electrodes, wherein said controller is configured to:
      i. activate at least a subset of said plurality of electrodes with a voltage to alter a wetting characteristic of said surface;
   wherein said array does not comprise a permanent reference electrode.
142. The system of any one of the preceding paragraphs, wherein said voltage is a time-varying voltage.
143. The system of any one of the preceding paragraphs, wherein said system does not comprise an overlying electrode.
144. The system of any one of the preceding paragraphs, wherein said plurality of electrodes comprises at least one electrode comprising a cross-section or overlap with said droplet sufficient to generate a current-return path adjacent to said electrode and an adjacent electrode.
145. The system of any one of the preceding paragraphs, wherein said overlap of said droplet with at least one electrode is sufficient to generate a minimized energy state or state of equilibrium in said droplet.
146. The system of any one of the preceding paragraphs, wherein said plurality of electrodes are co-planar.
147. The system of any one of the preceding paragraphs, wherein said time-varying voltage is bipolar.
148. The system of any one of the preceding paragraphs, wherein said time-varying voltage is from about 1 Hz to about 20 kHz.
149. The system of any one of the preceding paragraphs, wherein, upon activation of at least said subset of said plurality of electrodes, the system further comprises a current-return path adjacent to said droplet and one or more inactive electrodes.
150. The system of any one of the preceding paragraphs, wherein said activation of at least said subset of said plurality of electrodes generates an antagonistic current driving scheme in one or more adjacent electrodes.
151. The system of any one of the preceding paragraphs, further comprising a dielectric layer.
152. The system of any one of the preceding paragraphs, wherein said dielectric layer comprises a thickness, wherein said thickness is sufficient to ground an electric current generated by said plurality of electrodes.
153. The system of any one of the preceding paragraphs, wherein said dielectric layer comprises a thickness, wherein said thickness is sufficient to act as at least a partial electrical barrier.
154. The system of any one of the preceding paragraphs, wherein said thickness is 0.025 micrometer (μm) to 10,000 μm.
155. The system of any one of the preceding paragraphs, wherein said dielectric layer comprises a natural polymeric material, a synthetic polymeric material, a fluorinated material, a surface modification, or any combination thereof.
156. The system of any one of the preceding paragraphs, wherein said natural polymeric material comprises shellac, amber, wool, silk, natural rubber, cellulose, wax, chiton, or any combination thereof.
157. The system of any one of the preceding paragraphs, wherein said synthetic polymeric material comprises polyethylene, polypropylene, polystyrene, polyetheretherketone (PEEK), polyimide, polyacetal, polysilfone, polyphenulene ether, polyphenylene Sulfide (PPS), polyvinyl chloride, synthetic rubber, neoprene, nylon, polyacrylonitrile, polyvinyl butyral, silicone, parafilm, polyethylene terephthalate, polybutylene terephthalate, polyamides, polyoxymethlyene, polycarbonate, polymethylpentene, polyphenylene oxide (Polyphenyl ether), polyphthalamide (PPA), polylactic acid, synthetic cellulose ethers (e.g., methyl cellulose, ethyl cellulose, propyl cellulose, hydroxyethyl cellulose, hydroxyethyl cellulose, hydroxypropyl cellulose (HPC), hydroxyethyl methyl cellulose, hydroxypropyl methyl cellulose (HPMC), ethyl hydroxyethyl cellulose), paraffins, microcrystalline wax, epoxy, or any combination thereof.
158. The system of any one of the preceding paragraphs, wherein said fluorinated material comprises polytetrafluoroethylene (PTFE), tetrafluoroethylene (TFE), fluorinated ethylenepropylene copolymer (FEP), polyvinylidene fluoride (PVDF), perfluoroalkoxytetrafluoroethylene copolymer (PFA), perfluoromethyl vinylether copolymer (MFA), ethylenechlorotrifluoroethylene copolymer (ECTFE), ethylene-tetrafluoroethylene copolymer (ETFE), perfluoropolyether (PFPE), polychlorotetrafluoroethylene (PCTFE), or any combination thereof.
159. The system of any one of the preceding paragraphs, wherein said surface modification comprises silicone, silane, fluoro-polymer treatment, parylene coating, any other suitable surface chemistry modification process, ceramic, clay minerals, bentonite, kaolinite, vermiculite, graphite, molybdenum disulfide, mica, boron nitride, sodium formate, sodium oleate, sodium palmitate, sodium sulfate, sodium alginate, or any combination thereof.
160. The system of any one of the preceding paragraphs, wherein said surface comprises a liquid layer.
161. The system of any one of the preceding paragraphs, wherein said liquid layer comprises silicone oils, fluorinated oils, ionic liquids, mineral oils, ferrofluids, polyphenyl ether, vegetable oil, esters of saturated fatty and dibasic acids, grease, fatty acids, triglycerides, polyalphaolefin, polyglycol hydrocarbons, other Non-hydrocarbon synthetic oils, or any combination thereof.
162. The system of any one of the preceding paragraphs, wherein said liquid layer further comprises surfactants, electrolytes, rheology modifier, wax, graphite, graphene, molybdenum disulfide, PTFE particles, or any combination thereof.
163. The system of any one of the preceding paragraphs, further comprising a liquid disposed in an interspace adjacent to said dielectric layer and said plurality of electrodes.
164. The system of any one of the preceding paragraphs, wherein said liquid generates adhesion between said plurality of electrodes and said dielectric layer.
165. The system of any one of the preceding paragraphs, wherein said liquid comprises a dielectric material.
166. The system of any one of the preceding paragraphs, wherein said liquid prevents or reduces electrical conductivity of air disposed in said interspace.
167. The system of any one of the preceding paragraphs, wherein said liquid comprises silicone oils, fluorinated oils, ionic liquids, mineral oils, ferrofluids, polyphenyl ether, vegetable oil, esters of saturated fatty and dibasic acids, grease, fatty acids, triglycerides, polyalphaolefin, polyglycol hydrocarbons, other Non-hydrocarbon synthetic oils, or any combination thereof.
168. The system of any one of the preceding paragraphs, wherein said liquid further comprises surfactants, electrolytes, rheology modifier, wax, graphite, graphene, molybdenum disulfide, PTFE particles, or any combination thereof.
169. A method for motioning a droplet over an array, wherein said array comprises a plurality of electrodes, wherein no electrode of said plurality of electrodes is permanently grounded; and a surface configured to support said droplet comprising said sample, the method comprising:
   a. activating at least a subset of said plurality of electrodes with a time-varying voltage to alter a wetting characteristic of said surface;
   wherein said time-varying voltage generates a current-return path adjacent to said droplet and one or more inactive electrodes, thereby inducing motion of said droplet.
170. The method of any one of the preceding paragraphs, wherein said plurality of electrodes are co-planar.
171. The method of any one of the preceding paragraphs, wherein said time-varying voltage is bipolar.
172. The method of any one of the preceding paragraphs, wherein said time-varying voltage is from about 1 Hz to about 20 kHz.
173. The method of any one of the preceding paragraphs, wherein, upon activation of at least said subset of said plurality of electrodes, the system further comprises a current-return path adjacent to said droplet and one or more inactive electrodes.
174. The method of any one of the preceding paragraphs, wherein said activation of at least said subset of said plurality of electrodes generates an antagonistic current driving scheme in one or more adjacent electrodes.
175. A method for motioning a droplet over an array, wherein said array comprises a plurality of electrodes, wherein no electrode of said plurality of electrodes is permanently grounded; and a surface configured to support said droplet comprising said sample, the method comprising:
   a. activating at least a subset of said plurality of electrodes with a voltage to alter a wetting characteristic of said surface;
   wherein said array does not comprise a permanent reference electrode.
176. wherein said time-varying voltage generates a current-return path adjacent to said droplet and one or more inactive electrodes, thereby inducing motion of said droplet.
177. The method of any one of the preceding paragraphs, wherein said plurality of electrodes are co-planar.
178. The method of any one of the preceding paragraphs, wherein said time-varying voltage is bipolar.
179. The method of any one of the preceding paragraphs, wherein said time-varying voltage is from about 1 Hz to about 20 kHz.
180. The method of any one of the preceding paragraphs, wherein, upon activation of at least said subset of said plurality of electrodes, the system further comprises a current-return path adjacent to said droplet and one or more inactive electrodes.
181. The method of any one of the preceding paragraphs, wherein said activation of at least said subset of said plurality of electrodes generates an antagonistic current driving scheme in one or more adjacent electrodes.
182. A system for processing a sample, comprising:
   a. a plurality of electrodes;
   b. a dielectric layer disposed over said plurality of electrodes, wherein said dielectric layer comprises a surface configured to support a droplet comprising said sample;
   c. a liquid disposed in an interspace adjacent to said plurality of electrodes and said dielectric layer.
183. The system of any one of the preceding paragraphs, wherein said liquid generates adhesion between said plurality of electrodes and said dielectric layer.
184. The system of any one of the preceding paragraphs, wherein said liquid comprises a dielectric material.
185. The system of any one of the preceding paragraphs, wherein said liquid prevents or reduces electrical conductivity of air disposed in said interspace.
186. The system of any one of the preceding paragraphs, wherein said dielectric layer comprises a natural polymeric material, a synthetic polymeric material, a fluorinated material, a surface modification, or any combination thereof.
187. The system of any one of the preceding paragraphs, wherein said natural polymeric material comprises shellac, amber, wool, silk, natural rubber, cellulose, wax, chiton, or any combination thereof.
188. The system of any one of the preceding paragraphs, wherein said synthetic polymeric material comprises polyethylene, polypropylene, polystyrene, polyetheretherketone (PEEK), polyimide, polyacetal, polysilfone, polyphenulene ether, polyphenylene Sulfide (PPS), polyvinyl chloride, synthetic rubber, neoprene, nylon, polyacrylonitrile, polyvinyl butyral, silicone, parafilm, polyethylene terephthalate, polybutylene terephthalate, polyamides, polyoxymethlyene, polycarbonate, polymethylpentene, polyphenylene oxide (Polyphenyl ether), polyphthalamide (PPA), polylactic acid, synthetic cellulose ethers (e.g., methyl cellulose, ethyl cellulose, propyl cellulose, hydroxyethyl cellulose, hydroxyethyl cellulose, hydroxypropyl cellulose (HPC), hydroxyethyl methyl cellulose, hydroxypropyl methyl cellulose (HPMC), ethyl hydroxyethyl cellulose), paraffins, microcrystalline wax, epoxy, or any combination thereof.
189. The system of any one of the preceding paragraphs, wherein said fluorinated material comprises polytetrafluoroethylene (PTFE), tetrafluoroethylene (TFE), fluorinated ethylenepropylene copolymer (FEP), polyvinylidene fluoride (PVDF), perfluoroalkoxytetrafluoroethylene copolymer (PFA), perfluoromethyl vinylether copolymer (MFA), ethylenechlorotrifluoroethylene copolymer (ECTFE), ethylene-tetrafluoroethylene copolymer (ETFE), perfluoropolyether (PFPE), polychlorotetrafluoroethylene (PCTFE), or any combination thereof.
190. The system of any one of the preceding paragraphs, wherein said surface modification comprises silicone, silane, fluoro-polymer treatment, parylene coating, any other suitable surface chemistry modification process, ceramic, clay minerals, bentonite, kaolinite, vermiculite, graphite, molybdenum disulfide, mica, boron nitride, sodium formate, sodium oleate, sodium palmitate, sodium sulfate, sodium alginate, or any combination thereof.
191. The system of any one of the preceding paragraphs, wherein said liquid comprises silicone oils, fluorinated oils, ionic liquids, mineral oils, ferrofluids, polyphenyl ether, vegetable oil, esters of saturated fatty and dibasic acids, grease, fatty acids, triglycerides, polyalphaolefin, polyglycol hydrocarbons, other Non-hydrocarbon synthetic oils, or any combination thereof.
192. The system of any one of the preceding paragraphs, wherein said liquid further comprises surfactants, electrolytes, rheology modifier, wax, graphite, graphene, molybdenum disulfide, PTFE particles, or any combination thereof.
193. The system of any one of the preceding paragraphs, wherein said surface comprises a liquid layer.
194. The system of any one of the preceding paragraphs, wherein said liquid layer comprises silicone oils, fluorinated oils, ionic liquids, mineral oils, ferrofluids, polyphenyl ether, vegetable oil, esters of saturated fatty and dibasic acids, grease, fatty acids, triglycerides, polyalphaolefin, polyglycol hydrocarbons, other Non-hydrocarbon synthetic oils, or any combination thereof.
195. The system of any one of the preceding paragraphs, wherein said liquid layer further comprises surfactants, electrolytes, rheology modifier, wax, graphite, graphene, molybdenum disulfide, PTFE particles, or any combination thereof.
196. The system of any one of the preceding paragraphs, wherein said dielectric layer is removable.
197. A system for processing a sample, comprising:
   a. a plurality of electrodes;
   b. a dielectric layer disposed over said plurality of electrodes, wherein said dielectric layer comprises a surface configured to support a droplet comprising said sample;
   c. a liquid adjacent to said surface, wherein said liquid comprises a chemical affinity to said surface, and wherein said chemical affinity is sufficient to immobilize said liquid onto said surface and wherein said liquid is resistant to gravity.
198. The system of any one of the preceding paragraphs, wherein said dielectric layer comprises a natural polymeric material, a synthetic polymeric material, a fluorinated material, a surface modification, or any combination thereof.
199. The system of any one of the preceding paragraphs, wherein said natural polymeric material comprises shellac, amber, wool, silk, natural rubber, cellulose, wax, chiton, or any combination thereof.
200. The system of any one of the preceding paragraphs, wherein said synthetic polymeric material comprises polyethylene, polypropylene, polystyrene, polyetheretherketone (PEEK), polyimide, polyacetal, polysilfone, polyphenulene ether, polyphenylene Sulfide (PPS), polyvinyl chloride, synthetic rubber, neoprene, nylon, polyacrylonitrile, polyvinyl butyral, silicone, parafilm, polyethylene terephthalate, polybutylene terephthalate, polyamides, polyoxymethlyene, polycarbonate, polymethylpentene, polyphenylene oxide (Polyphenyl ether), polyphthalamide (PPA), polylactic acid, synthetic cellulose ethers (e.g., methyl cellulose, ethyl cellulose, propyl cellulose, hydroxyethyl cellulose, hydroxyethyl cellulose, hydroxypropyl cellulose (HPC), hydroxyethyl methyl cellulose, hydroxypropyl methyl cellulose (HPMC), ethyl hydroxyethyl cellulose), paraffins, microcrystalline wax, epoxy, or any combination thereof.
201. The system of any one of the preceding paragraphs, wherein said fluorinated material comprises polytetrafluoroethylene (PTFE), tetrafluoroethylene (TFE), fluorinated ethylenepropylene copolymer (FEP), polyvinylidene fluoride (PVDF), perfluoroalkoxytetrafluoroethylene copolymer (PFA), perfluoromethyl vinylether copolymer (MFA), ethylenechlorotrifluoroethylene copolymer (ECTFE), ethylene-tetrafluoroethylene copolymer (ETFE), perfluoropolyether (PFPE), polychlorotetrafluoroethylene (PCTFE), or any combination thereof.
202. The system of any one of the preceding paragraphs, wherein said surface modification comprises silicone, silane, fluoro-polymer treatment, parylene coating, any other suitable surface chemistry modification process, ceramic, clay minerals, bentonite, kaolinite, vermiculite, graphite, molybdenum disulfide, mica, boron nitride, sodium formate, sodium oleate, sodium palmitate, sodium sulfate, sodium alginate, or any combination thereof.
203. The system of any one of the preceding paragraphs, wherein said liquid comprises silicone oils, fluorinated oils, ionic liquids, mineral oils, ferrofluids, polyphenyl ether, vegetable oil, esters of saturated fatty and dibasic acids, grease, fatty acids, triglycerides, polyalphaolefin, polyglycol hydrocarbons, other Non-hydrocarbon synthetic oils, or any combination thereof.
204. The system of any one of the preceding paragraphs, wherein said liquid further comprises surfactants, electrolytes, rheology modifier, wax, graphite, graphene, molybdenum disulfide, PTFE particles, or any combination thereof.
205. The system of any one of the preceding paragraphs, wherein said surface comprises a liquid layer.
206. The system of any one of the preceding paragraphs, wherein said liquid layer comprises silicone oils, fluorinated oils, ionic liquids, mineral oils, ferrofluids, polyphenyl ether, vegetable oil, esters of saturated fatty and dibasic acids, grease, fatty acids, triglycerides, polyalphaolefin, polyglycol hydrocarbons, other Non-hydrocarbon synthetic oils, or any combination thereof.
207. The system of any one of the preceding paragraphs, wherein said liquid layer further comprises surfactants, electrolytes, rheology modifier, wax, graphite, graphene, molybdenum disulfide, PTFE particles, or any combination thereof.
208. The system of any one of the preceding paragraphs, wherein said dielectric layer is removable.

### Embodiment 2

1. A method of generating a biopolymer, comprising:
   a. providing a plurality of droplets adjacent to a surface, wherein said plurality of droplets comprises a first droplet comprising a first reagent and a second droplet comprising a second reagent;
   b. subjecting said first droplet and said second droplet to motion relative to one another to
      (i) bring said first droplet in contact with said second droplet and (ii) form a merged droplet comprising said first reagent and said second reagent; and
   c. in said merged droplet, using at least (i) said first reagent and (ii) said second reagent to form at least a portion of said biopolymer,
   wherein (b)-(c) are performed in a time period of 10 minutes or less.
2. The method of paragraph 1, wherein said biopolymer is a polynucleotide.
3. The method of paragraph 1, wherein said biopolymer is a polypeptide.
4. The method of any one of paragraphs 1 or 2, wherein said polynucleotide comprises about 10 to about 250 bases.
5. The method of any one of paragraphs 1 to 3, where said polynucleotide comprises about 260 to about 1 kb.
6. The method of any one of paragraphs 1 to 3, where said polynucleotide comprises about 1 kb to about 10,000 kb.
7. The method of any one of paragraphs 1 to 6, wherein a vibration is applied to said synthesis droplet during (b), (c), or both.
8. The method of any one of paragraphs 1 to 7, wherein the method further comprises, one or more washing steps comprising subjecting a wash droplet to motion to contact said merged droplet.
9. The method of paragraph 8, wherein a vibration is applied to said one or more washing steps.
10. The method of any one of paragraphs 1 to 9, wherein said surface is dielectric.
11. The method of any one of paragraphs 1 to 9, wherein said surface comprises a dielectric layer disposed over one or more electrodes.
12. The method of any one of paragraphs 1 to 9, wherein said surface is the surface of a polymeric film.
13. The method of any one of paragraphs 1 to 9, wherein the surface comprises one or more oligonucleotides bound to the surface.
14. The method of any one of paragraphs 1 to 9, wherein said surface is the surface of a lubricating liquid layer.
15. The method of any one of paragraphs 1 to 14, wherein said plurality of droplets comprises a third droplet comprising a third reagent.
16. The method of any one of paragraphs 1 to 15, wherein said first reagent, said second reagent, said third reagent, or any combination thereof, comprises one or more functionalized beads.
17. The method of paragraph 16, wherein said functional beads comprise one or more oligonucleotides immobilized thereto.
18. The method of any one of paragraphs 1 to 17, wherein a vibration is applied to either said first droplet, said second droplet, said third droplet, a wash droplet, or the mixtures thereof
19. The method of any one of paragraphs 1 to 18, wherein said first reagent, said second reagent, said third reagent or any combination thereof comprises a polymerase.
20. The method of any one of paragraphs 1 to 19, wherein said first reagent, said second reagent, said third reagent or any combination thereof comprises a bio-monomer.
21. The method of paragraph 20, wherein said bio-monomer is an amino acid.
22. The method of paragraph 20, wherein said bio-monomer is a nucleic acid molecule.
23. The method of paragraph 22, wherein said nucleic acid molecule comprises of adenine, cytosine, guanine, thymine, or uracil.
24. The method of any one of paragraphs 1 to 23, wherein said first reagent, said second reagent, said third reagent, or any combination thereof, comprises one or more functionalized discs.
25. The method of paragraph 24, wherein said functionalized disc comprise one or more oligonucleotides immobilized thereto.
26. The method of any one of paragraphs 1 to 25, wherein said first reagent, said second reagent, said third reagent, or any combination thereof comprises an enzyme that mediates synthesis or polymerization.
27. The method of paragraph 26, wherein said enzyme is from the group consisting of Polynucleotide Phosphorylase (PNPase), Terminal Denucleotidyl Transferas (TdT), DNA polymerase Beta, DNA polymerase lambda, DNA polymerase mu and other enzymes from X family of DNA polymerases.
28. The method of any one of paragraphs 1 to 27, wherein at least one nucleic acid molecule of said polynucleotide is generated in 20 minutes or less within said merged droplet.
29. The method of any one of paragraphs 1 to 28, wherein at least one nucleic acid molecule of said polynucleotide is generated in 15 minutes or less within said merged droplet.
30. The method of any one of paragraphs 1 to 29, wherein at least one nucleic acid molecule of said polynucleotide is generated in 10 minutes or less within said merged droplet.
31. The method of any one of paragraphs 1 to 30, wherein at least one nucleic acid molecule of said polynucleotide is generated in 1 minute or less within said merged droplet.
32. The method of any one of paragraphs 1 to 31, wherein said merged droplet is temperature-controlled.
33. The method of any one of paragraphs 1 to 32, wherein said first droplet, said second droplet, said third droplet, or said merged droplet is subjected to a magnetic field.
34. The method of any one of paragraphs 1 to 33, wherein said first droplet, said second droplet, said third droplet, or said merged droplet is subjected to light.
35. The method of any one of paragraphs 1 to 34, wherein said first droplet, said second droplet, said third droplet, or said merged droplet is subjected to pH change.
36. The method of any one of paragraphs 1 to 35, wherein said first droplet, said second droplet, said third droplet, or said merged droplet comprises of deoxynucleoside triphosphate (dNTP).
37. The method of paragraph 36, wherein said deoxynucleoside triphosphate may have a protective group.
38. The method of paragraph 37, wherein said protective group can be removed during the reaction.
39. The method of any one of paragraphs 1 to 38, wherein said first droplet, said second droplet, said third droplet, or said merged droplet make contact with a surface only on one side.
40. The method of any one of paragraphs 1 to 39, wherein volumes of said first droplet, said second droplet, said third droplet, or said merged droplet is between 1 nanoliter (1 n1) and 500 microliters (500 μl).
41. The method of any one of paragraphs 1 to 40, wherein volumes of said first droplet, said second droplet, said third droplet, or said merged droplet is between 1 microliter (1 μl) and 500 microliters (500 μl).
42. The method of any one of paragraphs 1 to 41, wherein volumes of said first droplet, said second droplet, said third droplet, or said merged droplet is between 1 microliter (1 μl) and 200 microliters (200 μl).
43. The method of any one of paragraphs 1 to 42, wherein the method further comprises ligating said biopolymer to a second biopolymer.
44. The method of paragraph 43, wherein said second biopolymer was generated using the method of any one of paragraphs 1 to 43.
45. A method of generating a biopolymer, comprising:
   a. providing a plurality of droplets adjacent to a surface, wherein said plurality of droplets comprises a first droplet comprising a first reagent and a second droplet comprising a second reagent;
   b. subjecting said first droplet and said second droplet to motion relative to one another to
      (i) bring said first droplet in contact with said second droplet and (ii) form a merged droplet comprising said first reagent and said second reagent; and
   c. in said merged droplet, using at least (i) said first reagent and (ii) said second reagent to form at least a portion of said biopolymer,
   wherein a vibration is applied to (b), (c), or both.
46. The method of paragraph 45, wherein said biopolymer is a polynucleotide.
47. The method of paragraph 45 or 46, wherein said biopolymer is a polypeptide.
48. The method of any one of paragraphs 45 to 47, wherein said polynucleotide comprises 2 to 10,000,000 nucleic acid molecules.
49. The method of any one of paragraphs 45 to 48, wherein the method further comprises, one or more washing steps comprising subjecting a wash droplet to motion to contact said merged droplet.
50. The method of paragraph 49, wherein a vibration is applied to said one or more washing steps.
51. The method of any one of paragraphs 45 to 50, wherein at least one nucleic acid molecule of said polynucleotide is generated in 30 minutes or less within said merged droplet.
52. The method of any one of paragraphs 45 to 51, wherein said surface is dielectric.
53. The method of any one of paragraphs 45 to 51, wherein said surface comprises a dielectric layer disposed over one or more electrodes.
54. The method of any one of paragraphs 45 to 51, wherein said surface is the surface of a polymeric film.
55. The method of any one of paragraphs 45 to 51, wherein the surface comprises one or more oligonucleotides bound to the surface.
56. The method of any one of paragraphs 45 to 51, wherein said surface is the surface of a lubricating liquid layer.
57. The method of any one of paragraphs 45 to 56, wherein said plurality of droplets comprises a third droplet comprising a third reagent.
58. The method of any one of paragraphs 45 to 57, wherein said first reagent, said second reagent, said third reagent, or any combination thereof comprises one or more functionalized beads.
59. The method of any one of paragraphs 45 to 58, wherein said functional beads comprise one or more oligonucleotides immobilized thereto.
60. The method of any one of paragraphs 45 to 59, wherein said first reagent, said second reagent, said third reagent, or any combination thereof comprises a polymerase.
61. The method of any one of paragraphs 45 to 60, wherein said first reagent, said second reagent, said third reagent or any combination thereof comprises a bio-monomer.
62. The method of paragraph 61, wherein said bio-monomer is an amino acid.
63. The method of paragraph 61, wherein said bio-monomer is a nucleic acid molecule.
64. The method of paragraph 63, wherein said nucleic acid molecule is adenine, cytosine, guanine, thymine, or uracil.
65. The method of any one of paragraphs 45 to 64, wherein said first reagent comprises one or more functionalized discs.
66. The method of any one of paragraphs 45 to 65, wherein said functionalized disc comprise one or more oligonucleotides immobilized thereto.
67. The method of any one of paragraphs 45 to 66, wherein said first droplet, second droplet, third droplet, or both comprises an enzyme that mediate synthesis or polymerization.
68. The method of paragraph 67, wherein said enzyme is from the group consisting of Polynucleotide Phosphorylase (PNPase), Terminal Denucleotidyl Transferas (TdT), DNA polymerase Beta, DNA polymerase lambda, DNA polymerase mu and other enzymes from X family of DNA polymerases.
69. The method of any one of paragraphs 45 to 68, wherein at least one nucleic acid molecule of said polynucleotide is generated in 20 minutes or less within said merged droplet.
70. The method of any one of paragraphs 45 to 69, wherein at least one nucleic acid molecule of said polynucleotide is generated in 15 minutes or less within said merged droplet.
71. The method of any one of paragraphs 45 to 70, wherein at least one nucleic acid molecule of said polynucleotide is generated in 10 minutes or less within said merged droplet.
72. The method of any one of paragraphs 45 to 71, wherein said merged droplet is heated.
73. The method of any one of paragraphs 45 to 71, wherein said first droplet, said second droplet, said third droplet, or said merged droplet is subjected to magnetic field.
74. The method of any one of paragraphs 45 to 71, wherein said first droplet, said second droplet, said third droplet, or said merged droplet is subjected to light.
75. The method of any one of paragraphs 45 to 71, wherein said first droplet, said second droplet, said third droplet, or said merged droplet is subjected to pH change.
76. The method of any one of paragraphs 45 to 75, wherein said first droplet, said second droplet, said third droplet, or said merged droplet comprises of deoxynucleoside triphosphate (dNTP).
77. The method of paragraph 76, wherein said deoxynucleoside triphosphate may have a protective group.
78. The method of paragraph 77, wherein said protective group can be removed during the reaction.
79. The method of any one of paragraphs 45 to 78, wherein said first droplet, said second droplet, said third droplet, or said merged droplet makes contact with a surface only on one side.
80. The method of any one of paragraphs 45 to 79, wherein volumes of said first droplet, said second droplet, said third droplet, or said merged droplet is between 1 nanoliter (1 n1) and 500 microliters (500 μl).
81. The method of any one of paragraphs 45 to 80, wherein volumes of said first droplet, said second droplet, said third droplet, or said merged droplet is between 1 microliter (1 μl) and 500 microliters (500 μl).
82. The method of any one of paragraphs 45 to 81, wherein volumes of said first droplet, said second droplet, said third droplet, or said merged droplet is between 1 microliter (1 μl) and 200 microliters (200 μl).

### Embodiment 3

1. A method for circularizing a nucleic acid sample, comprising:
   (a) providing a droplet adjacent to an electrowetting array, wherein said sample droplet comprises said nucleic acid sample; and
   (b) using said electrowetting array to process said droplet to circularize said nucleic acid sample.
2. The method of paragraph 1, wherein said electrowetting array comprises a dielectric substrate.
3. The method of paragraph 1, wherein said electrowetting array further comprises one or more reagent droplets.
4. The method of paragraph 1, wherein said one or more reagent droplets comprises one or more reagents for circularizing said nucleic acid sample.
5. The method of paragraph 4, further comprising:
   (a) combining said sample droplet with said one or more reagent droplets;
   (b) separating said sample droplet from said one or more reagent droplets; and
   (c) combining said one or more reagent droplets with a second droplet.
6. The method of paragraph 1, wherein said droplet comprises one or more reagents for circularizing said nucleic acid sample.
7. The method of paragraph 4, wherein (b) further comprises performing one or more droplet operations on said electrowetting array to process said droplet, wherein said one or more droplet operations comprise contacting said one or more reagent droplets with said droplet.
8. The method of paragraph 3, wherein said electrowetting array comprises one or more electrodes beneath a surface of said electrowetting array, and wherein said one or more droplet operations comprise applying a voltage to at least one electrode of said one or more electrodes to manipulate said one or more reagent droplets, said sample droplet, or both.
9. The method of paragraph 7, wherein said one or more droplet operations comprise applying a vibration to said one or more reagent droplets, said sample droplet, or both.
10. The method of paragraph 7, wherein said one or more droplet operations comprise applying a vibration to said electrowetting array.
11. The method of paragraph 1, further comprising using a single polymerizing enzyme to subject said nucleic acid sample to a sequencing reaction.
12. The method of paragraph 1, further comprising yielding a sequencing read having a length of at least 70 kilobase (kb).
13. The method of paragraph 1, further comprising yielding a sequencing read having a length of at least 80 kilobase (kb).
14. The method of paragraph 1, further comprising yielding a sequencing read having a length of about 200 kilobase (kb).
15. The method of paragraph 1, wherein at least 100 (Gb) of sequencing data is produced.
16. The method of paragraph 15, wherein at least 500 Gb of sequencing data is produced.
17. The method of paragraph of paragraph 16, wherein at least 512 Gb of sequencing data is produced.
18. The method of paragraph 1, wherein at least 10 Gb of data is produced.
19. The method of paragraph 18, wherein at least 30 Gb of data is produced.
20. The method of paragraph 11, wherein said sequencing reaction comprises repeated passes.
21. The method of paragraph 12, wherein one or more subreads of said sequencing read are produced.
22. The method of paragraph 21, wherein a consensus sequence is produced from said subreads of sequencing reads.
23. The method of paragraph 13, wherein said sequencing read comprises an A260/A280 ratio of less than about 1.84.
24. The method of paragraph 1, further comprising generating a circularized nucleic acid sample. 25. The method of paragraph 24, wherein said circularized nucleic acid sample comprises a target sequence.
26. The method of paragraph 24, wherein said circularized nucleic acid sample comprises a plurality of sequences comprising said target sequence.
27. The method of paragraph 25, wherein at least 80% of said plurality of sequences comprises said target sequence.
28. The method of paragraph 1, wherein the method further comprises, prior to (a), deriving said nucleic acid sample from a biological sample on said electrowetting array.
29. A method of sequencing a nucleic acid sample, comprising (a) providing a droplet adjacent to an electrowetting array, which droplet comprises said nucleic acid sample, (b) using said electrowetting array to process said droplet to circularize said nucleic acid sample, and (c) using a single polymerizing enzyme to subject said circularized nucleic acid sample to a sequencing reaction.
30. A method for sequencing a circular nucleic acid sample, comprising using a single polymerizing enzyme to subject said nucleic acid sample to a sequencing reaction to yield a sequencing read having a length of at least 70 kilobase.
31. The method of paragraph 29, further comprising using a waveguide to detect bases incorporated into said nucleic acid sample during said sequencing reaction.
32. A method of producing a circularized nucleic acid sample with a longer insert size, comprising (a) providing a droplet adjacent to an electrowetting array, which droplet comprises said nucleic acid sample, (b) using said electrowetting array to process said droplet to circularize said nucleic acid sample, and (c) using a single polymerizing enzyme to subject said circularized nucleic acid sample to a sequencing reaction.
33. A method for generating a sequencing library, comprising (a) providing a nucleic acid sample comprising a plurality of nucleic acid molecules comprising a plurality of sequences, and (b) using said nucleic acid sample to generate said sequencing library, wherein said sequencing library comprises at least 80% of said plurality of sequences of complements thereof.
34. A method for circularizing a nucleic acid sample, comprising:
   (a) providing a droplet adjacent to an electrowetting array, wherein said droplet comprises said nucleic acid sample;
   (b) combining said droplet with one or more reagent droplets;
   (c) using said electrowetting array to process said droplet to circularize said nucleic acid sample;
   (d) separating said droplet from said one or more reagent droplets; and
   (e) combining said one or more reagent droplets with said sample droplet to yield a circularized nucleic acid sample.
35. The method of any one of the previous paragraphs, wherein said electrowetting array comprises a dielectric substrate.
36. The method of any one of the previous paragraphs, wherein said electrowetting array further comprises one or more reagent droplets.
37. The method of any one of the previous paragraphs, wherein said one or more reagent droplets comprises one or more reagents for circularizing said nucleic acid sample.
38. The method of any one of the previous paragraphs, further comprising:
   (a) combining said sample droplet with said one or more reagent droplets;
   (b) separating said sample droplet from said one or more reagent droplets; and
   (c) combining said one or more reagent droplets with a second droplet.
39. The method of any one of the previous paragraphs, wherein said droplet comprises one or more reagents for circularizing said nucleic acid sample.
40. The method of any one of the previous paragraphs, wherein (b) further comprises performing one or more droplet operations on said electrowetting array to process said droplet, wherein said one or more droplet operations comprise contacting said one or more reagent droplets with said droplet.
41. The method of any one of the previous paragraphs, wherein said electrowetting array comprises one or more electrodes beneath a surface of said electrowetting array, and wherein said one or more droplet operations comprise applying a voltage to at least one electrode of said one or more electrodes to manipulate said one or more reagent droplets, said sample droplet, or both.
42. The method of any one of the previous paragraphs, wherein said one or more droplet operations comprise applying a vibration to said one or more reagent droplets, said sample droplet, or both.
43. The method of any one of the previous paragraphs, wherein said one or more droplet operations comprise applying a vibration to said electrowetting array.
44. The method of any one of the previous paragraphs, further comprising using a single polymerizing enzyme to subject said nucleic acid sample to a sequencing reaction.
45. The method of any one of the previous paragraphs, further comprising yielding a sequencing read having a length of at least 70 kilobase (kb).
46. The method of any one of the previous paragraphs, further comprising yielding a sequencing read having a length of at least 80 kilobase (kb).
47. The method of any one of the previous paragraphs, further comprising yielding a sequencing read having a length of about 200 kilobase (kb).
48. The method of any one of the previous paragraphs, wherein at least 100 (Gb) of sequencing data is produced.
49. The method of any one of the previous paragraphs, wherein at least 500 Gb of sequencing data is produced.
50. The method of paragraph of paragraph 16, wherein at least 512 Gb of sequencing data is produced.
51. The method of any one of the previous paragraphs, wherein at least 10 Gb of data is produced.
52. The method of any one of the previous paragraphs, wherein at least 30 Gb of data is produced.
53. The method of any one of the previous paragraphs, wherein said sequencing reaction comprises repeated passes.
54. The method of any one of the previous paragraphs, wherein one or more subreads of said sequencing read are produced.
55. The method of any one of the previous paragraphs, wherein a consensus sequence is produced from said subreads of sequencing reads.
56. The method of any one of the previous paragraphs, wherein said sequencing read comprises an A260/A280 ratio of less than about 1.84.
57. The method of any one of the previous paragraphs, further comprising generating a circularized nucleic acid sample.
58. The method of any one of the previous paragraphs, wherein said circularized nucleic acid sample comprises a target sequence.
59. The method of any one of the previous paragraphs, wherein said circularized nucleic acid sample comprises a plurality of sequences comprising said target sequence.
60. The method of any one of the previous paragraphs, wherein at least 80% of said plurality of sequences comprises said target sequence.
61. The method of any one of the previous paragraphs, wherein the method further comprises, prior to (a), deriving said nucleic acid sample from a biological sample on said electrowetting array.
62. A method for processing a nucleic acid sample, comprising:
   (a) providing a biological sample adjacent to an electrowetting array, wherein said sample droplet comprises said nucleic acid sample; and
   (b) extracting said nucleic acid sample from said biological sample adjacent to said electrowetting array
   wherein said nucleic acid sample comprises a sequencing read having a length of at least about 70 kilobases (kb)
63. The method of paragraph 61, wherein said length is at least about 80 kilobases (kb).
64. The method of paragraph 61, wherein said length is at least about 200 kilobases (kb).
65. The method of paragraph 61, wherein said sequencing read comprises an A260/A280 ratio of less than about 1.84.

1. A method of generating a biopolymer, comprising:
a. providing a plurality of droplets adjacent to a surface, wherein said plurality of droplets comprises a first droplet comprising a first reagent and a second droplet comprising a second reagent;
b. subjecting said first droplet and/or said second droplet to motion to (i) bring said first droplet in contact with said second droplet and (ii) form a merged droplet comprising said first reagent and said second reagent; and
c. using at least (i) said first reagent and (ii) said second reagent to form at least a portion of said biopolymer in said merged droplet,
wherein generating at least said portion of said biopolymer is performed in a time period of 10 minutes or less.

2. (canceled)

3. (canceled)

4. (canceled)

5. (canceled)

6. (canceled)

7. The method of claim 1, wherein a vibration is applied to said merged droplet during (b), (c), or both.

8. The method of claim 1, wherein the method further comprises subjecting a wash droplet to motion to contact said merged droplet.

9. (canceled)

10. (canceled)

11. The method of claim 1, wherein said surface comprises a dielectric layer disposed over one or more electrodes.

12. (canceled)

13. (canceled)

14. The method of claim 1, wherein said surface comprises a lubricating liquid layer.

15. (canceled)

16. (canceled)

17. (canceled)

18. The method of claim 1, wherein a vibration is applied to either said first droplet, said second droplet, a wash droplet, or the mixtures thereof.

19. The method of claim 1, wherein said first reagent, said second reagent, both comprises a polymerase.

20. (canceled)

21. (canceled)

22. (canceled)

23. (canceled)

24. (canceled)

25. (canceled)

26. The method of claim 1, wherein said first reagent, said second reagent, or both comprises an enzyme that mediates synthesis or polymerization.

27. (canceled)

28. The method of claim 1, wherein at least one nucleic acid molecule of said biopolymer is generated in 20 minutes or less within said merged droplet.

29-38. (canceled)

39. The method of claim 1, wherein said first droplet, said second droplet, or said merged droplet make contact with only one side of the surface.

40. The method of claim 1, wherein a volume of said first droplet, said second droplet, or said merged droplet is between 1 nanoliter (1 nl) and 500 microliters (500 μl).

41. (canceled)

42. (canceled)

43. The method of claim 1, wherein the method further comprises ligating said biopolymer to a second biopolymer.

44. (canceled)

45. A method for processing a sample comprising:
a. providing an array comprising:
i. a plurality of electrodes; and
ii. a surface configured to support said sample;
wherein said array is coupled to an electro-mechanical actuator and said electro-mechanical actuator is configured to vibrate said array;
b. introducing a droplet comprising said sample to said surface; and
c. directing said electro-mechanical actuator to apply a frequency of vibration to said array.

46. (canceled)

47. (canceled)

48. (canceled)

49. (canceled)

50. (canceled)

51. The method of claim 45, further comprising directing at least a subset of said plurality of electrodes to supply an electric field to alter a wetting characteristic of said surface.

52. (canceled)

53. (canceled)

54. (canceled)

55. (canceled)

56. The method of claim 45, wherein said electromagnetic actuator comprises electromagnetic voice coil actuators.

57. The method of claim 45, wherein said frequency of vibration comprises a gradient.

58. (canceled)

59. (canceled)

60. (canceled)

61. (canceled)

62. The method of claim 45, wherein a top of said surface comprises a dielectric wherein said dielectric is disposed over said plurality of electrodes.

63. The method of claim 45, wherein said surface comprises a layer disposed over a dielectric wherein said dielectric is disposed over said plurality of electrodes.

64. (canceled)

65. (canceled)

66. (canceled)

67. (canceled)

68. (canceled)

69. (canceled)

70. (canceled)

71. (canceled)

72. (canceled)

73. (canceled)

74. (canceled)

75. (canceled)

76. (canceled)

77. The method of claim 45, wherein said frequency of vibration displaces said surface or a portion of said surface from 0.05 millimeters (mm) to 10 mm.

78. (canceled)

79-143. (canceled)

144. A system configured to generate a biopolymer, comprising:
a plurality of droplets adjacent to a surface, wherein said plurality of droplets comprises a first droplet comprising a first reagent and a second droplet comprising a second reagent; wherein the system is configured to:
a. subject said first droplet and said second droplet to motion relative to one another to (i) bring said first droplet in contact with said second droplet and (ii) form a merged droplet comprising said first reagent and said second reagent; and
b. using at least (i) said first reagent and (ii) said second reagent to form at least a portion of said biopolymer in said merged droplet,
wherein generating at least said portion of said biopolymer is performed in a time period of 10 minutes or less.
